(19) United States
(12) Patent Application Publication
(10) Pub. No.: US 2026/0193187 A1
(43) Pub. Date: Jul. 9, 2026

(54) MATERIALS FOR ORGANIC ELECTRONIC DEVICES

(71) Applicant: Merck Patent GmbH, Darmstadt (DE)

(72) Inventors: Christian WIRGES, Darmstadt (DE); Teresa MUJICA-FERNAUD, Darmstadt (DE); Elvira MONTENEGRO, Darmstadt (DE); Frank VOGES, Darmstadt (DE); Florian MAIER-FLAIG, Darmstadt (DE); Thomas EBERLE, Darmstadt (DE)

(21) Appl. No.: 19/554,025

(22) Filed: Mar. 2, 2026

Related U.S. Application Data
(63) Continuation of application No. 16/489,752, filed on Jan. 9, 2018.

(30) Foreign Application Priority Data
Mar. 2, 2017 (EP) 17158932.8

(51) Int. Cl. C07D 213/38; C07D 239/26; C07D 251/24; C07D 307/91; C07D 333/76; C07D 403/04; C07D 405/12; C07D 409/12; H10K 50/11; H10K 50/15; H10K 50/18; H10K 85/60; H10K 101/10

(52) U.S. Cl. CPC C07D 213/38; C07D 239/26; C07D 251/24; C07D 307/91; C07D 333/76; C07D 403/04; C07D 405/12; C07D 409/12; H10K 85/624; H10K 85/633; H10K 85/636; H10K 85/654; H10K 85/6572; H10K 85/6574; H10K 85/6576; H10K 50/11; H10K 50/15; H10K 50/181; H10K 2101/10

(57) ABSTRACT

The present application relates to triarylamine compounds of a defined formula. The present application further relates to processes for preparing the compounds, to the use of the compounds in electronic devices, and to electronic devices comprising the compounds.

The present application relates to triarylamine compounds of a formula (I) defined further down. These compounds are suitable for use in electronic devices. The present application further relates to processes for preparing the compounds mentioned, and to electronic devices comprising the compounds mentioned.

Electronic devices in the context of this application are understood to mean what are called organic electronic devices, which contain organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The construction and general principle of function of OLEDs are known to those skilled in the art.

In electronic devices, especially OLEDs, there is great interest in an improvement in the performance data, especially lifetime, efficiency and operating voltage. In these aspects, it has not yet been possible to find any entirely satisfactory solution.

In addition, materials having a high refractive index are being sought, especially for use in hole-transporting layers of OLEDs, very particularly for use in electronic blocking layers of OLEDs.

A great influence on the performance data of electronic devices is possessed by emission layers and layers having a hole-transporting function. Novel compounds are also being sought for use in these layers, especially hole-transporting compounds and compounds that can serve as matrix material, especially for phosphorescent emitters, in an emitting layer. Compounds that combine hole- and electron-transporting properties in one compound also being sought. Compounds of this kind are referred to as bipolar compounds. It is preferable here that the hole-transporting properties are localized in one part of the compound, and the electron-transporting properties in another part of the compound.

In the prior art, various triarylamine compounds are known as hole transport materials for electronic devices. Likewise known is the use of particular triarylamine compounds as matrix materials in emitting layers.

However, there is still a need for alternative compounds suitable for use in electronic devices.

There is also a need for improvement with regard to the performance data in use in electronic devices, especially with regard to lifetime and efficiency, and with regard to refractive index.

It has now been found that particular triarylamine compounds are of excellent suitability for use in electronic devices, especially for use in OLEDs, even more especially for use therein as hole transport materials and for use as matrix materials for phosphorescent emitters. The materials preferably fulfil the abovementioned desirable properties with regard to lifetime, efficiency and refractive index.

The present application thus provides compounds of a formula (I)

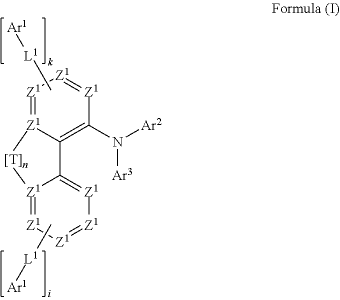

where the variables that occur are as follows:
Z¹ is the same or different at each instance and is selected from CR¹ and N, where Z¹ is C when an Ar¹ or T group is attached;
Ar¹ is the same or different at each instance and is a heteroaryl group which has 5 to 30 aromatic ring atoms and may be substituted by one or more R² radicals;
L¹ is a single bond, or an aromatic ring system which has 6 to 30 aromatic ring atoms and may be substituted by one or more R² radicals, or a heteroaromatic ring system which has 5 to 30 aromatic ring atoms and may be substituted by one or more R² radicals;
Ar² corresponds to a formula (A), (B) or (C)

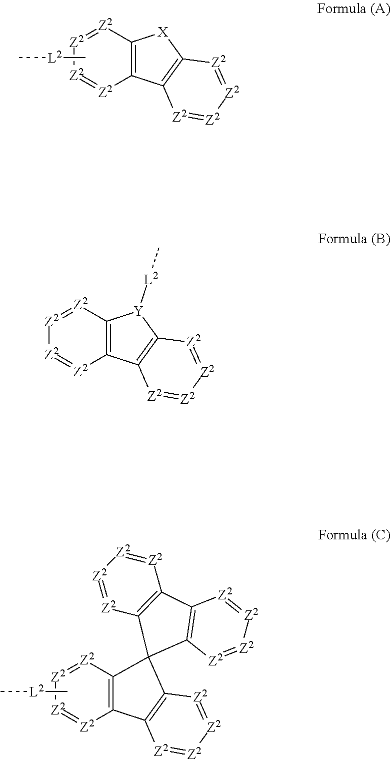

Z² is the same or different at each instance and is CR³ or N, where Z² is C when an L² group is bonded thereto;
L² is a single bond, or an aromatic ring system which has 6 to 30 aromatic ring atoms and may be substituted by one or more R³ radicals, or a heteroaromatic ring system which has 5 to 30 aromatic ring atoms and may be substituted by one or more R³ radicals;
X is selected from C(R³)₂, NR³, O and S;
Y is selected from CR³ and N;
Ar³ corresponds to a formula (A), a formula (B) or a formula (C), or is an aromatic ring system which has 6 to 30 aromatic ring atoms and may be substituted by one or more R⁴ radicals, or a heteroaromatic ring system which has 5 to 30 aromatic ring atoms and may be substituted by one or more R⁴ radicals;
T is selected from C(R¹)₂, NR¹, O and S;
R¹, R², R³, R⁴ are the same or different at each instance and are selected from H, D, F, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ or R² or R³ or R⁴ radicals may be joined to one another and may form a ring; where the alkyl, alkoxy, alkenyl and alkynyl groups mentioned and the aromatic ring systems and heteroaromatic ring systems mentioned may each be substituted by one or more R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned may each be substituted by one or more R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or S02;
R⁶ is the same or different at each instance and is selected from H, D, F, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁶ radicals may be joined to one another and may form a ring; and where the alkyl, alkoxy, alkenyl and alkynyl groups, aromatic ring systems and heteroaromatic ring systems mentioned may be substituted by F or CN;
n is 0 or 1, where the T group is absent when n is 0;
i is 0, 1, 2, 3, 4 or 5, where the -L¹-Ar¹ group having the index i is absent when i is 0;
k is 0, 1, 2, 3 or 4, where the -L¹-Ar¹ group having the index k is absent when k is 0;
where the sum of k and i is at least 1,
excluding:

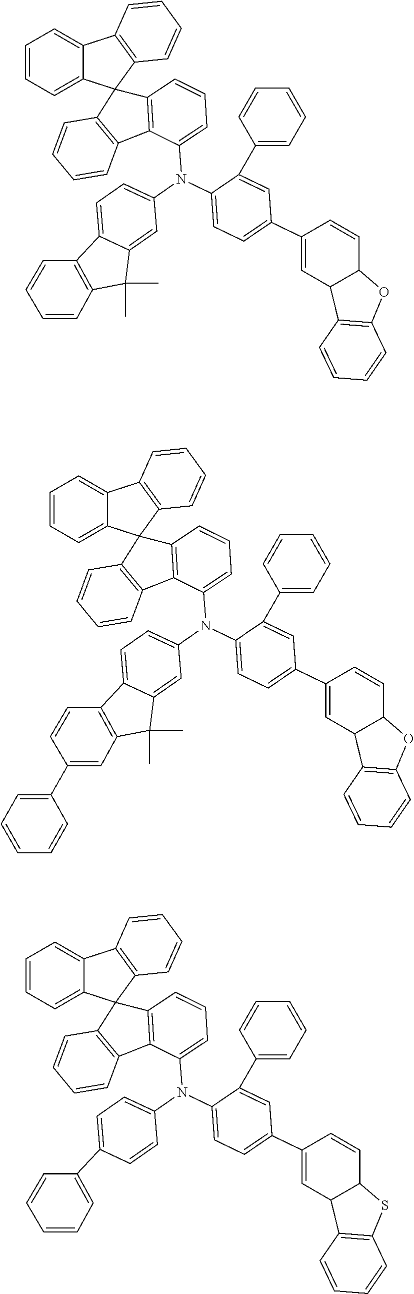 [SMILES: CC1(C)c2ccccc2-c2ccc(N(c3ccc(C4=CC5c6ccccc6OC5C=C4)cc3-c3ccccc3)c3cccc4c3-c3ccccc3C43c4ccccc4-c4ccccc43)cc21 ; CC1(C)c2cc(-c3ccccc3)ccc2-c2ccc(N(c3ccc(C4=CC5c6ccccc6OC5C=C4)cc3-c3ccccc3)c3cccc4c3-c3ccccc3C43c4ccccc4-c4ccccc43)cc21 ; C1=CC2Sc3ccccc3C2C=C1c1ccc(N(c2ccc(-c3ccccc3)cc2)c2cccc3c2-c2ccccc2C32c3ccccc3-c3ccccc32)c(-c2ccccc2)c1]

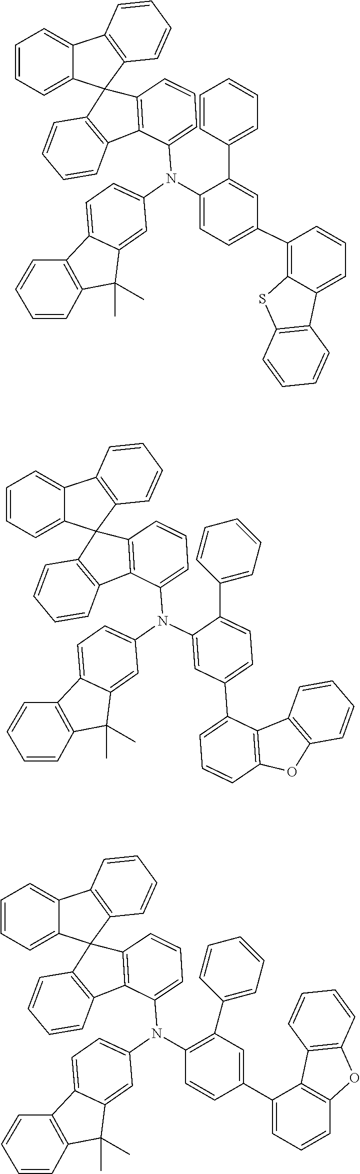 [SMILES: CC1(C)c2ccccc2-c2ccc(N(c3ccc(-c4cccc5c4sc4ccccc45)cc3-c3ccccc3)c3cccc4c3-c3ccccc3C43c4ccccc4-c4ccccc43)cc21 ; CC1(C)c2ccccc2-c2ccc(N(c3cc(-c4cccc5oc6ccccc6c45)ccc3-c3ccccc3)c3cccc4c3-c3ccccc3C43c4ccccc4-c4ccccc43)cc21 ; CC1(C)c2ccccc2-c2ccc(N(c3ccc(-c4cccc5oc6ccccc6c45)cc3-c3ccccc3)c3cccc4c3-c3ccccc3C43c4ccccc4-c4ccccc43)cc21]

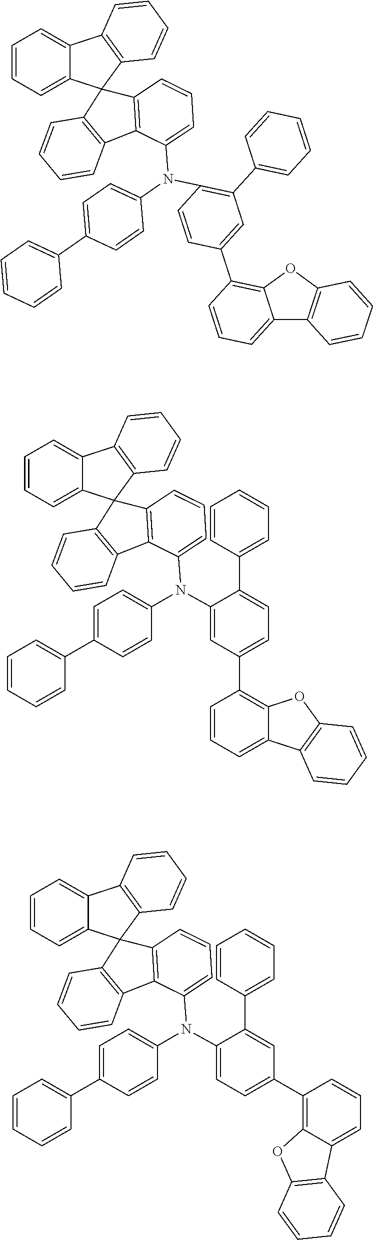 [SMILES: c1ccc(-c2ccc(N(c3ccc(-c4cccc5c4oc4ccccc45)cc3-c3ccccc3)c3cccc4c3-c3ccccc3C43c4ccccc4-c4ccccc43)cc2)cc1 ; c1ccc(-c2ccc(N(c3cc(-c4cccc5c4oc4ccccc45)ccc3-c3ccccc3)c3cccc4c3-c3ccccc3C43c4ccccc4-c4ccccc43)cc2)cc1 ; c1ccc(-c2ccc(N(c3ccc(-c4cccc5c4oc4ccccc45)cc3-c3ccccc3)c3cccc4c3-c3ccccc3C43c4ccccc4-c4ccccc43)cc2)cc1]

An aryl group in the context of this invention contains 6 to 40 aromatic ring atoms of which none is a heteroatom. An aryl group in the context of this invention is understood to mean either a simple aromatic cycle, i.e. benzene, or a fused aromatic polycycle, for example naphthalene, phenanthrene or anthracene. A fused aromatic polycycle in the context of the present application consists of two or more simple aromatic cycles fused to one another. Fusion between cycles is understood here to mean that the cycles share at least one edge with one another.

A heteroaryl group in the context of this invention contains 5 to 40 aromatic ring atoms of which at least one is a heteroatom. The heteroatoms of the heteroaryl group are preferably selected from N, O and S. A heteroaryl group in the context of this invention is understood to mean either a simple heteroaromatic cycle, for example pyridine, pyrimidine or thiophene, or a fused heteroaromatic polycycle, for example quinoline or carbazole. A fused heteroaromatic polycycle in the context of the present application consists of two or more simple heteroaromatic cycles fused to one another. Fusion between cycles is understood here to mean that the cycles share at least one edge with one another.

An aryl or heteroaryl group, each of which may be substituted by the abovementioned radicals and which may be joined to the aromatic or heteroaromatic system via any desired positions, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention contains 6 to 40 carbon atoms in the ring system and does not include any heteroatoms as aromatic ring atoms. An aromatic ring system in the context of this invention therefore does not contain any heteroaryl groups. An aromatic ring system in the context of this invention shall be understood to mean a system which does not necessarily contain only aryl groups but in which it is also possible for a plurality of aryl groups to be bonded by a single bond or by a non-aromatic unit, for example one or more optionally substituted C, Si, N, O or S atoms. In this case, the non-aromatic unit comprises preferably less than 10% of the atoms other than H, based on the total number of atoms other than H in the system. For example, systems such as 9,9′-spirobifluorene, 9,9′-diarylfluorene, triarylamine, diaryl ethers and stilbene are also to be regarded as aromatic ring systems in the context of this invention, and likewise systems in which two or more aryl groups are joined, for example, by a linear or cyclic alkyl, alkenyl or alkynyl group or by a silyl group. In addition, systems in which two or more aryl groups are joined to one another via single bonds are also regarded as aromatic ring systems in the context of this invention, for example systems such as biphenyl and terphenyl.

A heteroaromatic ring system in the context of this invention contains 5 to 40 aromatic ring atoms, at least one of which is a heteroatom. The heteroatoms of the heteroaromatic ring system are preferably selected from N, O and/or S. A heteroaromatic ring system corresponds to the abovementioned definition of an aromatic ring system, but has at least one heteroatom as one of the aromatic ring atoms. In this way, it differs from an aromatic ring system in the sense of the definition of the present application, which, according to this definition, cannot contain any heteroatom as aromatic ring atom.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be replaced by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

When T is selected from O and S, L¹ preferably does not contain any carbazole unit, and Ar¹, including its substituents, preferably does not contain any carbazole unit. This means that L¹ and Ar¹ also do not have any groups derived from carbazole by fusion of rings, for example benzocarbazole.

When T is selected from O and S, L¹ is preferably selected from a single bond and an aromatic ring system which has 6 to 30 aromatic ring atoms and may be substituted by one or more R² radicals, and Ar¹ is selected from a group of the formula (Ar¹-A) shown below.

Preferably, Z¹ is CR¹, where Z¹ is C when an Ar¹ or T group is bonded thereto.

Preferably Ar¹ is the same or different at each instance and is a heteroaryl group which has 6 to 20 aromatic ring atoms and may be substituted by one or more R² radicals. More preferably, Ar¹ is the same or different at each instance and is selected from groups of the following formulae:

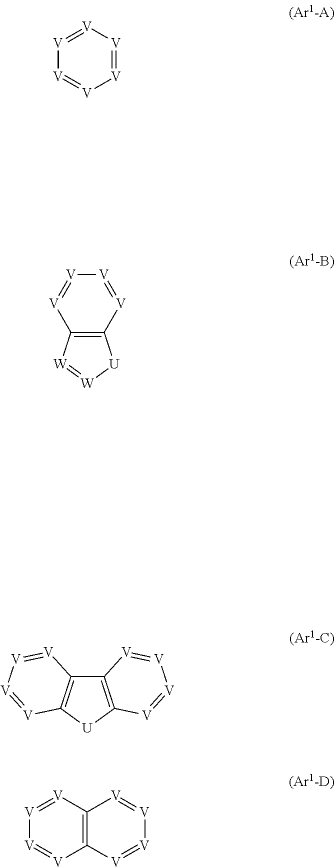

where the variables that occur are defined as follows:
V is the same or different at each instance and is N or CR², where at least one V group in each of formulae (Ar¹-A) and (Ar¹-D) is N;
W is the same or different at each instance and is N or CR²;
U is O, S or NR²;
where at least one R² group per formula is replaced by the bond to the L¹ group.

Among the abovementioned groups of the formulae (Ar¹-A) to (Ar¹-D), preference is given to groups of the formulae (Ar¹-A).

Most preferably, Ar¹ is the same or different at each instance and is selected from pyridine, pyrimidine, pyridazine, pyrazine, triazine, dibenzofuran, dibenzothiophene, carbazole, benzimidazole, benzoxazole and benzothiazole, even more preferably selected from pyridine, pyrimidine, triazine, dibenzothiophene, dibenzofuran and carbazole, even more preferably still selected from pyridine, pyrimidine, triazine, dibenzothiophene and dibenzofuran, most preferably selected from pyridine, pyrimidine and triazine, where the groups mentioned may each be substituted by one or more R² radicals.

Examples of preferred substructures of the formula (I)

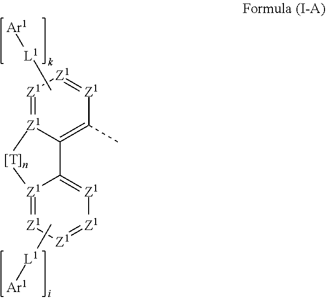

where the dotted bond indicates the bond to the rest of the formula (I) are depicted below:

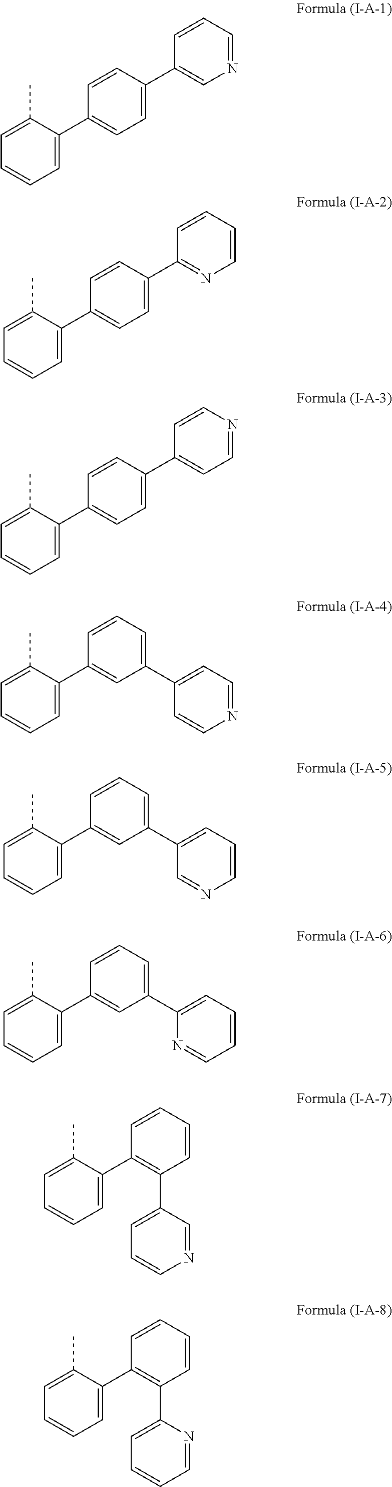

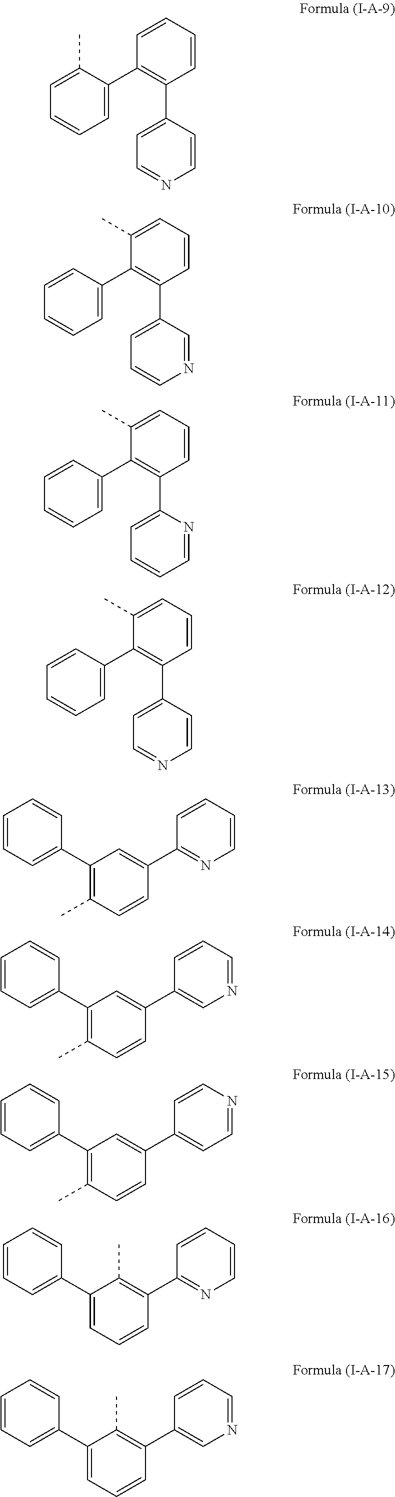

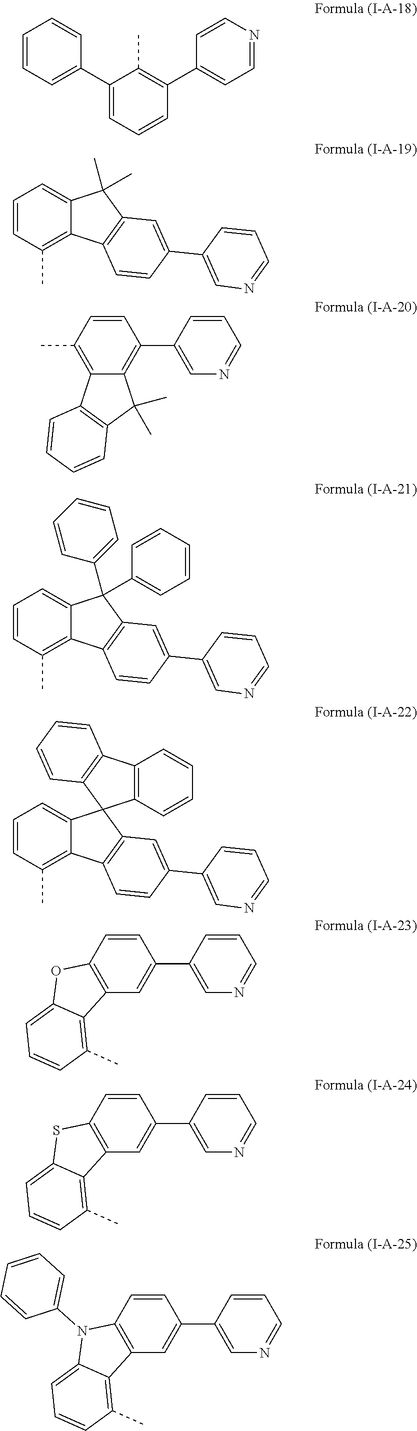

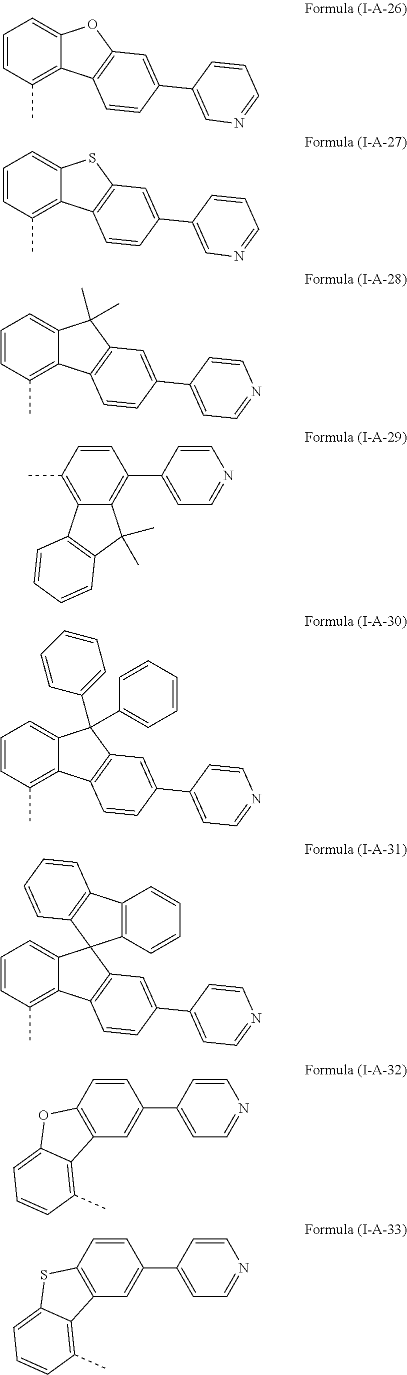

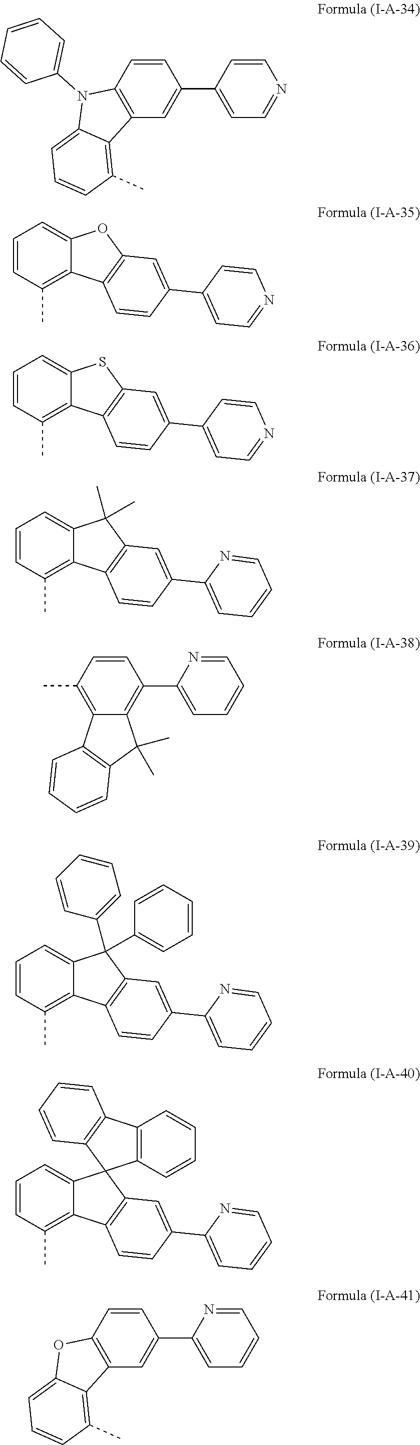

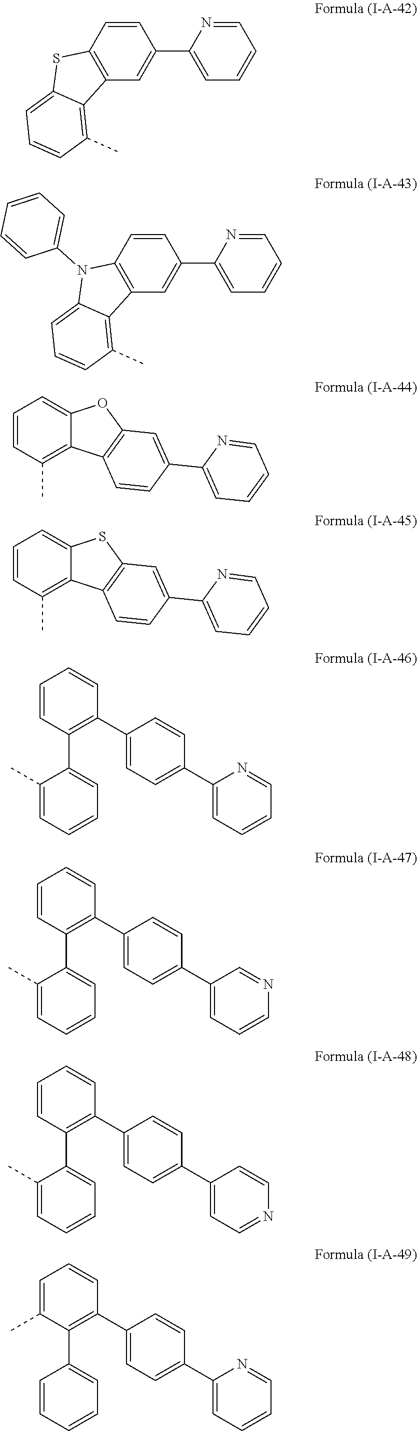

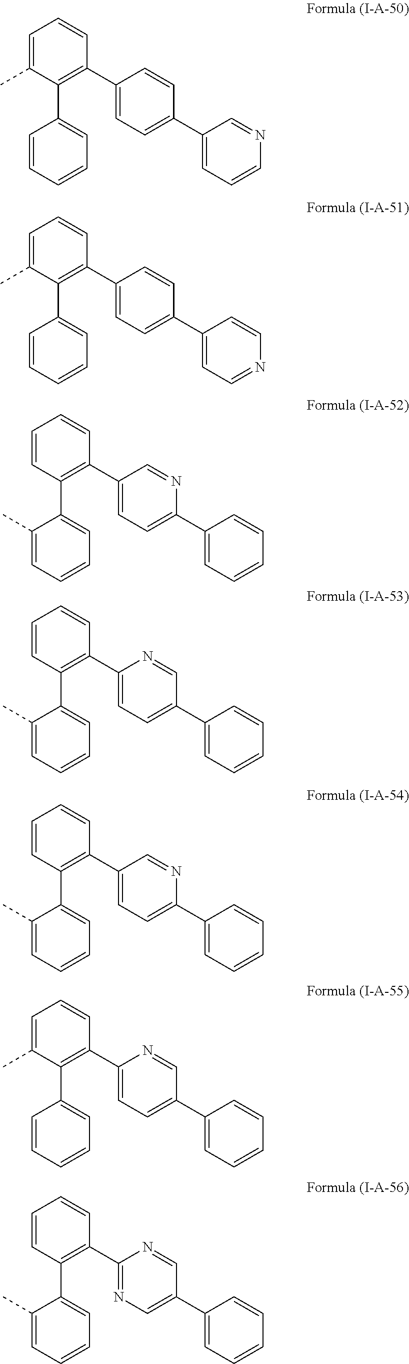

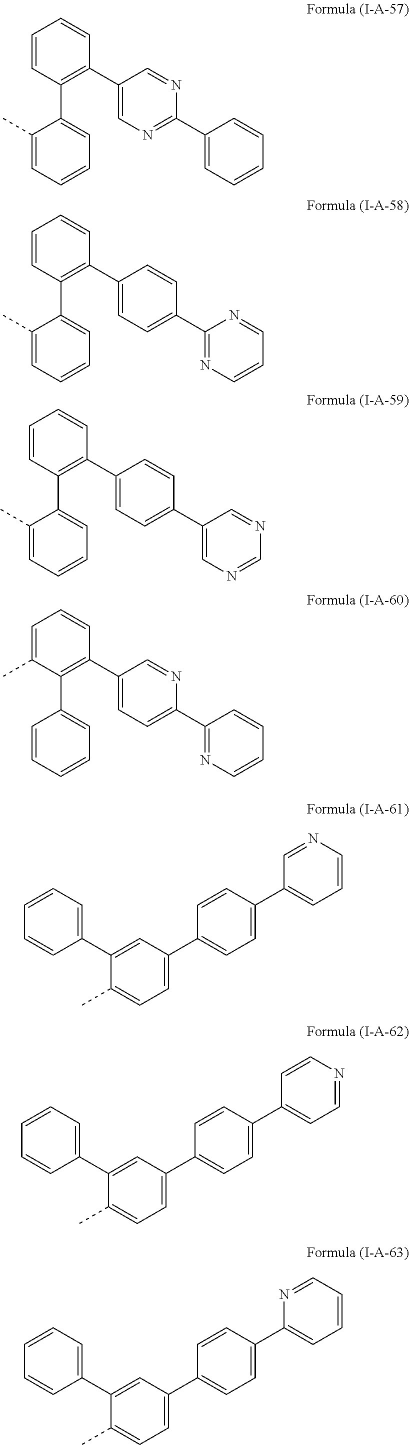

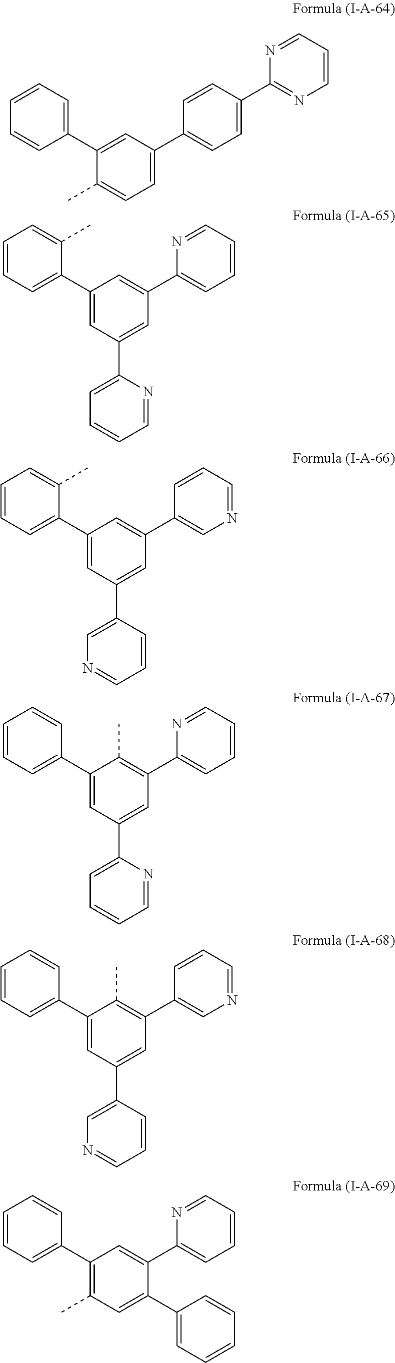

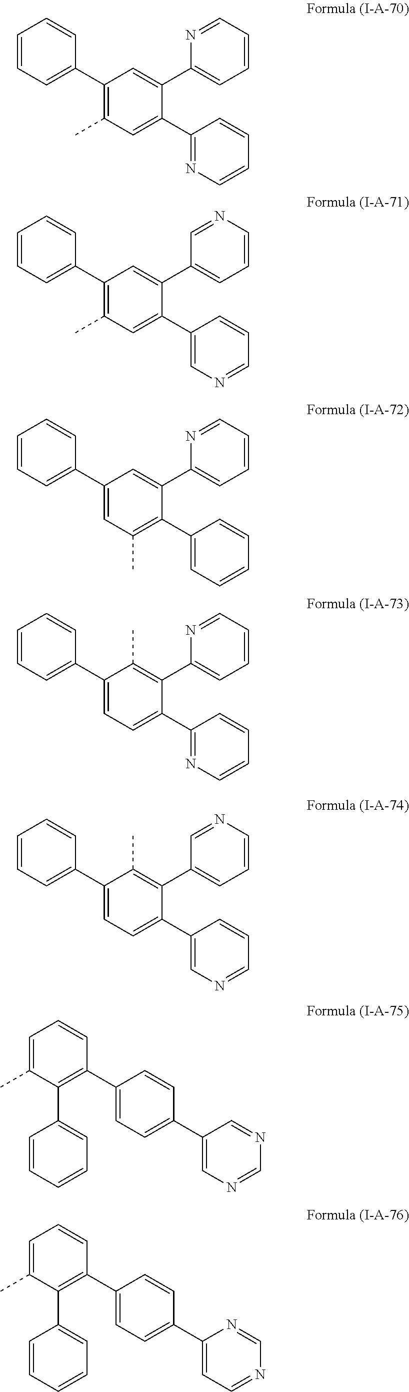

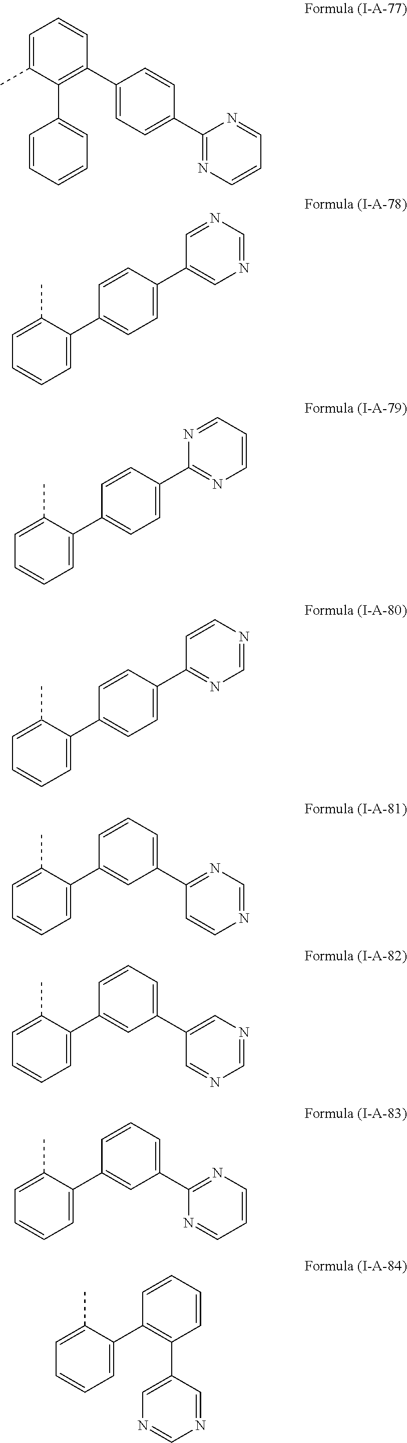

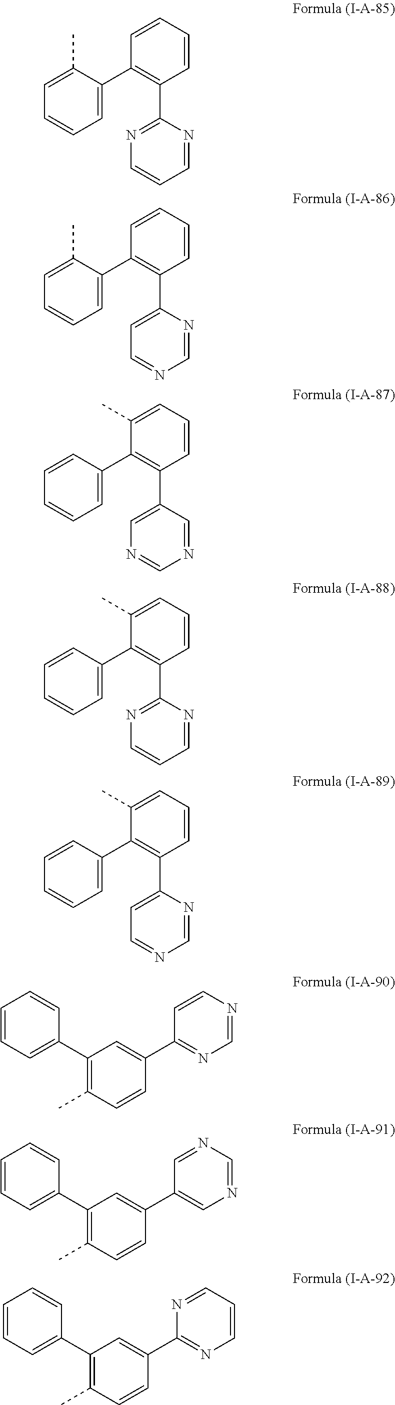

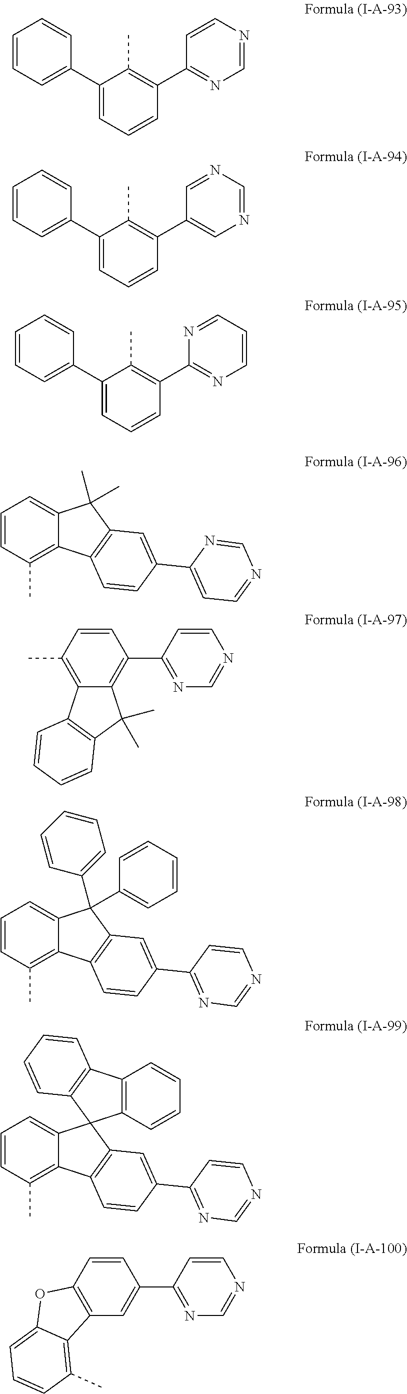

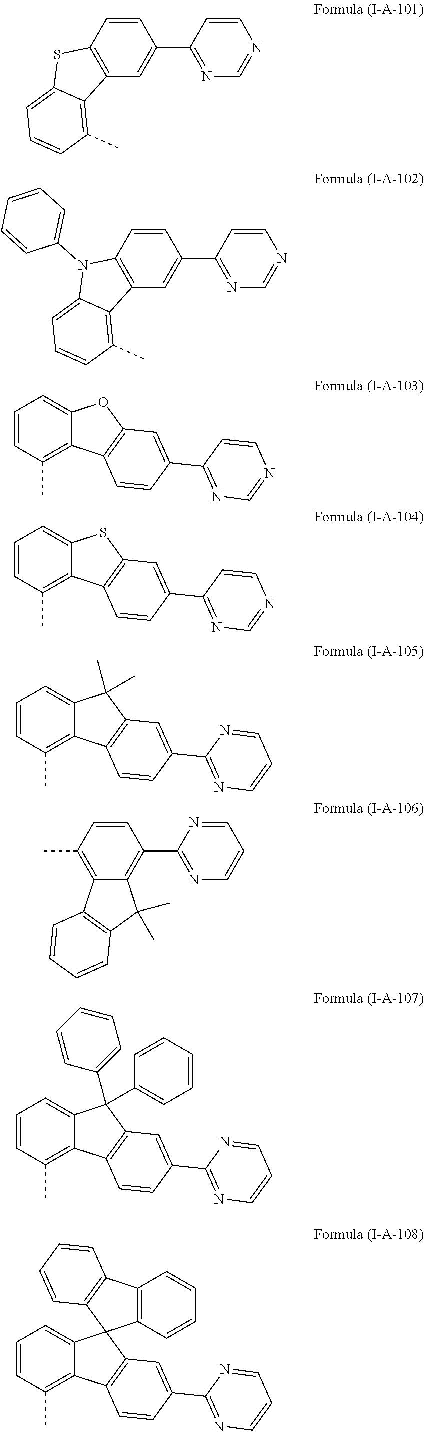

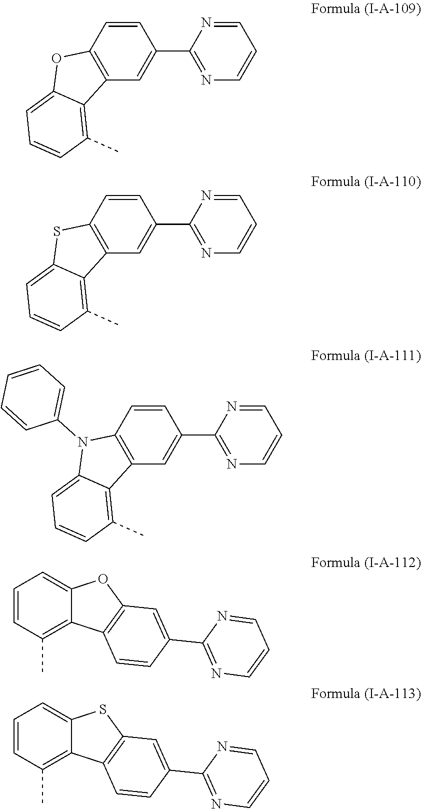

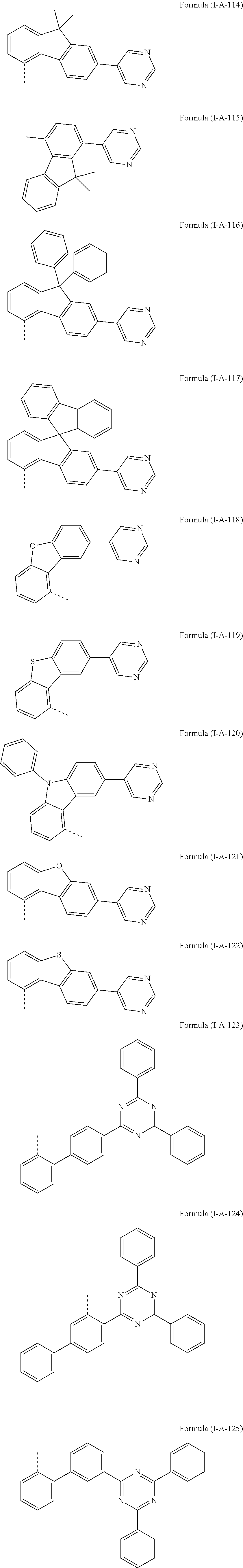

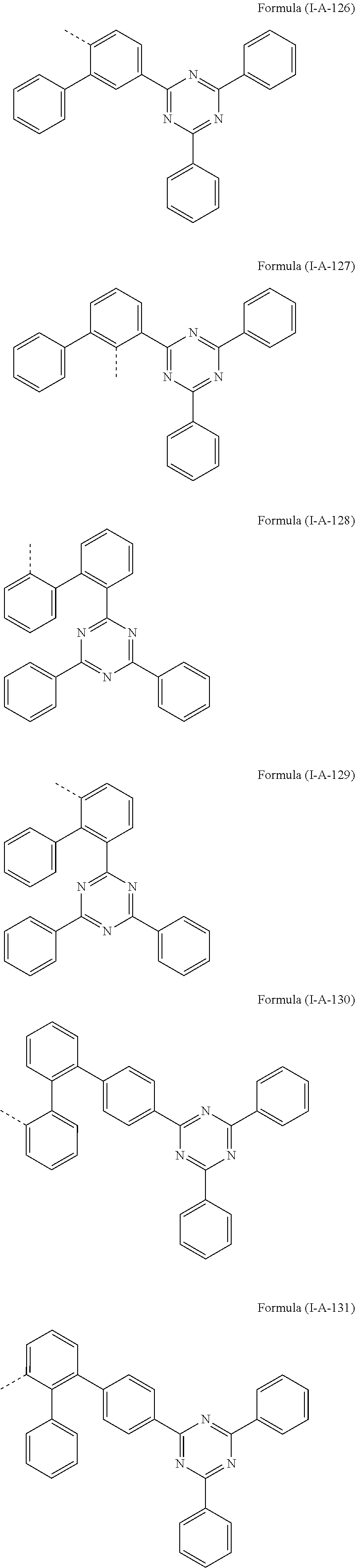

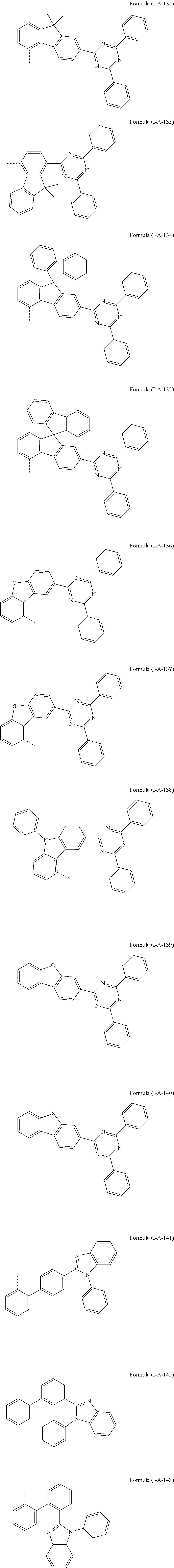

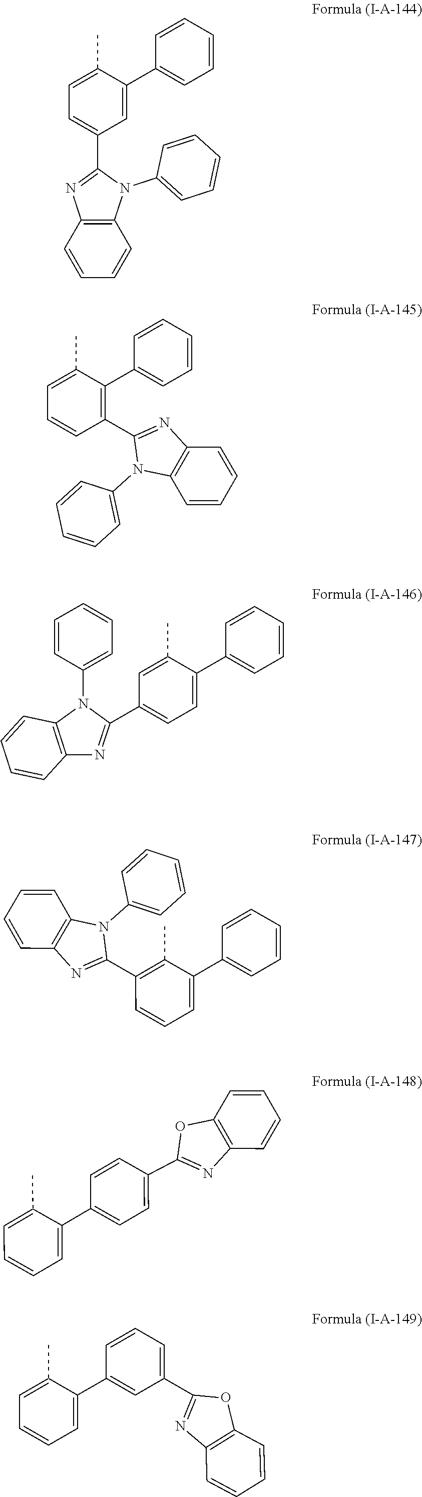

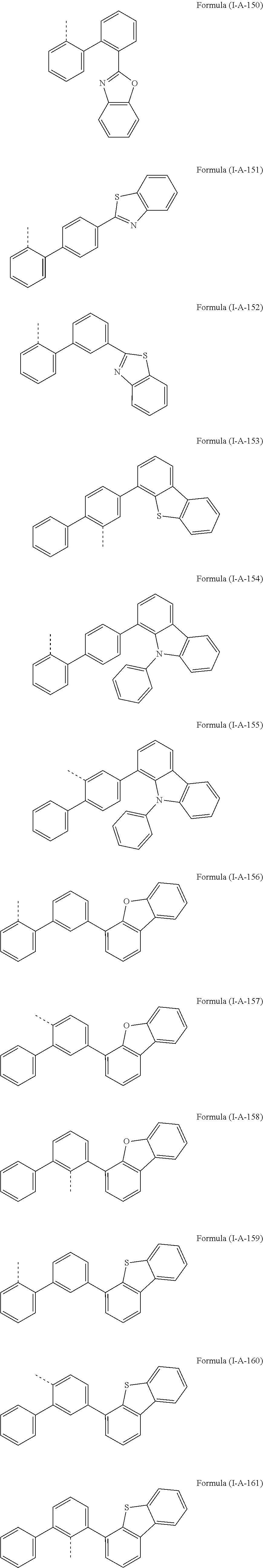

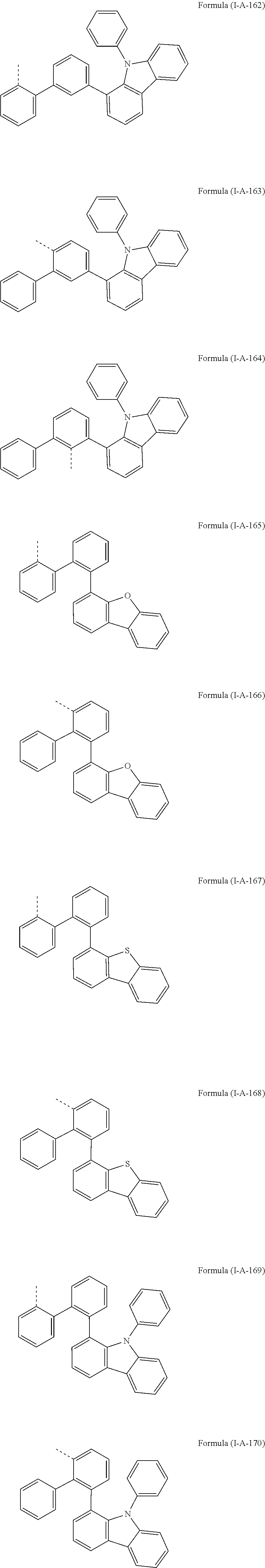

[chemical structure]

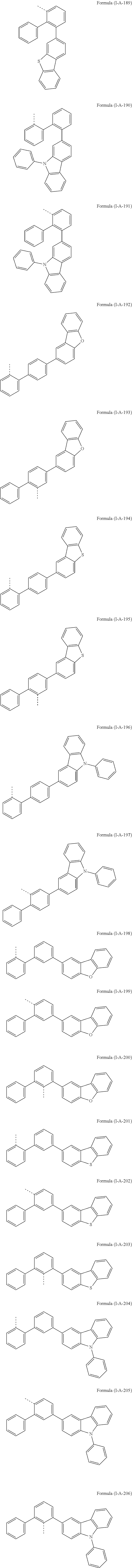

[chemical structure]

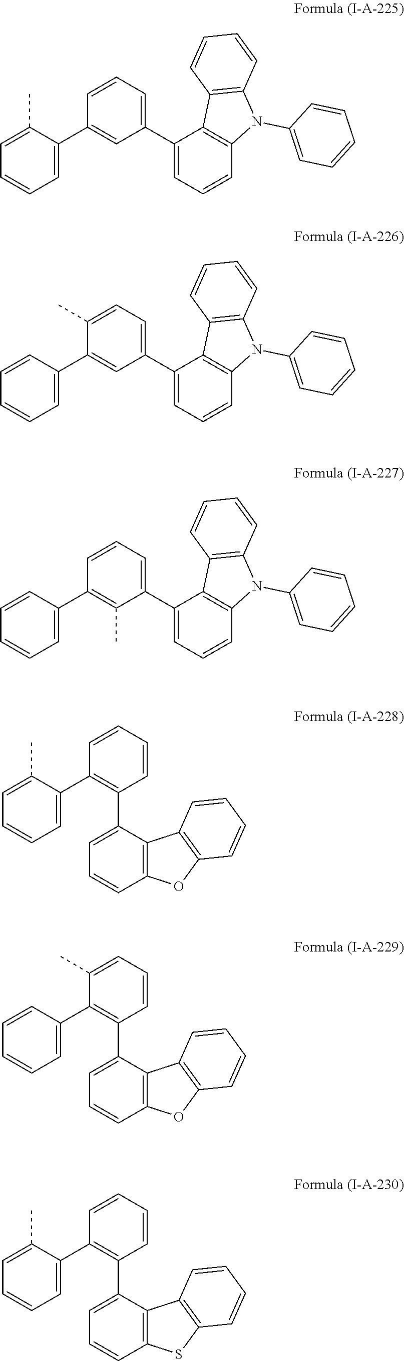

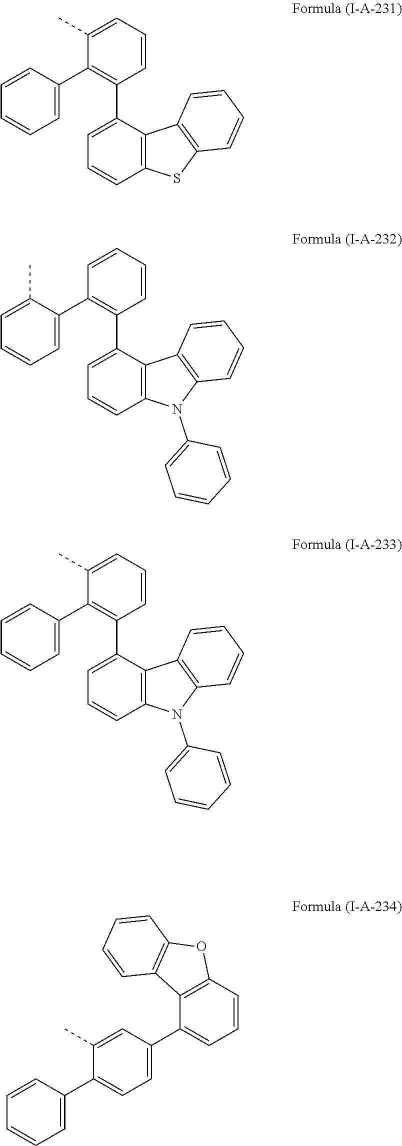

Among the abovementioned groups, particular preference is given to the following: formula (I-A-1), formula (I-A-2), formula (I-A-3), formula (I-A-19), formula (I-A-20), formula (I-A-21), formula (I-A-22), formula (I-A-78), formula (I-A-79), formula (I-A-80), formula (I-A-105), formula (I-A-106), formula (I-A-107), formula (I-A-108), formula (I-A-123), formula (I-A-126), formula (I-A-132), formula (I-A-133), formula (I-A-134), formula (I-A-135).

Preferably, L¹ is a single bond or a divalent group selected from phenylene, biphenylene, terphenylene, naphthylene, dibenzofuran, dibenzothiophene, carbazole and fluorene, where the divalent group may be substituted by one or more R² radicals. More preferably, L¹ is a single bond. Preference is given to the embodiment where L¹ is a single bond, for all the preferred embodiments of the formula (I) that are specified hereinafter.

Preferably, Ar² corresponds to the formula (A) or (C), more preferably to the formula (A).

Preferred embodiments of the formula (C) correspond to the following formulae:

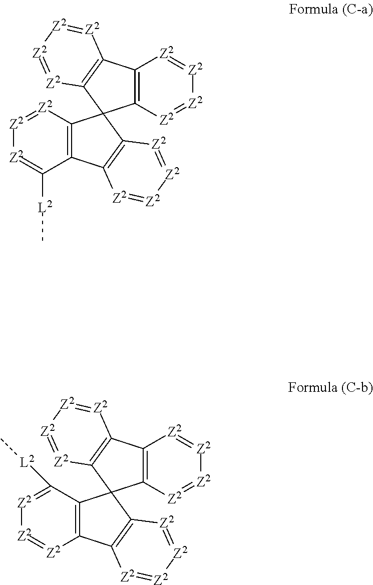

where Z² is CR³, and where L² is as defined above.

Preferred Ar² groups of the formulae (A), (B) and (C) are depicted below:

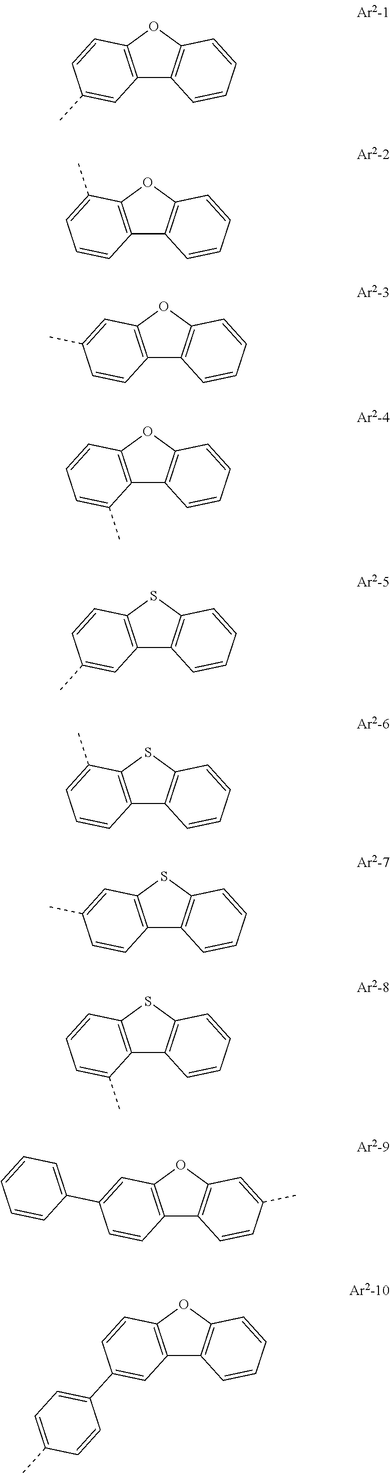

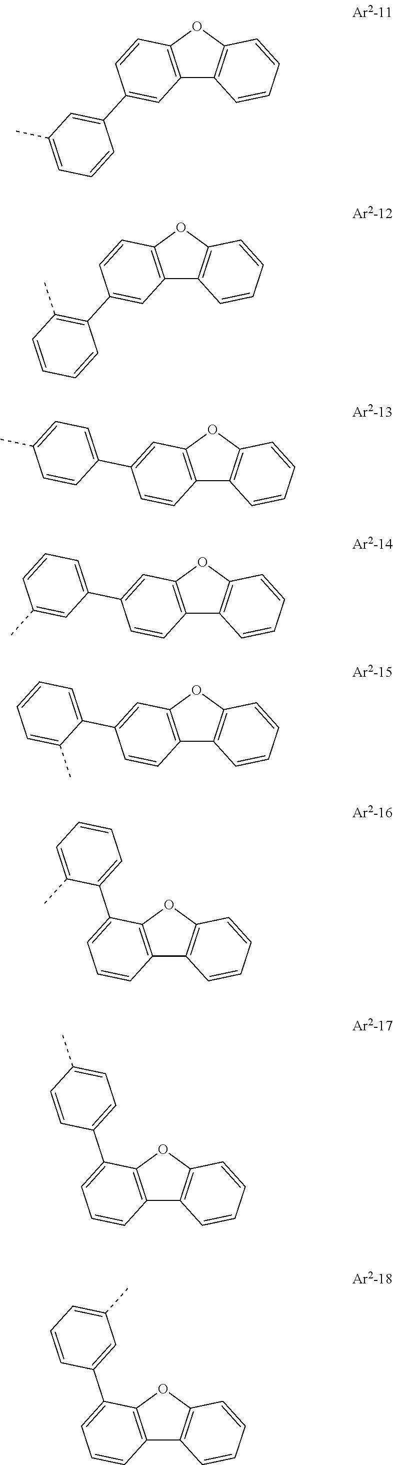

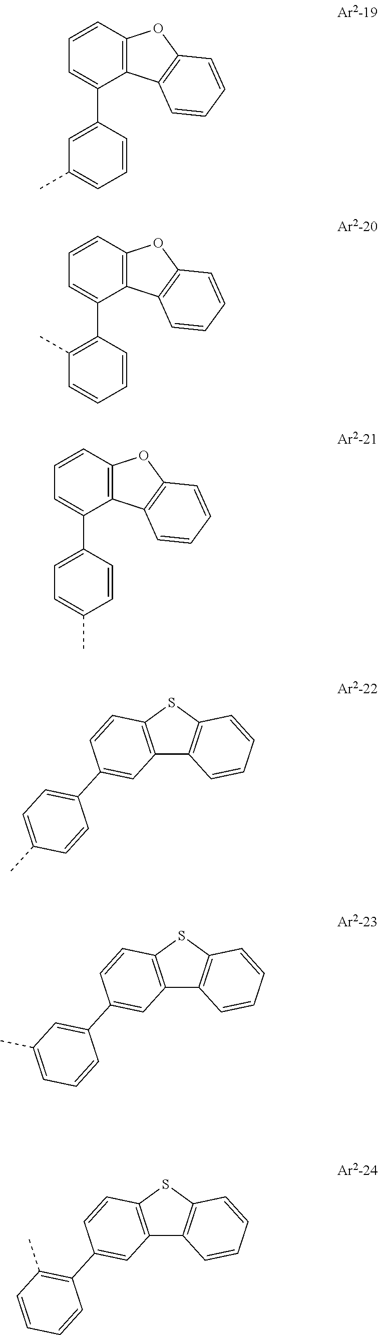

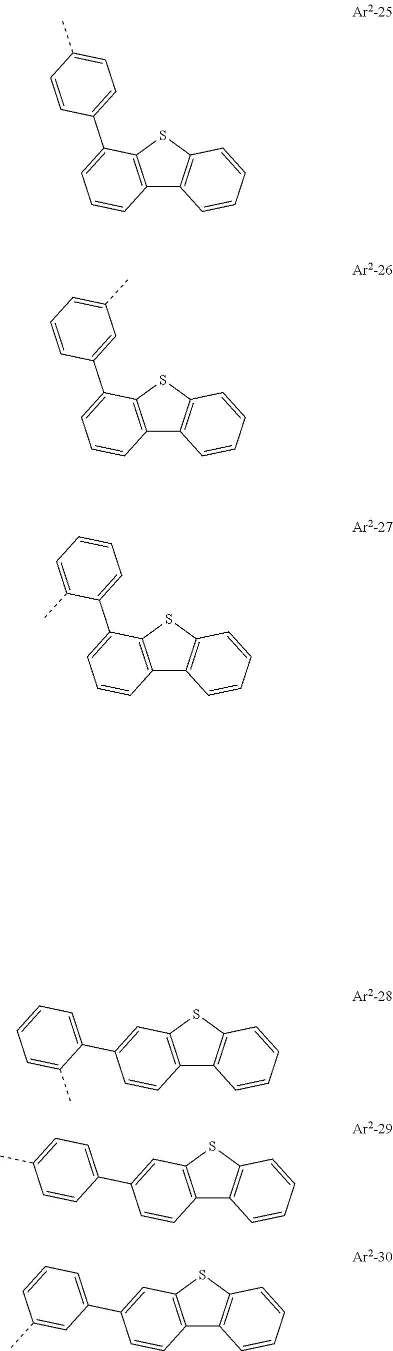

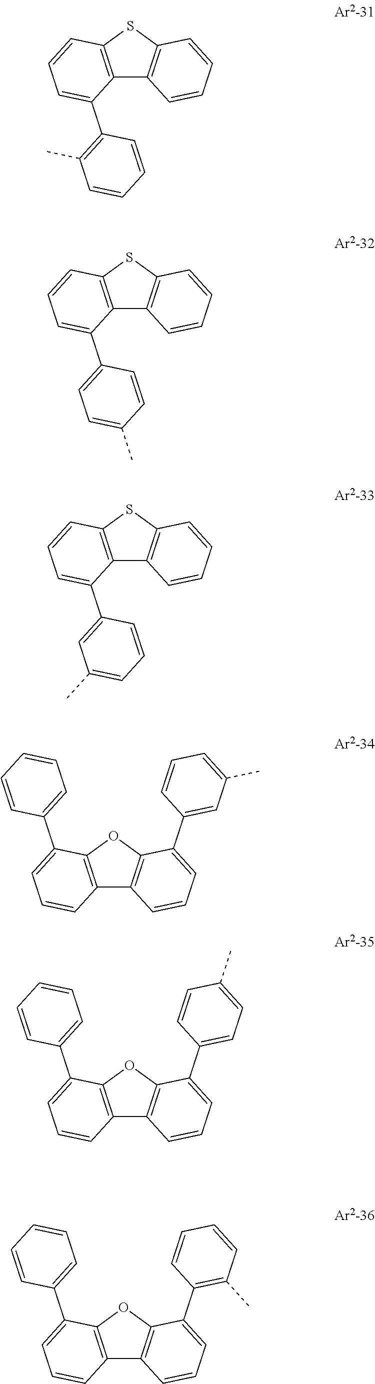

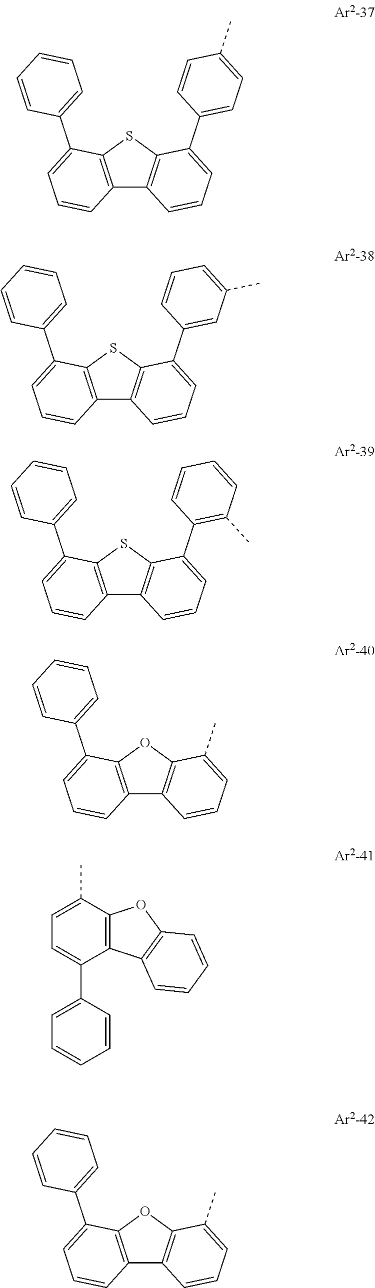

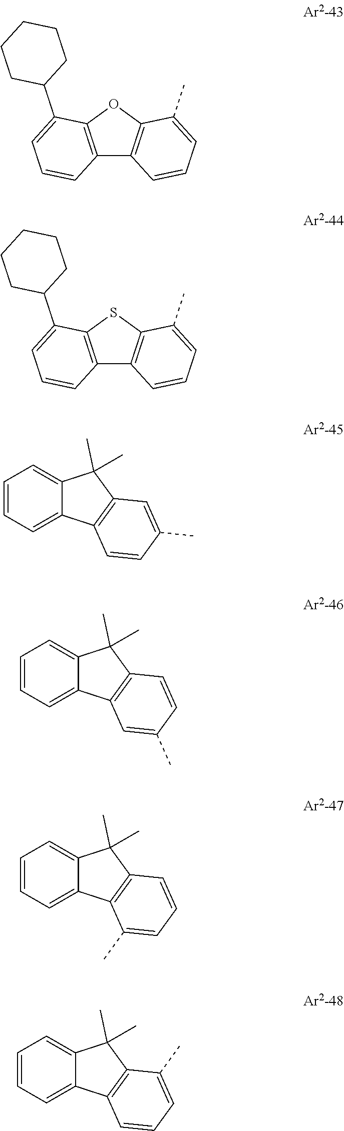

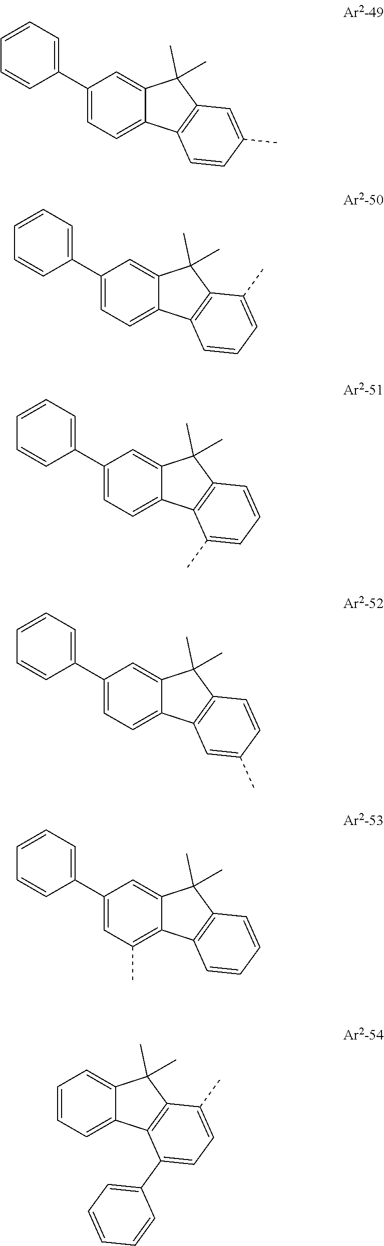

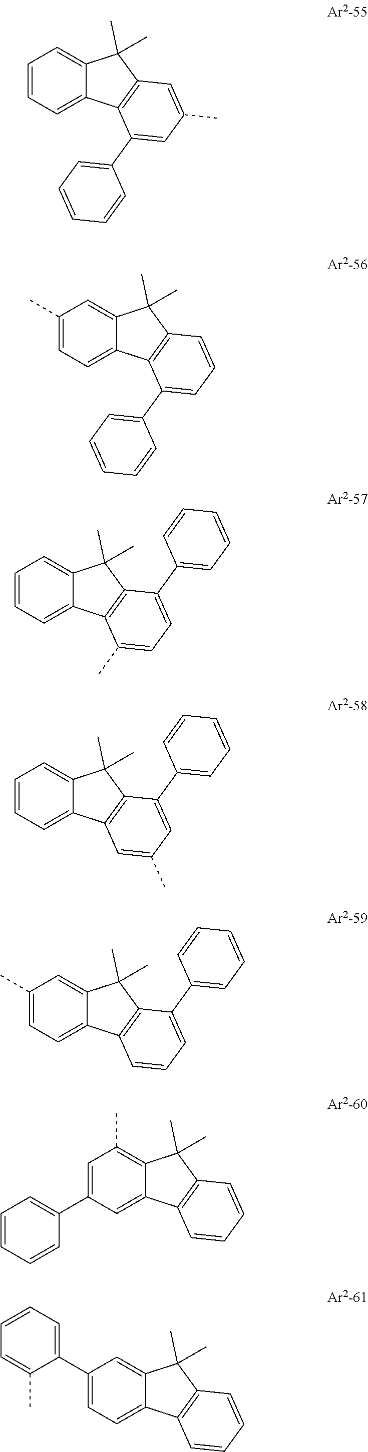

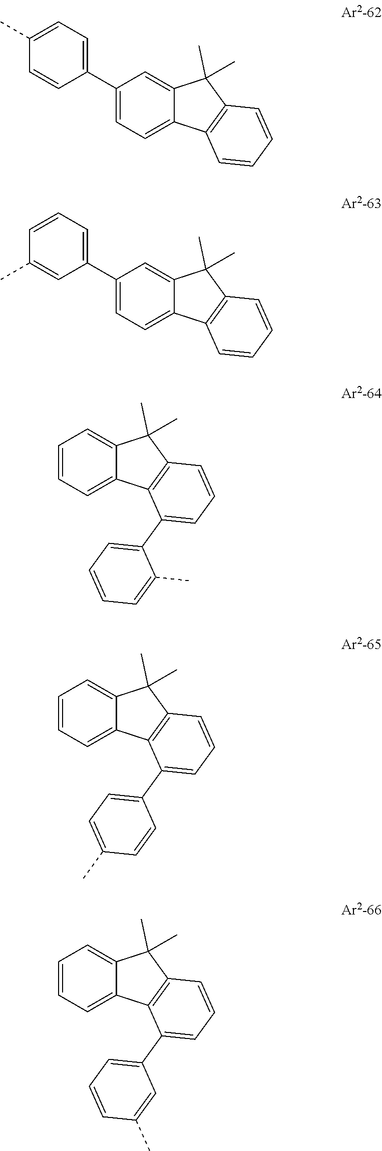

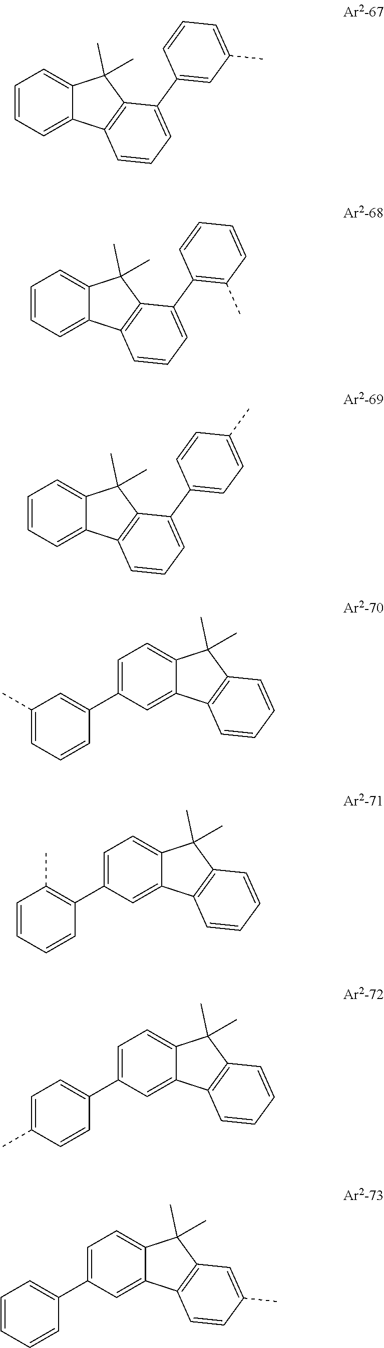

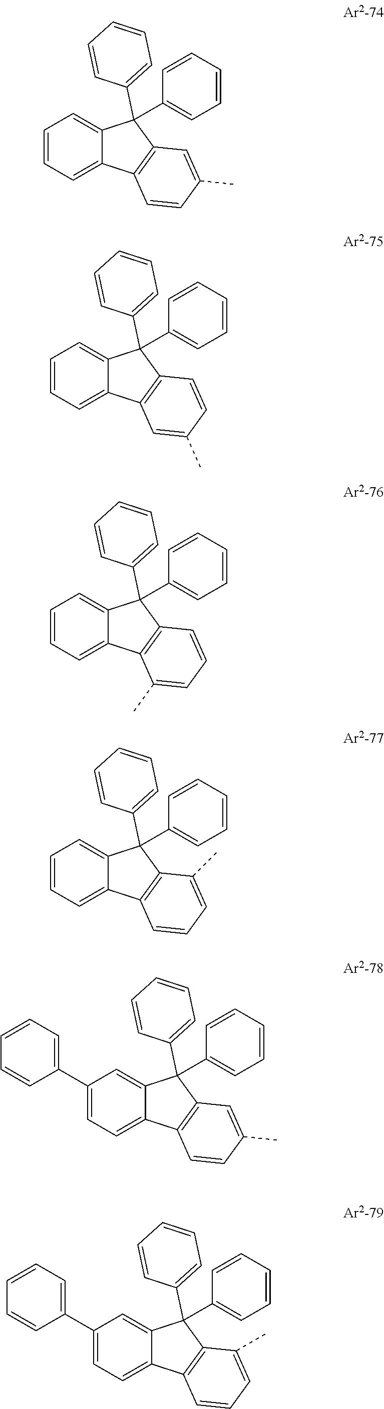

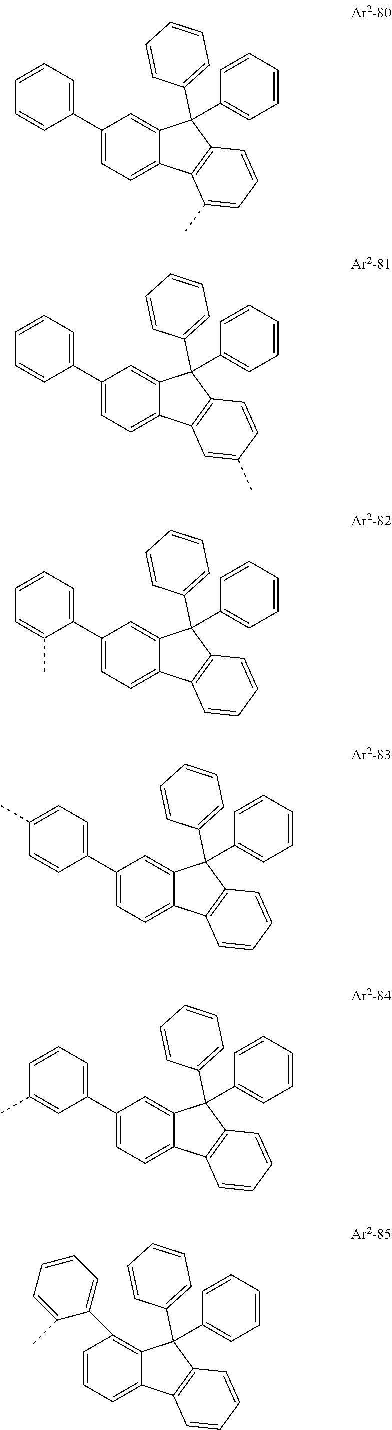

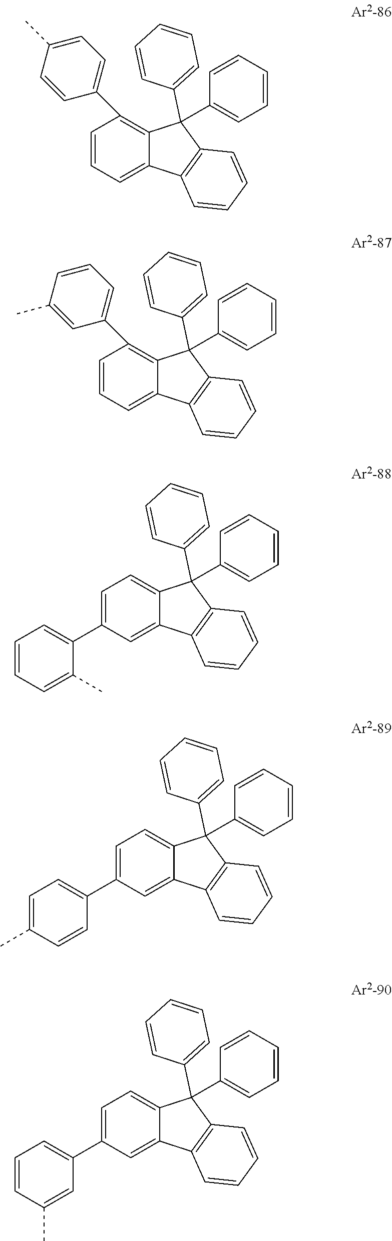

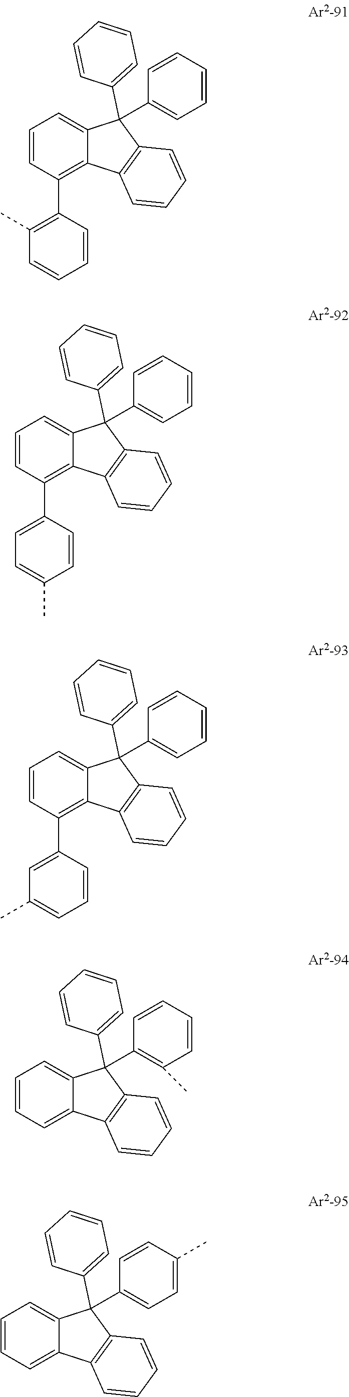

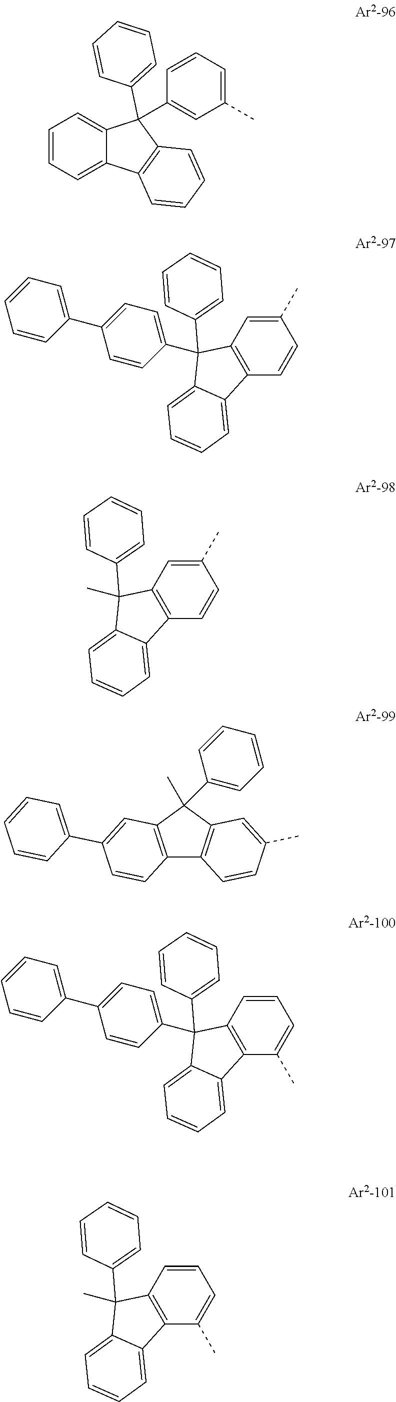

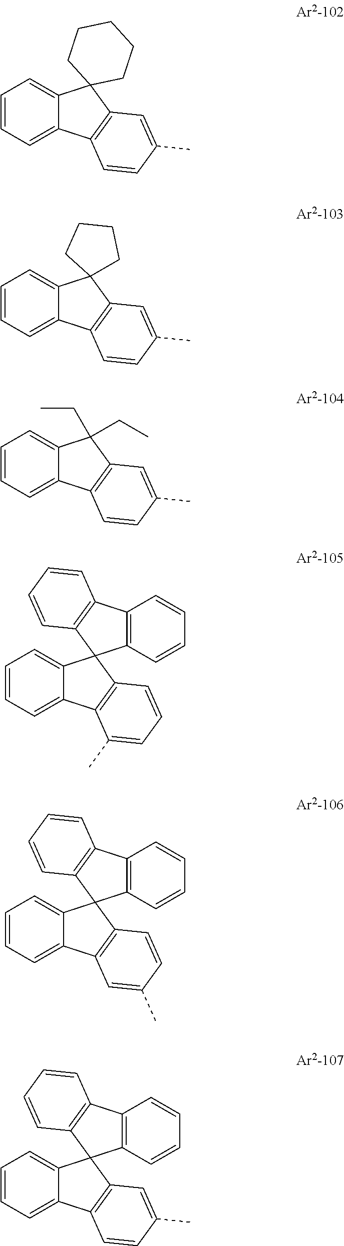

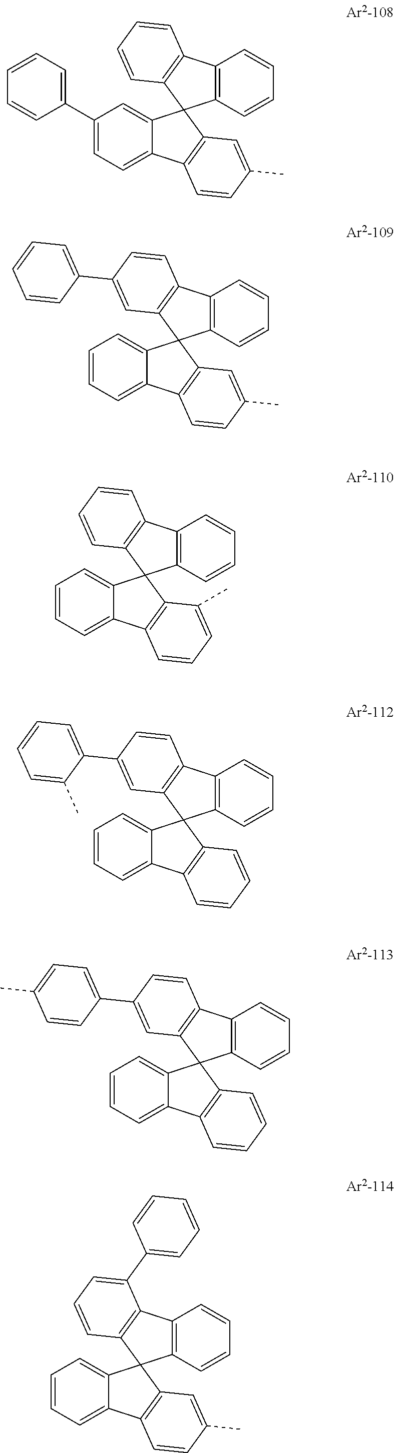

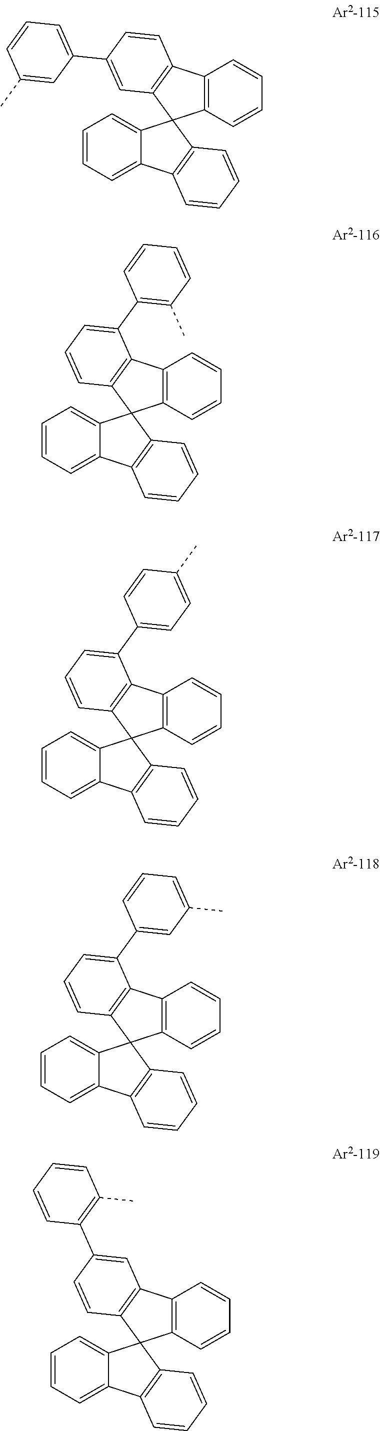

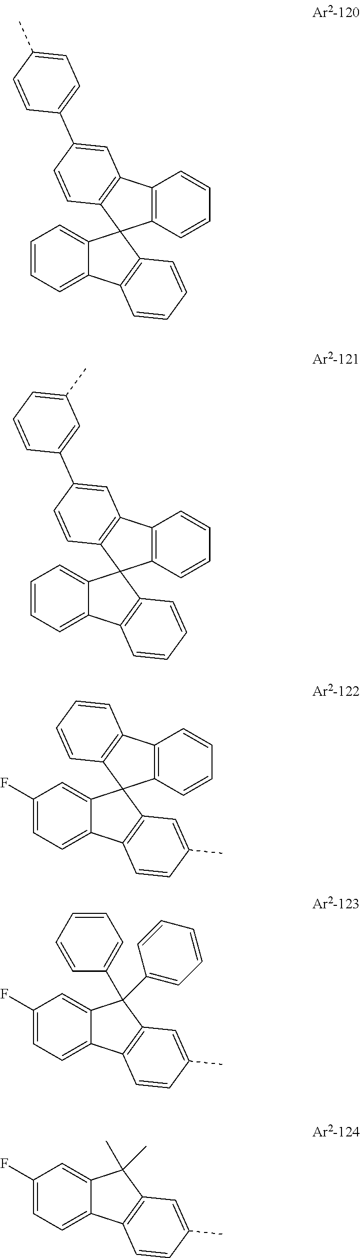

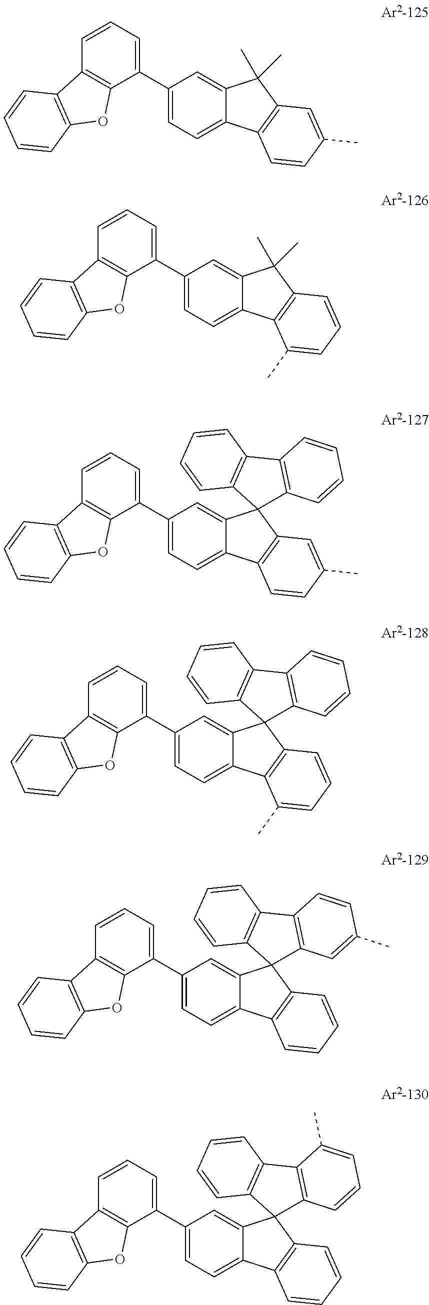

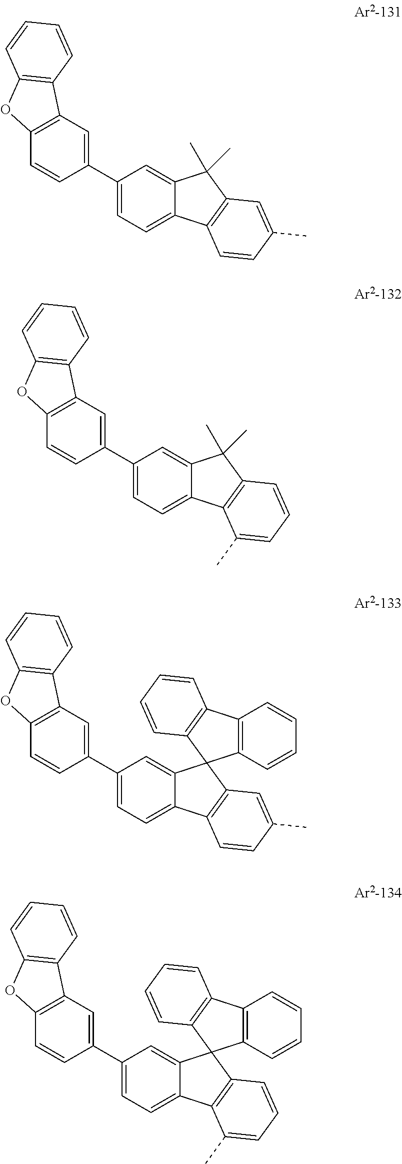

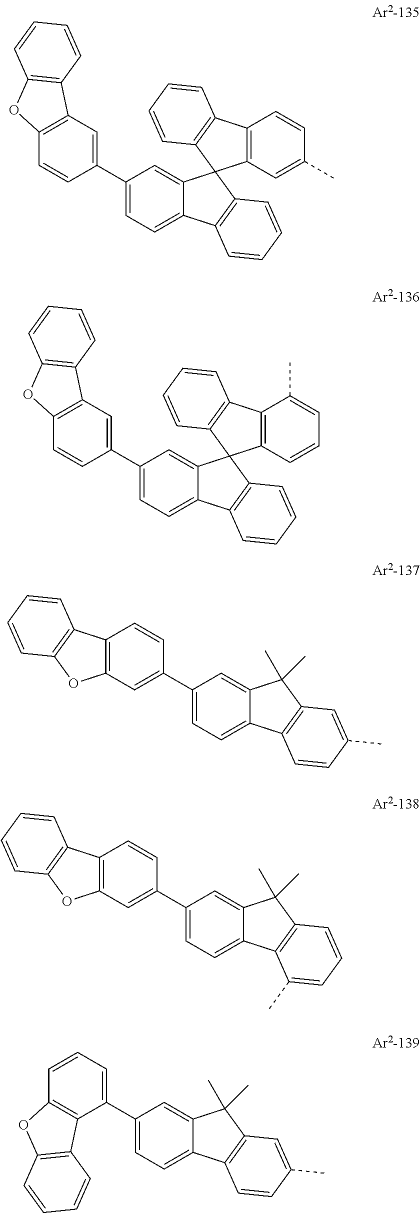

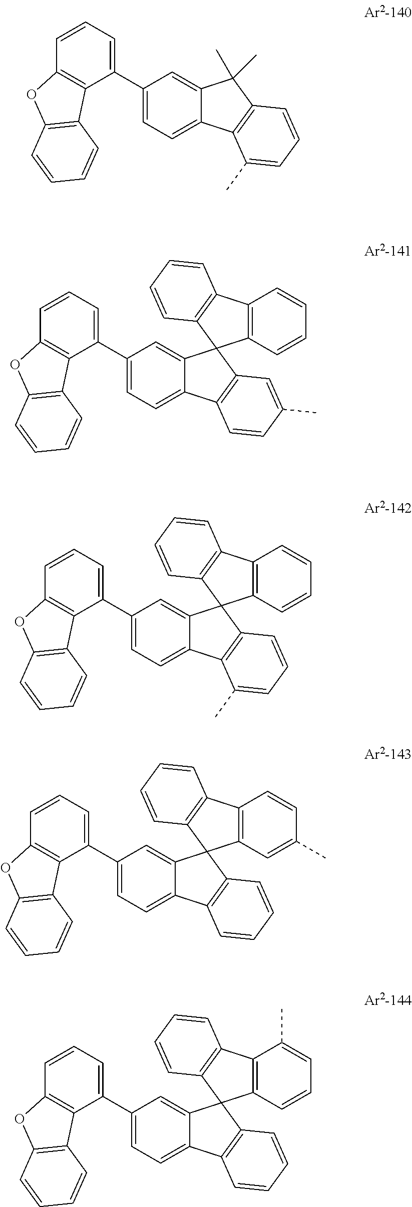

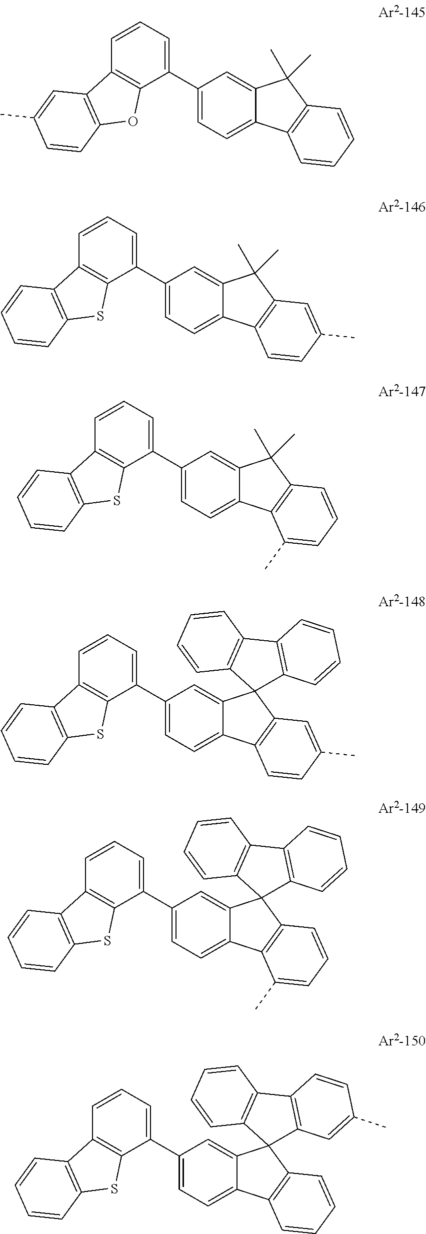

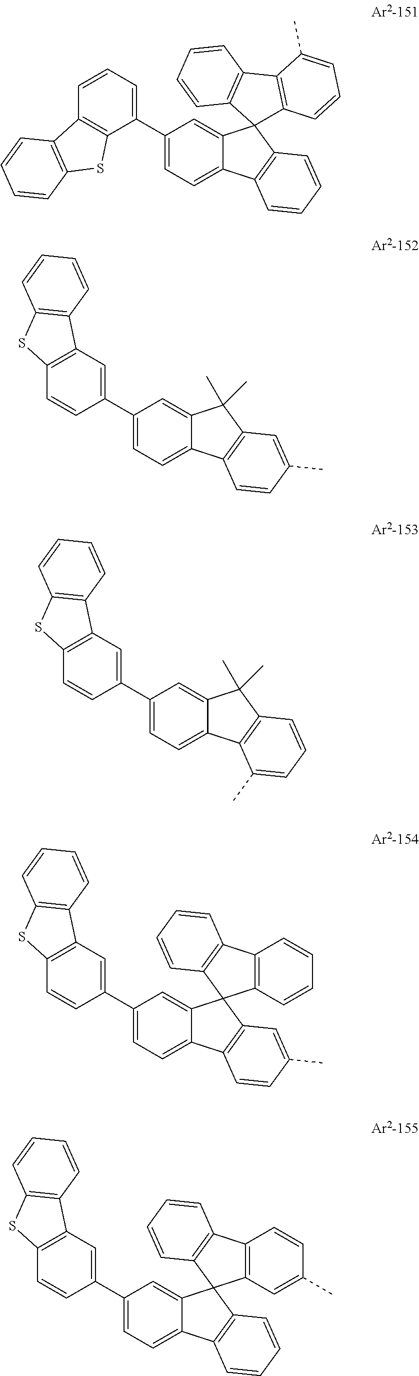

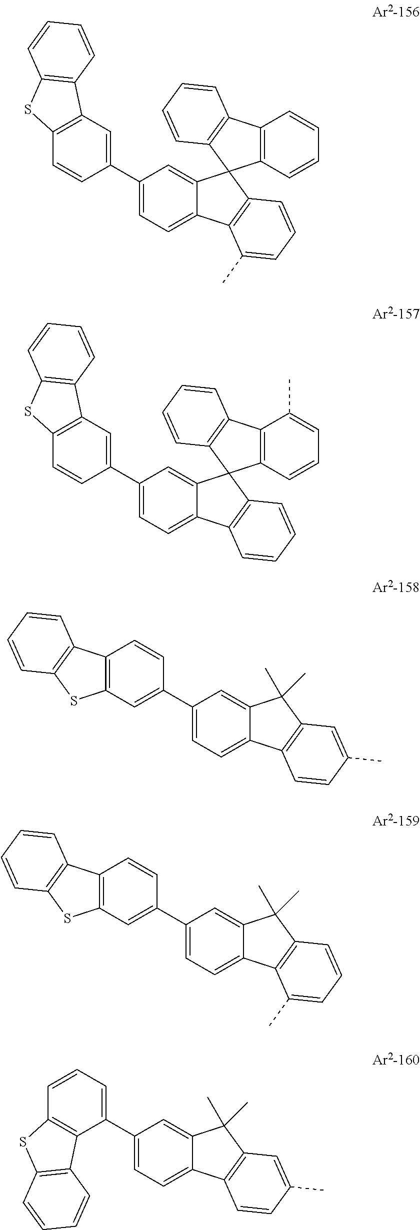

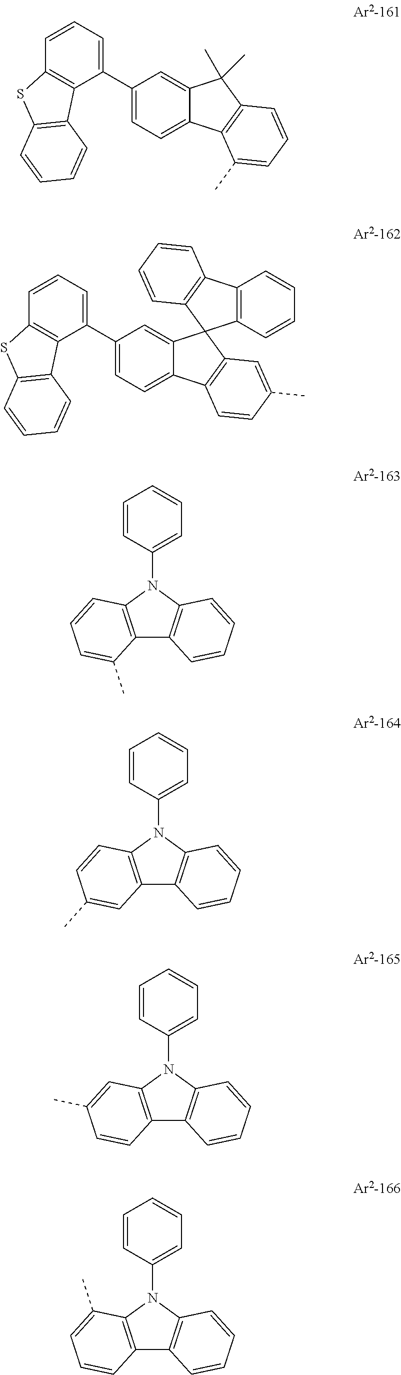

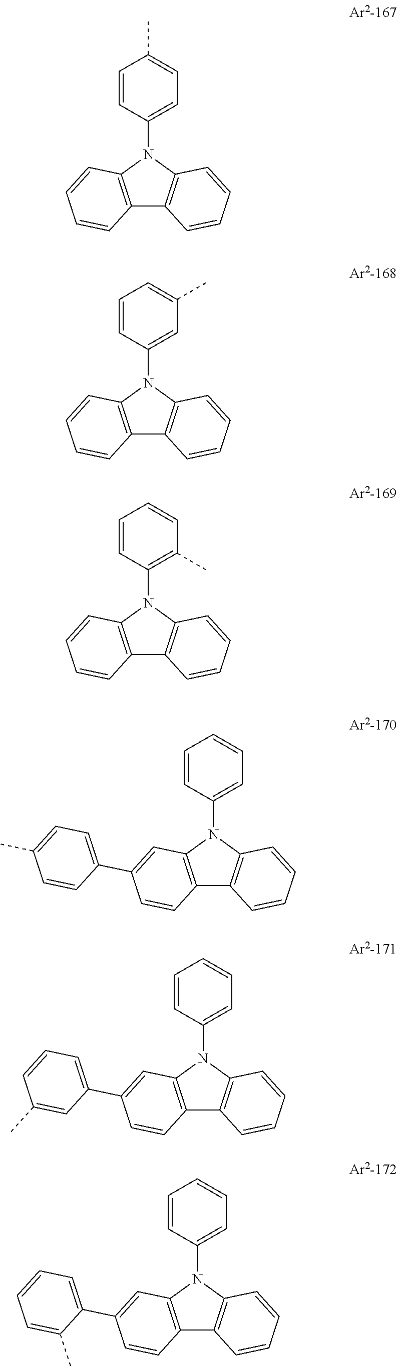

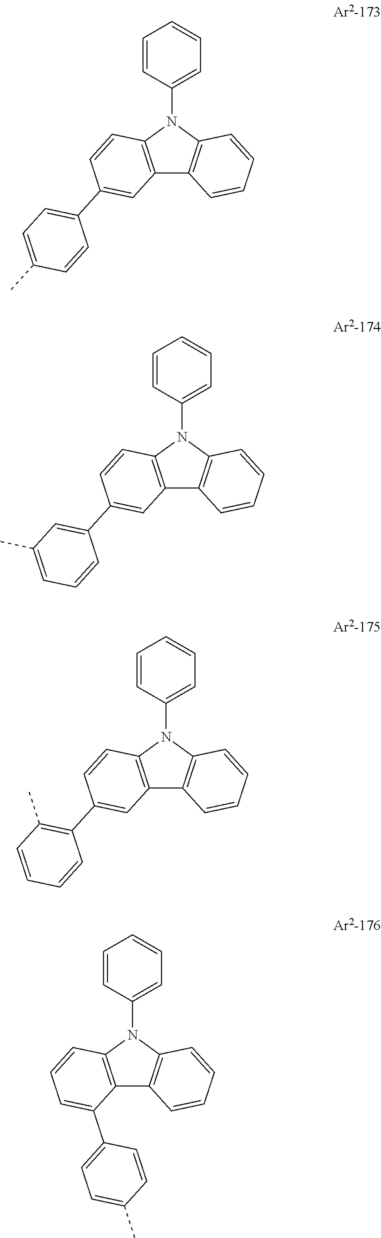

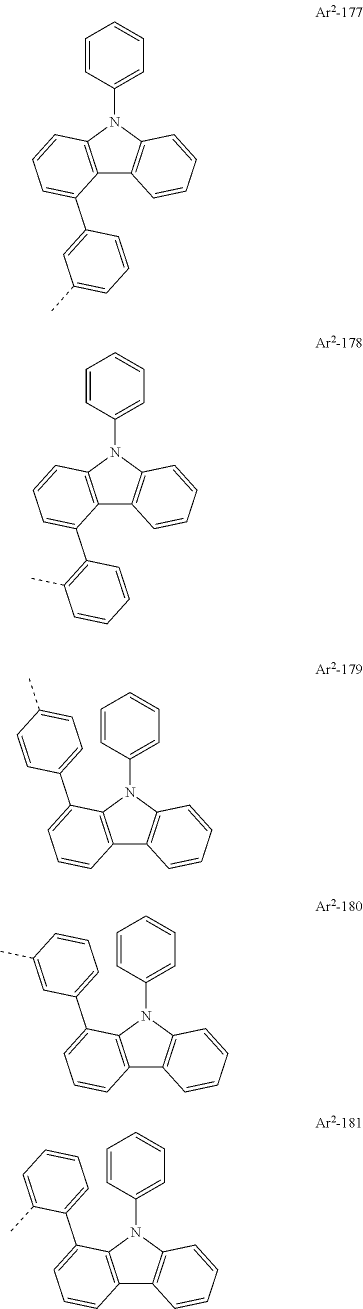

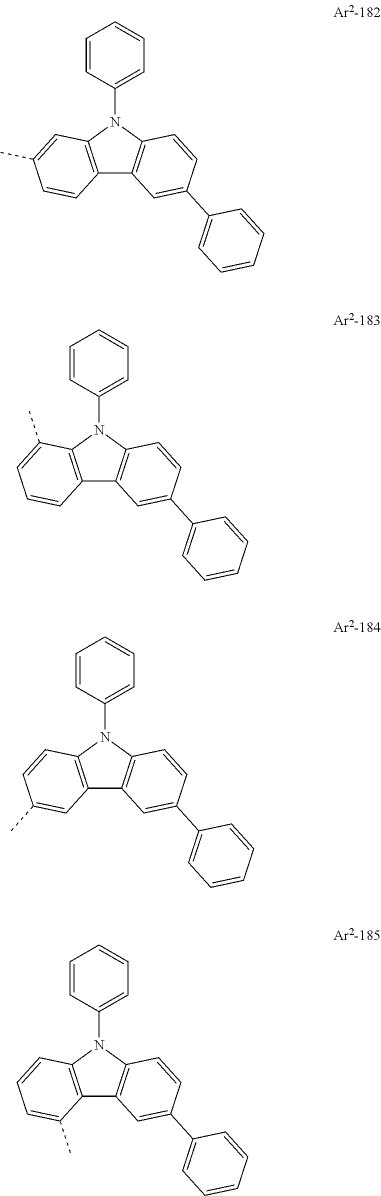

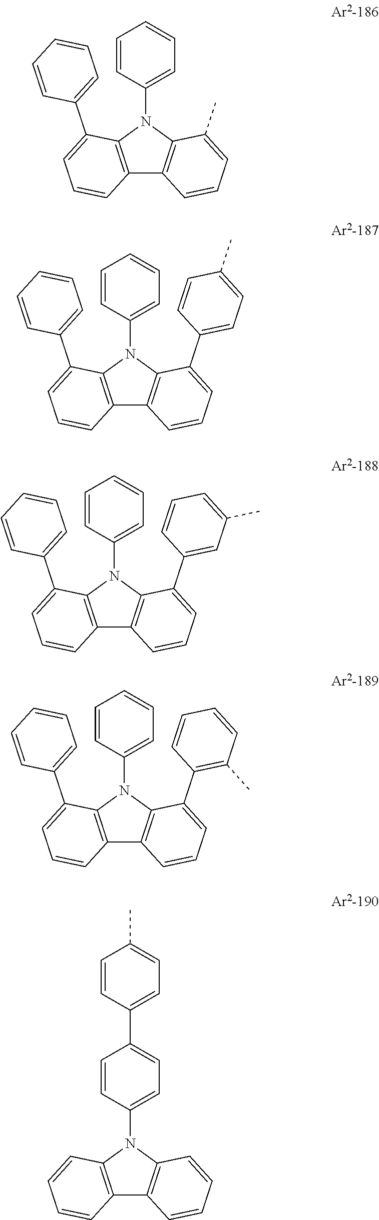

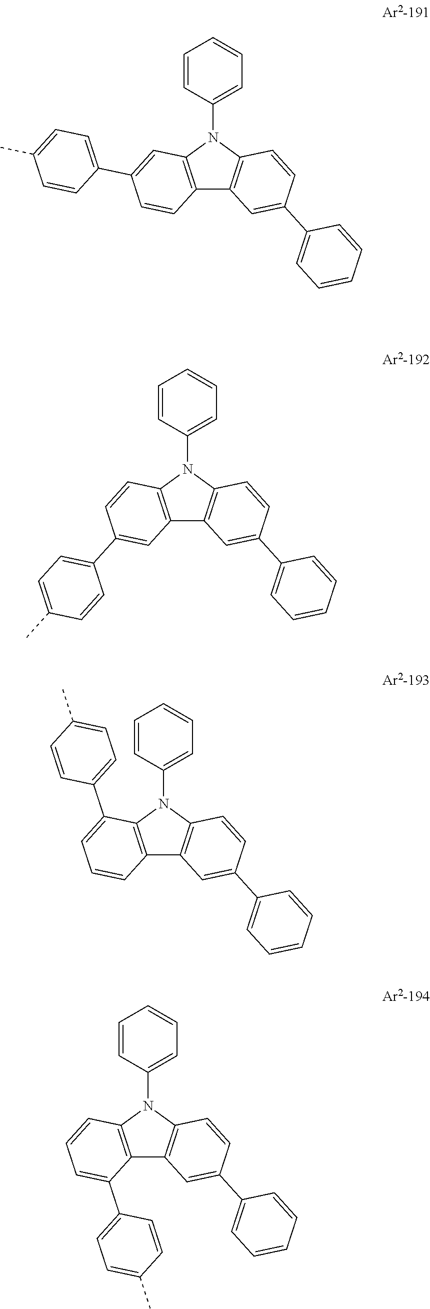

Among the abovementioned groups, particular preference is given to the following: Ar²-2, Ar²-6, Ar²-17, Ar²-25, Ar²-45, Ar²-65, Ar²-74, Ar²-105, Ar²-165, Ar²-173.

Preferably, Z² is CR³, where Z² is C when an L² group is bonded thereto.

Preferably, L² is selected from a single bond and an aromatic ring system which has 6 to 20 aromatic ring atoms and may be substituted by one or more R³ radicals. Aromatic ring systems particularly preferred for L² are divalent groups selected from phenylene, biphenylene, terphenylene, naphthylene, dibenzofuran, dibenzothiophene, carbazole and fluorene, where the divalent groups may each be substituted by one or more R³ radicals. Even more preferably, L² is a single bond or a phenylene group which may be substituted by one or more R³ radicals. A preferred phenylene group is a 1,4-phenylene group which may be substituted by one or more R³ radicals. Most preferably, L² is a single bond.

Preferred divalent L² groups are depicted below:

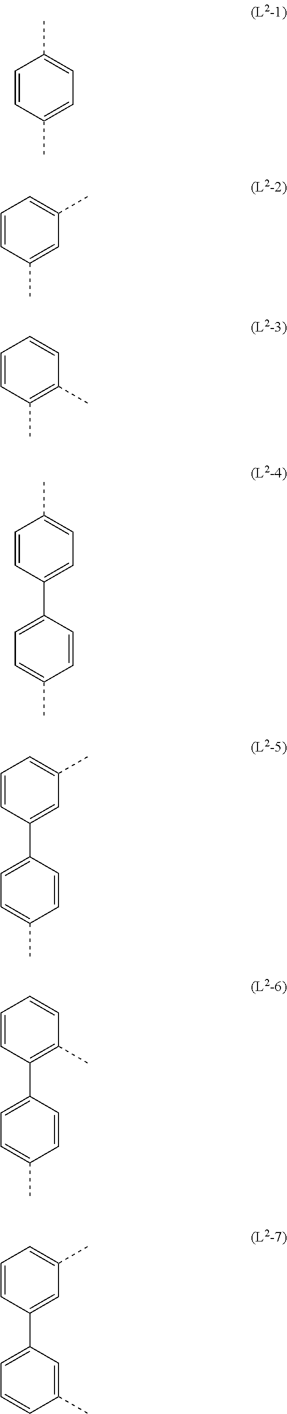

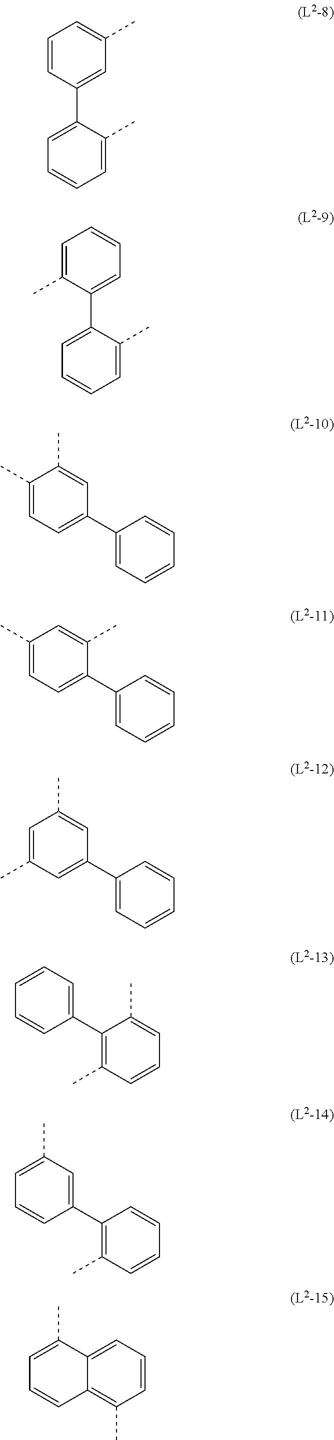

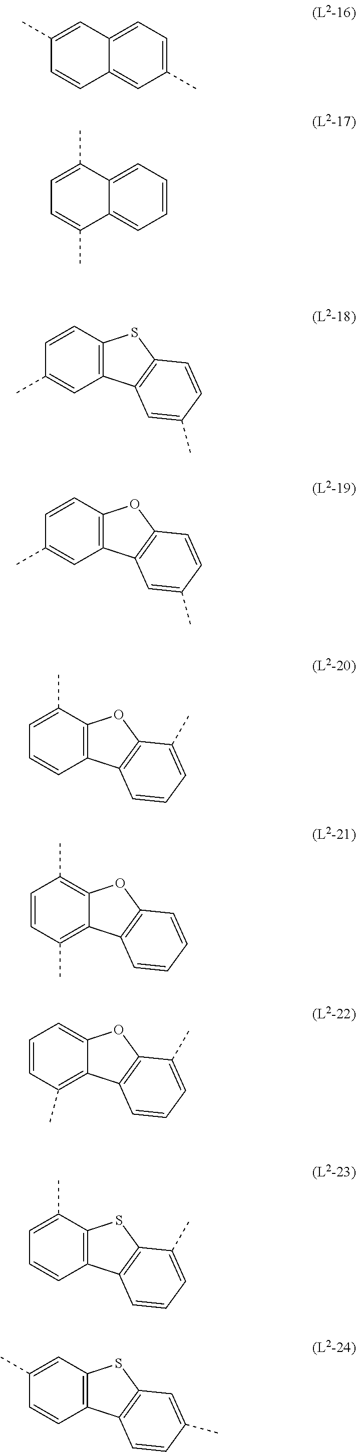

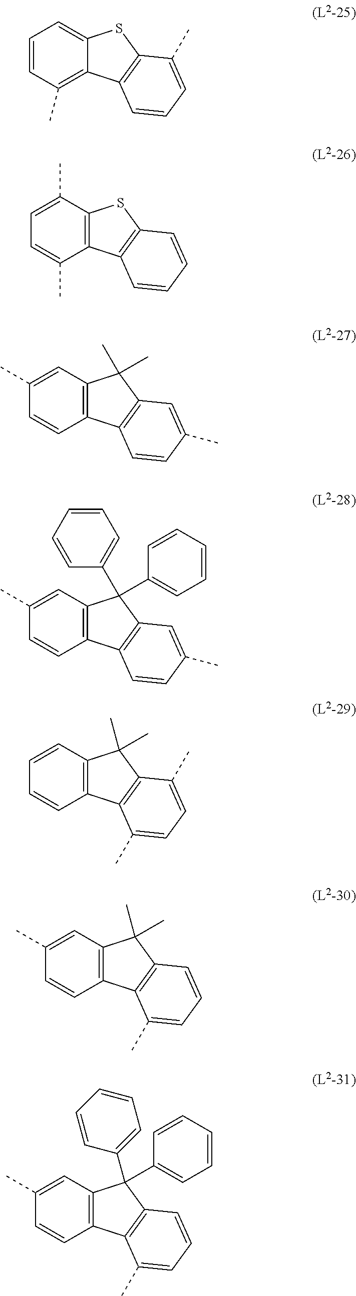

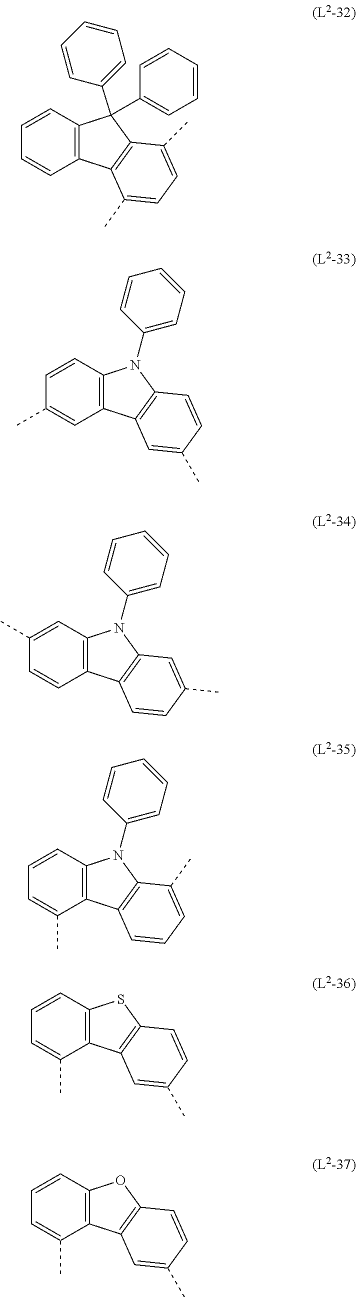

where the dotted bonds indicate the bonds of the divalent group to the rest of the compound, and where the groups at positions shown as unsubstituted may each be substituted by an R³ radical, but are preferably unsubstituted at these positions.

### Preferably, Y is N.

Ar³ preferably does not correspond to one of the formulae (A), (B) and (C).

Ar³ is preferably an aromatic ring system which has 6 to 20 aromatic ring atoms and may be substituted by one or more R⁴ radicals. Ar³ is more preferably selected from phenyl, biphenyl, terphenyl, fluorenyl, naphthyl, spirobifluorenyl, pyridyl, pyrimidyl, triazinyl, dibenzofuranyl, benzofused dibenzofuranyl, dibenzothiophenyl, benzofused dibenzothiophenyl, carbazolyl, and benzofused carbazolyl, and combinations of two, three or four of these groups, where all the groups mentioned may each be substituted by one or more R⁴ radicals.

Preferred embodiments of Ar³ are depicted below:

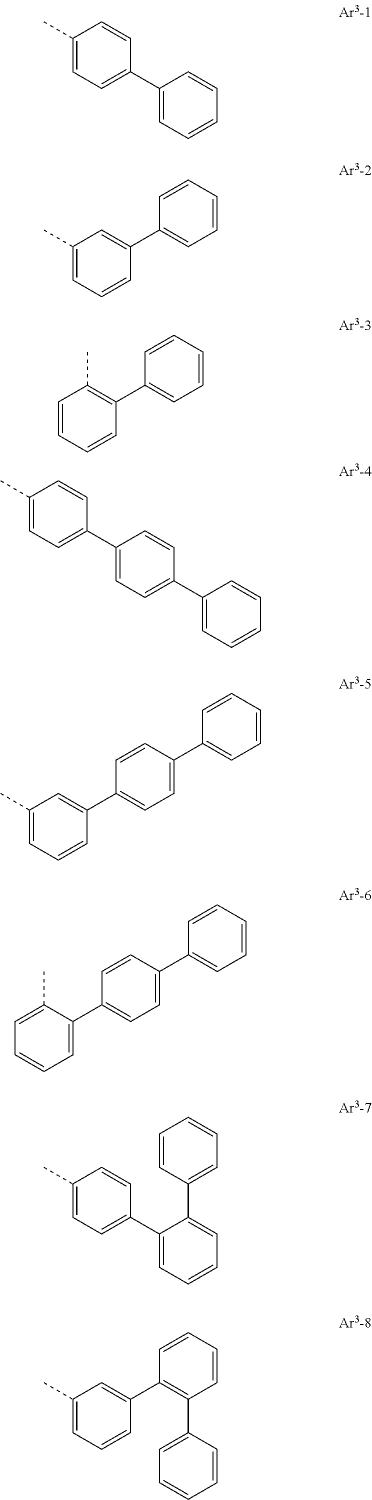

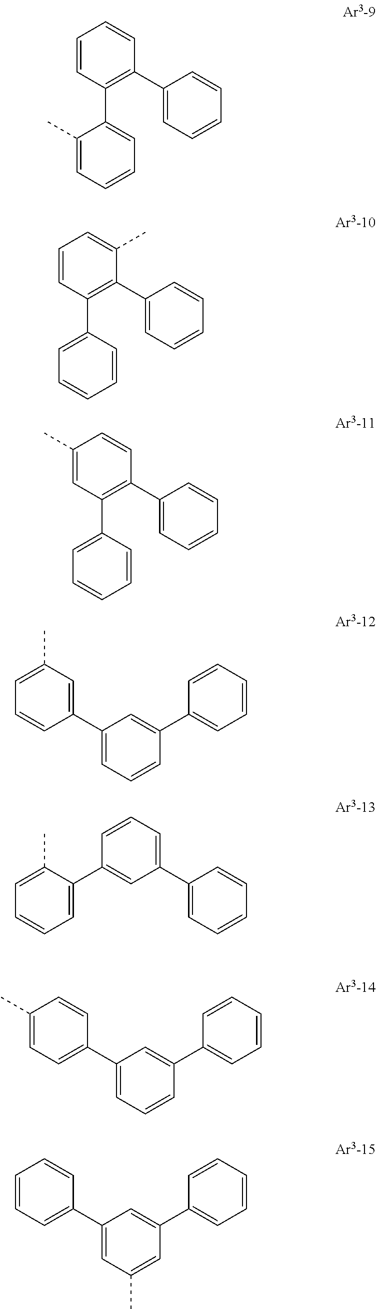

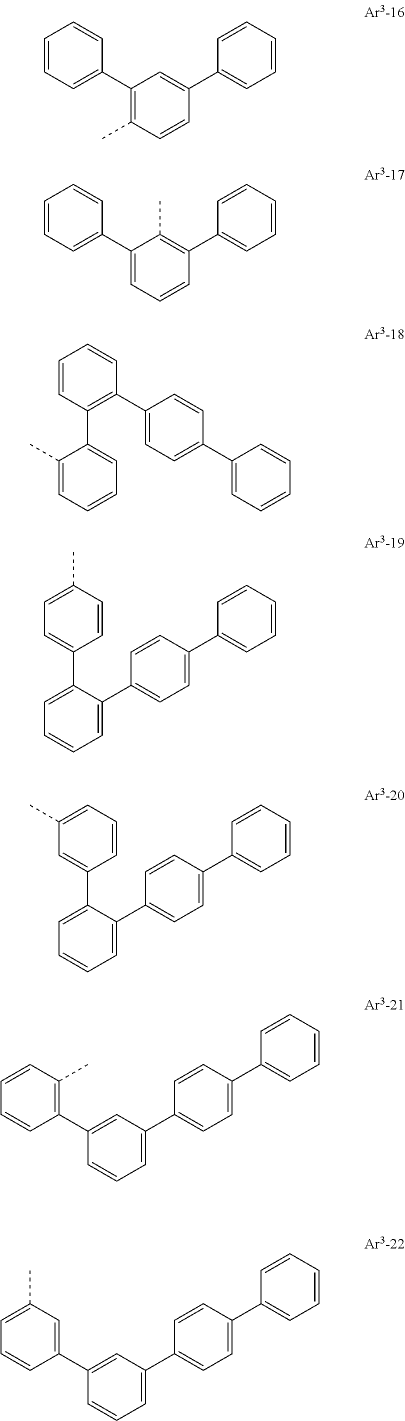

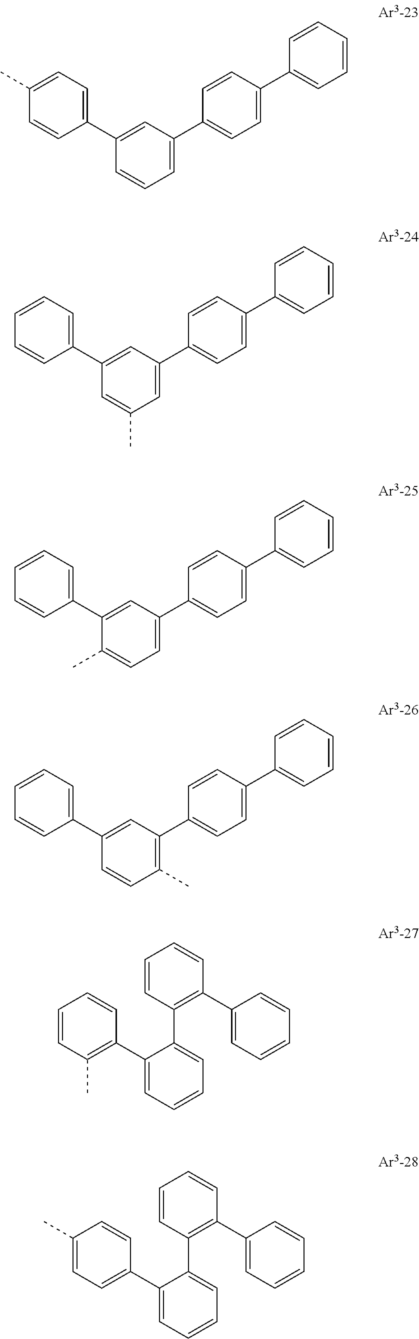

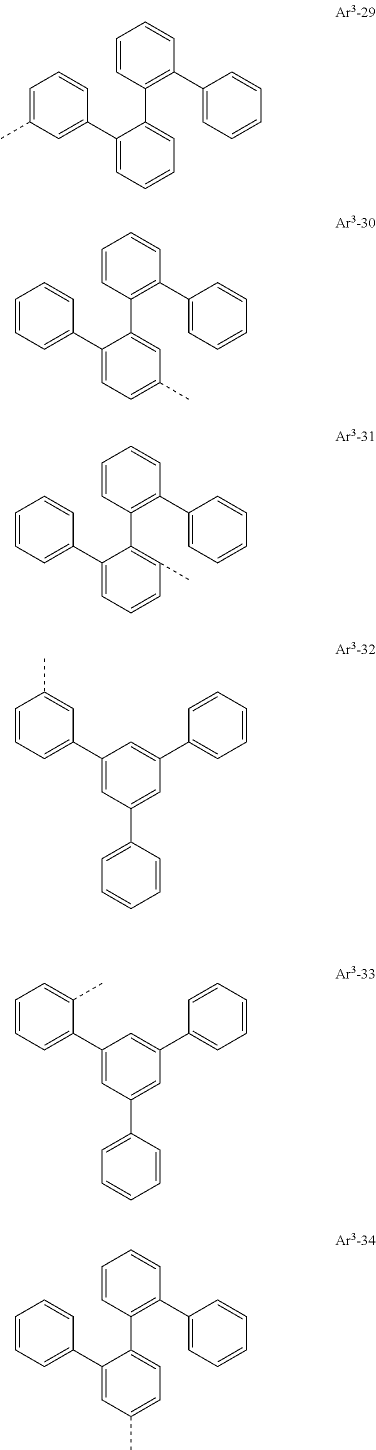

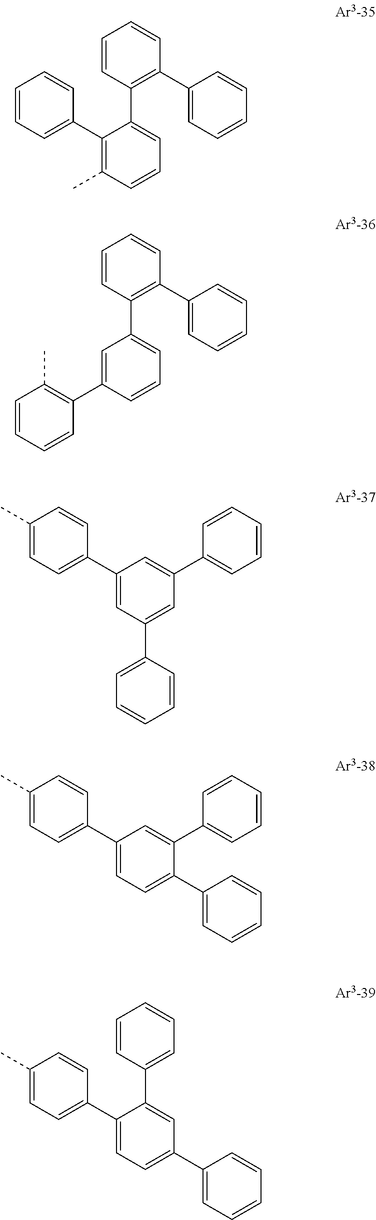

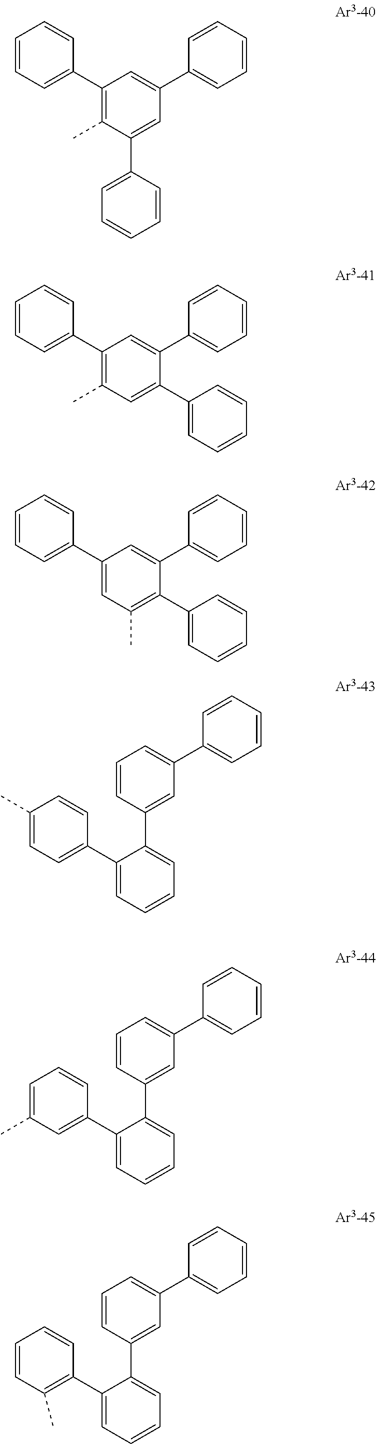

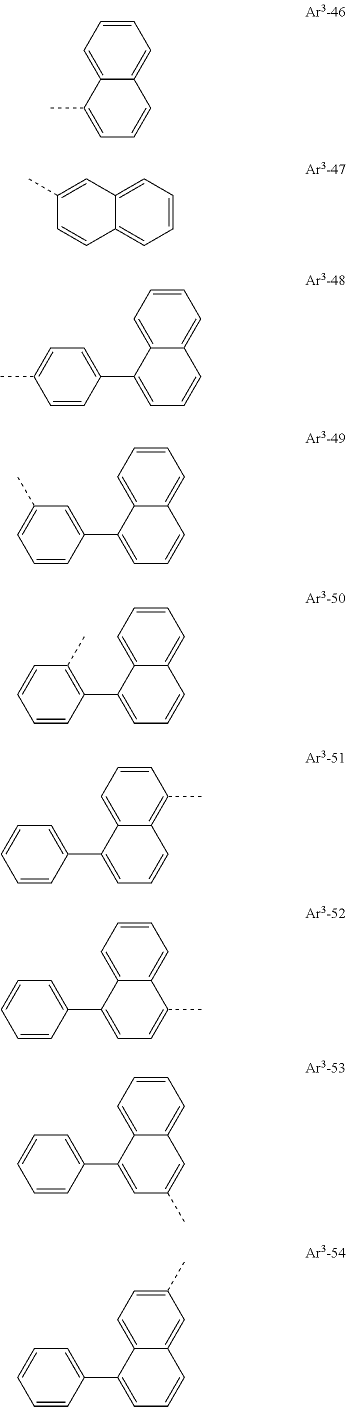

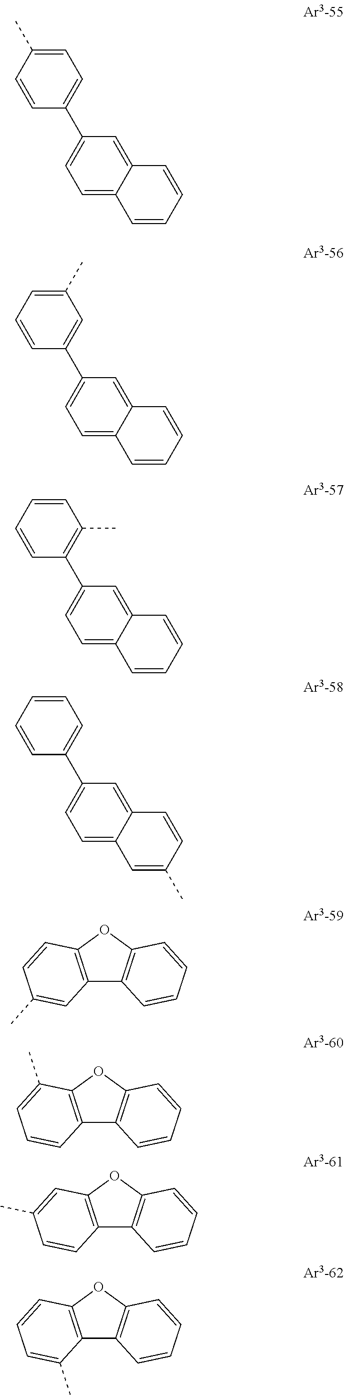

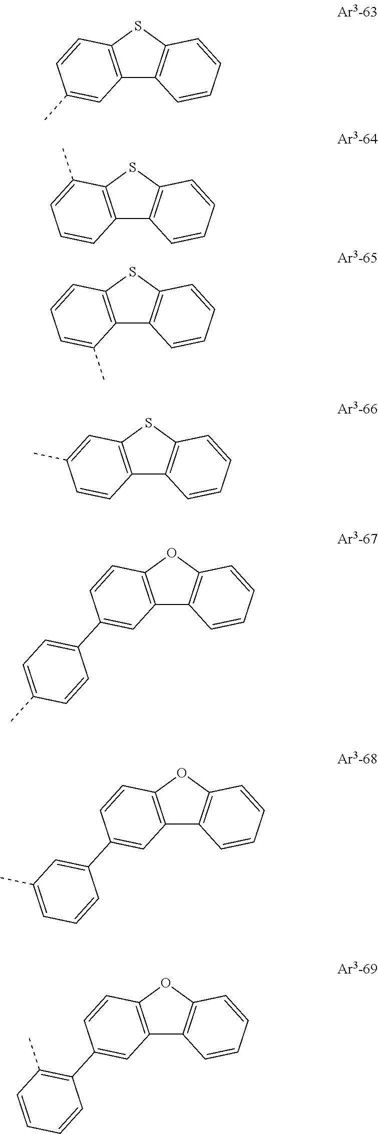

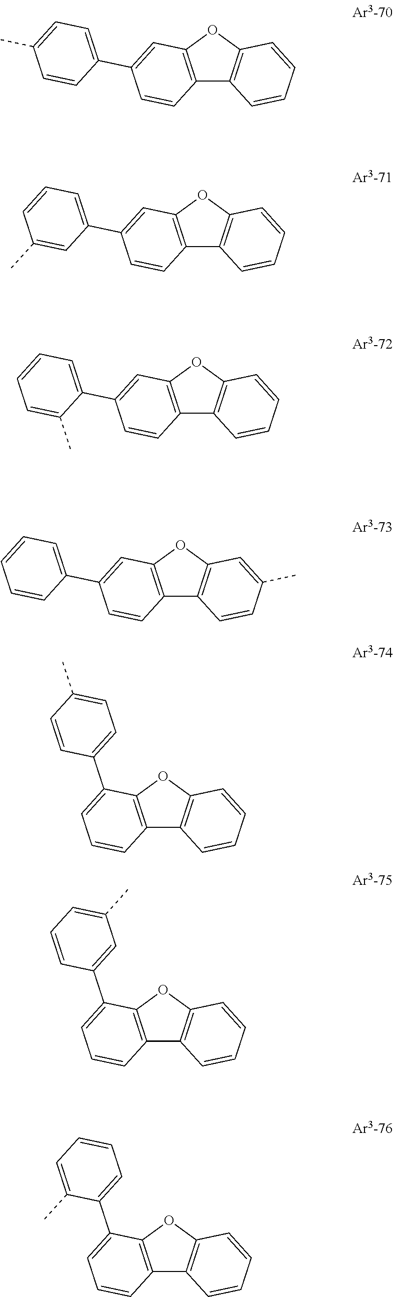

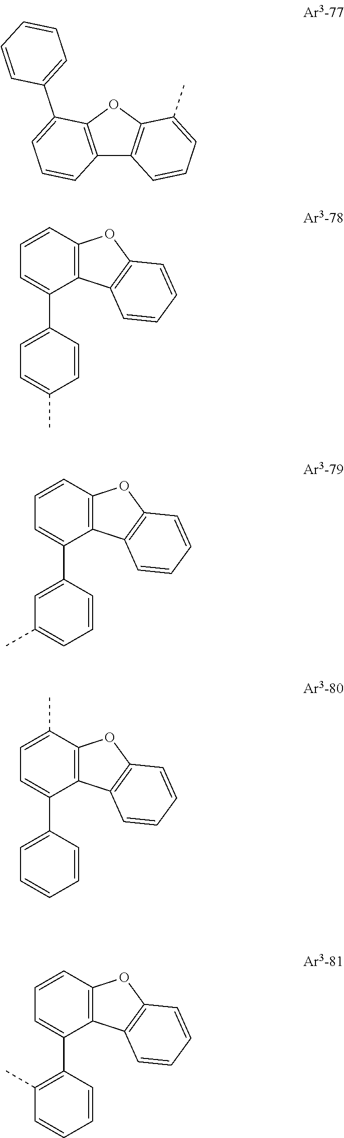

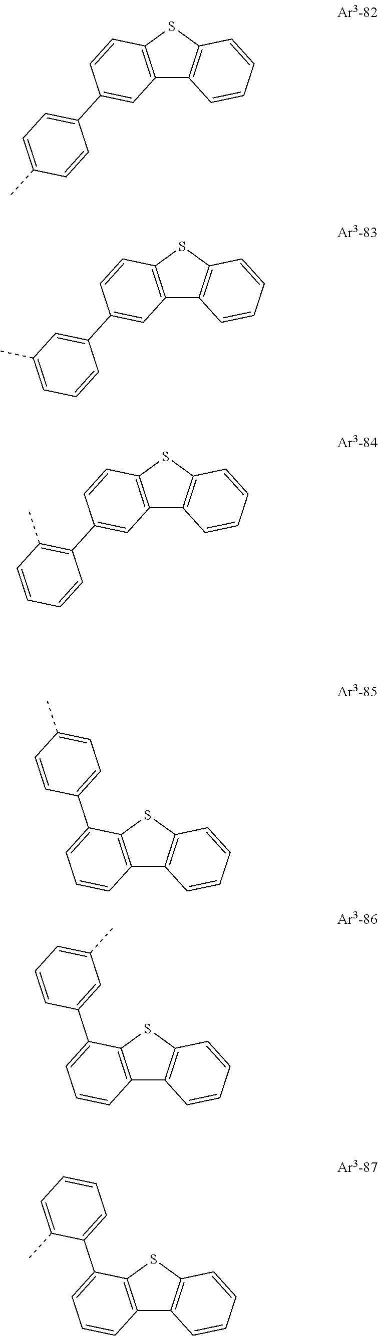

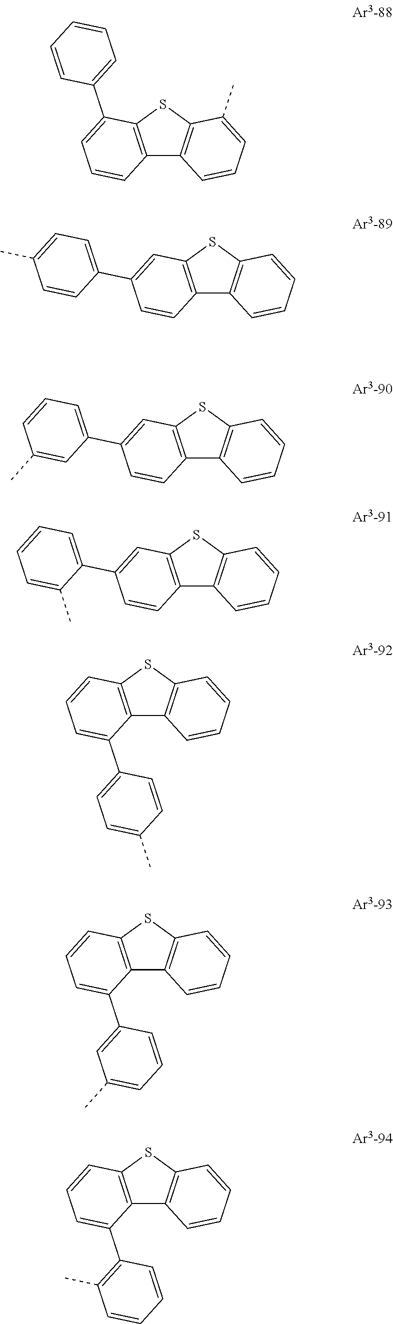

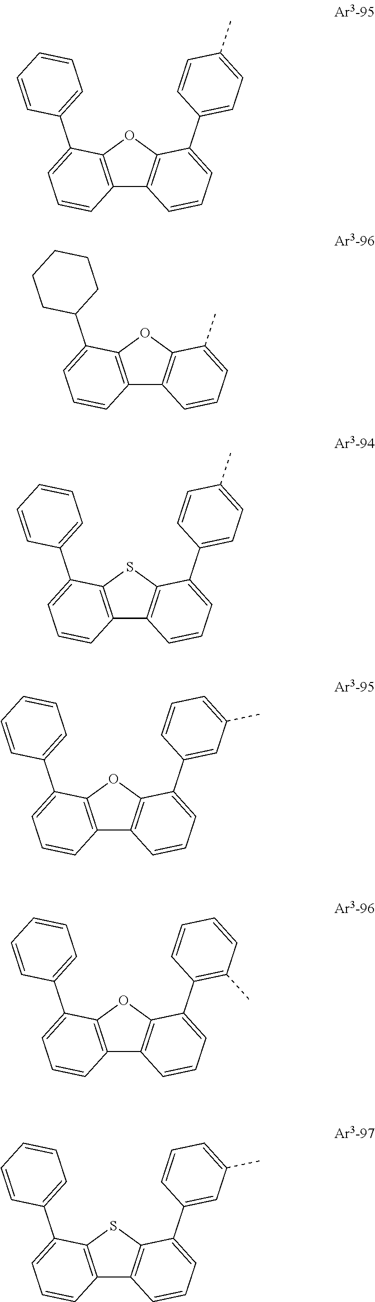

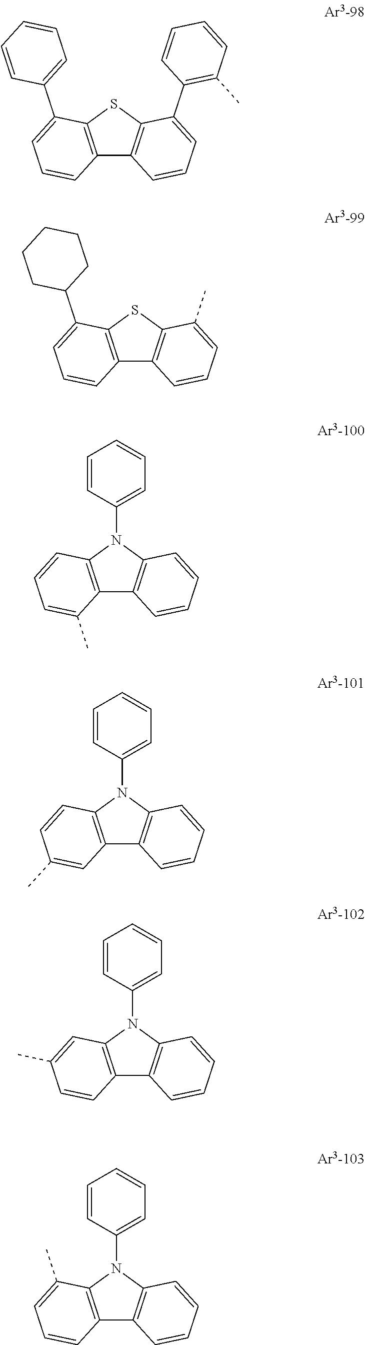

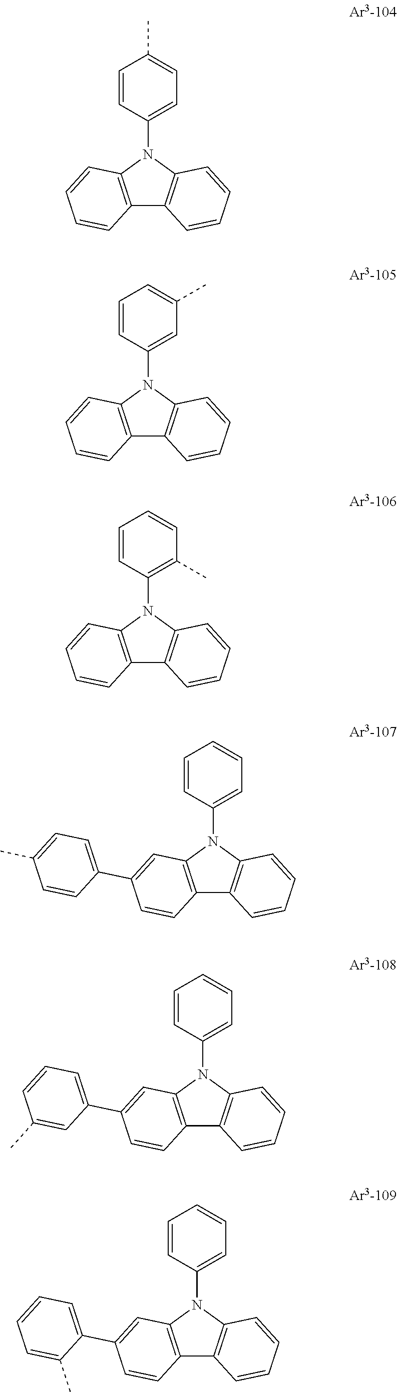

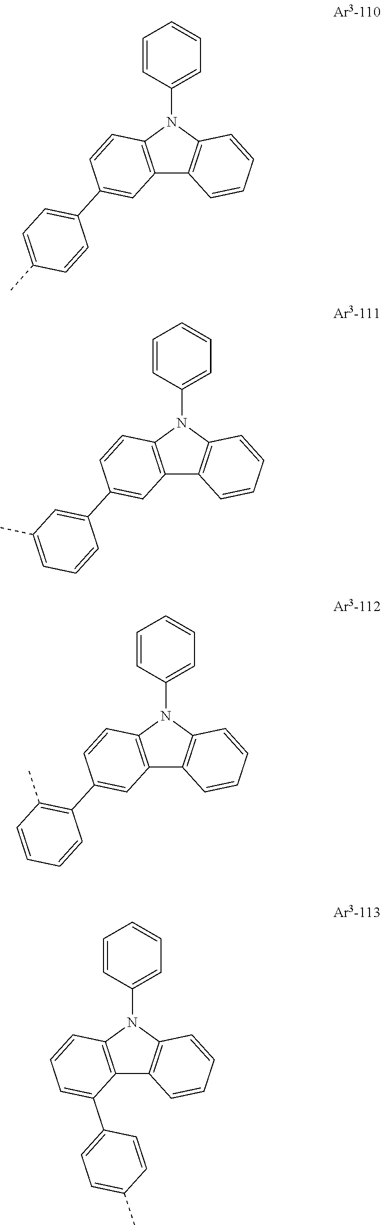

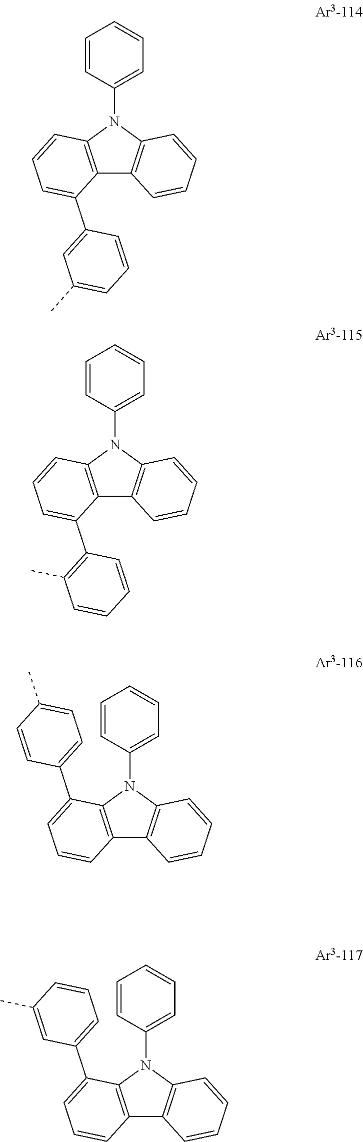

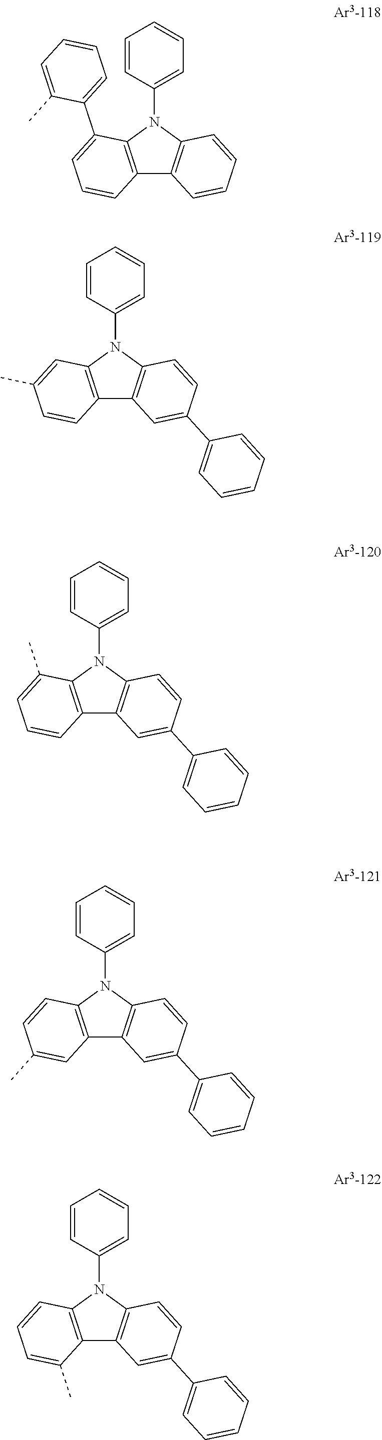

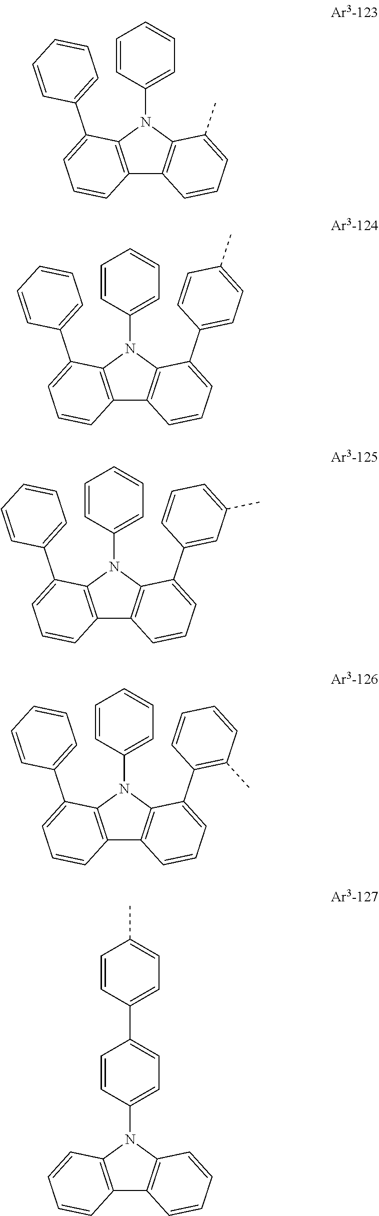

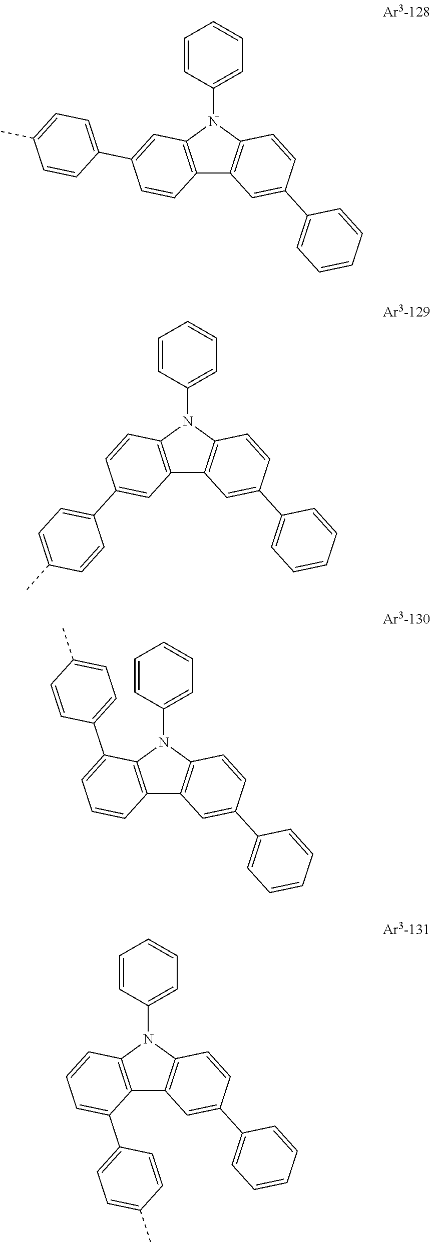

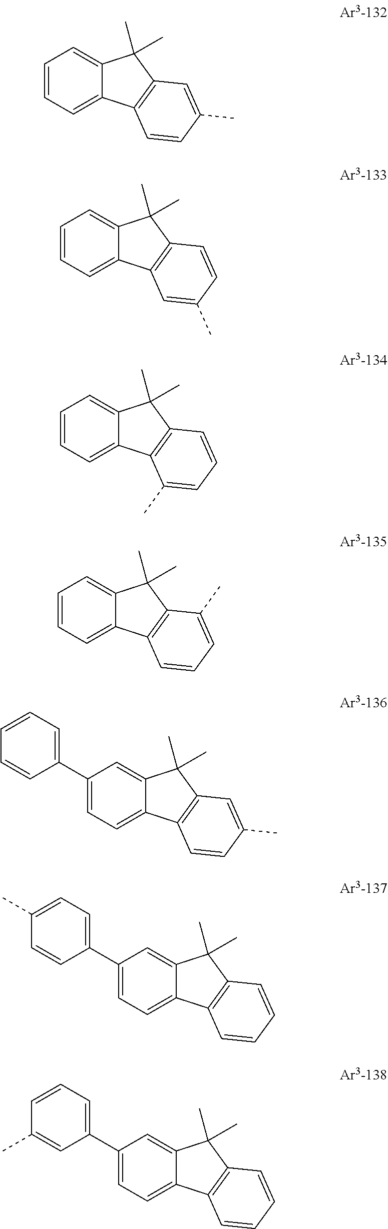

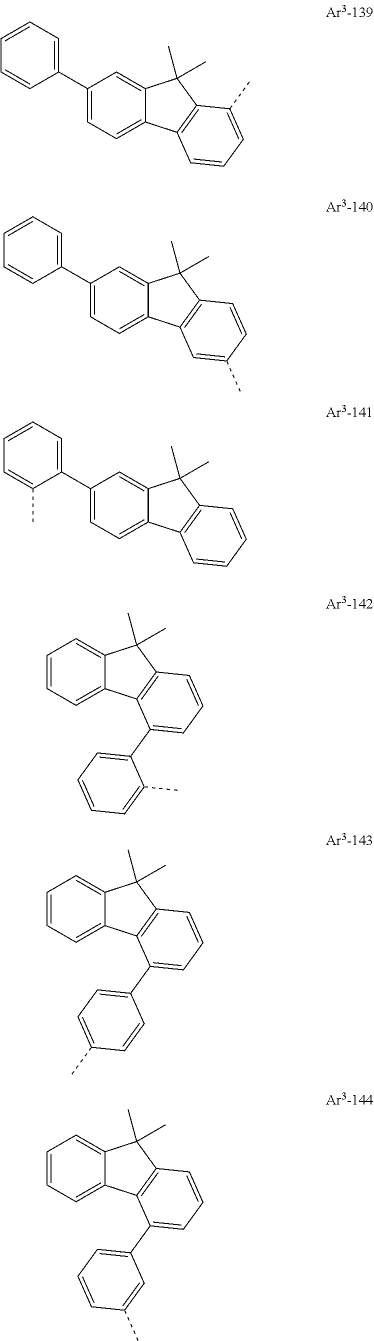

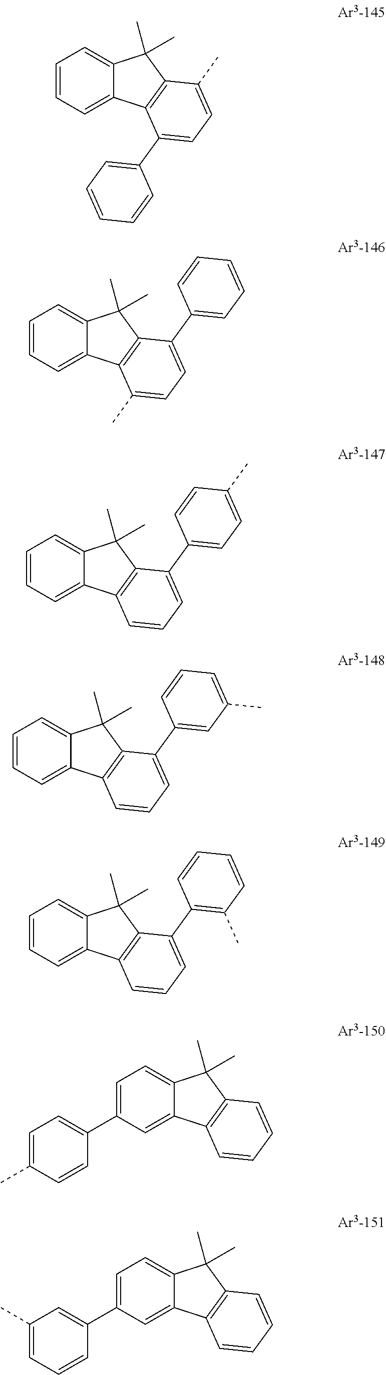

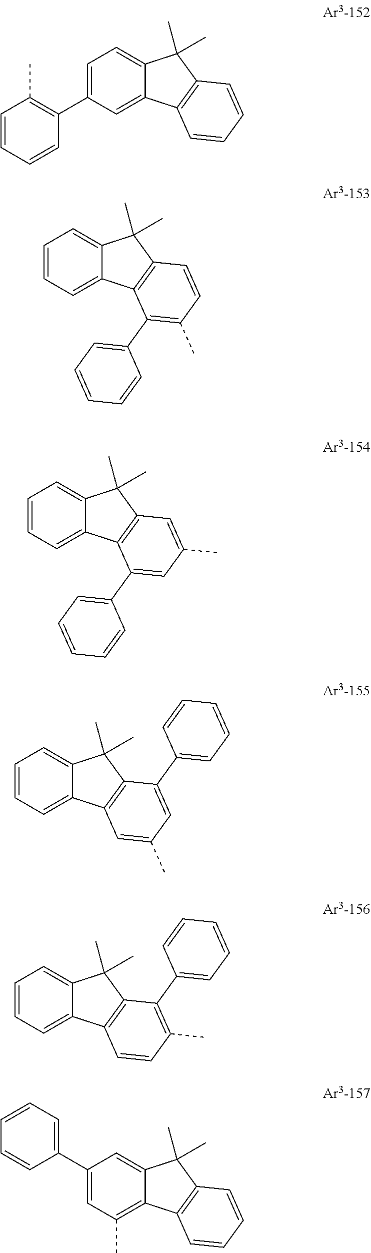

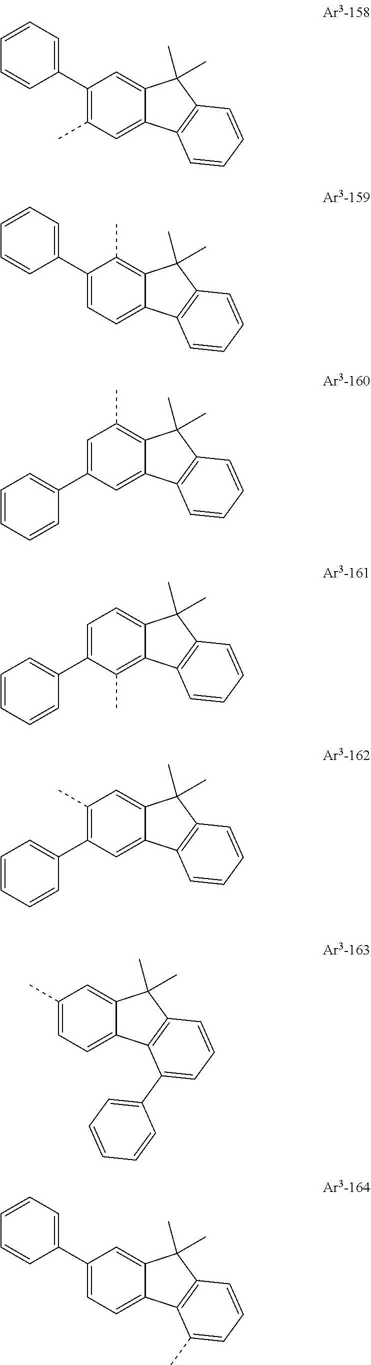

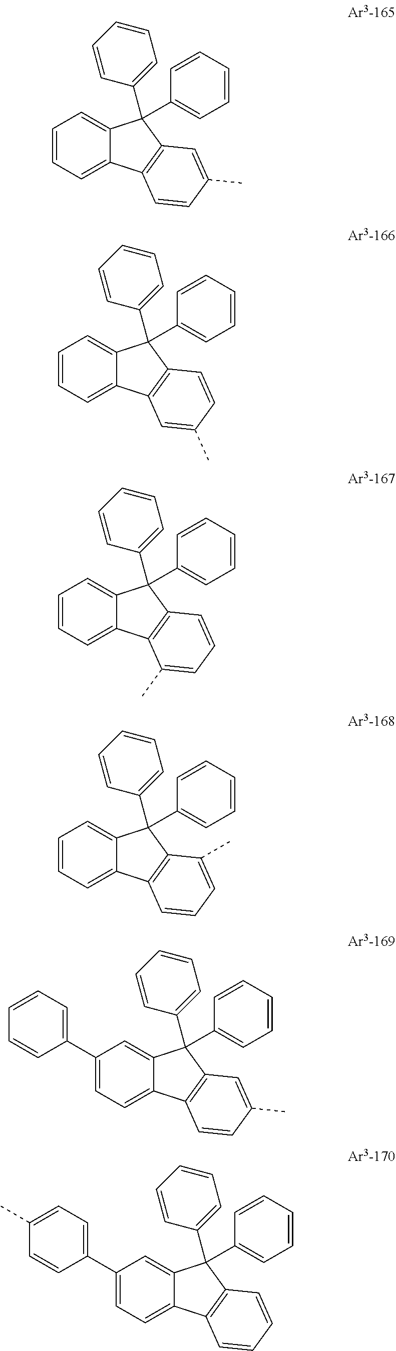

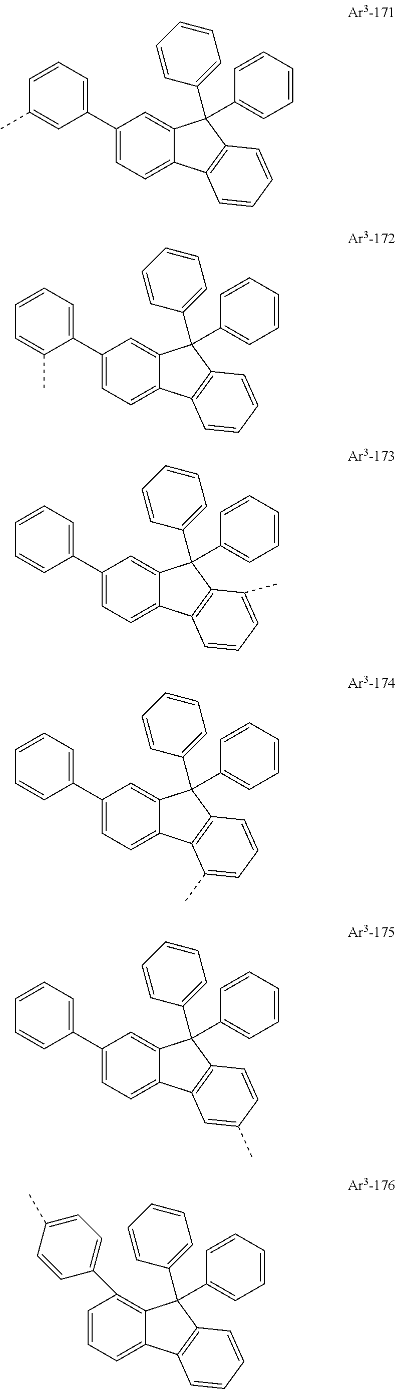

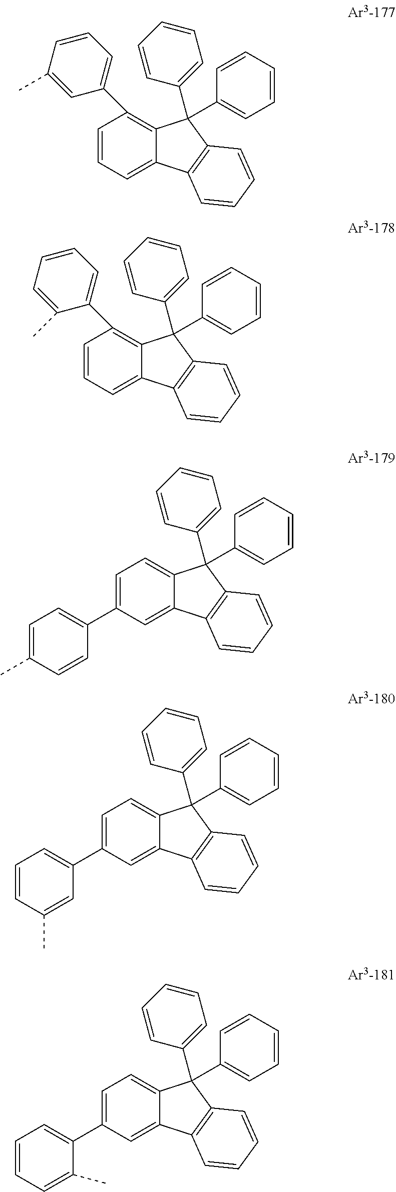

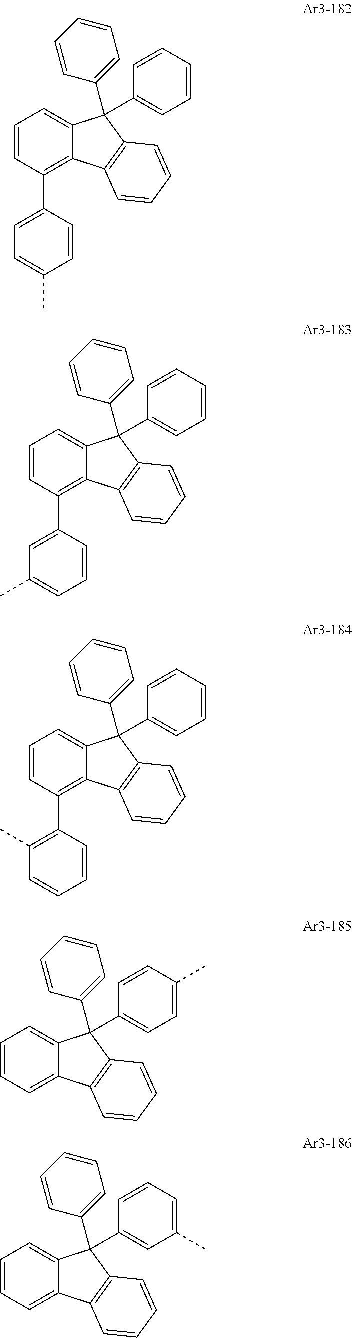

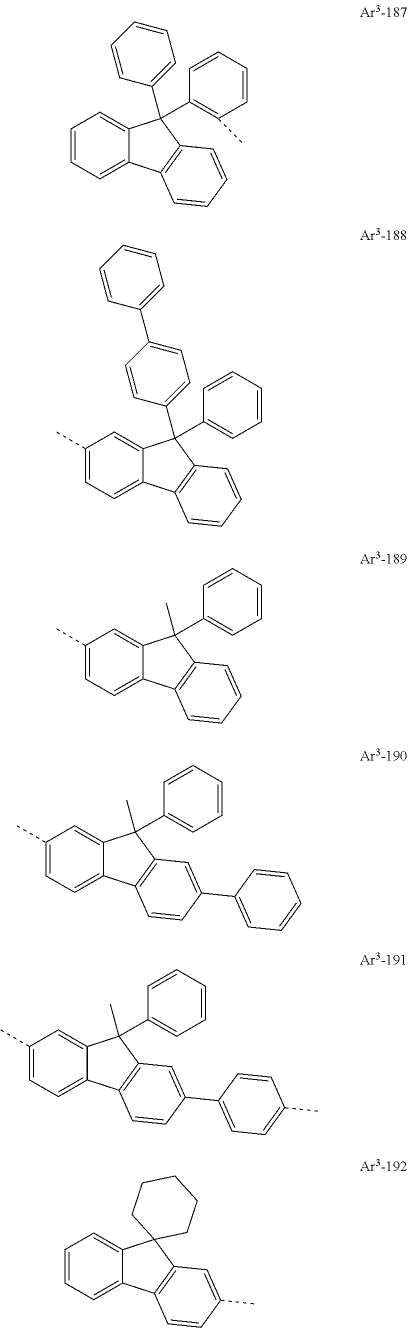

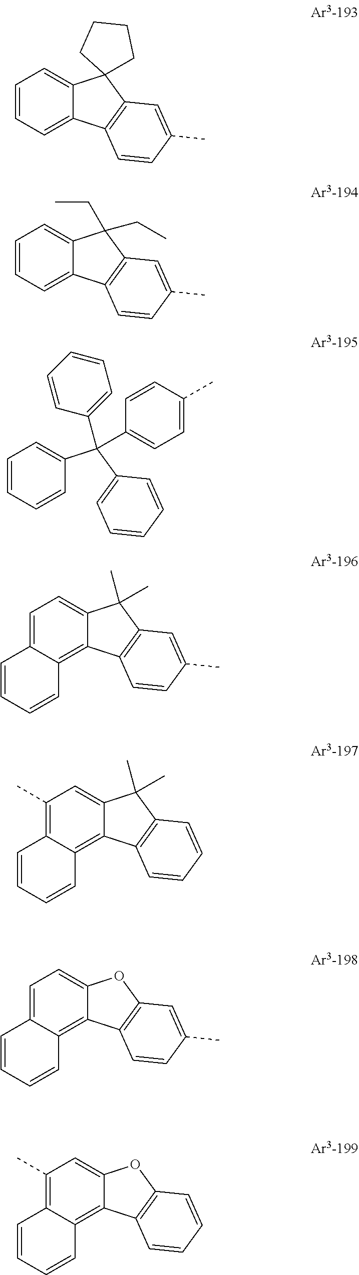

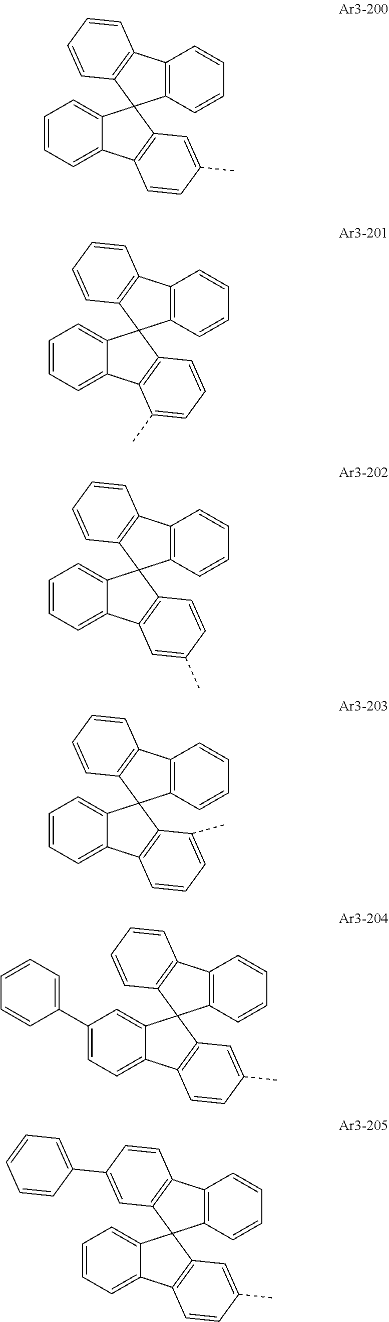

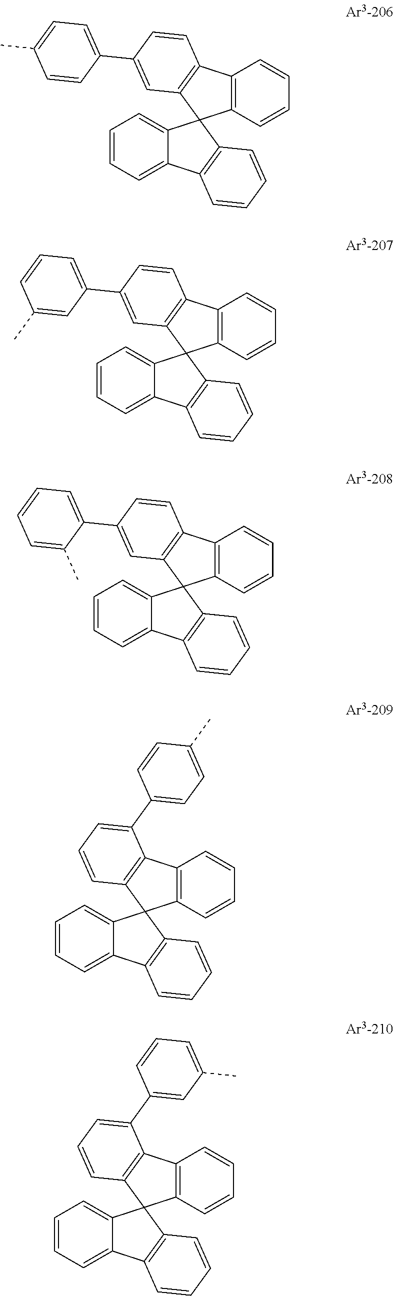

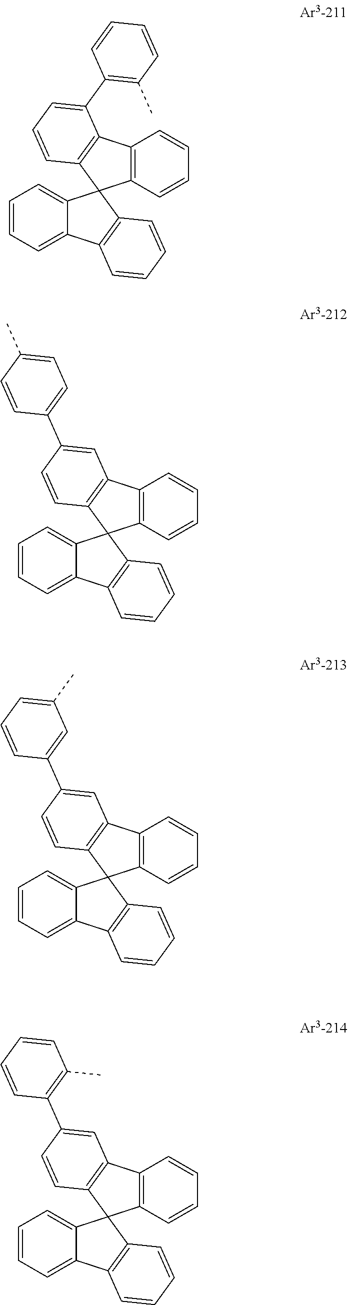

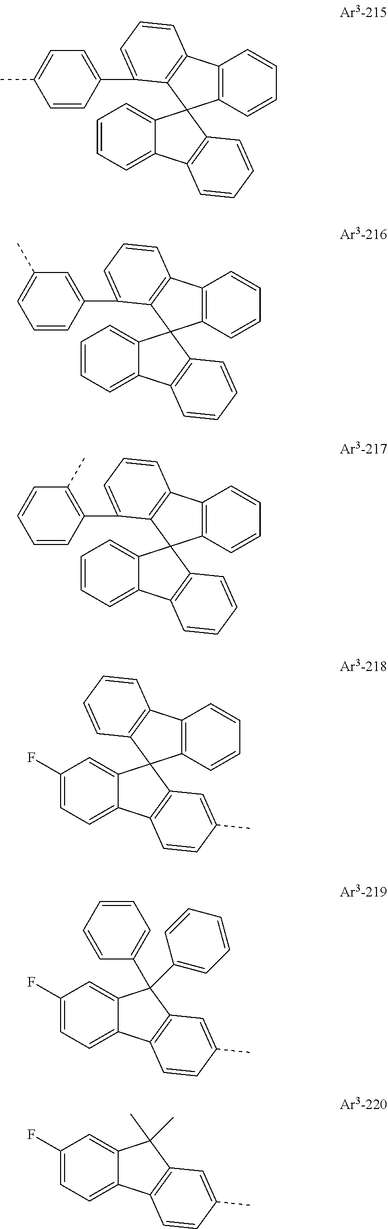

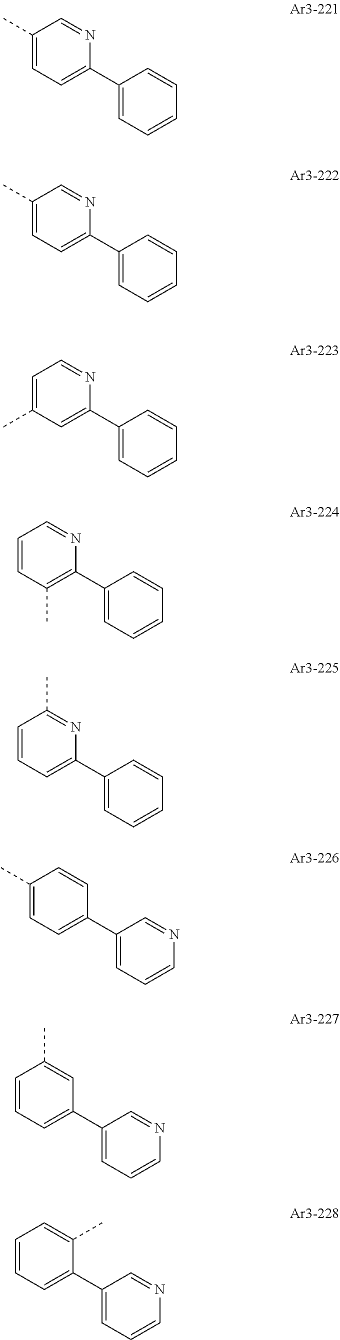

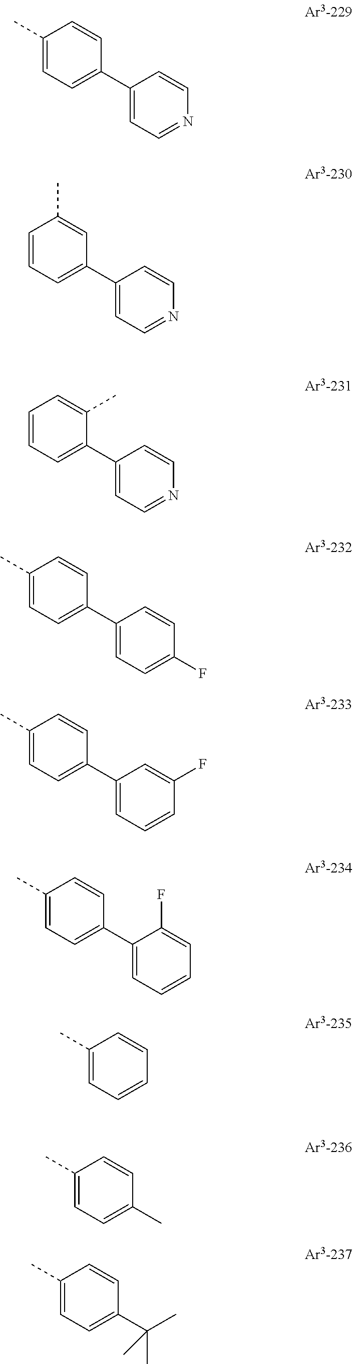

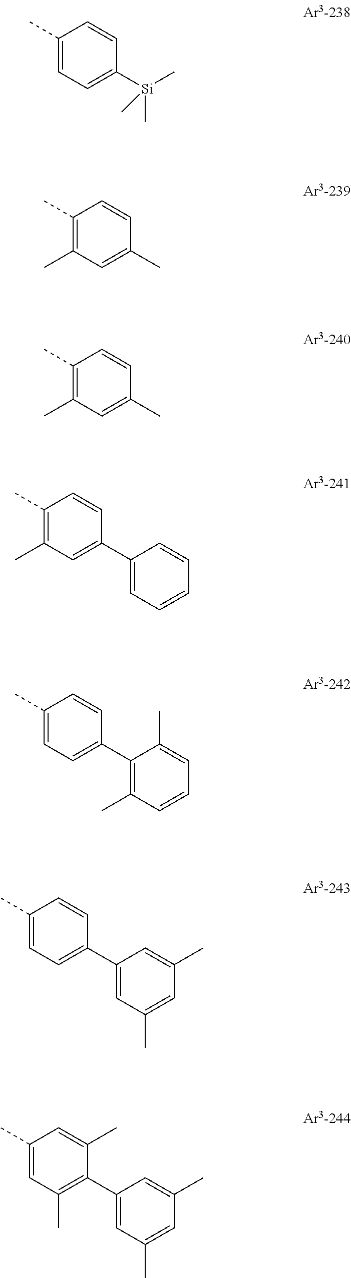

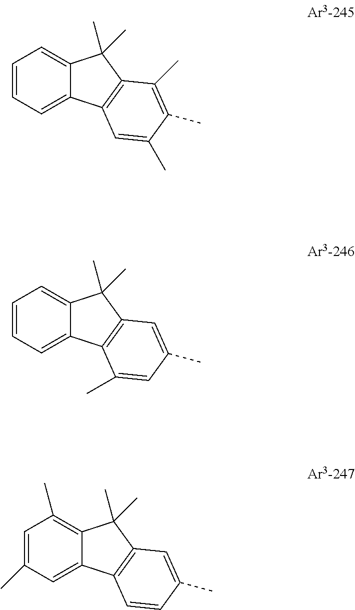

Among the abovementioned groups, preference is given to the following groups: Ar³-1, Ar³-2, Ar³-3, Ar³-4, Ar³-74, Ar³-85, Ar³-110, Ar³-132, Ar³-165, Ar³-235.

Preferably, R¹, R², R³ and R⁴ are the same or different at each instance and are selected from H, D, F, CN, Si(R⁵)₃, N(R⁵)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms, where the alkyl and alkoxy groups mentioned, the aromatic ring systems mentioned and the heteroaromatic ring systems mentioned may each be substituted by one or more R⁵ radicals, and where one or more CH₂ groups in the alkyl or alkoxy groups mentioned may be replaced by —C≡C—, —R⁵C═CR⁵—, Si(R⁵)₂, C═O, C═NR⁵, —NR⁵—, —O—, —S—, —C(═O)O— or —C(═O)NR⁵—.

More preferably, R¹ is H, with the exception of R¹ groups bonded to a T group which is C(R¹)₂ or NR¹. In this case, R¹ is preferably selected from alkyl groups having 1 to 20 carbon atoms and aromatic ring systems having 5 to 40 aromatic ring atoms, where the alkyl groups mentioned and the aromatic ring systems mentioned may each be substituted by one or more R⁵ radicals.

More preferably, R² is H.

More preferably, R³ is H, with the exception of R³ groups bonded to an X group which is C(R³)₂ or NR³. In this case, R³ is preferably selected from alkyl groups having 1 to 20 carbon atoms and aromatic ring systems having 5 to 40 aromatic ring atoms, where the alkyl groups mentioned and the aromatic ring systems mentioned may each be substituted by one or more R⁵ radicals.

More preferably, R⁴ is H.

Preferably, R⁵ is the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms, where the alkyl and alkoxy groups mentioned, the aromatic ring systems mentioned and the heteroaromatic ring systems mentioned may each be substituted by one or more R⁶ radicals, and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—. More preferably, R⁵ is H.

Preferably, n is 0.

Preferably, i is 0 or 1.

Preferably, k is 0 or 1.

Preferably, the sum total of i and k is 1.

Preferably, T is selected from C(R¹)₂ and NR¹.

Preferred embodiments of the formula (I) correspond to one of the following formulae:

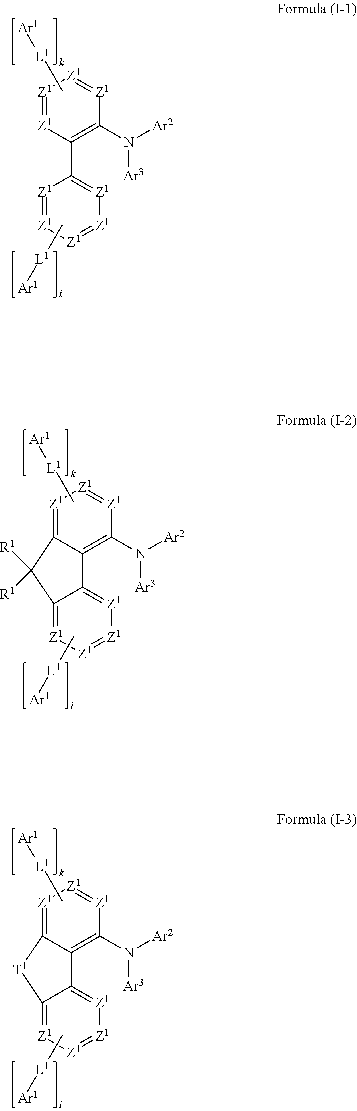

where the variables that occur are as defined above, and where T¹ is selected from O, S and NR¹.

Preferably, in formulae (I-1) to (I-3), Z¹ is CR¹, where Z¹ is C when an -L¹-Ar¹ group is bonded thereto. Further preferably, in formulae (I-1) to (I-3), the sum total of i and k is 1.

In a particularly preferred embodiment, the formulae (I-1) to (I-3) correspond to the following formulae:

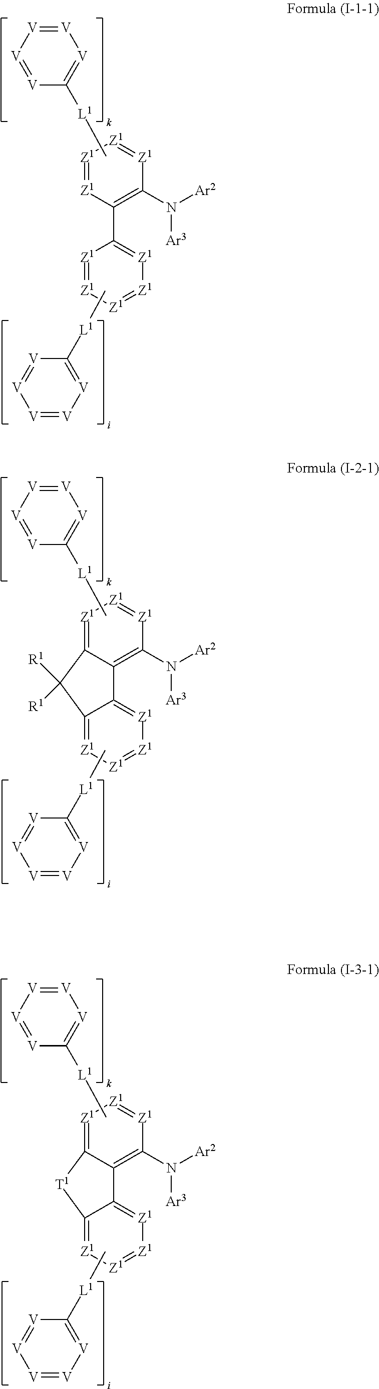

where the variables that occur are as defined above, and where T¹ is selected from O, S and NR¹, and where at least one V group per formula is N.

Preferably, in formulae (I-1-1), (I-2-1) and (I-3-1), Z¹ is CR¹, where Z¹ is C when a group having the index k or i is bonded thereto. Further preferably, in formulae (I-1-1), (I-2-1) and (I-3-1), the sum total of i and k is 1. It is further preferable that one, two or three V groups per formula are N. More preferably, the group

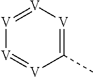

in each case is selected from pyridyl, pyrimidyl and triazinyl.

In a particularly preferred embodiment, the formulae (I-1-1), (I-2-1) and (I-3-1) correspond to the following formulae:

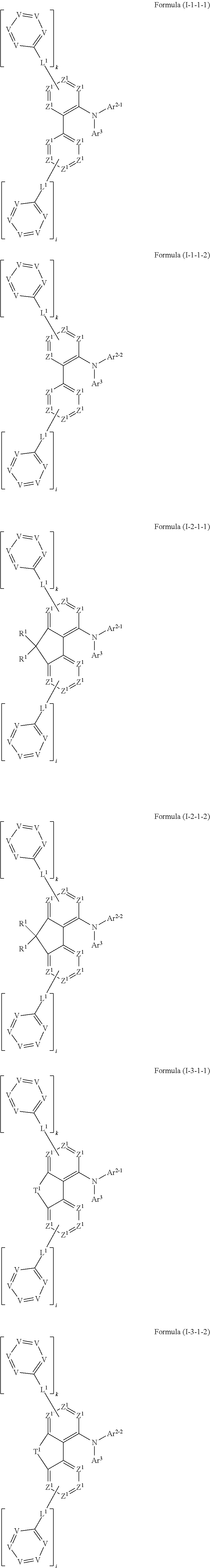

where the variables that occur are as defined above, and where T¹ is selected from O, S and NR¹, and where at least one V group per formula is N, and where, in addition:
Ar²-1′ is selected from formulae (A-1) and (B-1)

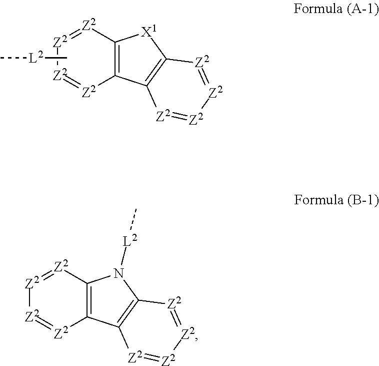

where Z² and L² are as defined above, and where X¹ is selected from NR³, O and S;
Ar²⁻¹² is selected from formulae (A-2), (B-2) and (C)

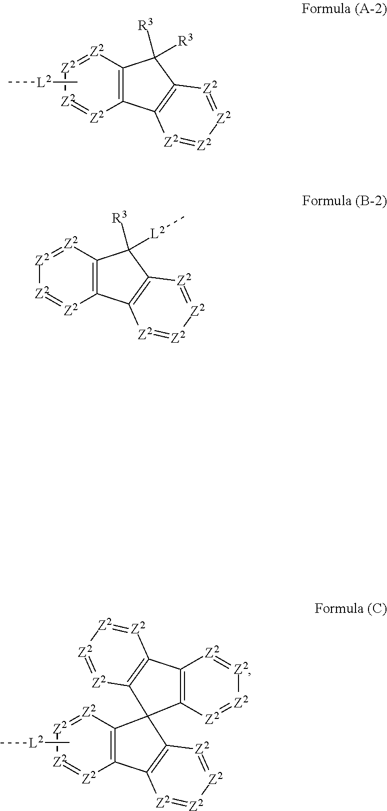

where L² and Z² are as defined above.

More preferably, Ar²⁻¹¹ in the abovementioned formulae corresponds to the formula (A-1). More preferably, Ar²⁻¹² in the abovementioned formulae corresponds to the formula (A-2) or (C), and among these more preferably to the formula (A-2).

Preferably, in the abovementioned formulae, Z¹ is CR¹, where Z¹ is C when a group having the index k or i is bonded thereto. Further preferably, in the abovementioned formulae, the sum total of i and k is 1. It is further preferable that one, two or three V groups per formula are N. More preferably, the group

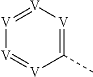

in each case is selected from pyridyl, pyrimidyl and triazinyl.

In a particularly preferred embodiment, the formulae (I-1) to (I-3) correspond to the following formulae:

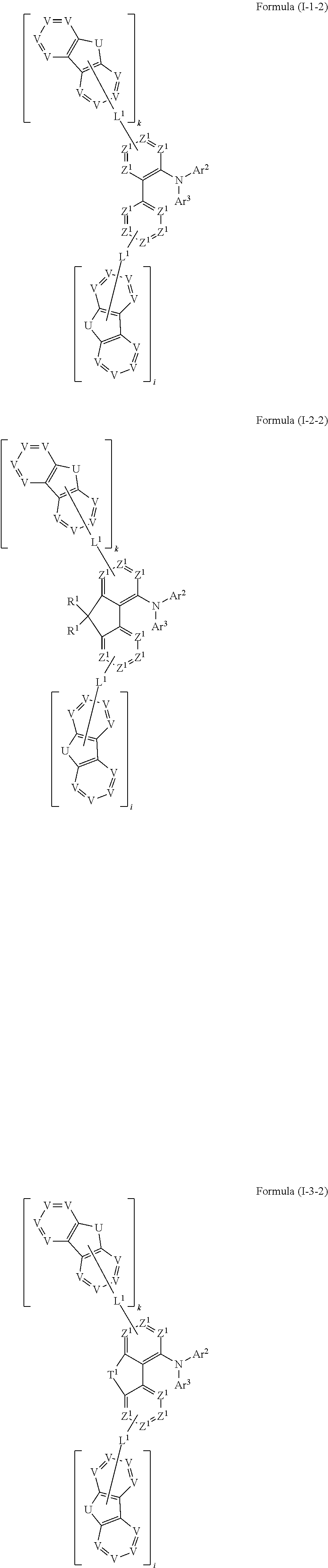

where the variables that occur are as defined above, and where T¹ is selected from O, S and NR¹, and where V is the same or different at each instance and is selected from CR² and N, where V is C when an L¹ group is bonded thereto, and where U is O, S or NR², where U is N when an L group is bonded thereto.

Preferably, in formulae (I-1-2), (I-2-2) and (I-3-2), Z¹ is CR¹, where Z¹ is C when a group having the index k or i is bonded thereto. Further preferably, in formulae (I-1-2), (I-2-2) and (I-3-2), the sum total of i and k is 1. More preferably, the group

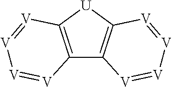

in each case is selected from dibenzofuran, dibenzothiophene and carbazole, where carbazole may be bonded via the nitrogen atom or via a bonding site on one of the six-membered rings. Very particular preference is given to dibenzofuran and dibenzothiophene.

In a particularly preferred embodiment, the formulae (I-1-2), (I-2-2) and (I-3-2) correspond to the following formulae:

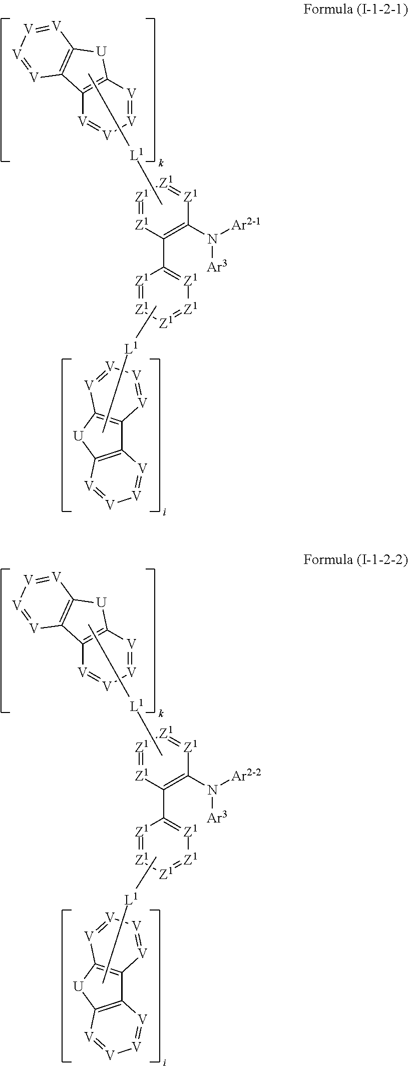

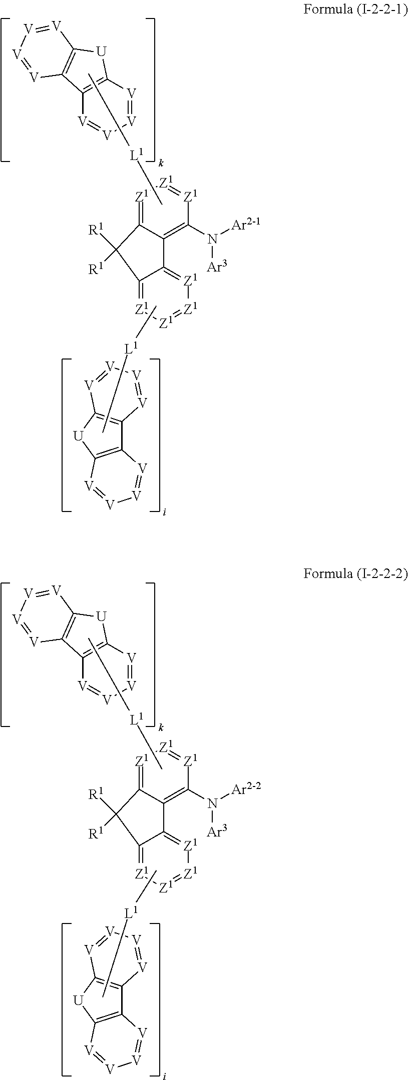

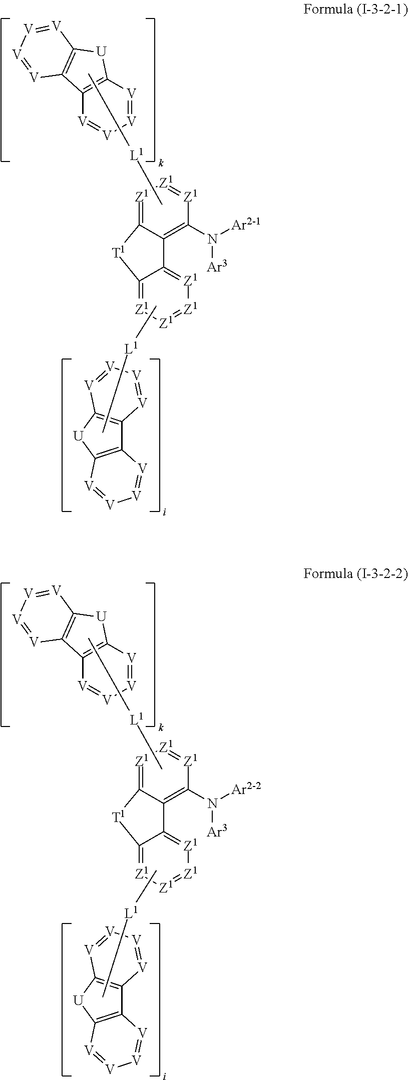

where the variables that occur are as defined above, and where T¹ is selected from O, S and NR¹, and where V is the same or different at each instance and is selected from CR² and N, where V is C when an L¹ group is bonded thereto, and where U is O, S or NR², where U is N when an L¹ group is bonded thereto, and where, in addition:
Ar²⁻¹ is selected from formulae (A-1) and (B-1)

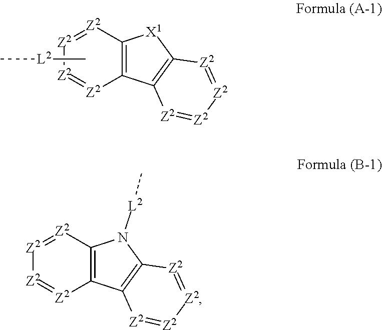

where Z² and L² are as defined above, and where X¹ is selected from NR³, O and S;
Ar²⁻¹² is selected from formulae (A-2), (B-2) and (C)

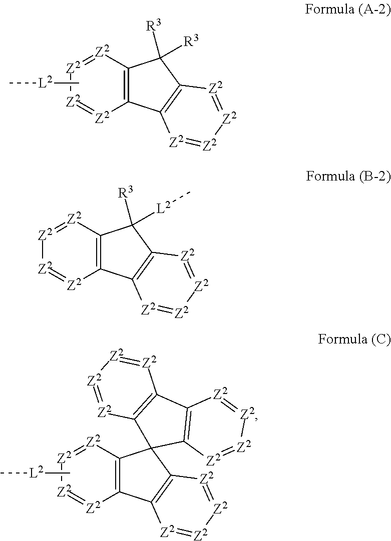

where L² and Z² are as defined above.

More preferably, Ar²⁻¹¹ in the abovementioned formulae corresponds to the formula (A-1). More preferably, Ar²⁻¹² in the abovementioned formulae corresponds to the formula (A-2) or (C), and among these most preferably to the formula (A-2).

Preferably, in the abovementioned formulae, Z¹ is CR¹, where Z¹ is C when a group having the index k or i is bonded thereto. Further preferably, in the abovementioned formulae, the sum total of i and k is 1. More preferably, the group

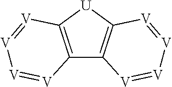

in each case is selected from dibenzofuran, dibenzothiophene and carbazole, where carbazole may be bonded via the nitrogen atom or via a bonding site on one of the six-membered rings. Very particular preference is given to dibenzofuran and dibenzothiophene.

For the abovementioned formulae, it is preferable that L² is selected from a single bond and an aromatic ring system which has 10 to 30 aromatic ring atoms and may be substituted by one or more R³ radicals. More preferably, L² in this case is a single bond. This is especially true of the formulae (I-1-2-1) and (I-1-2-2).

It is further preferable for the abovementioned formulae that Ar² corresponds to a formula (A-1), (A-2), (B-1) or (B-2), more preferably to a formula (A-1) or (A-2), most preferably to a formula (A-1). This is especially true of the formulae (I-1-2-1) and (I-1-2-2).

It is further preferable for the abovementioned formulae that Ar³ corresponds to a formula (A-1), (A-2), (B-1) or (B-2), or that Ar³ is selected from aromatic ring systems which have 6 to 18 aromatic ring atoms and may each be substituted by one or more R⁴ radicals and heteroaromatic ring systems which have 5 to 30 aromatic ring atoms and may each be substituted by one or more R⁴ radicals. This is especially true of the formulae (I-1-2-1) and (I-1-2-2).

Preferred compounds of the formula (I) are listed below. In these compounds, the unit of the formula (I-A) corresponds to one of the preferred embodiments listed in the table below, the Ar² group corresponds to one of the preferred embodiments listed in the table below, and the Ar³ group corresponds to one of the preferred embodiments listed in the table below:

| Formula (I-A) | Ar² | Ar³ |
|---|---|---|
| Formula (I-A-1) | Ar²-2 | Ar³-1 |
| Formula (I-A-2) | Ar²-6 | Ar³-2 |
| Formula (I-A-3) | Ar²-17 | Ar³-3 |
| Formula (I-A-19) | Ar²-25 | Ar³-4 |
| Formula (I-A-20) | Ar²-45 | Ar³-74 |
| Formula (I-A-21) | Ar²-65 | Ar³-85 |
| Formula (I-A-22) | Ar²-74 | Ar³-110 |
| Formula (I-A-78) | Ar²-105 | Ar³-132 |
| Formula (I-A-79) | Ar²-165 | Ar³-165 |
| Formula (I-A-80) | Ar²-173 | Ar³-235 |
| Formula (I-A-105) | | |
| Formula (I-A-106) | | |
| Formula (I-A-107) | | |
| Formula (I-A-108) | | |
| Formula (I-A-123) | | |
| Formula (I-A-126) | | |
| Formula (I-A-132) | | |
| Formula (I-A-133) | | |
| Formula (I-A-134) | | |
| Formula (I-A-135) | | |

The abovementioned groups do not bear any further substituents apart from those shown explicitly.

The following compounds are preferred embodiments of the compounds of the formula (I):

| | Formula | | |
|---|---|---|---|
| Nr. | (I-A- | Ar²- | Ar³- |
| 1 | 1 | 2 | 1 |
| 2 | 1 | 2 | 2 |
| 3 | 1 | 2 | 3 |
| 4 | 1 | 2 | 4 |
| 5 | 1 | 2 | 74 |
| 6 | 1 | 2 | 85 |
| 7 | 1 | 2 | 110 |
| 8 | 1 | 2 | 132 |
| 9 | 1 | 2 | 165 |
| 10 | 1 | 2 | 235 |
| 11 | 1 | 6 | 1 |
| 12 | 1 | 6 | 2 |
| 13 | 1 | 6 | 3 |
| 14 | 1 | 6 | 4 |
| 15 | 1 | 6 | 74 |
| 16 | 1 | 6 | 85 |
| 17 | 1 | 6 | 110 |
| 18 | 1 | 6 | 132 |
| 19 | 1 | 6 | 165 |
| 20 | 1 | 6 | 235 |
| 21 | 1 | 17 | 1 |
| 22 | 1 | 17 | 2 |
| 23 | 1 | 17 | 3 |
| 24 | 1 | 17 | 4 |
| 25 | 1 | 17 | 74 |
| 26 | 1 | 17 | 85 |
| 27 | 1 | 17 | 110 |
| 28 | 1 | 17 | 132 |
| 29 | 1 | 17 | 165 |
| 30 | 1 | 17 | 235 |
| 31 | 1 | 25 | 1 |
| 32 | 1 | 25 | 2 |
| 33 | 1 | 25 | 3 |
| 34 | 1 | 25 | 4 |
| 35 | 1 | 25 | 74 |
| 36 | 1 | 25 | 85 |
| 37 | 1 | 25 | 110 |
| 38 | 1 | 25 | 132 |
| 39 | 1 | 25 | 165 |
| 40 | 1 | 25 | 235 |
| 41 | 1 | 45 | 1 |
| 42 | 1 | 45 | 2 |
| 43 | 1 | 45 | 3 |
| 44 | 1 | 45 | 4 |
| 45 | 1 | 45 | 74 |
| 46 | 1 | 45 | 85 |
| 47 | 1 | 45 | 110 |
| 48 | 1 | 45 | 132 |
| 49 | 1 | 45 | 165 |
| 50 | 1 | 45 | 235 |
| 51 | 1 | 65 | 1 |
| 52 | 1 | 65 | 2 |
| 53 | 1 | 65 | 3 |
| 54 | 1 | 65 | 4 |
| 55 | 1 | 65 | 74 |
| 56 | 1 | 65 | 85 |
| 57 | 1 | 65 | 110 |
| 58 | 1 | 65 | 132 |
| 59 | 1 | 65 | 165 |
| 60 | 1 | 65 | 235 |
| 61 | 1 | 74 | 1 |
| 62 | 1 | 74 | 2 |
| 63 | 1 | 74 | 3 |
| 64 | 1 | 74 | 4 |
| 65 | 1 | 74 | 74 |
| 66 | 1 | 74 | 85 |
| 67 | 1 | 74 | 110 |
| 68 | 1 | 74 | 132 |
| 69 | 1 | 74 | 165 |
| 70 | 1 | 74 | 235 |
| 71 | 1 | 105 | 1 |
| 72 | 1 | 105 | 2 |
| 73 | 1 | 105 | 3 |
| 74 | 1 | 105 | 4 |
| 75 | 1 | 105 | 74 |
| 76 | 1 | 105 | 85 |
| 77 | 1 | 105 | 110 |
| 78 | 1 | 105 | 132 |
| 79 | 1 | 105 | 165 |
| 80 | 1 | 105 | 235 |
| 81 | 1 | 165 | 1 |
| 82 | 1 | 165 | 2 |
| 83 | 1 | 165 | 3 |
| 84 | 1 | 165 | 4 |
| 85 | 1 | 165 | 74 |
| 86 | 1 | 165 | 85 |
| 87 | 1 | 165 | 110 |
| 88 | 1 | 165 | 132 |
| 89 | 1 | 165 | 165 |
| 90 | 1 | 165 | 235 |
| 91 | 1 | 173 | 1 |
| 92 | 1 | 173 | 2 |
| 93 | 1 | 173 | 3 |
| 94 | 1 | 173 | 4 |
| 95 | 1 | 173 | 74 |
| 96 | 1 | 173 | 85 |
| 97 | 1 | 173 | 110 |
| 98 | 1 | 173 | 132 |
| 99 | 1 | 173 | 165 |
| 100 | 1 | 173 | 235 |
| 101 | 2 | 2 | 1 |
| 102 | 2 | 2 | 2 |
| 103 | 2 | 2 | 3 |
| 104 | 2 | 2 | 4 |
| 105 | 2 | 2 | 74 |
| 106 | 2 | 2 | 85 |
| 107 | 2 | 2 | 110 |
| 108 | 2 | 2 | 132 |
| 109 | 2 | 2 | 165 |
| 110 | 2 | 2 | 235 |
| 111 | 2 | 6 | 1 |
| 112 | 2 | 6 | 2 |
| 113 | 2 | 6 | 3 |
| 114 | 2 | 6 | 4 |
| 115 | 2 | 6 | 74 |
| 116 | 2 | 6 | 85 |
| 117 | 2 | 6 | 110 |
| 118 | 2 | 6 | 132 |
| 119 | 2 | 6 | 165 |
| 120 | 2 | 6 | 235 |
| 121 | 2 | 17 | 1 |
| 122 | 2 | 17 | 2 |
| 123 | 2 | 17 | 3 |
| 124 | 2 | 17 | 4 |
| 125 | 2 | 17 | 74 |
| 126 | 2 | 17 | 85 |
| 127 | 2 | 17 | 110 |
| 128 | 2 | 17 | 132 |
| 129 | 2 | 17 | 165 |
| 130 | 2 | 17 | 235 |
| 131 | 2 | 25 | 1 |
| 132 | 2 | 25 | 2 |
| 133 | 2 | 25 | 3 |
| 134 | 2 | 25 | 4 |
| 135 | 2 | 25 | 74 |
| 136 | 2 | 25 | 85 |
| 137 | 2 | 25 | 110 |
| 138 | 2 | 25 | 132 |
| 139 | 2 | 25 | 165 |
| 140 | 2 | 25 | 235 |
| 141 | 2 | 45 | 1 |
| 142 | 2 | 45 | 2 |
| 143 | 2 | 45 | 3 |
| 144 | 2 | 45 | 4 |
| 145 | 2 | 45 | 74 |
| 146 | 2 | 45 | 85 |
| 147 | 2 | 45 | 110 |
| 148 | 2 | 45 | 132 |
| 149 | 2 | 45 | 165 |
| 150 | 2 | 45 | 235 |
| 151 | 2 | 65 | 1 |
| 152 | 2 | 65 | 2 |
| 153 | 2 | 65 | 3 |
| 154 | 2 | 65 | 4 |
| 155 | 2 | 65 | 74 |
| 156 | 2 | 65 | 85 |
| 157 | 2 | 65 | 110 |
| 158 | 2 | 65 | 132 |
| 159 | 2 | 65 | 165 |
| 160 | 2 | 65 | 235 |
| 161 | 2 | 74 | 1 |
| 162 | 2 | 74 | 2 |
| 163 | 2 | 74 | 3 |
| 164 | 2 | 74 | 4 |
| 165 | 2 | 74 | 74 |
| 166 | 2 | 74 | 85 |
| 167 | 2 | 74 | 110 |
| 168 | 2 | 74 | 132 |
| 169 | 2 | 74 | 165 |
| 170 | 2 | 74 | 235 |
| 171 | 2 | 105 | 1 |
| 172 | 2 | 105 | 2 |
| 173 | 2 | 105 | 3 |
| 174 | 2 | 105 | 4 |
| 175 | 2 | 105 | 74 |
| 176 | 2 | 105 | 85 |
| 177 | 2 | 105 | 110 |
| 178 | 2 | 105 | 132 |
| 179 | 2 | 105 | 165 |
| 180 | 2 | 105 | 235 |
| 181 | 2 | 165 | 1 |
| 182 | 2 | 165 | 2 |
| 183 | 2 | 165 | 3 |
| 184 | 2 | 165 | 4 |
| 185 | 2 | 165 | 74 |
| 186 | 2 | 165 | 85 |
| 187 | 2 | 165 | 110 |
| 188 | 2 | 165 | 132 |
| 189 | 2 | 165 | 165 |
| 190 | 2 | 165 | 235 |
| 191 | 2 | 173 | 1 |
| 193 | 2 | 173 | 2 |
| 192 | 2 | 173 | 3 |
| 196 | 2 | 173 | 4 |
| 194 | 2 | 173 | 74 |
| 195 | 2 | 173 | 85 |
| 196 | 2 | 173 | 110 |
| 197 | 2 | 173 | 132 |
| 199 | 2 | 173 | 165 |
| 200 | 2 | 173 | 235 |
| 201 | 3 | 2 | 1 |
| 202 | 3 | 2 | 2 |
| 203 | 3 | 2 | 3 |
| 204 | 3 | 2 | 4 |
| 205 | 3 | 2 | 74 |
| 206 | 3 | 2 | 85 |
| 207 | 3 | 2 | 110 |
| 208 | 3 | 2 | 132 |
| 209 | 3 | 2 | 165 |
| 210 | 3 | 2 | 235 |
| 211 | 3 | 6 | 1 |
| 212 | 3 | 6 | 2 |
| 213 | 3 | 6 | 3 |
| 214 | 3 | 6 | 4 |
| 215 | 3 | 6 | 74 |
| 216 | 3 | 6 | 85 |
| 217 | 3 | 6 | 110 |
| 218 | 3 | 6 | 132 |
| 219 | 3 | 6 | 165 |
| 220 | 3 | 6 | 235 |
| 221 | 3 | 17 | 1 |
| 222 | 3 | 17 | 2 |
| 223 | 3 | 17 | 3 |
| 224 | 3 | 17 | 4 |
| 225 | 3 | 17 | 74 |
| 226 | 3 | 17 | 85 |
| 227 | 3 | 17 | 110 |
| 228 | 3 | 17 | 132 |
| 229 | 3 | 17 | 165 |
| 230 | 3 | 17 | 235 |
| 231 | 3 | 25 | 1 |
| 232 | 3 | 25 | 2 |
| 233 | 3 | 25 | 3 |
| 234 | 3 | 25 | 4 |
| 235 | 3 | 25 | 74 |
| 236 | 3 | 25 | 85 |
| 237 | 3 | 25 | 110 |
| 238 | 3 | 25 | 132 |
| 239 | 3 | 25 | 165 |
| 240 | 3 | 25 | 235 |
| 241 | 3 | 45 | 1 |
| 242 | 3 | 45 | 2 |
| 243 | 3 | 45 | 3 |
| 244 | 3 | 45 | 4 |
| 245 | 3 | 45 | 74 |
| 246 | 3 | 45 | 85 |
| 247 | 3 | 45 | 110 |
| 248 | 3 | 45 | 132 |
| 249 | 3 | 45 | 165 |
| 250 | 3 | 45 | 235 |
| 251 | 3 | 65 | 1 |
| 252 | 3 | 65 | 2 |
| 253 | 3 | 65 | 3 |
| 254 | 3 | 65 | 4 |
| 255 | 3 | 65 | 74 |
| 256 | 3 | 65 | 85 |
| 257 | 3 | 65 | 110 |
| 258 | 3 | 65 | 132 |
| 259 | 3 | 65 | 165 |
| 260 | 3 | 65 | 235 |
| 261 | 3 | 74 | 1 |
| 262 | 3 | 74 | 2 |
| 263 | 3 | 74 | 3 |
| 264 | 3 | 74 | 4 |
| 265 | 3 | 74 | 74 |
| 266 | 3 | 74 | 85 |
| 267 | 3 | 74 | 110 |
| 268 | 3 | 74 | 132 |
| 269 | 3 | 74 | 165 |
| 270 | 3 | 74 | 235 |
| 271 | 3 | 105 | 1 |
| 272 | 3 | 105 | 2 |
| 273 | 3 | 105 | 3 |
| 274 | 3 | 105 | 4 |
| 275 | 3 | 105 | 74 |
| 276 | 3 | 105 | 85 |
| 277 | 3 | 105 | 110 |
| 278 | 3 | 105 | 132 |
| 279 | 3 | 105 | 165 |
| 280 | 3 | 105 | 235 |
| 281 | 3 | 165 | 1 |
| 282 | 3 | 165 | 2 |
| 283 | 3 | 165 | 3 |
| 284 | 3 | 165 | 4 |
| 285 | 3 | 165 | 74 |
| 286 | 3 | 165 | 85 |
| 287 | 3 | 165 | 110 |
| 288 | 3 | 165 | 132 |
| 289 | 3 | 165 | 165 |
| 290 | 3 | 165 | 235 |
| 291 | 3 | 173 | 1 |
| 292 | 3 | 173 | 2 |
| 293 | 3 | 173 | 3 |
| 294 | 3 | 173 | 4 |
| 295 | 3 | 173 | 74 |
| 296 | 3 | 173 | 85 |
| 297 | 3 | 173 | 110 |
| 298 | 3 | 173 | 132 |
| 299 | 3 | 173 | 165 |
| 300 | 3 | 173 | 235 |
| 301 | 19 | 2 | 1 |
| 302 | 19 | 2 | 2 |
| 303 | 19 | 2 | 3 |
| 304 | 19 | 2 | 4 |
| 305 | 19 | 2 | 74 |
| 306 | 19 | 2 | 85 |
| 307 | 19 | 2 | 110 |
| 308 | 19 | 2 | 132 |
| 309 | 19 | 2 | 165 |
| 310 | 19 | 2 | 235 |
| 311 | 19 | 6 | 1 |
| 312 | 19 | 6 | 2 |
| 313 | 19 | 6 | 3 |
| 314 | 19 | 6 | 4 |
| 315 | 19 | 6 | 74 |
| 316 | 19 | 6 | 85 |
| 317 | 19 | 6 | 110 |
| 318 | 19 | 6 | 132 |
| 319 | 19 | 6 | 165 |
| 320 | 19 | 6 | 235 |
| 321 | 19 | 17 | 1 |
| 322 | 19 | 17 | 2 |
| 323 | 19 | 17 | 3 |
| 324 | 19 | 17 | 4 |
| 325 | 19 | 17 | 74 |
| 326 | 19 | 17 | 85 |
| 327 | 19 | 17 | 110 |
| 328 | 19 | 17 | 132 |
| 329 | 19 | 17 | 165 |
| 330 | 19 | 17 | 235 |
| 331 | 19 | 25 | 1 |
| 332 | 19 | 25 | 2 |
| 333 | 19 | 25 | 3 |
| 334 | 19 | 25 | 4 |
| 335 | 19 | 25 | 74 |
| 336 | 19 | 25 | 85 |
| 337 | 19 | 25 | 110 |
| 338 | 19 | 25 | 132 |
| 339 | 19 | 25 | 165 |
| 340 | 19 | 25 | 235 |
| 341 | 19 | 45 | 1 |
| 342 | 19 | 45 | 2 |
| 343 | 19 | 45 | 3 |
| 344 | 19 | 45 | 4 |
| 345 | 19 | 45 | 74 |
| 346 | 19 | 45 | 85 |
| 347 | 19 | 45 | 110 |
| 348 | 19 | 45 | 132 |
| 349 | 19 | 45 | 165 |
| 350 | 19 | 45 | 235 |
| 351 | 19 | 65 | 1 |
| 352 | 19 | 65 | 2 |
| 353 | 19 | 65 | 3 |
| 354 | 19 | 65 | 4 |
| 355 | 19 | 65 | 74 |
| 356 | 19 | 65 | 85 |
| 357 | 19 | 65 | 110 |
| 358 | 19 | 65 | 132 |
| 359 | 19 | 65 | 165 |
| 360 | 19 | 65 | 235 |
| 361 | 19 | 74 | 1 |
| 362 | 19 | 74 | 2 |
| 363 | 19 | 74 | 3 |
| 364 | 19 | 74 | 4 |
| 365 | 19 | 74 | 74 |
| 366 | 19 | 74 | 85 |
| 367 | 19 | 74 | 110 |
| 368 | 19 | 74 | 132 |
| 369 | 19 | 74 | 165 |
| 370 | 19 | 74 | 235 |
| 371 | 19 | 105 | 1 |
| 372 | 19 | 105 | 2 |
| 373 | 19 | 105 | 3 |
| 374 | 19 | 105 | 4 |
| 375 | 19 | 105 | 74 |
| 376 | 19 | 105 | 85 |
| 377 | 19 | 105 | 110 |
| 378 | 19 | 105 | 132 |
| 379 | 19 | 105 | 165 |
| 380 | 19 | 105 | 235 |
| 381 | 19 | 165 | 1 |
| 382 | 19 | 165 | 2 |
| 383 | 19 | 165 | 3 |
| 384 | 19 | 165 | 4 |
| 385 | 19 | 165 | 74 |
| 386 | 19 | 165 | 85 |
| 387 | 19 | 165 | 110 |
| 388 | 19 | 165 | 132 |
| 389 | 19 | 165 | 165 |
| 390 | 19 | 165 | 235 |
| 391 | 19 | 173 | 1 |
| 392 | 19 | 173 | 2 |
| 393 | 19 | 173 | 3 |
| 394 | 19 | 173 | 4 |
| 395 | 19 | 173 | 74 |
| 396 | 19 | 173 | 85 |
| 397 | 19 | 173 | 110 |
| 398 | 19 | 173 | 132 |
| 399 | 19 | 173 | 165 |
| 400 | 19 | 173 | 235 |
| 401 | 20 | 2 | 1 |
| 402 | 20 | 2 | 2 |
| 403 | 20 | 2 | 3 |
| 404 | 20 | 2 | 4 |
| 405 | 20 | 2 | 74 |
| 406 | 20 | 2 | 85 |
| 407 | 20 | 2 | 110 |
| 408 | 20 | 2 | 132 |
| 409 | 20 | 2 | 165 |
| 410 | 20 | 2 | 235 |
| 411 | 20 | 6 | 1 |
| 412 | 20 | 6 | 2 |
| 413 | 20 | 6 | 3 |
| 414 | 20 | 6 | 4 |
| 415 | 20 | 6 | 74 |
| 416 | 20 | 6 | 85 |
| 417 | 20 | 6 | 110 |
| 418 | 20 | 6 | 132 |
| 419 | 20 | 6 | 165 |
| 420 | 20 | 6 | 235 |
| 421 | 20 | 17 | 1 |
| 422 | 20 | 17 | 2 |
| 423 | 20 | 17 | 3 |
| 424 | 20 | 17 | 4 |
| 425 | 20 | 17 | 74 |
| 426 | 20 | 17 | 85 |
| 427 | 20 | 17 | 110 |
| 428 | 20 | 17 | 132 |
| 429 | 20 | 17 | 165 |
| 430 | 20 | 17 | 235 |
| 431 | 20 | 25 | 1 |
| 432 | 20 | 25 | 2 |
| 433 | 20 | 25 | 3 |
| 434 | 20 | 25 | 4 |
| 435 | 20 | 25 | 74 |
| 436 | 20 | 25 | 85 |
| 437 | 20 | 25 | 110 |
| 438 | 20 | 25 | 132 |
| 439 | 20 | 25 | 165 |
| 440 | 20 | 25 | 235 |
| 441 | 20 | 45 | 1 |
| 442 | 20 | 45 | 2 |
| 443 | 20 | 45 | 3 |
| 444 | 20 | 45 | 4 |
| 445 | 20 | 45 | 74 |
| 446 | 20 | 45 | 85 |
| 447 | 20 | 45 | 110 |
| 448 | 20 | 45 | 132 |
| 449 | 20 | 45 | 165 |
| 450 | 20 | 45 | 235 |
| 451 | 20 | 65 | 1 |
| 452 | 20 | 65 | 2 |
| 453 | 20 | 65 | 3 |
| 454 | 20 | 65 | 4 |
| 455 | 20 | 65 | 74 |
| 456 | 20 | 65 | 85 |
| 457 | 20 | 65 | 110 |
| 458 | 20 | 65 | 132 |
| 459 | 20 | 65 | 165 |
| 460 | 20 | 65 | 235 |
| 461 | 20 | 74 | 1 |
| 462 | 20 | 74 | 2 |
| 463 | 20 | 74 | 3 |
| 464 | 20 | 74 | 4 |
| 465 | 20 | 74 | 74 |
| 466 | 20 | 74 | 85 |
| 467 | 20 | 74 | 110 |
| 468 | 20 | 74 | 132 |
| 469 | 20 | 74 | 165 |
| 470 | 20 | 74 | 235 |
| 471 | 20 | 105 | 1 |
| 472 | 20 | 105 | 2 |
| 473 | 20 | 105 | 3 |
| 474 | 20 | 105 | 4 |
| 475 | 20 | 105 | 74 |
| 476 | 20 | 105 | 85 |
| 477 | 20 | 105 | 110 |
| 478 | 20 | 105 | 132 |
| 479 | 20 | 105 | 165 |
| 480 | 20 | 105 | 235 |
| 481 | 20 | 165 | 1 |
| 482 | 20 | 165 | 2 |
| 483 | 20 | 165 | 3 |
| 484 | 20 | 165 | 4 |
| 485 | 20 | 165 | 74 |
| 486 | 20 | 165 | 85 |
| 487 | 20 | 165 | 110 |
| 488 | 20 | 165 | 132 |
| 489 | 20 | 165 | 165 |
| 490 | 20 | 165 | 235 |
| 491 | 20 | 173 | 1 |
| 492 | 20 | 173 | 2 |
| 493 | 20 | 173 | 3 |
| 494 | 20 | 173 | 4 |
| 495 | 20 | 173 | 74 |
| 496 | 20 | 173 | 85 |
| 497 | 20 | 173 | 110 |
| 498 | 20 | 173 | 132 |
| 499 | 20 | 173 | 165 |
| 500 | 20 | 173 | 235 |
| 501 | 21 | 2 | 1 |
| 502 | 21 | 2 | 2 |
| 503 | 21 | 2 | 3 |
| 504 | 21 | 2 | 4 |
| 505 | 21 | 2 | 74 |
| 506 | 21 | 2 | 85 |
| 507 | 21 | 2 | 110 |
| 508 | 21 | 2 | 132 |
| 509 | 21 | 2 | 165 |
| 510 | 21 | 2 | 235 |
| 511 | 21 | 6 | 1 |
| 512 | 21 | 6 | 2 |
| 513 | 21 | 6 | 3 |
| 514 | 21 | 6 | 4 |
| 515 | 21 | 6 | 74 |
| 516 | 21 | 6 | 85 |
| 517 | 21 | 6 | 110 |
| 518 | 21 | 6 | 132 |
| 519 | 21 | 6 | 165 |
| 520 | 21 | 6 | 235 |
| 521 | 21 | 17 | 1 |
| 522 | 21 | 17 | 2 |
| 523 | 21 | 17 | 3 |
| 524 | 21 | 17 | 4 |
| 525 | 21 | 17 | 74 |
| 526 | 21 | 17 | 85 |
| 527 | 21 | 17 | 110 |
| 528 | 21 | 17 | 132 |
| 529 | 21 | 17 | 165 |
| 530 | 21 | 17 | 235 |
| 531 | 21 | 25 | 1 |
| 532 | 21 | 25 | 2 |
| 533 | 21 | 25 | 3 |
| 534 | 21 | 25 | 4 |
| 535 | 21 | 25 | 74 |
| 536 | 21 | 25 | 85 |
| 537 | 21 | 25 | 110 |
| 538 | 21 | 25 | 132 |
| 539 | 21 | 25 | 165 |
| 540 | 21 | 25 | 235 |
| 541 | 21 | 45 | 1 |
| 542 | 21 | 45 | 2 |
| 543 | 21 | 45 | 3 |
| 544 | 21 | 45 | 4 |
| 545 | 21 | 45 | 74 |
| 546 | 21 | 45 | 85 |
| 547 | 21 | 45 | 110 |
| 548 | 21 | 45 | 132 |
| 549 | 21 | 45 | 165 |
| 550 | 21 | 45 | 235 |
| 551 | 21 | 65 | 1 |
| 552 | 21 | 65 | 2 |
| 553 | 21 | 65 | 3 |
| 554 | 21 | 65 | 4 |
| 555 | 21 | 65 | 74 |
| 556 | 21 | 65 | 85 |
| 557 | 21 | 65 | 110 |
| 558 | 21 | 65 | 132 |
| 559 | 21 | 65 | 165 |
| 560 | 21 | 65 | 235 |
| 561 | 21 | 74 | 1 |
| 562 | 21 | 74 | 2 |
| 563 | 21 | 74 | 3 |
| 564 | 21 | 74 | 4 |
| 565 | 21 | 74 | 74 |
| 566 | 21 | 74 | 85 |
| 567 | 21 | 74 | 110 |
| 568 | 21 | 74 | 132 |
| 569 | 21 | 74 | 165 |
| 570 | 21 | 74 | 235 |
| 571 | 21 | 105 | 1 |
| 572 | 21 | 105 | 2 |
| 573 | 21 | 105 | 3 |
| 574 | 21 | 105 | 4 |
| 575 | 21 | 105 | 74 |
| 576 | 21 | 105 | 85 |
| 577 | 21 | 105 | 110 |
| 578 | 21 | 105 | 132 |
| 579 | 21 | 105 | 165 |
| 580 | 21 | 105 | 235 |
| 581 | 21 | 165 | 1 |
| 582 | 21 | 165 | 2 |
| 583 | 21 | 165 | 3 |
| 584 | 21 | 165 | 4 |
| 585 | 21 | 165 | 74 |
| 586 | 21 | 165 | 85 |
| 587 | 21 | 165 | 110 |
| 588 | 21 | 165 | 132 |
| 589 | 21 | 165 | 165 |
| 590 | 21 | 165 | 235 |
| 591 | 21 | 173 | 1 |
| 592 | 21 | 173 | 2 |
| 593 | 21 | 173 | 3 |
| 594 | 21 | 173 | 4 |
| 595 | 21 | 173 | 74 |
| 596 | 21 | 173 | 85 |
| 597 | 21 | 173 | 110 |
| 598 | 21 | 173 | 132 |
| 599 | 21 | 173 | 165 |
| 600 | 21 | 173 | 235 |
| 601 | 22 | 2 | 1 |
| 602 | 22 | 2 | 2 |
| 603 | 22 | 2 | 3 |
| 604 | 22 | 2 | 4 |
| 605 | 22 | 2 | 74 |
| 606 | 22 | 2 | 85 |
| 607 | 22 | 2 | 110 |
| 608 | 22 | 2 | 132 |
| 609 | 22 | 2 | 165 |
| 610 | 22 | 2 | 235 |
| 611 | 22 | 6 | 1 |
| 612 | 22 | 6 | 2 |
| 613 | 22 | 6 | 3 |
| 614 | 22 | 6 | 4 |
| 615 | 22 | 6 | 74 |
| 616 | 22 | 6 | 85 |
| 617 | 22 | 6 | 110 |
| 618 | 22 | 6 | 132 |
| 619 | 22 | 6 | 165 |
| 620 | 22 | 6 | 235 |
| 621 | 22 | 17 | 1 |
| 622 | 22 | 17 | 2 |
| 623 | 22 | 17 | 3 |
| 624 | 22 | 17 | 4 |
| 625 | 22 | 17 | 74 |
| 626 | 22 | 17 | 85 |
| 627 | 22 | 17 | 110 |
| 628 | 22 | 17 | 132 |
| 629 | 22 | 17 | 165 |
| 630 | 22 | 17 | 235 |
| 631 | 22 | 25 | 1 |
| 632 | 22 | 25 | 2 |
| 633 | 22 | 25 | 3 |
| 634 | 22 | 25 | 4 |
| 635 | 22 | 25 | 74 |
| 636 | 22 | 25 | 85 |
| 637 | 22 | 25 | 110 |
| 638 | 22 | 25 | 132 |
| 639 | 22 | 25 | 165 |
| 640 | 22 | 25 | 235 |
| 641 | 22 | 45 | 1 |
| 642 | 22 | 45 | 2 |
| 643 | 22 | 45 | 3 |
| 644 | 22 | 45 | 4 |
| 645 | 22 | 45 | 74 |
| 646 | 22 | 45 | 85 |
| 647 | 22 | 45 | 110 |
| 648 | 22 | 45 | 132 |
| 649 | 22 | 45 | 165 |
| 650 | 22 | 45 | 235 |
| 651 | 22 | 65 | 1 |
| 652 | 22 | 65 | 2 |
| 653 | 22 | 65 | 3 |
| 654 | 22 | 65 | 4 |
| 655 | 22 | 65 | 74 |
| 656 | 22 | 65 | 85 |
| 657 | 22 | 65 | 110 |
| 658 | 22 | 65 | 132 |
| 659 | 22 | 65 | 165 |
| 660 | 22 | 65 | 235 |
| 661 | 22 | 74 | 1 |
| 662 | 22 | 74 | 2 |
| 663 | 22 | 74 | 3 |
| 664 | 22 | 74 | 4 |
| 665 | 22 | 74 | 74 |
| 666 | 22 | 74 | 85 |
| 667 | 22 | 74 | 110 |
| 668 | 22 | 74 | 132 |
| 669 | 22 | 74 | 165 |
| 670 | 22 | 74 | 235 |
| 671 | 22 | 105 | 1 |
| 672 | 22 | 105 | 2 |
| 673 | 22 | 105 | 3 |
| 674 | 22 | 105 | 4 |
| 675 | 22 | 105 | 74 |
| 676 | 22 | 105 | 85 |
| 677 | 22 | 105 | 110 |
| 678 | 22 | 105 | 132 |
| 679 | 22 | 105 | 165 |
| 680 | 22 | 105 | 235 |
| 681 | 22 | 165 | 1 |
| 682 | 22 | 165 | 2 |
| 683 | 22 | 165 | 3 |
| 684 | 22 | 165 | 4 |
| 685 | 22 | 165 | 74 |
| 686 | 22 | 165 | 85 |
| 687 | 22 | 165 | 110 |
| 688 | 22 | 165 | 132 |
| 689 | 22 | 165 | 165 |
| 690 | 22 | 165 | 235 |
| 691 | 22 | 173 | 1 |
| 692 | 22 | 173 | 2 |
| 693 | 22 | 173 | 3 |
| 694 | 22 | 173 | 4 |
| 695 | 22 | 173 | 74 |
| 696 | 22 | 173 | 85 |
| 697 | 22 | 173 | 110 |
| 698 | 22 | 173 | 132 |
| 699 | 22 | 173 | 165 |
| 700 | 22 | 173 | 235 |
| 701 | 78 | 2 | 1 |
| 702 | 78 | 2 | 2 |
| 703 | 78 | 2 | 3 |
| 704 | 78 | 2 | 4 |
| 705 | 78 | 2 | 74 |
| 706 | 78 | 2 | 85 |
| 707 | 78 | 2 | 110 |
| 708 | 78 | 2 | 132 |
| 709 | 78 | 2 | 165 |
| 710 | 78 | 2 | 235 |
| 711 | 78 | 6 | 1 |
| 712 | 78 | 6 | 2 |
| 713 | 78 | 6 | 3 |
| 714 | 78 | 6 | 4 |
| 715 | 78 | 6 | 74 |
| 716 | 78 | 6 | 85 |
| 717 | 78 | 6 | 110 |
| 718 | 78 | 6 | 132 |
| 719 | 78 | 6 | 165 |
| 720 | 78 | 6 | 235 |
| 721 | 78 | 17 | 1 |
| 722 | 78 | 17 | 2 |
| 723 | 78 | 17 | 3 |
| 724 | 78 | 17 | 4 |
| 725 | 78 | 17 | 74 |
| 726 | 78 | 17 | 85 |
| 727 | 78 | 17 | 110 |
| 728 | 78 | 17 | 132 |
| 729 | 78 | 17 | 165 |
| 730 | 78 | 17 | 235 |
| 731 | 78 | 25 | 1 |
| 732 | 78 | 25 | 2 |
| 733 | 78 | 25 | 3 |
| 734 | 78 | 25 | 4 |
| 735 | 78 | 25 | 74 |
| 736 | 78 | 25 | 85 |
| 737 | 78 | 25 | 110 |
| 738 | 78 | 25 | 132 |
| 739 | 78 | 25 | 165 |
| 740 | 78 | 25 | 235 |
| 741 | 78 | 45 | 1 |
| 742 | 78 | 45 | 2 |
| 743 | 78 | 45 | 3 |
| 744 | 78 | 45 | 4 |
| 745 | 78 | 45 | 74 |
| 746 | 78 | 45 | 85 |
| 747 | 78 | 45 | 110 |
| 748 | 78 | 45 | 132 |
| 749 | 78 | 45 | 165 |
| 750 | 78 | 45 | 235 |
| 751 | 78 | 65 | 1 |
| 752 | 78 | 65 | 2 |
| 753 | 78 | 65 | 3 |
| 754 | 78 | 65 | 4 |
| 755 | 78 | 65 | 74 |
| 756 | 78 | 65 | 85 |
| 757 | 78 | 65 | 110 |
| 758 | 78 | 65 | 132 |
| 759 | 78 | 65 | 165 |
| 760 | 78 | 65 | 235 |
| 761 | 78 | 74 | 1 |
| 762 | 78 | 74 | 2 |
| 763 | 78 | 74 | 3 |
| 764 | 78 | 74 | 4 |
| 765 | 78 | 74 | 74 |
| 766 | 78 | 74 | 85 |
| 767 | 78 | 74 | 110 |
| 768 | 78 | 74 | 132 |
| 769 | 78 | 74 | 165 |
| 770 | 78 | 74 | 235 |
| 771 | 78 | 105 | 1 |
| 772 | 78 | 105 | 2 |
| 773 | 78 | 105 | 3 |
| 774 | 78 | 105 | 4 |
| 775 | 78 | 105 | 74 |
| 776 | 78 | 105 | 85 |
| 777 | 78 | 105 | 110 |
| 778 | 78 | 105 | 132 |
| 779 | 78 | 105 | 165 |
| 780 | 78 | 105 | 235 |
| 781 | 78 | 165 | 1 |
| 782 | 78 | 165 | 2 |
| 783 | 78 | 165 | 3 |
| 784 | 78 | 165 | 4 |
| 785 | 78 | 165 | 74 |
| 786 | 78 | 165 | 85 |
| 787 | 78 | 165 | 110 |
| 788 | 78 | 165 | 132 |
| 789 | 78 | 165 | 165 |
| 790 | 78 | 165 | 235 |
| 791 | 78 | 173 | 1 |
| 792 | 78 | 173 | 2 |
| 793 | 78 | 173 | 3 |
| 794 | 78 | 173 | 4 |
| 795 | 78 | 173 | 74 |
| 796 | 78 | 173 | 85 |
| 797 | 78 | 173 | 110 |
| 798 | 78 | 173 | 132 |
| 799 | 78 | 173 | 165 |
| 800 | 78 | 173 | 235 |
| 801 | 79 | 2 | 1 |
| 802 | 79 | 2 | 2 |
| 803 | 79 | 2 | 3 |
| 804 | 79 | 2 | 4 |
| 805 | 79 | 2 | 74 |
| 806 | 79 | 2 | 85 |
| 807 | 79 | 2 | 110 |
| 808 | 79 | 2 | 132 |
| 809 | 79 | 2 | 165 |
| 810 | 79 | 2 | 235 |
| 811 | 79 | 6 | 1 |
| 812 | 79 | 6 | 2 |
| 813 | 79 | 6 | 3 |
| 814 | 79 | 6 | 4 |
| 815 | 79 | 6 | 74 |
| 816 | 79 | 6 | 85 |
| 817 | 79 | 6 | 110 |
| 818 | 79 | 6 | 132 |
| 819 | 79 | 6 | 165 |
| 820 | 79 | 6 | 235 |
| 821 | 79 | 17 | 1 |
| 822 | 79 | 17 | 2 |
| 823 | 79 | 17 | 3 |
| 824 | 79 | 17 | 4 |
| 825 | 79 | 17 | 74 |
| 826 | 79 | 17 | 85 |
| 827 | 79 | 17 | 110 |
| 828 | 79 | 17 | 132 |
| 829 | 79 | 17 | 165 |
| 830 | 79 | 17 | 235 |
| 831 | 79 | 25 | 1 |
| 832 | 79 | 25 | 2 |
| 833 | 79 | 25 | 3 |
| 834 | 79 | 25 | 4 |
| 835 | 79 | 25 | 74 |
| 836 | 79 | 25 | 85 |
| 837 | 79 | 25 | 110 |
| 838 | 79 | 25 | 132 |
| 839 | 79 | 25 | 165 |
| 840 | 79 | 25 | 235 |
| 841 | 79 | 45 | 1 |
| 842 | 79 | 45 | 2 |
| 843 | 79 | 45 | 3 |
| 844 | 79 | 45 | 4 |
| 845 | 79 | 45 | 74 |
| 846 | 79 | 45 | 85 |
| 847 | 79 | 45 | 110 |
| 848 | 79 | 45 | 132 |
| 849 | 79 | 45 | 165 |
| 850 | 79 | 45 | 235 |
| 851 | 79 | 65 | 1 |
| 852 | 79 | 65 | 2 |
| 853 | 79 | 65 | 3 |
| 854 | 79 | 65 | 4 |
| 855 | 79 | 65 | 74 |
| 856 | 79 | 65 | 85 |
| 857 | 79 | 65 | 110 |
| 858 | 79 | 65 | 132 |
| 859 | 79 | 65 | 165 |
| 860 | 79 | 65 | 235 |
| 861 | 79 | 74 | 1 |
| 862 | 79 | 74 | 2 |
| 863 | 79 | 74 | 3 |
| 864 | 79 | 74 | 4 |
| 865 | 79 | 74 | 74 |
| 866 | 79 | 74 | 85 |
| 867 | 79 | 74 | 110 |
| 868 | 79 | 74 | 132 |
| 869 | 79 | 74 | 165 |
| 870 | 79 | 74 | 235 |
| 871 | 79 | 105 | 1 |
| 872 | 79 | 105 | 2 |
| 873 | 79 | 105 | 3 |
| 874 | 79 | 105 | 4 |
| 875 | 79 | 105 | 74 |
| 876 | 79 | 105 | 85 |
| 877 | 79 | 105 | 110 |
| 878 | 79 | 105 | 132 |
| 879 | 79 | 105 | 165 |
| 880 | 79 | 105 | 235 |
| 881 | 79 | 165 | 1 |
| 882 | 79 | 165 | 2 |
| 883 | 79 | 165 | 3 |
| 884 | 79 | 165 | 4 |
| 885 | 79 | 165 | 74 |
| 886 | 79 | 165 | 85 |
| 887 | 79 | 165 | 110 |
| 888 | 79 | 165 | 132 |
| 889 | 79 | 165 | 165 |
| 890 | 79 | 165 | 235 |
| 891 | 79 | 173 | 1 |
| 892 | 79 | 173 | 2 |
| 893 | 79 | 173 | 3 |
| 894 | 79 | 173 | 4 |
| 895 | 79 | 173 | 74 |
| 896 | 79 | 173 | 85 |
| 897 | 79 | 173 | 110 |
| 898 | 79 | 173 | 132 |
| 899 | 79 | 173 | 165 |
| 900 | 79 | 173 | 235 |
| 901 | 80 | 2 | 1 |
| 902 | 80 | 2 | 2 |
| 903 | 80 | 2 | 3 |
| 904 | 80 | 2 | 4 |
| 905 | 80 | 2 | 74 |
| 906 | 80 | 2 | 85 |
| 907 | 80 | 2 | 110 |
| 908 | 80 | 2 | 132 |
| 909 | 80 | 2 | 165 |
| 910 | 80 | 2 | 235 |
| 911 | 80 | 6 | 1 |
| 912 | 80 | 6 | 2 |
| 913 | 80 | 6 | 3 |
| 914 | 80 | 6 | 4 |
| 915 | 80 | 6 | 74 |
| 916 | 80 | 6 | 85 |
| 917 | 80 | 6 | 110 |
| 918 | 80 | 6 | 132 |
| 919 | 80 | 6 | 165 |
| 920 | 80 | 6 | 235 |
| 921 | 80 | 17 | 1 |
| 922 | 80 | 17 | 2 |
| 923 | 80 | 17 | 3 |
| 924 | 80 | 17 | 4 |
| 925 | 80 | 17 | 74 |
| 926 | 80 | 17 | 85 |
| 927 | 80 | 17 | 110 |
| 928 | 80 | 17 | 132 |
| 929 | 90 | 17 | 165 |
| 930 | 80 | 17 | 235 |
| 931 | 80 | 25 | 1 |
| 932 | 80 | 25 | 2 |
| 933 | 80 | 25 | 3 |
| 934 | 80 | 25 | 4 |
| 935 | 80 | 25 | 74 |
| 936 | 80 | 25 | 85 |
| 937 | 80 | 25 | 110 |
| 938 | 80 | 25 | 132 |
| 939 | 80 | 25 | 165 |
| 940 | 80 | 25 | 235 |
| 941 | 80 | 45 | 1 |
| 942 | 80 | 45 | 2 |
| 943 | 80 | 45 | 3 |
| 944 | 80 | 45 | 4 |
| 945 | 80 | 45 | 74 |
| 946 | 80 | 45 | 85 |
| 947 | 80 | 45 | 110 |
| 948 | 80 | 45 | 132 |
| 949 | 80 | 45 | 165 |
| 950 | 80 | 45 | 235 |
| 951 | 80 | 65 | 1 |
| 952 | 80 | 65 | 2 |
| 953 | 80 | 65 | 3 |
| 954 | 80 | 65 | 4 |
| 955 | 80 | 65 | 74 |
| 956 | 80 | 65 | 85 |
| 957 | 80 | 65 | 110 |
| 958 | 90 | 65 | 132 |
| 959 | 80 | 65 | 165 |
| 960 | 80 | 65 | 235 |
| 961 | 80 | 74 | 1 |
| 962 | 80 | 74 | 2 |
| 963 | 80 | 74 | 3 |
| 964 | 80 | 74 | 4 |
| 965 | 80 | 74 | 74 |
| 966 | 80 | 74 | 85 |
| 967 | 80 | 74 | 110 |
| 968 | 80 | 74 | 132 |
| 969 | 80 | 74 | 165 |
| 970 | 80 | 74 | 235 |
| 971 | 80 | 105 | 1 |
| 972 | 80 | 105 | 2 |
| 973 | 80 | 105 | 3 |
| 974 | 80 | 105 | 4 |
| 975 | 80 | 105 | 74 |
| 976 | 80 | 105 | 85 |
| 977 | 80 | 105 | 110 |
| 978 | 80 | 105 | 132 |
| 978 | 80 | 105 | 165 |
| 980 | 80 | 105 | 235 |
| 981 | 80 | 165 | 1 |
| 982 | 80 | 165 | 2 |
| 983 | 80 | 165 | 3 |
| 984 | 80 | 165 | 4 |
| 985 | 80 | 165 | 74 |
| 986 | 80 | 165 | 85 |
| 987 | 80 | 165 | 110 |
| 988 | 80 | 165 | 132 |
| 989 | 80 | 165 | 165 |
| 990 | 80 | 165 | 235 |
| 991 | 80 | 173 | 1 |
| 992 | 80 | 173 | 2 |
| 993 | 80 | 173 | 3 |
| 994 | 80 | 173 | 4 |
| 995 | 80 | 173 | 74 |
| 996 | 80 | 173 | 85 |
| 997 | 80 | 173 | 110 |
| 998 | 80 | 173 | 132 |
| 999 | 80 | 173 | 165 |
| 1000 | 80 | 173 | 235 |
| 1001 | 105 | 2 | 1 |
| 1002 | 105 | 2 | 2 |
| 1003 | 105 | 2 | 3 |
| 1004 | 105 | 2 | 4 |
| 1005 | 105 | 2 | 74 |
| 1006 | 105 | 2 | 85 |
| 1007 | 105 | 2 | 110 |
| 1008 | 105 | 2 | 132 |
| 1009 | 105 | 2 | 165 |
| 1010 | 105 | 2 | 235 |
| 1011 | 105 | 6 | 1 |
| 1012 | 105 | 6 | 2 |
| 1013 | 105 | 6 | 3 |
| 1014 | 105 | 6 | 4 |
| 1015 | 105 | 6 | 74 |
| 1016 | 105 | 6 | 85 |
| 1017 | 105 | 6 | 110 |
| 1018 | 105 | 6 | 132 |
| 1019 | 105 | 6 | 165 |
| 1020 | 105 | 6 | 235 |
| 1021 | 105 | 17 | 1 |
| 1022 | 105 | 17 | 2 |
| 1023 | 105 | 17 | 3 |
| 1024 | 105 | 17 | 4 |
| 1025 | 105 | 17 | 74 |
| 1026 | 105 | 17 | 85 |
| 1027 | 105 | 17 | 110 |
| 1028 | 105 | 17 | 132 |
| 1029 | 105 | 17 | 165 |
| 1030 | 105 | 17 | 235 |
| 1031 | 105 | 25 | 1 |
| 1032 | 105 | 25 | 2 |
| 1033 | 105 | 25 | 3 |
| 1034 | 105 | 25 | 4 |
| 1035 | 105 | 25 | 74 |
| 1036 | 105 | 25 | 85 |
| 1037 | 105 | 25 | 110 |
| 1038 | 105 | 25 | 132 |
| 1039 | 105 | 25 | 165 |
| 1040 | 105 | 25 | 235 |
| 1041 | 105 | 45 | 1 |
| 1042 | 105 | 45 | 2 |
| 1043 | 105 | 45 | 3 |
| 1044 | 105 | 45 | 4 |
| 1045 | 105 | 45 | 74 |
| 1046 | 105 | 45 | 85 |
| 1047 | 105 | 45 | 110 |
| 1048 | 105 | 45 | 132 |
| 1049 | 105 | 45 | 165 |
| 1050 | 105 | 45 | 235 |
| 1051 | 105 | 65 | 1 |
| 1052 | 105 | 65 | 2 |
| 1053 | 105 | 65 | 3 |
| 1054 | 105 | 65 | 4 |
| 1055 | 105 | 65 | 74 |
| 1056 | 105 | 65 | 85 |
| 1057 | 105 | 65 | 110 |
| 1058 | 105 | 65 | 132 |
| 1059 | 105 | 65 | 165 |
| 1060 | 105 | 65 | 235 |
| 1061 | 105 | 74 | 1 |
| 1062 | 105 | 74 | 2 |
| 1063 | 105 | 74 | 3 |
| 1064 | 105 | 74 | 4 |
| 1065 | 105 | 74 | 74 |
| 1066 | 105 | 74 | 85 |
| 1067 | 105 | 74 | 110 |
| 1068 | 105 | 74 | 132 |
| 1069 | 105 | 74 | 165 |
| 1070 | 105 | 74 | 235 |
| 1071 | 105 | 105 | 1 |
| 1072 | 105 | 105 | 2 |
| 1073 | 105 | 105 | 3 |
| 1074 | 105 | 105 | 4 |
| 1075 | 105 | 105 | 74 |
| 1076 | 105 | 105 | 85 |
| 1077 | 105 | 105 | 110 |
| 1078 | 105 | 105 | 132 |
| 1079 | 105 | 105 | 165 |
| 1080 | 105 | 105 | 235 |
| 1081 | 105 | 165 | 1 |
| 1082 | 105 | 165 | 2 |
| 1083 | 105 | 165 | 3 |
| 1084 | 105 | 165 | 4 |
| 1085 | 105 | 165 | 74 |
| 1086 | 105 | 165 | 85 |
| 1087 | 105 | 165 | 110 |
| 1088 | 105 | 165 | 132 |
| 1089 | 105 | 165 | 165 |
| 1090 | 105 | 165 | 235 |
| 1091 | 105 | 173 | 1 |
| 1092 | 105 | 173 | 2 |
| 1093 | 105 | 173 | 3 |
| 1094 | 105 | 173 | 4 |
| 1095 | 105 | 173 | 74 |
| 1096 | 105 | 173 | 85 |
| 1097 | 105 | 173 | 110 |
| 1098 | 105 | 173 | 132 |
| 1099 | 105 | 173 | 165 |
| 1100 | 105 | 173 | 235 |
| 1101 | 106 | 2 | 1 |
| 1102 | 106 | 2 | 2 |
| 1103 | 106 | 2 | 3 |
| 1104 | 106 | 2 | 4 |
| 1105 | 106 | 2 | 74 |
| 1106 | 106 | 2 | 85 |
| 1107 | 106 | 2 | 110 |
| 1108 | 106 | 2 | 132 |
| 1109 | 106 | 2 | 165 |
| 1110 | 106 | 2 | 235 |
| 1111 | 106 | 2 | 1 |
| 1112 | 106 | 2 | 2 |
| 1113 | 106 | 6 | 3 |
| 1114 | 106 | 6 | 4 |
| 1115 | 106 | 6 | 74 |
| 1116 | 106 | 6 | 85 |
| 1117 | 106 | 6 | 110 |
| 1118 | 106 | 6 | 132 |
| 1119 | 106 | 6 | 165 |
| 1120 | 106 | 6 | 235 |
| 1121 | 106 | 17 | 1 |
| 1122 | 106 | 17 | 2 |
| 1123 | 106 | 17 | 3 |
| 1124 | 106 | 17 | 4 |
| 1125 | 106 | 17 | 74 |
| 1126 | 106 | 17 | 85 |
| 1127 | 106 | 17 | 110 |
| 1128 | 106 | 17 | 132 |
| 1129 | 106 | 17 | 165 |
| 1130 | 106 | 17 | 235 |
| 1131 | 106 | 25 | 1 |
| 1132 | 106 | 25 | 2 |
| 1133 | 106 | 25 | 3 |
| 1134 | 106 | 25 | 4 |
| 1135 | 106 | 25 | 74 |
| 1136 | 106 | 25 | 85 |
| 1137 | 106 | 25 | 110 |
| 1138 | 106 | 25 | 132 |
| 1139 | 106 | 25 | 165 |
| 1140 | 106 | 25 | 235 |
| 1141 | 106 | 45 | 1 |
| 1142 | 106 | 45 | 2 |
| 1143 | 106 | 45 | 3 |
| 1144 | 106 | 45 | 4 |
| 1145 | 106 | 45 | 74 |
| 1146 | 106 | 45 | 85 |
| 1147 | 106 | 45 | 110 |
| 1148 | 106 | 45 | 132 |
| 1149 | 106 | 45 | 165 |
| 1150 | 106 | 45 | 235 |
| 1151 | 106 | 65 | 1 |
| 1152 | 106 | 65 | 2 |
| 1153 | 106 | 65 | 3 |
| 1154 | 106 | 65 | 4 |
| 1155 | 106 | 65 | 74 |
| 1156 | 106 | 65 | 85 |
| 1157 | 106 | 65 | 110 |
| 1158 | 106 | 65 | 132 |
| 1159 | 106 | 65 | 165 |
| 1160 | 106 | 65 | 235 |
| 1161 | 106 | 74 | 1 |
| 1162 | 106 | 74 | 2 |
| 1163 | 106 | 74 | 3 |
| 1164 | 106 | 74 | 4 |
| 1165 | 106 | 74 | 74 |
| 1166 | 106 | 74 | 85 |
| 1167 | 106 | 74 | 110 |
| 1168 | 106 | 74 | 132 |
| 1169 | 106 | 74 | 165 |
| 1170 | 106 | 74 | 235 |
| 1171 | 106 | 105 | 1 |
| 1172 | 106 | 105 | 2 |
| 1173 | 106 | 105 | 3 |
| 1174 | 106 | 105 | 4 |
| 1175 | 106 | 105 | 74 |
| 1176 | 106 | 105 | 85 |
| 1177 | 106 | 105 | 110 |
| 1178 | 106 | 105 | 132 |
| 1179 | 106 | 105 | 165 |
| 1180 | 106 | 105 | 235 |
| 1181 | 106 | 165 | 1 |
| 1182 | 106 | 165 | 2 |
| 1183 | 106 | 165 | 3 |
| 1184 | 106 | 165 | 4 |
| 1185 | 106 | 165 | 74 |
| 1186 | 106 | 165 | 85 |
| 1187 | 106 | 165 | 110 |
| 1188 | 106 | 165 | 132 |
| 1189 | 106 | 165 | 165 |
| 1190 | 106 | 165 | 235 |
| 1191 | 106 | 173 | 1 |
| 1192 | 106 | 173 | 2 |
| 1193 | 106 | 173 | 3 |
| 1194 | 106 | 173 | 4 |
| 1195 | 106 | 173 | 74 |
| 1196 | 106 | 173 | 85 |
| 1197 | 106 | 173 | 110 |
| 1198 | 106 | 173 | 132 |
| 1199 | 106 | 173 | 165 |
| 1200 | 106 | 173 | 235 |
| 1201 | 107 | 2 | 1 |
| 1202 | 107 | 2 | 2 |
| 1203 | 107 | 2 | 3 |
| 1204 | 107 | 2 | 4 |
| 1205 | 107 | 2 | 74 |
| 1206 | 107 | 2 | 85 |
| 1207 | 107 | 2 | 110 |
| 1208 | 107 | 2 | 132 |
| 1209 | 107 | 2 | 165 |
| 1210 | 107 | 2 | 235 |
| 1211 | 107 | 6 | 1 |
| 1212 | 107 | 6 | 2 |
| 1213 | 107 | 6 | 3 |
| 1214 | 107 | 6 | 4 |
| 1215 | 107 | 6 | 74 |
| 1216 | 107 | 6 | 85 |
| 1217 | 107 | 6 | 110 |
| 1218 | 107 | 6 | 132 |
| 1219 | 107 | 6 | 165 |
| 1220 | 107 | 6 | 235 |
| 1221 | 107 | 17 | 1 |
| 1222 | 107 | 17 | 2 |
| 1223 | 107 | 17 | 3 |
| 1224 | 107 | 17 | 4 |
| 1225 | 107 | 17 | 74 |
| 1226 | 107 | 17 | 85 |
| 1227 | 107 | 17 | 110 |
| 1228 | 107 | 17 | 132 |
| 1229 | 107 | 17 | 165 |
| 1230 | 107 | 17 | 235 |
| 1231 | 107 | 25 | 1 |
| 1232 | 107 | 25 | 2 |
| 1233 | 107 | 25 | 3 |
| 1234 | 107 | 25 | 4 |
| 1235 | 107 | 25 | 74 |
| 1236 | 107 | 25 | 85 |
| 1237 | 107 | 25 | 110 |
| 1238 | 107 | 25 | 132 |
| 1239 | 107 | 25 | 165 |
| 1240 | 107 | 25 | 235 |
| 1241 | 107 | 45 | 1 |
| 1242 | 107 | 45 | 2 |
| 1243 | 107 | 45 | 3 |
| 1244 | 107 | 45 | 4 |
| 1245 | 107 | 45 | 74 |
| 1246 | 107 | 45 | 85 |
| 1247 | 107 | 45 | 110 |
| 1248 | 107 | 45 | 132 |
| 1249 | 107 | 45 | 165 |
| 1250 | 107 | 45 | 235 |
| 1251 | 107 | 65 | 1 |
| 1252 | 107 | 65 | 2 |
| 1253 | 107 | 65 | 3 |
| 1254 | 107 | 65 | 4 |
| 1255 | 107 | 65 | 74 |
| 1256 | 107 | 65 | 85 |
| 1257 | 107 | 65 | 110 |
| 1258 | 107 | 65 | 132 |
| 1259 | 107 | 65 | 165 |
| 1260 | 107 | 65 | 235 |
| 1261 | 107 | 74 | 1 |
| 1262 | 107 | 74 | 2 |
| 1263 | 107 | 74 | 3 |
| 1264 | 107 | 74 | 4 |
| 1265 | 107 | 74 | 74 |
| 1266 | 107 | 74 | 85 |
| 1267 | 107 | 74 | 110 |
| 1268 | 107 | 74 | 132 |
| 1269 | 107 | 74 | 165 |
| 1270 | 107 | 74 | 235 |
| 1271 | 107 | 105 | 1 |
| 1272 | 107 | 105 | 2 |
| 1273 | 107 | 105 | 3 |
| 1274 | 107 | 105 | 4 |
| 1275 | 107 | 105 | 74 |
| 1276 | 107 | 105 | 85 |
| 1277 | 107 | 105 | 110 |
| 1278 | 107 | 105 | 132 |
| 1279 | 107 | 105 | 165 |
| 1280 | 107 | 105 | 235 |
| 1281 | 107 | 165 | 1 |
| 1282 | 107 | 165 | 2 |
| 1283 | 107 | 165 | 3 |
| 1284 | 107 | 165 | 4 |
| 1285 | 107 | 165 | 74 |
| 1286 | 107 | 165 | 85 |
| 1287 | 107 | 165 | 110 |
| 1288 | 107 | 165 | 132 |
| 1289 | 107 | 165 | 165 |
| 1290 | 107 | 165 | 235 |
| 1291 | 107 | 173 | 1 |
| 1292 | 107 | 173 | 2 |
| 1293 | 107 | 173 | 3 |
| 1294 | 107 | 173 | 4 |
| 1295 | 107 | 173 | 74 |
| 1296 | 107 | 173 | 85 |
| 1297 | 107 | 173 | 110 |
| 1298 | 107 | 173 | 132 |
| 1299 | 107 | 173 | 165 |
| 1300 | 107 | 173 | 235 |
| 1301 | 108 | 2 | 1 |
| 1302 | 108 | 2 | 2 |
| 1303 | 108 | 2 | 3 |
| 1304 | 108 | 2 | 4 |
| 1305 | 108 | 2 | 74 |
| 1306 | 108 | 2 | 85 |
| 1307 | 108 | 2 | 110 |
| 1308 | 108 | 2 | 132 |
| 1309 | 108 | 2 | 165 |
| 1310 | 108 | 2 | 235 |
| 1311 | 108 | 6 | 1 |
| 1312 | 108 | 6 | 2 |
| 1313 | 108 | 6 | 3 |
| 1314 | 108 | 6 | 4 |
| 1315 | 108 | 6 | 74 |
| 1316 | 108 | 6 | 85 |
| 1317 | 108 | 6 | 110 |
| 1318 | 108 | 6 | 132 |
| 1319 | 108 | 6 | 165 |
| 1320 | 108 | 6 | 235 |
| 1321 | 108 | 17 | 1 |
| 1322 | 108 | 17 | 2 |
| 1323 | 108 | 17 | 3 |
| 1324 | 108 | 17 | 4 |
| 1325 | 108 | 17 | 74 |
| 1326 | 108 | 17 | 85 |
| 1327 | 108 | 17 | 110 |
| 1328 | 108 | 17 | 132 |
| 1329 | 108 | 17 | 165 |
| 1330 | 108 | 17 | 235 |
| 1331 | 108 | 25 | 1 |
| 1332 | 108 | 25 | 2 |
| 1333 | 108 | 25 | 3 |
| 1334 | 108 | 25 | 4 |
| 1335 | 108 | 25 | 74 |
| 1336 | 108 | 25 | 85 |
| 1337 | 108 | 25 | 110 |
| 1338 | 108 | 25 | 132 |
| 1339 | 108 | 25 | 165 |
| 1340 | 108 | 25 | 235 |
| 1341 | 108 | 45 | 1 |
| 1342 | 108 | 45 | 2 |
| 1343 | 108 | 45 | 3 |
| 1344 | 108 | 45 | 4 |
| 1345 | 108 | 45 | 74 |
| 1346 | 108 | 45 | 85 |
| 1347 | 108 | 45 | 110 |
| 1348 | 108 | 45 | 132 |
| 1349 | 108 | 45 | 165 |
| 1350 | 108 | 45 | 235 |
| 1351 | 108 | 65 | 1 |
| 1352 | 108 | 65 | 2 |
| 1353 | 108 | 65 | 3 |
| 1354 | 108 | 65 | 4 |
| 1355 | 108 | 65 | 74 |
| 1356 | 108 | 65 | 85 |
| 1357 | 108 | 65 | 110 |
| 1358 | 108 | 65 | 132 |
| 1359 | 108 | 65 | 165 |
| 1360 | 108 | 65 | 235 |
| 1361 | 108 | 74 | 1 |
| 1362 | 108 | 74 | 2 |
| 1363 | 108 | 74 | 3 |
| 1364 | 108 | 74 | 4 |
| 1365 | 108 | 74 | 74 |
| 1366 | 108 | 74 | 85 |
| 1367 | 108 | 74 | 110 |
| 1368 | 108 | 74 | 132 |
| 1369 | 108 | 74 | 165 |
| 1370 | 108 | 74 | 235 |
| 1371 | 108 | 105 | 1 |
| 1372 | 108 | 105 | 2 |
| 1373 | 108 | 105 | 3 |
| 1374 | 108 | 105 | 4 |
| 1375 | 108 | 105 | 74 |
| 1376 | 108 | 105 | 85 |
| 1377 | 108 | 105 | 110 |
| 1378 | 108 | 105 | 132 |
| 1379 | 108 | 105 | 165 |
| 1380 | 108 | 105 | 235 |
| 1381 | 108 | 165 | 1 |
| 1382 | 108 | 165 | 2 |
| 1383 | 108 | 165 | 3 |
| 1384 | 108 | 165 | 4 |
| 1385 | 108 | 165 | 74 |
| 1386 | 108 | 165 | 85 |
| 1387 | 108 | 165 | 110 |
| 1388 | 108 | 165 | 132 |
| 1389 | 108 | 165 | 165 |
| 1390 | 108 | 165 | 235 |
| 1391 | 108 | 173 | 1 |
| 1392 | 108 | 173 | 2 |
| 1393 | 108 | 173 | 3 |
| 1394 | 108 | 173 | 4 |
| 1395 | 108 | 173 | 74 |
| 1396 | 108 | 173 | 85 |
| 1397 | 108 | 173 | 110 |
| 1398 | 108 | 173 | 132 |
| 1399 | 108 | 173 | 165 |
| 1400 | 108 | 173 | 235 |
| 1401 | 123 | 2 | 1 |
| 1402 | 123 | 2 | 2 |
| 1403 | 123 | 2 | 3 |
| 1404 | 123 | 2 | 4 |
| 1405 | 123 | 2 | 74 |
| 1406 | 123 | 2 | 85 |
| 1407 | 123 | 2 | 110 |
| 1408 | 123 | 2 | 132 |
| 1409 | 123 | 2 | 165 |
| 1410 | 123 | 2 | 235 |
| 1411 | 123 | 6 | 1 |
| 1412 | 123 | 6 | 2 |
| 1413 | 123 | 6 | 3 |
| 1414 | 123 | 6 | 4 |
| 1415 | 123 | 6 | 74 |
| 1416 | 123 | 6 | 85 |
| 1417 | 123 | 6 | 110 |
| 1418 | 123 | 6 | 132 |
| 1419 | 123 | 6 | 165 |
| 1420 | 123 | 6 | 235 |
| 1421 | 123 | 17 | 1 |
| 1422 | 123 | 17 | 2 |
| 1423 | 123 | 17 | 3 |
| 1424 | 123 | 17 | 4 |
| 1425 | 123 | 17 | 74 |
| 1426 | 123 | 17 | 85 |
| 1427 | 123 | 17 | 110 |
| 1428 | 123 | 17 | 132 |
| 1429 | 123 | 17 | 165 |
| 1430 | 123 | 17 | 235 |
| 1431 | 123 | 25 | 1 |
| 1432 | 123 | 25 | 2 |
| 1433 | 123 | 25 | 3 |
| 1434 | 123 | 25 | 4 |
| 1435 | 123 | 25 | 74 |
| 1436 | 123 | 25 | 85 |
| 1437 | 123 | 25 | 110 |
| 1438 | 123 | 25 | 132 |
| 1439 | 123 | 25 | 165 |
| 1440 | 123 | 25 | 235 |
| 1441 | 123 | 45 | 1 |
| 1442 | 123 | 45 | 2 |
| 1443 | 123 | 45 | 3 |
| 1444 | 123 | 45 | 4 |
| 1445 | 123 | 45 | 74 |
| 1446 | 123 | 45 | 85 |
| 1447 | 123 | 45 | 110 |
| 1448 | 123 | 45 | 132 |
| 1449 | 123 | 45 | 165 |
| 1450 | 123 | 45 | 235 |
| 1451 | 123 | 65 | 1 |
| 1452 | 123 | 65 | 2 |
| 1453 | 123 | 65 | 3 |
| 1454 | 123 | 65 | 4 |
| 1455 | 123 | 65 | 74 |
| 1456 | 123 | 65 | 85 |
| 1457 | 123 | 65 | 110 |
| 1458 | 123 | 65 | 132 |
| 1459 | 123 | 65 | 165 |
| 1460 | 123 | 65 | 235 |
| 1461 | 123 | 74 | 1 |
| 1462 | 123 | 74 | 2 |
| 1463 | 123 | 74 | 3 |
| 1464 | 123 | 74 | 4 |
| 1465 | 123 | 74 | 74 |
| 1466 | 123 | 74 | 85 |
| 1467 | 123 | 74 | 110 |
| 1468 | 123 | 74 | 132 |
| 1469 | 123 | 74 | 165 |
| 1470 | 123 | 74 | 235 |
| 1471 | 123 | 105 | 1 |
| 1472 | 123 | 105 | 2 |
| 1473 | 123 | 105 | 3 |
| 1474 | 123 | 105 | 4 |
| 1475 | 123 | 105 | 74 |
| 1476 | 123 | 105 | 85 |
| 1477 | 123 | 105 | 110 |
| 1478 | 123 | 105 | 132 |
| 1479 | 123 | 105 | 165 |
| 1480 | 123 | 105 | 235 |
| 1481 | 123 | 165 | 1 |
| 1482 | 123 | 165 | 2 |
| 1483 | 123 | 165 | 3 |
| 1484 | 123 | 165 | 4 |
| 1485 | 123 | 165 | 74 |
| 1486 | 123 | 165 | 85 |
| 1487 | 123 | 165 | 110 |
| 1488 | 123 | 165 | 132 |
| 1489 | 123 | 165 | 165 |
| 1490 | 123 | 165 | 235 |
| 1491 | 123 | 173 | 1 |
| 1492 | 123 | 173 | 2 |
| 1493 | 123 | 173 | 3 |
| 1494 | 123 | 173 | 4 |
| 1495 | 123 | 173 | 74 |
| 1496 | 123 | 173 | 85 |
| 1497 | 123 | 173 | 110 |
| 1498 | 123 | 173 | 132 |
| 1499 | 123 | 173 | 165 |
| 1500 | 123 | 173 | 235 |
| 1501 | 126 | 2 | 1 |
| 1502 | 126 | 2 | 2 |
| 1503 | 126 | 2 | 3 |
| 1504 | 126 | 2 | 4 |
| 1505 | 126 | 2 | 74 |
| 1506 | 126 | 2 | 85 |
| 1507 | 126 | 2 | 110 |
| 1508 | 126 | 2 | 132 |
| 1509 | 126 | 2 | 165 |
| 1510 | 126 | 2 | 235 |
| 1511 | 126 | 6 | 1 |
| 1512 | 126 | 6 | 2 |
| 1513 | 126 | 6 | 3 |
| 1514 | 126 | 6 | 4 |
| 1515 | 126 | 6 | 74 |
| 1516 | 126 | 6 | 85 |
| 1517 | 126 | 6 | 110 |
| 1518 | 126 | 6 | 132 |
| 1519 | 126 | 6 | 165 |
| 1520 | 126 | 6 | 235 |
| 1521 | 126 | 17 | 1 |
| 1522 | 126 | 17 | 2 |
| 1523 | 126 | 17 | 3 |
| 1524 | 126 | 17 | 4 |
| 1525 | 126 | 17 | 74 |
| 1526 | 126 | 17 | 85 |
| 1527 | 126 | 17 | 110 |
| 1528 | 126 | 17 | 132 |
| 1529 | 126 | 17 | 165 |
| 1530 | 126 | 17 | 235 |
| 1531 | 126 | 25 | 1 |
| 1532 | 126 | 25 | 2 |
| 1533 | 126 | 25 | 3 |
| 1534 | 126 | 25 | 4 |
| 1535 | 126 | 25 | 74 |
| 1536 | 126 | 25 | 85 |
| 1537 | 126 | 25 | 110 |
| 1538 | 126 | 25 | 132 |
| 1539 | 126 | 25 | 165 |
| 1540 | 126 | 25 | 235 |
| 1541 | 126 | 45 | 1 |
| 1542 | 126 | 45 | 2 |
| 1543 | 126 | 45 | 3 |
| 1544 | 126 | 45 | 4 |
| 1545 | 126 | 45 | 74 |
| 1546 | 126 | 45 | 85 |
| 1547 | 126 | 45 | 110 |
| 1548 | 126 | 45 | 132 |
| 1549 | 126 | 45 | 165 |
| 1550 | 126 | 45 | 235 |
| 1551 | 126 | 65 | 1 |
| 1552 | 126 | 65 | 2 |
| 1553 | 126 | 65 | 3 |
| 1554 | 126 | 65 | 4 |
| 1555 | 126 | 65 | 74 |
| 1556 | 126 | 65 | 85 |
| 1557 | 126 | 65 | 110 |
| 1558 | 126 | 65 | 132 |
| 1559 | 126 | 65 | 165 |
| 1560 | 126 | 65 | 235 |
| 1561 | 126 | 74 | 1 |
| 1562 | 126 | 74 | 2 |
| 1563 | 126 | 74 | 3 |
| 1564 | 126 | 74 | 4 |
| 1565 | 126 | 74 | 74 |
| 1566 | 126 | 74 | 85 |
| 1567 | 126 | 74 | 110 |
| 1568 | 126 | 74 | 132 |
| 1569 | 126 | 74 | 165 |
| 1570 | 126 | 74 | 235 |
| 1571 | 126 | 105 | 1 |
| 1572 | 126 | 105 | 2 |
| 1573 | 126 | 105 | 3 |
| 1574 | 126 | 105 | 4 |
| 1575 | 126 | 105 | 74 |
| 1576 | 126 | 105 | 85 |
| 1577 | 126 | 105 | 110 |
| 1578 | 126 | 105 | 132 |
| 1579 | 126 | 105 | 165 |
| 1580 | 126 | 105 | 235 |
| 1581 | 126 | 165 | 1 |
| 1582 | 126 | 165 | 2 |
| 1583 | 126 | 165 | 3 |
| 1584 | 126 | 165 | 4 |
| 1585 | 126 | 165 | 74 |
| 1586 | 126 | 165 | 85 |
| 1587 | 126 | 165 | 110 |
| 1588 | 126 | 165 | 132 |
| 1589 | 126 | 165 | 165 |
| 1590 | 126 | 165 | 235 |
| 1591 | 126 | 173 | 1 |
| 1592 | 126 | 173 | 2 |
| 1593 | 126 | 173 | 3 |
| 1594 | 126 | 173 | 4 |
| 1595 | 126 | 173 | 74 |
| 1596 | 126 | 173 | 85 |
| 1597 | 126 | 173 | 110 |
| 1598 | 126 | 173 | 132 |
| 1599 | 126 | 173 | 165 |
| 1600 | 126 | 173 | 235 |
| 1601 | 132 | 2 | 1 |
| 1602 | 132 | 2 | 2 |
| 1603 | 132 | 2 | 3 |
| 1604 | 132 | 2 | 4 |
| 1605 | 132 | 2 | 74 |
| 1606 | 132 | 2 | 85 |
| 1607 | 132 | 2 | 110 |
| 1608 | 132 | 2 | 132 |
| 1609 | 132 | 2 | 165 |
| 1610 | 132 | 2 | 235 |
| 1611 | 132 | 6 | 1 |
| 1612 | 132 | 6 | 2 |
| 1613 | 132 | 6 | 3 |
| 1614 | 132 | 6 | 4 |
| 1615 | 132 | 6 | 74 |
| 1616 | 132 | 6 | 85 |
| 1617 | 132 | 6 | 110 |
| 1618 | 132 | 6 | 132 |
| 1619 | 132 | 6 | 165 |
| 1620 | 132 | 6 | 235 |
| 1621 | 132 | 17 | 1 |
| 1622 | 132 | 17 | 2 |
| 1623 | 132 | 17 | 3 |
| 1624 | 132 | 17 | 4 |
| 1625 | 132 | 17 | 74 |
| 1626 | 132 | 17 | 85 |
| 1627 | 132 | 17 | 110 |
| 1628 | 132 | 17 | 132 |
| 1629 | 132 | 17 | 165 |
| 1630 | 132 | 17 | 235 |
| 1631 | 132 | 25 | 1 |
| 1632 | 132 | 25 | 2 |
| 1633 | 132 | 25 | 3 |
| 1634 | 132 | 25 | 4 |
| 1635 | 132 | 25 | 74 |
| 1636 | 132 | 25 | 85 |
| 1637 | 132 | 25 | 110 |
| 1638 | 132 | 25 | 132 |
| 1639 | 132 | 25 | 165 |
| 1640 | 132 | 25 | 235 |
| 1641 | 132 | 45 | 1 |
| 1642 | 132 | 45 | 2 |
| 1643 | 132 | 45 | 3 |
| 1644 | 132 | 45 | 4 |
| 1645 | 132 | 45 | 74 |
| 1646 | 132 | 45 | 85 |
| 1647 | 132 | 45 | 110 |
| 1648 | 132 | 45 | 132 |
| 1649 | 132 | 45 | 165 |
| 1650 | 132 | 45 | 235 |
| 1651 | 132 | 65 | 1 |
| 1652 | 132 | 65 | 2 |
| 1653 | 132 | 65 | 3 |
| 1654 | 132 | 65 | 4 |
| 1655 | 132 | 65 | 74 |
| 1656 | 132 | 65 | 85 |
| 1657 | 132 | 65 | 110 |
| 1658 | 132 | 65 | 132 |
| 1659 | 132 | 65 | 165 |
| 1660 | 132 | 65 | 235 |
| 1661 | 132 | 74 | 1 |
| 1662 | 132 | 74 | 2 |
| 1663 | 132 | 74 | 3 |
| 1664 | 132 | 74 | 4 |
| 1665 | 132 | 74 | 74 |
| 1666 | 132 | 74 | 85 |
| 1667 | 132 | 74 | 110 |
| 1668 | 132 | 74 | 132 |
| 1669 | 132 | 74 | 165 |
| 1670 | 132 | 74 | 235 |
| 1671 | 132 | 105 | 1 |
| 1672 | 132 | 105 | 2 |
| 1673 | 132 | 105 | 3 |
| 1674 | 132 | 105 | 4 |
| 1675 | 132 | 105 | 74 |
| 1676 | 132 | 105 | 85 |
| 1677 | 132 | 105 | 110 |
| 1678 | 132 | 105 | 132 |
| 1679 | 132 | 105 | 165 |
| 1680 | 132 | 105 | 235 |
| 1681 | 132 | 165 | 1 |
| 1682 | 132 | 165 | 2 |
| 1683 | 132 | 165 | 3 |
| 1684 | 132 | 165 | 4 |
| 1685 | 132 | 165 | 74 |
| 1686 | 132 | 165 | 85 |
| 1687 | 132 | 165 | 110 |
| 1688 | 132 | 165 | 132 |
| 1689 | 132 | 165 | 165 |
| 1690 | 132 | 165 | 235 |
| 1691 | 132 | 173 | 1 |
| 1692 | 132 | 173 | 2 |
| 1693 | 132 | 173 | 3 |
| 1694 | 132 | 173 | 4 |
| 1695 | 132 | 173 | 74 |
| 1696 | 132 | 173 | 85 |
| 1697 | 132 | 173 | 110 |
| 1698 | 132 | 173 | 132 |
| 1699 | 132 | 173 | 165 |
| 1700 | 132 | 173 | 235 |
| 1701 | 133 | 2 | 1 |
| 1702 | 133 | 2 | 2 |
| 1703 | 133 | 2 | 3 |
| 1704 | 133 | 2 | 4 |
| 1705 | 133 | 2 | 74 |
| 1706 | 133 | 2 | 85 |
| 1707 | 133 | 2 | 110 |
| 1708 | 133 | 2 | 132 |
| 1709 | 133 | 2 | 165 |
| 1710 | 133 | 2 | 235 |
| 1711 | 133 | 6 | 1 |
| 1712 | 133 | 6 | 2 |
| 1713 | 133 | 6 | 3 |
| 1714 | 133 | 6 | 4 |
| 1715 | 133 | 6 | 74 |
| 1716 | 133 | 6 | 85 |
| 1717 | 133 | 6 | 110 |
| 1718 | 133 | 6 | 132 |
| 1719 | 133 | 6 | 165 |
| 1720 | 133 | 6 | 235 |
| 1721 | 133 | 17 | 1 |
| 1722 | 133 | 17 | 2 |
| 1723 | 133 | 17 | 3 |
| 1724 | 133 | 17 | 4 |
| 1725 | 133 | 17 | 74 |
| 1726 | 133 | 17 | 85 |
| 1727 | 133 | 17 | 110 |
| 1728 | 133 | 17 | 132 |
| 1729 | 133 | 17 | 165 |
| 1730 | 133 | 17 | 235 |
| 1731 | 133 | 25 | 1 |
| 1732 | 133 | 25 | 2 |
| 1733 | 133 | 25 | 3 |
| 1734 | 133 | 25 | 4 |
| 1735 | 133 | 25 | 74 |
| 1736 | 133 | 25 | 85 |
| 1737 | 133 | 25 | 110 |
| 1738 | 133 | 25 | 132 |
| 1739 | 133 | 25 | 165 |
| 1740 | 133 | 25 | 235 |
| 1741 | 133 | 45 | 1 |
| 1742 | 133 | 45 | 2 |
| 1743 | 133 | 45 | 3 |
| 1744 | 133 | 45 | 4 |
| 1745 | 133 | 45 | 74 |
| 1746 | 133 | 45 | 85 |
| 1747 | 133 | 45 | 110 |
| 1748 | 133 | 45 | 132 |
| 1749 | 133 | 45 | 165 |
| 1750 | 133 | 45 | 235 |
| 1751 | 133 | 65 | 1 |
| 1752 | 133 | 65 | 2 |
| 1753 | 133 | 65 | 3 |
| 1754 | 133 | 65 | 4 |
| 1755 | 133 | 65 | 74 |
| 1756 | 133 | 65 | 85 |
| 1757 | 133 | 65 | 110 |
| 1758 | 133 | 65 | 132 |
| 1759 | 133 | 65 | 165 |
| 1760 | 133 | 65 | 235 |
| 1761 | 133 | 74 | 1 |
| 1762 | 133 | 74 | 2 |
| 1763 | 133 | 74 | 3 |
| 1764 | 133 | 74 | 4 |
| 1765 | 133 | 74 | 74 |
| 1766 | 133 | 74 | 85 |
| 1767 | 133 | 74 | 110 |
| 1768 | 133 | 74 | 132 |
| 1769 | 133 | 74 | 165 |
| 1770 | 133 | 74 | 235 |
| 1771 | 133 | 105 | 1 |
| 1772 | 133 | 105 | 2 |
| 1773 | 133 | 105 | 3 |
| 1774 | 133 | 105 | 4 |
| 1775 | 133 | 105 | 74 |
| 1776 | 133 | 105 | 85 |
| 1777 | 133 | 105 | 110 |
| 1778 | 133 | 105 | 132 |
| 1779 | 133 | 105 | 165 |
| 1780 | 133 | 105 | 235 |
| 1781 | 133 | 165 | 1 |
| 1782 | 133 | 165 | 2 |
| 1783 | 133 | 165 | 3 |
| 1784 | 133 | 165 | 4 |
| 1785 | 133 | 165 | 74 |
| 1786 | 133 | 165 | 85 |
| 1787 | 133 | 165 | 110 |
| 1788 | 133 | 165 | 132 |
| 1789 | 133 | 165 | 165 |
| 1790 | 133 | 165 | 235 |
| 1791 | 133 | 173 | 1 |
| 1792 | 133 | 173 | 2 |
| 1793 | 133 | 173 | 3 |
| 1794 | 133 | 173 | 4 |
| 1795 | 133 | 173 | 74 |
| 1796 | 133 | 173 | 85 |
| 1797 | 133 | 173 | 110 |
| 1798 | 133 | 173 | 132 |
| 1799 | 133 | 173 | 165 |
| 1800 | 133 | 173 | 235 |
| 1801 | 134 | 2 | 1 |
| 1802 | 134 | 2 | 2 |
| 1803 | 134 | 2 | 3 |
| 1804 | 134 | 2 | 4 |
| 1805 | 134 | 2 | 74 |
| 1806 | 134 | 2 | 85 |
| 1807 | 134 | 2 | 110 |
| 1808 | 134 | 2 | 132 |
| 1809 | 134 | 2 | 165 |
| 1810 | 134 | 2 | 235 |
| 1811 | 134 | 6 | 1 |
| 1812 | 134 | 6 | 2 |
| 1813 | 134 | 6 | 3 |
| 1814 | 134 | 6 | 4 |
| 1815 | 134 | 6 | 74 |
| 1816 | 134 | 6 | 85 |
| 1817 | 134 | 6 | 110 |
| 1818 | 134 | 6 | 132 |
| 1819 | 134 | 6 | 165 |
| 1820 | 134 | 6 | 235 |
| 1821 | 134 | 17 | 1 |
| 1822 | 134 | 17 | 2 |
| 1823 | 134 | 17 | 3 |
| 1824 | 134 | 17 | 4 |
| 1825 | 134 | 17 | 74 |
| 1826 | 134 | 17 | 85 |
| 1827 | 134 | 17 | 110 |
| 1828 | 134 | 17 | 132 |
| 1829 | 134 | 17 | 165 |
| 1830 | 134 | 17 | 235 |
| 1831 | 134 | 25 | 1 |
| 1832 | 134 | 25 | 2 |
| 1833 | 134 | 25 | 3 |
| 1834 | 134 | 25 | 4 |
| 1835 | 134 | 25 | 74 |
| 1836 | 134 | 25 | 85 |
| 1837 | 134 | 25 | 110 |
| 1838 | 134 | 25 | 132 |
| 1839 | 134 | 25 | 165 |
| 1840 | 134 | 25 | 235 |
| 1841 | 134 | 45 | 1 |
| 1842 | 134 | 45 | 2 |
| 1843 | 134 | 45 | 3 |
| 1844 | 134 | 45 | 4 |
| 1845 | 134 | 45 | 74 |
| 1846 | 134 | 45 | 85 |
| 1847 | 134 | 45 | 110 |
| 1848 | 134 | 45 | 132 |
| 1849 | 134 | 45 | 165 |
| 1850 | 134 | 45 | 235 |
| 1851 | 134 | 65 | 1 |
| 1852 | 134 | 65 | 2 |
| 1853 | 134 | 65 | 3 |
| 1854 | 134 | 65 | 4 |
| 1855 | 134 | 65 | 74 |
| 1856 | 134 | 65 | 85 |
| 1857 | 134 | 65 | 110 |
| 1858 | 134 | 65 | 132 |
| 1859 | 134 | 65 | 165 |
| 1860 | 134 | 65 | 235 |
| 1861 | 134 | 74 | 1 |
| 1862 | 134 | 74 | 2 |
| 1863 | 134 | 74 | 3 |
| 1864 | 134 | 74 | 4 |
| 1865 | 134 | 74 | 74 |
| 1866 | 134 | 74 | 85 |
| 1867 | 134 | 74 | 110 |
| 1868 | 134 | 74 | 132 |
| 1869 | 134 | 74 | 165 |
| 1870 | 134 | 74 | 235 |
| 1871 | 134 | 105 | 1 |
| 1872 | 134 | 105 | 2 |
| 1873 | 134 | 105 | 3 |
| 1874 | 134 | 105 | 4 |
| 1875 | 134 | 105 | 74 |
| 1876 | 134 | 105 | 85 |
| 1877 | 134 | 105 | 110 |
| 1878 | 134 | 105 | 132 |
| 1879 | 134 | 105 | 165 |
| 1880 | 134 | 105 | 235 |
| 1881 | 134 | 165 | 1 |
| 1882 | 134 | 165 | 2 |
| 1883 | 134 | 165 | 3 |
| 1884 | 134 | 165 | 4 |
| 1885 | 134 | 165 | 74 |
| 1886 | 134 | 165 | 85 |
| 1887 | 134 | 165 | 110 |
| 1888 | 134 | 165 | 132 |
| 1889 | 134 | 165 | 165 |
| 1890 | 134 | 165 | 235 |
| 1891 | 134 | 173 | 1 |
| 1892 | 134 | 173 | 2 |
| 1893 | 134 | 173 | 3 |
| 1894 | 134 | 173 | 4 |
| 1895 | 134 | 173 | 74 |
| 1896 | 134 | 173 | 85 |
| 1897 | 134 | 173 | 110 |
| 1898 | 134 | 173 | 132 |
| 1899 | 134 | 173 | 165 |
| 1900 | 134 | 173 | 235 |
| 1901 | 135 | 2 | 1 |
| 1902 | 135 | 2 | 2 |
| 1903 | 135 | 2 | 3 |
| 1904 | 135 | 2 | 4 |
| 1905 | 135 | 2 | 74 |
| 1906 | 135 | 2 | 85 |
| 1907 | 135 | 2 | 110 |
| 1908 | 135 | 2 | 132 |
| 1909 | 135 | 2 | 165 |
| 1910 | 135 | 2 | 235 |
| 1911 | 135 | 6 | 1 |
| 1912 | 135 | 6 | 2 |
| 1913 | 135 | 6 | 3 |
| 1914 | 135 | 6 | 4 |
| 1915 | 135 | 6 | 74 |
| 1916 | 135 | 6 | 85 |
| 1917 | 135 | 6 | 110 |
| 1918 | 135 | 6 | 132 |
| 1919 | 135 | 6 | 165 |
| 1920 | 135 | 6 | 235 |
| 1921 | 135 | 17 | 1 |
| 1922 | 135 | 17 | 2 |
| 1923 | 135 | 17 | 3 |
| 1924 | 135 | 17 | 4 |
| 1925 | 135 | 17 | 74 |
| 1926 | 135 | 17 | 85 |
| 1927 | 135 | 17 | 110 |
| 1928 | 135 | 17 | 132 |
| 1929 | 135 | 17 | 165 |
| 1930 | 135 | 17 | 235 |
| 1931 | 135 | 25 | 1 |
| 1932 | 135 | 25 | 2 |
| 1933 | 135 | 25 | 3 |
| 1934 | 135 | 25 | 4 |
| 1935 | 135 | 25 | 74 |
| 1936 | 135 | 25 | 85 |
| 1937 | 135 | 25 | 110 |
| 1938 | 135 | 25 | 132 |
| 1936 | 135 | 25 | 165 |
| 1940 | 135 | 25 | 235 |
| 1941 | 135 | 45 | 1 |
| 1942 | 135 | 45 | 2 |
| 1943 | 135 | 45 | 3 |
| 1944 | 135 | 45 | 4 |
| 1945 | 135 | 45 | 74 |
| 1946 | 135 | 45 | 85 |
| 1947 | 135 | 45 | 110 |
| 1948 | 135 | 45 | 132 |
| 1949 | 135 | 45 | 165 |
| 1950 | 135 | 45 | 235 |
| 1951 | 135 | 65 | 1 |
| 1952 | 135 | 65 | 2 |
| 1953 | 135 | 65 | 3 |
| 1954 | 135 | 65 | 4 |
| 1955 | 135 | 65 | 74 |
| 1956 | 135 | 65 | 85 |
| 1957 | 135 | 65 | 110 |
| 1958 | 135 | 65 | 132 |
| 1959 | 135 | 65 | 165 |
| 1960 | 135 | 65 | 235 |
| 1961 | 135 | 74 | 1 |
| 1962 | 135 | 74 | 2 |
| 1963 | 135 | 74 | 3 |
| 1964 | 135 | 74 | 4 |
| 1965 | 135 | 74 | 74 |
| 1966 | 135 | 74 | 85 |
| 1967 | 135 | 74 | 110 |
| 1968 | 135 | 74 | 132 |
| 1969 | 135 | 74 | 165 |
| 1970 | 135 | 74 | 235 |
| 1971 | 135 | 105 | 1 |
| 1972 | 135 | 105 | 2 |
| 1973 | 135 | 105 | 3 |
| 1974 | 135 | 105 | 4 |
| 1975 | 135 | 105 | 74 |
| 1976 | 135 | 105 | 85 |
| 1977 | 135 | 105 | 110 |
| 1978 | 135 | 105 | 132 |
| 1979 | 135 | 105 | 165 |
| 1980 | 135 | 105 | 235 |
| 1981 | 135 | 165 | 1 |
| 1982 | 135 | 165 | 2 |
| 1983 | 135 | 165 | 3 |
| 1984 | 135 | 165 | 4 |
| 1985 | 135 | 165 | 74 |
| 1986 | 135 | 165 | 85 |
| 1987 | 135 | 165 | 110 |
| 1988 | 135 | 165 | 132 |
| 1989 | 135 | 165 | 165 |
| 1990 | 135 | 165 | 235 |
| 1991 | 135 | 173 | 1 |
| 1992 | 135 | 173 | 2 |
| 1993 | 135 | 173 | 3 |
| 1994 | 135 | 173 | 4 |
| 1995 | 135 | 173 | 74 |
| 1996 | 135 | 173 | 85 |
| 1997 | 135 | 173 | 110 |
| 1998 | 135 | 173 | 132 |
| 1999 | 135 | 173 | 165 |
| 2000 | 135 | 173 | 235 |

The compounds of the formula (I) can be prepared by customary methods of synthetic organic chemistry that are known to those skilled in the art. In the preparation of the compounds, transition metal-catalysed coupling reactions in particular are used, such as Buchwald coupling reactions and Suzuki coupling reactions, and also halogenation reactions.

The invention thus provides a process for preparing a compound of the formula (I) as defined above, characterized in that a diarylamine which is a secondary amine is reacted with a halogen-substituted aromatic or heteroaromatic ring system to give a triarylamine compound which is a tertiary amine. The reaction is preferably effected by a Buchwald coupling reaction.

The halogen-substituted aromatic or heteroaromatic ring system preferably corresponds to a formula (I-X)

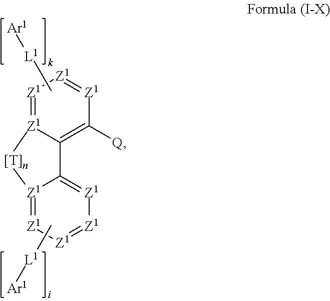

where the variables that occur are as defined above, and where Q is a halogen atom or a triflate or tosylate group, and is preferably Cl, Br or I, more preferably Cl or Br.

The diarylamine preferably corresponds to a formula (I-Y)

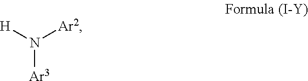

where the variables that occur are as defined above.

The above-described compounds of the formula (I), especially compounds substituted by reactive leaving groups, such as bromine, iodine, chlorine, boronic acid or boronic esters, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C—C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (I), wherein the bond(s) to the polymer, oligomer or dendrimer may be localized at any desired positions substituted by R¹, R², R³ or R⁴ in formula (I). According to the linkage of the compound of formula (I), the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or nonconjugated. The oligomers or polymers of the invention may be linear, branched or dendritic. In the structures having linear linkage, the units of formula (I) may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of formula (I) to be joined via a trivalent or higher-valency group, for example via a trivalent or higher-valency aromatic or heteroaromatic group, to give a branched or dendritic oligomer or polymer.

For the repeat units of formula (I) in oligomers, dendrimers and polymers, the same preferences apply as described above for compounds of formula (I).

For preparation of the oligomers or polymers, the monomers of the invention are homopolymerized or copolymerized with further monomers. Suitable and preferred comonomers are selected from fluorenes (for example according to EP 842208 or WO 2000/22026), spirobifluorenes (for example according to EP 707020, EP 894107 or WO 2006/061181), paraphenylenes (for example according to WO 1992/18552), carbazoles (for example according to WO 2004/070772 or WO 2004/113468), thiophenes (for example according to EP 1028136), dihydrophenanthrenes (for example according to WO 2005/014689 or WO 2007/006383), cis- and trans-indenofluorenes (for example according to WO 2004/041901 or WO 2004/113412), ketones (for example according to WO 2005/040302), phenanthrenes (for example according to WO 2005/104264 or WO 2007/017066) or else a plurality of these units. The polymers, oligomers and dendrimers typically contain still further units, for example emitting (fluorescent or phosphorescent) units, for example vinyltriarylamines (for example according to WO 2007/068325) or phosphorescent metal complexes (for example according to WO 2006/003000), and/or charge transport units, especially those based on triarylamines.

The polymers and oligomers of the invention are generally prepared by polymerization of one or more monomer types, of which at least one monomer leads to repeat units of the formula (I) in the polymer. Suitable polymerization reactions are known to those skilled in the art and are described in the literature. Particularly suitable and preferred polymerization reactions which lead to formation of C—C or C—N bonds are the Suzuki polymerization, the Yamamoto polymerization, the Stille polymerization and the Hartwig-Buchwald polymerization.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene heptylbenzene, octylbenzene, 1,1-bis(3,4-dimethyl phenyl)ethane or mixtures of these solvents.

The invention therefore further provides a formulation, especially a solution, dispersion or emulsion, comprising at least one compound of formula (I) and at least one solvent, preferably an organic solvent. The way in which such solutions can be prepared is known to those skilled in the art and is described, for example, in WO 2002/072714, WO 2003/019694 and the literature cited therein.

The compounds of the invention are suitable for use in electronic devices, especially in organic electroluminescent devices (OLEDs). Depending on the substitution, the compounds are used in different functions and layers.

The invention therefore further provides for the use of the compound of formula (I) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides, as already set out above, an electronic device comprising at least one compound of formula (I). This electronic device is preferably selected from the abovementioned devices.

It is more preferably an organic electroluminescent device (OLED) comprising anode, cathode and at least one emitting layer, characterized in that at least one organic layer, which may be an emitting layer, a hole-transporting layer or another layer, comprises at least one compound of formula (I).

Apart from the cathode, anode and emitting layer, the organic electroluminescent device may also comprise further layers. These are selected, for example, from in each case one or more hole injection layers, hole transport layers, hole blocker layers, electron transport layers, electron injection layers, electron blocker layers, exciton blocker layers, interlayers, charge generation layers (IDMC 2003, Taiwan; Session 21 OLED (5), T. Matsumoto, T. Nakada, J. Endo, K. Mori, N. Kawamura, A. Yokoi, J. Kido, Multiphoton Organic EL Device Having Charge Generation Layer) and/or organic or inorganic p/n junctions.

The sequence of the layers of the organic electroluminescent device comprising the compound of the formula (I) is preferably as follows: anode-hole injection layer-hole transport layer-optionally further hole transport layer(s)-optionally electron blocker layer-emitting layer-optionally hole blocker layer-electron transport layer-electron injection layer-cathode. It is additionally possible for further layers to be present in the OLED.

The organic electroluminescent device of the invention may contain two or more emitting layers. More preferably, these emission layers in this case have several emission maxima between 380 nm and 750 nm overall, such that the overall result is white emission; in other words, various emitting compounds which may fluoresce or phosphoresce and which emit blue, green, yellow, orange or red light are used in the emitting layers. Especially preferred are three-layer systems, i.e. systems having three emitting layers, where the three layers show blue, green and orange or red emission (for the basic construction see, for example, WO 2005/011013).

The compounds of the invention are preferably present here in a hole transport layer, hole injection layer, electron blocker layer, emitting layer, hole-blocking layer and/or electron-transporting layer, more preferably in an emitting layer as matrix material, in a hole blocker layer and/or in an electron transport layer.

It is preferable in accordance with the invention when the compound of formula (I) is used in an electronic device comprising one or more phosphorescent emitting compounds. In this case, the compound may be present in different layers, preferably in a hole transport layer, an electron blocker layer, a hole injection layer, an emitting layer, a hole blocker layer and/or an electron transport layer. More preferably, it is present in an emitting layer in combination with a phosphorescent emitting compound.

The term “phosphorescent emitting compounds” typically encompasses compounds where the emission of light is effected through a spin-forbidden transition, for example a transition from an excited triplet state or a state having a higher spin quantum number, for example a quintet state.

Suitable phosphorescent emitting compounds (=triplet emitters) are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitting compounds, compounds containing copper, molybdenum, tungsten, rhenium, ruthenium, osmium, rhodium, iridium, palladium, platinum, silver, gold or europium, especially compounds containing iridium, platinum or copper. In the context of the present invention, all luminescent iridium, platinum or copper complexes are considered to be phosphorescent emitting compounds.

Examples of the above-described emitting compounds can be found in applications WO 00/70655, WO 01/41512, WO 02/02714, WO 02/15645, EP 1191613, EP 1191612, EP 1191614, WO 05/033244, WO 05/019373 and US 2005/0258742. In general, all phosphorescent complexes as used for phosphorescent OLEDs according to the prior art and as known to those skilled in the art in the field of organic electroluminescent devices are suitable. It is also possible for the person skilled in the art, without exercising inventive skill, to use further phosphorescent complexes in combination with the compounds of formula (I) in organic electroluminescent devices. Further examples are listed in the following table:

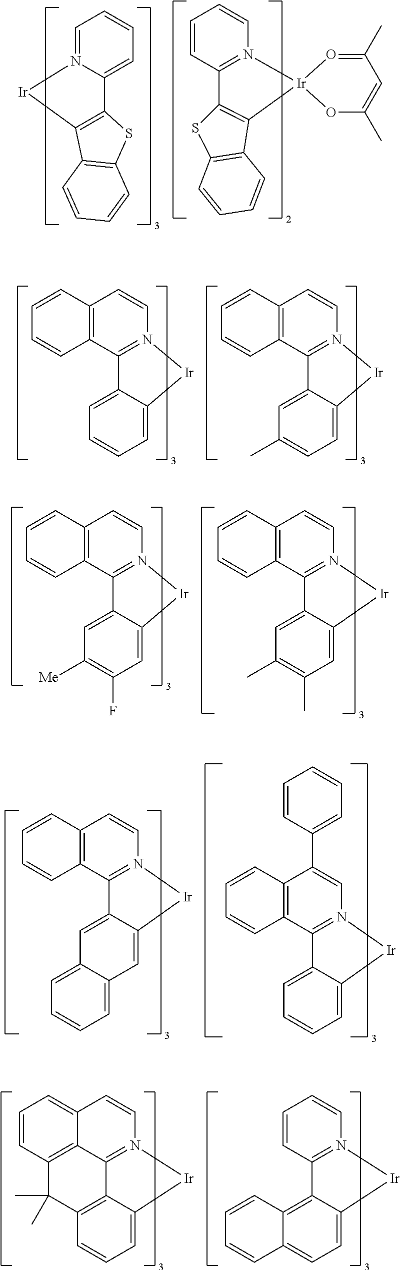

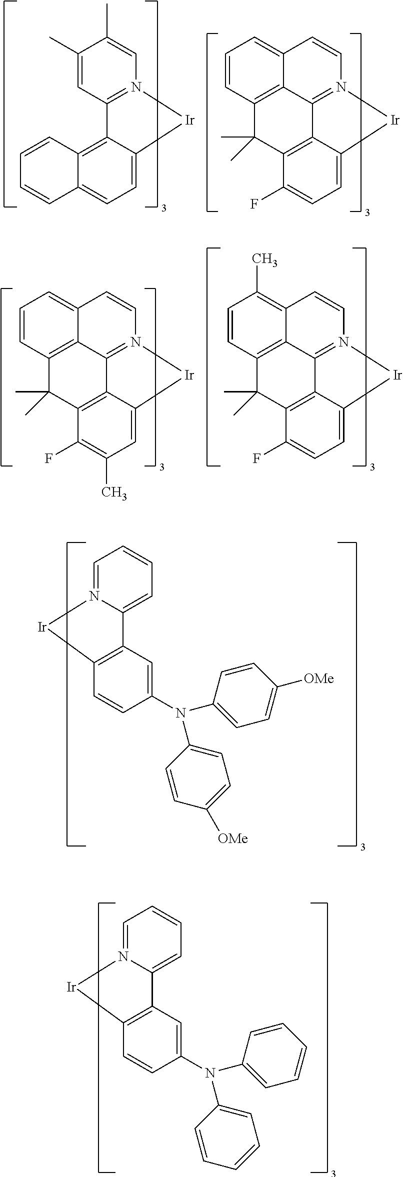

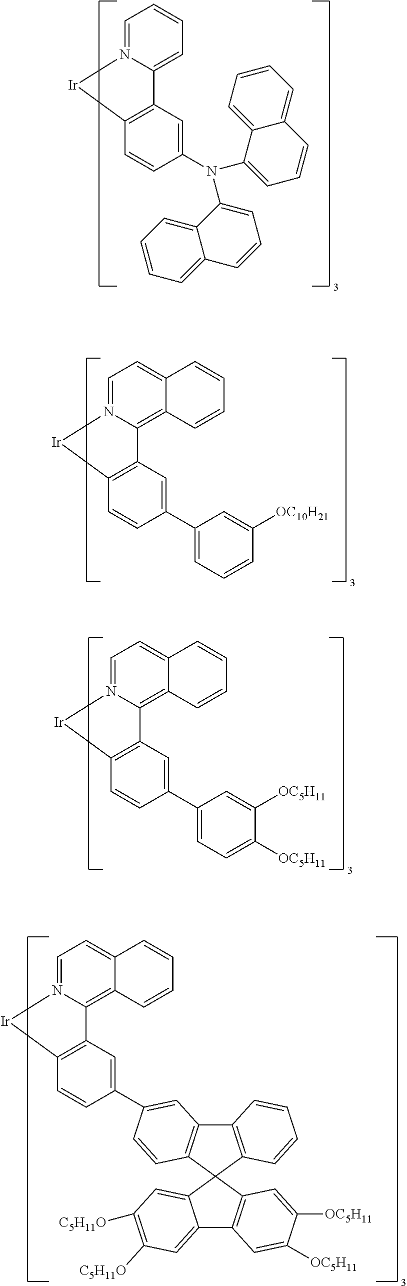

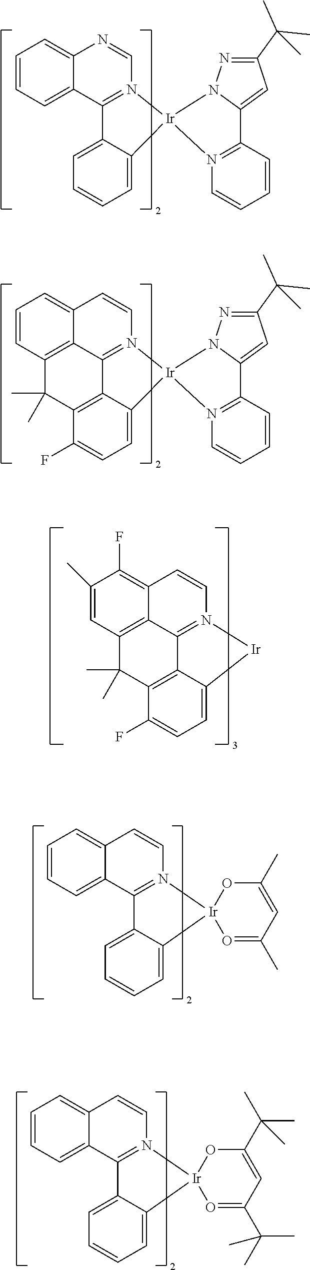

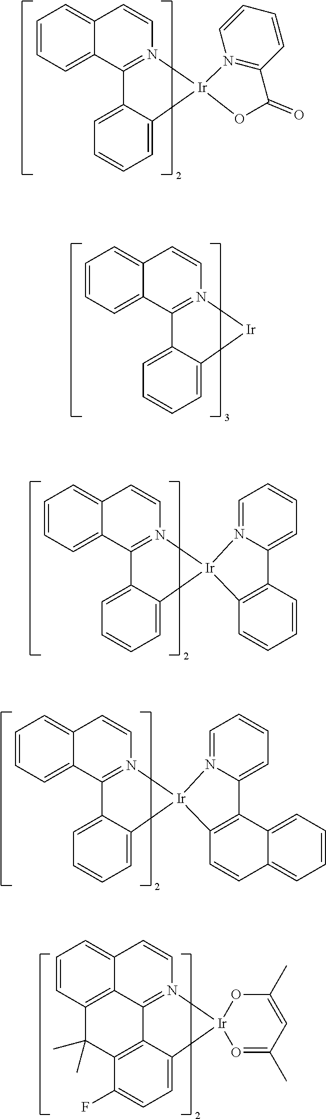

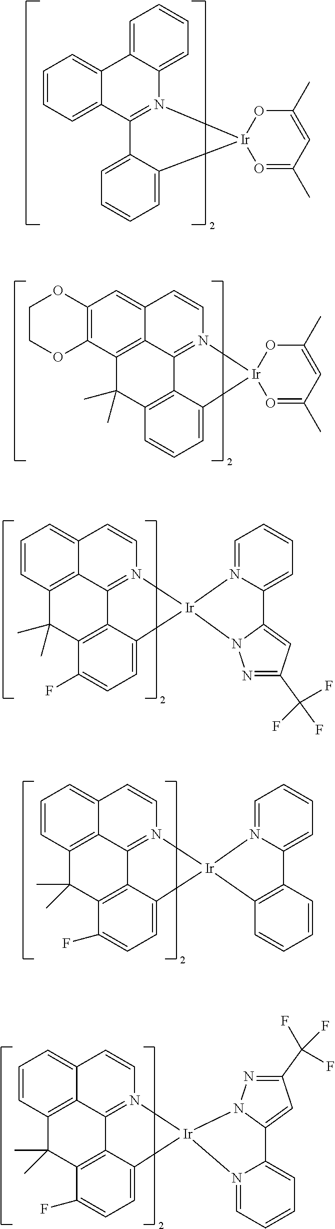

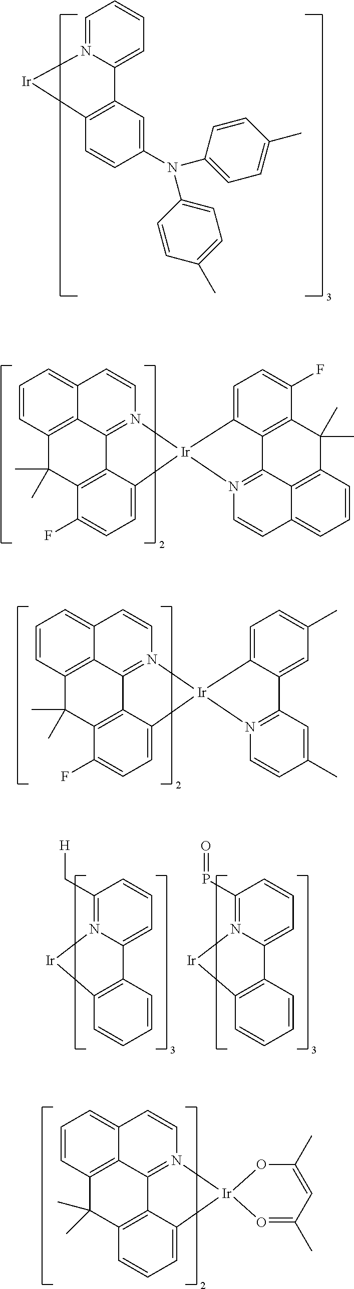

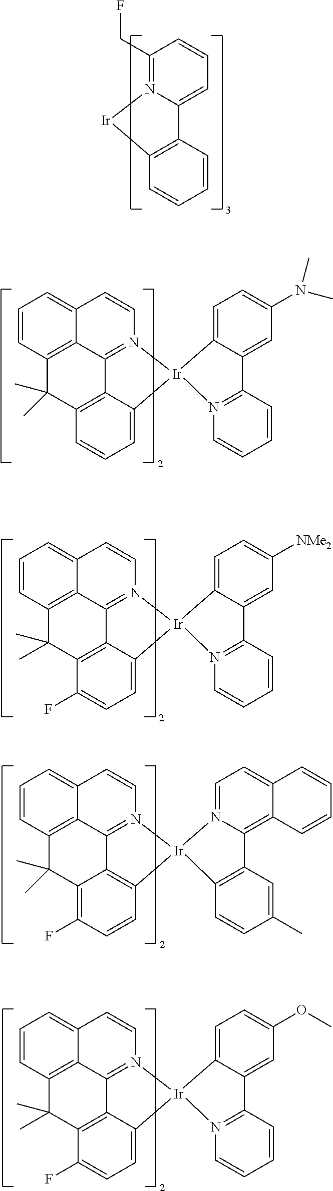

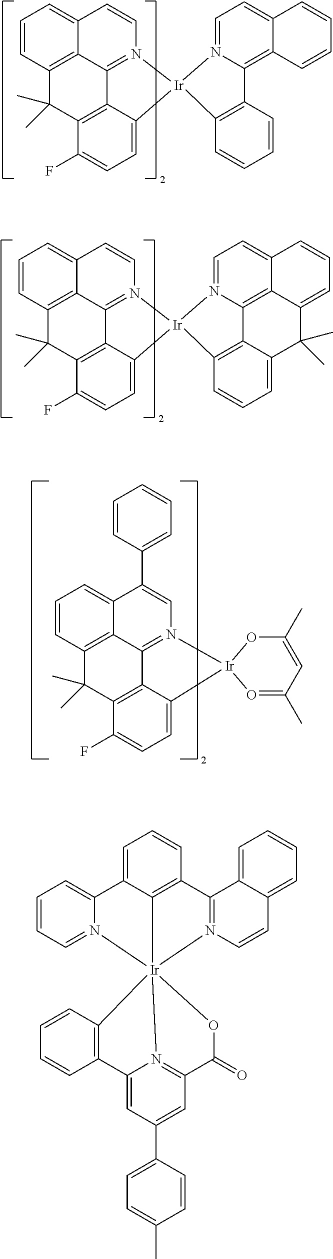

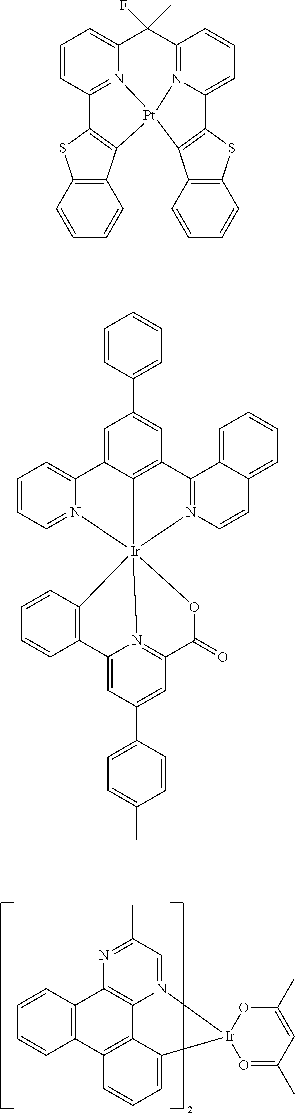

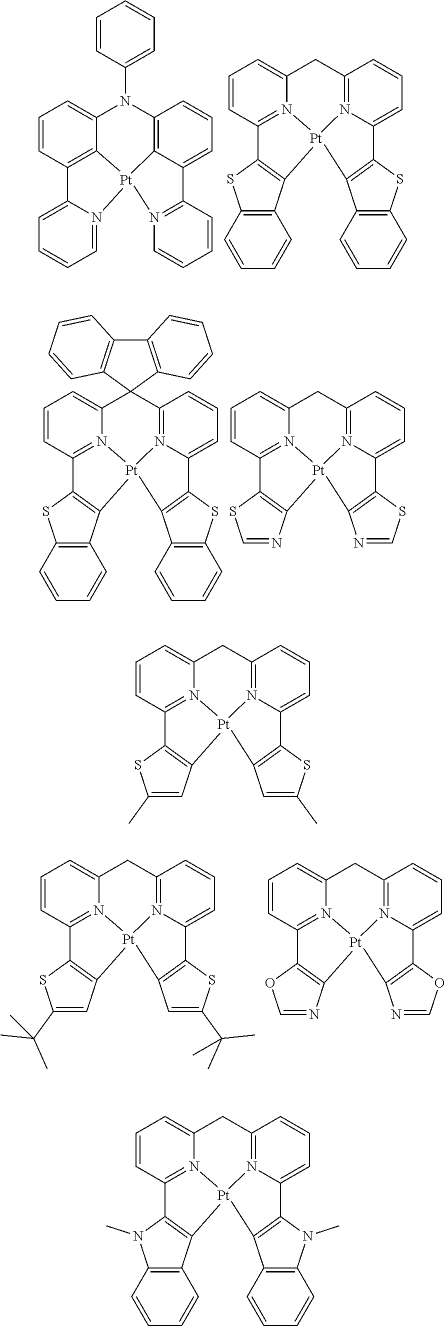

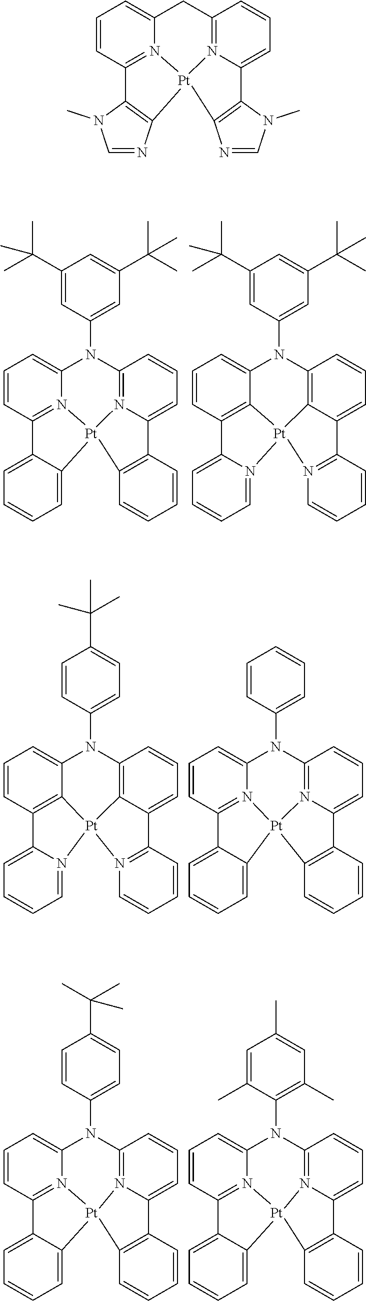

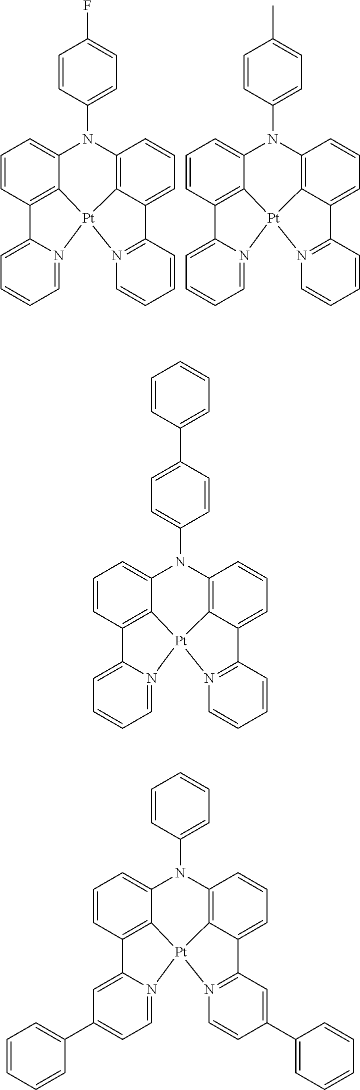

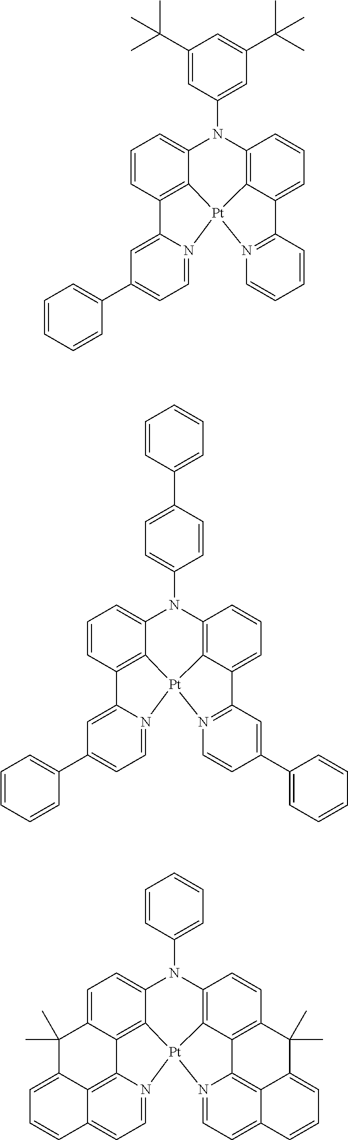

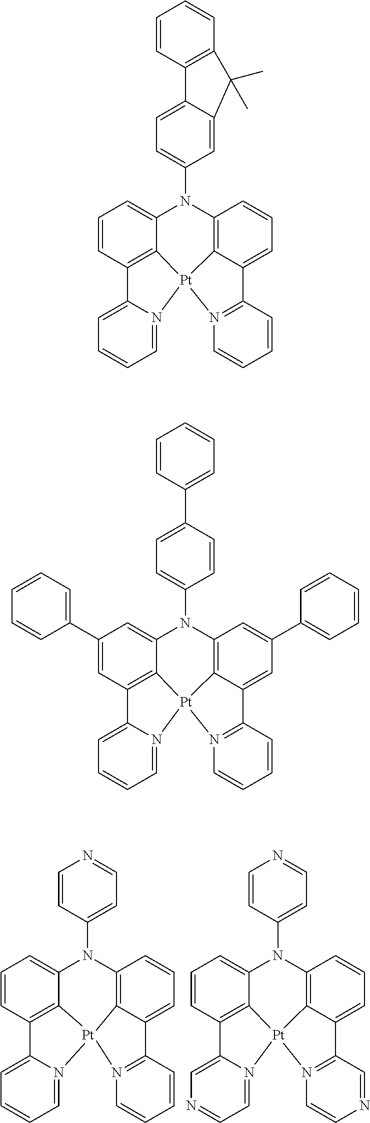

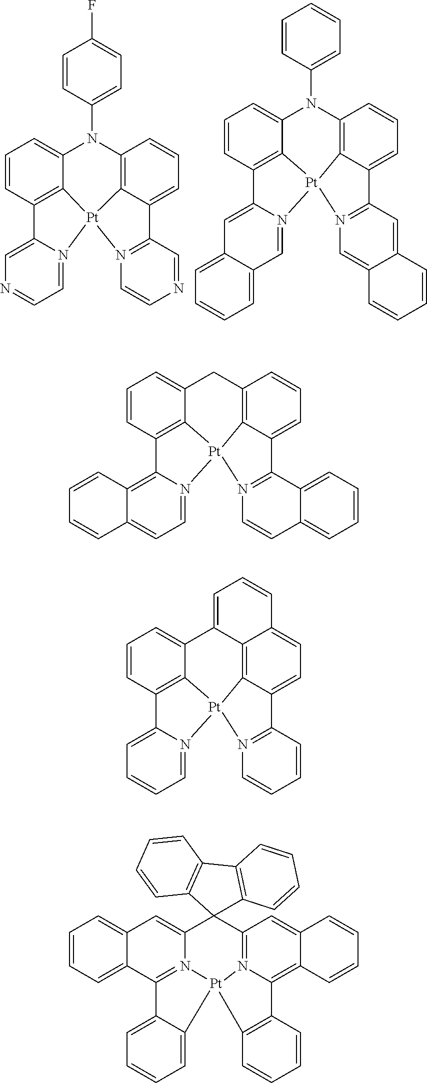

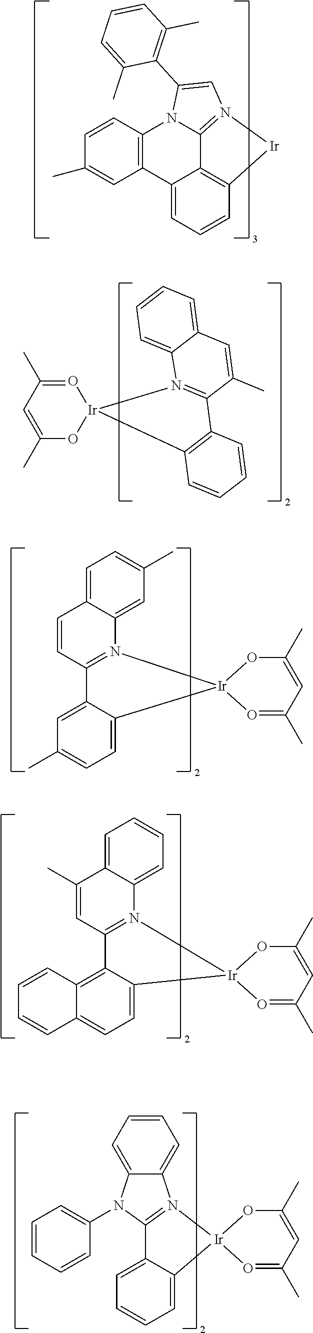

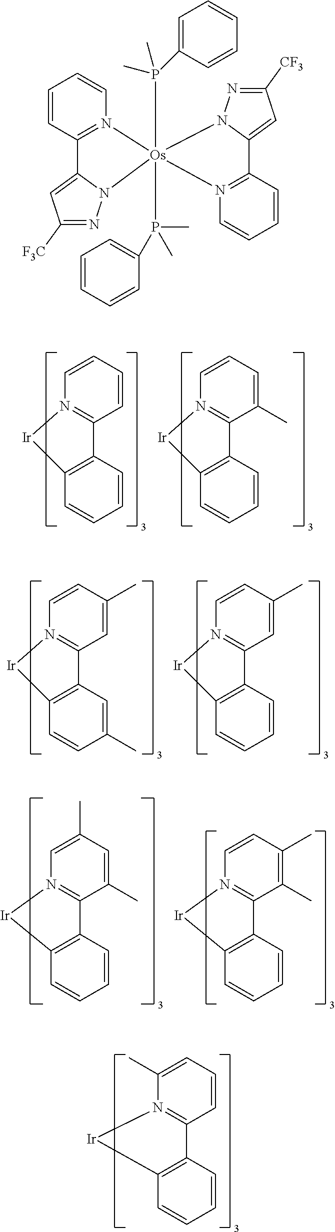

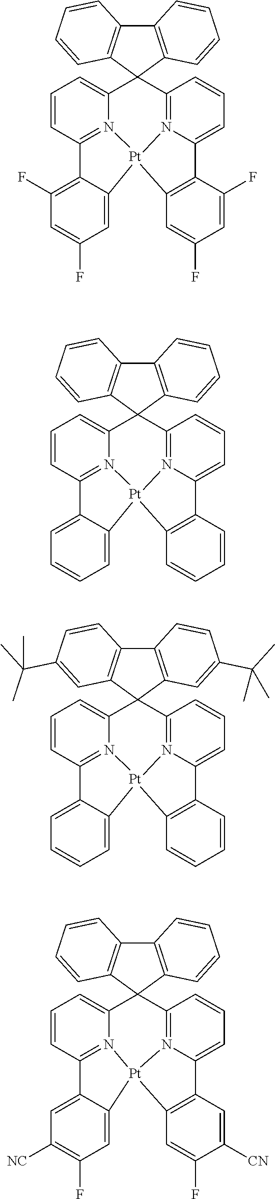

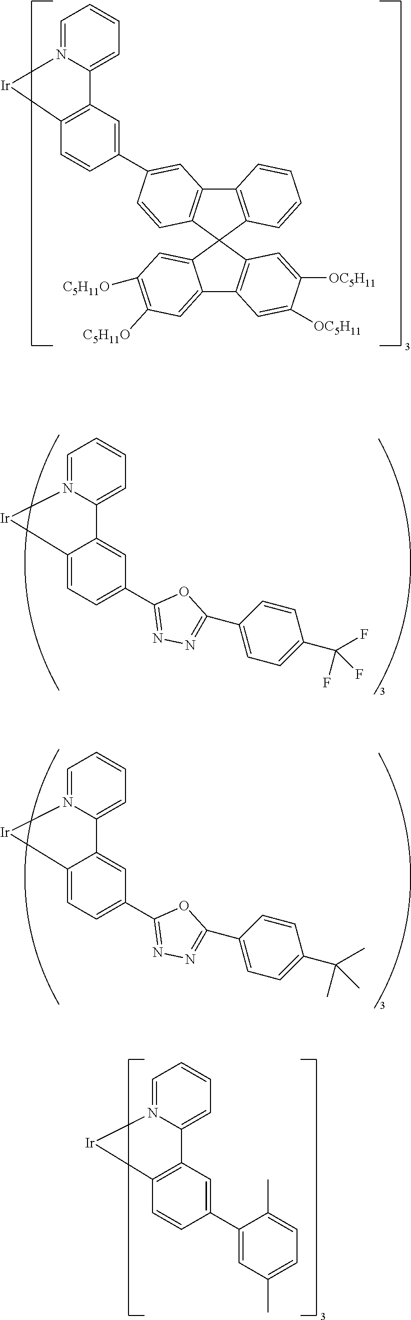

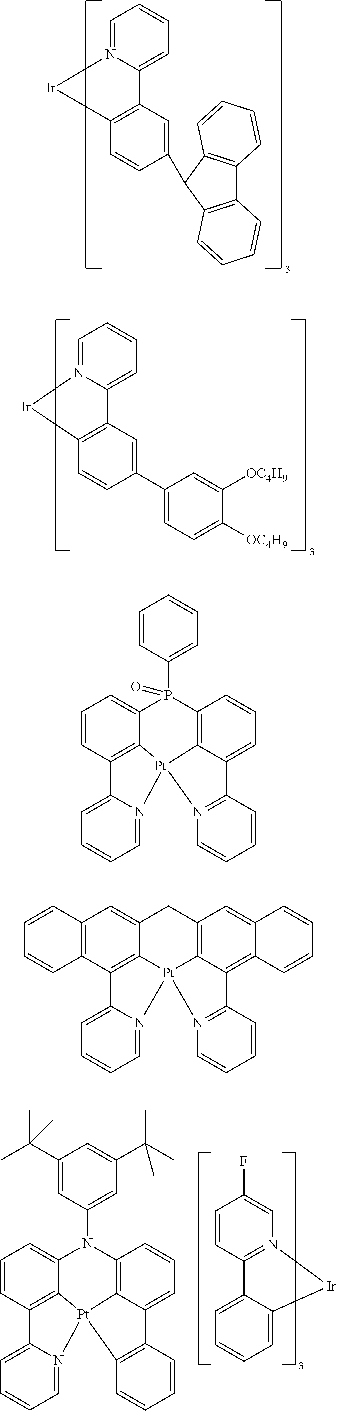

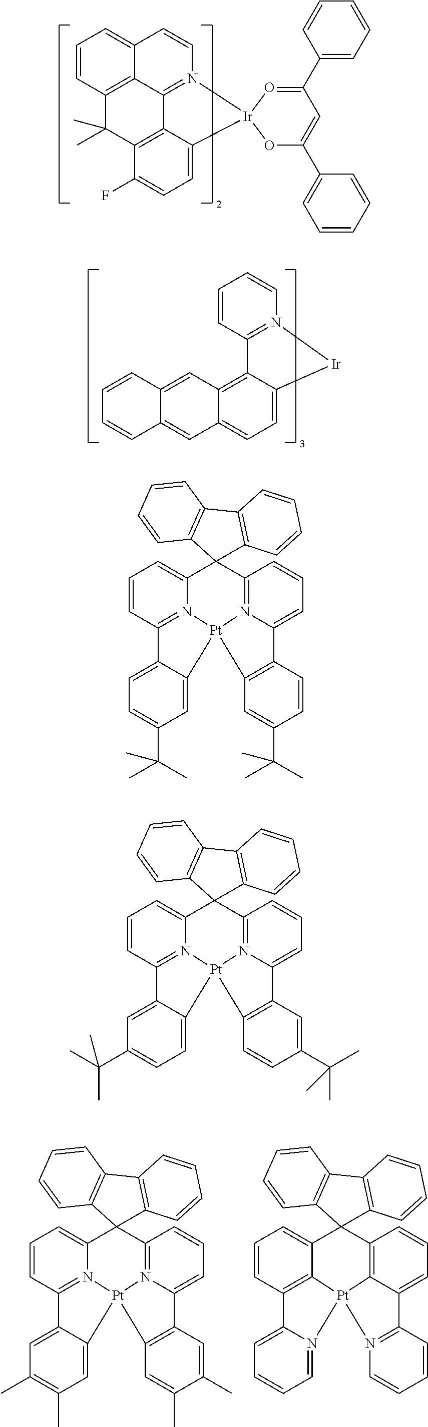

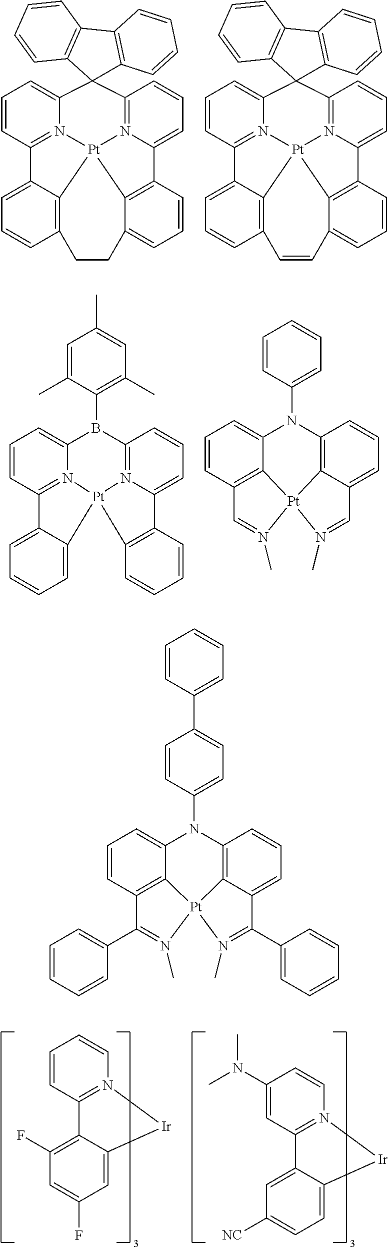

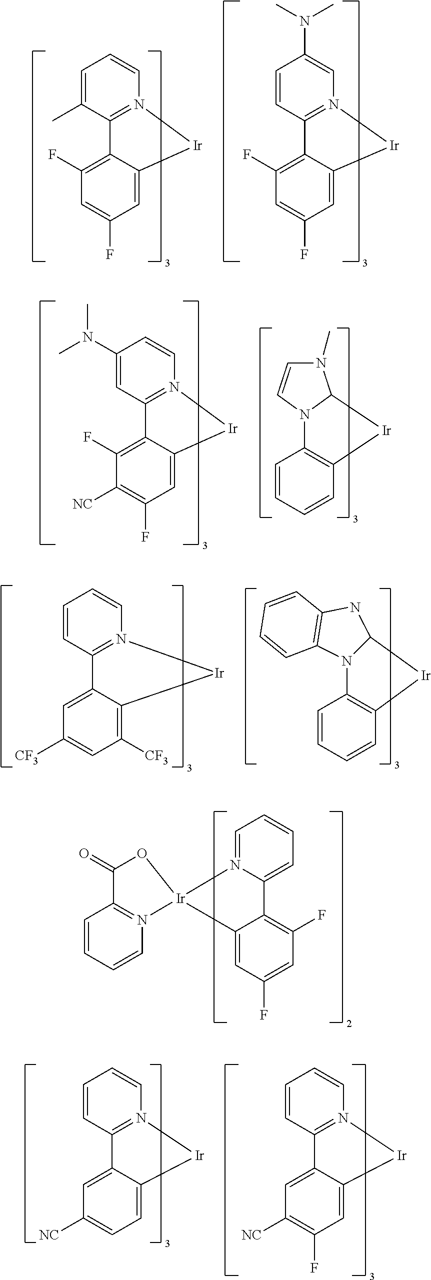

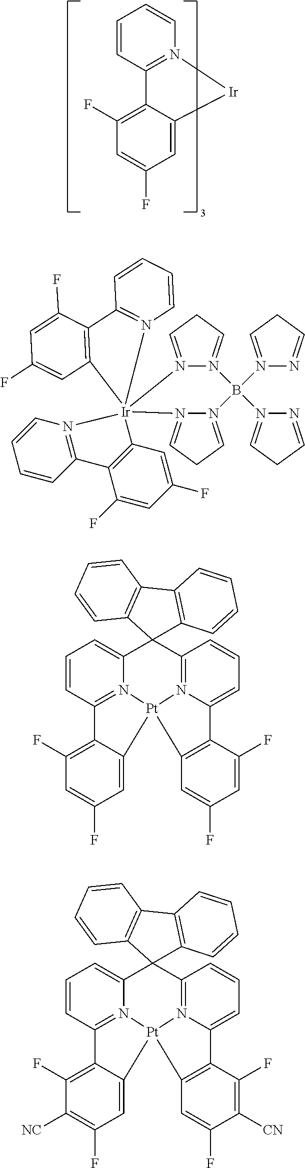

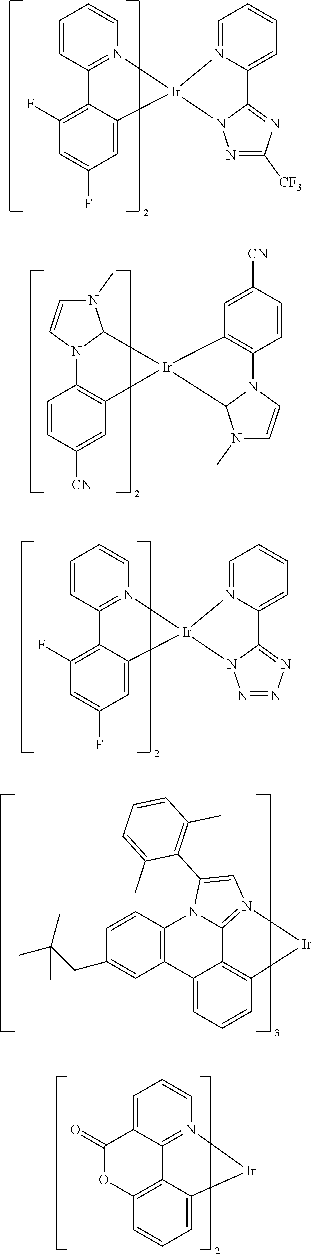

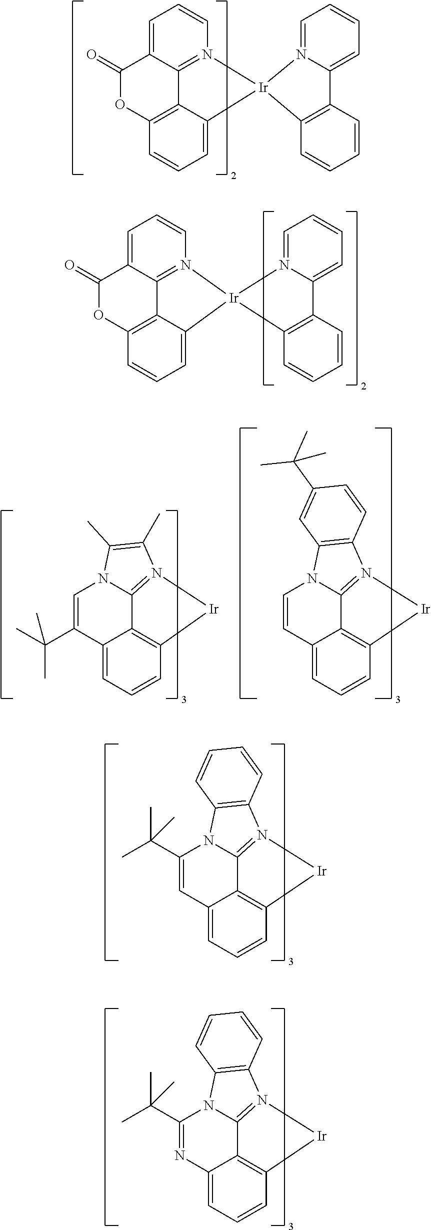

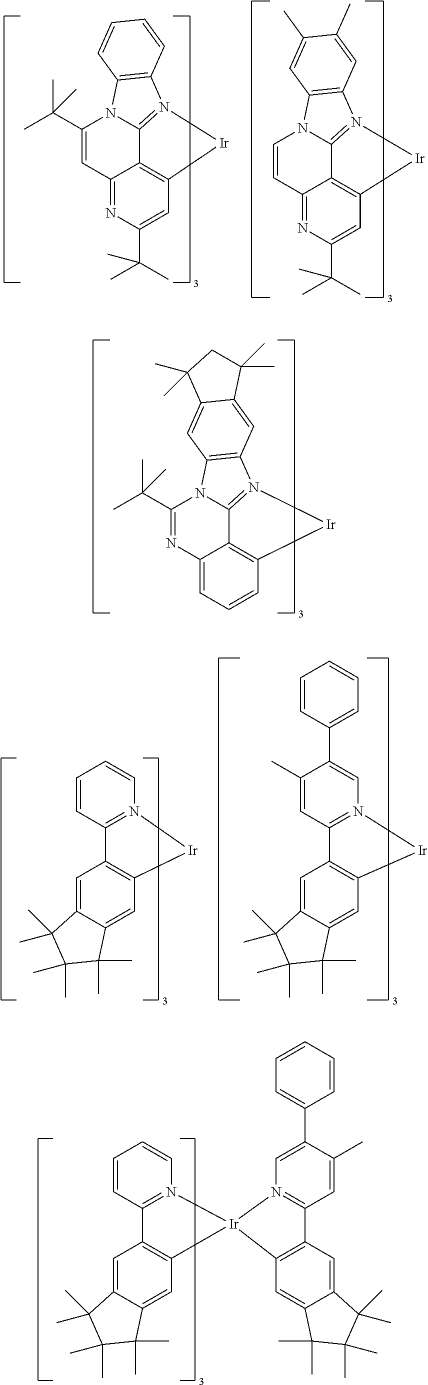

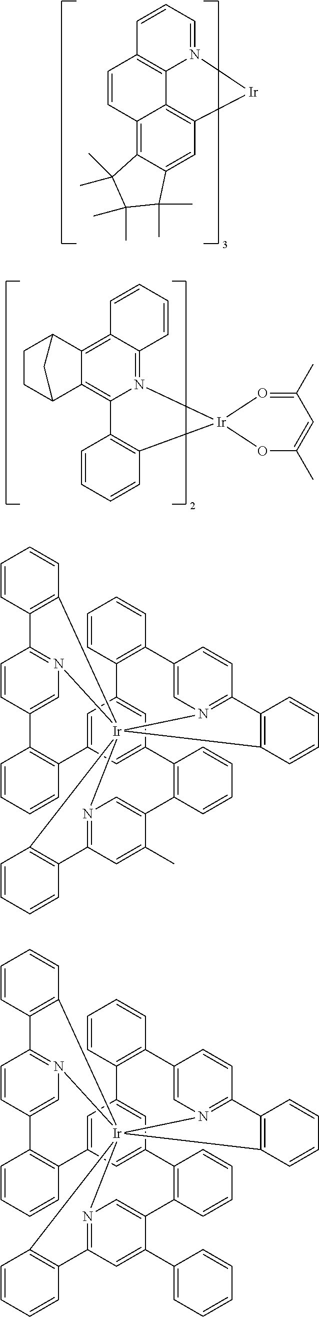

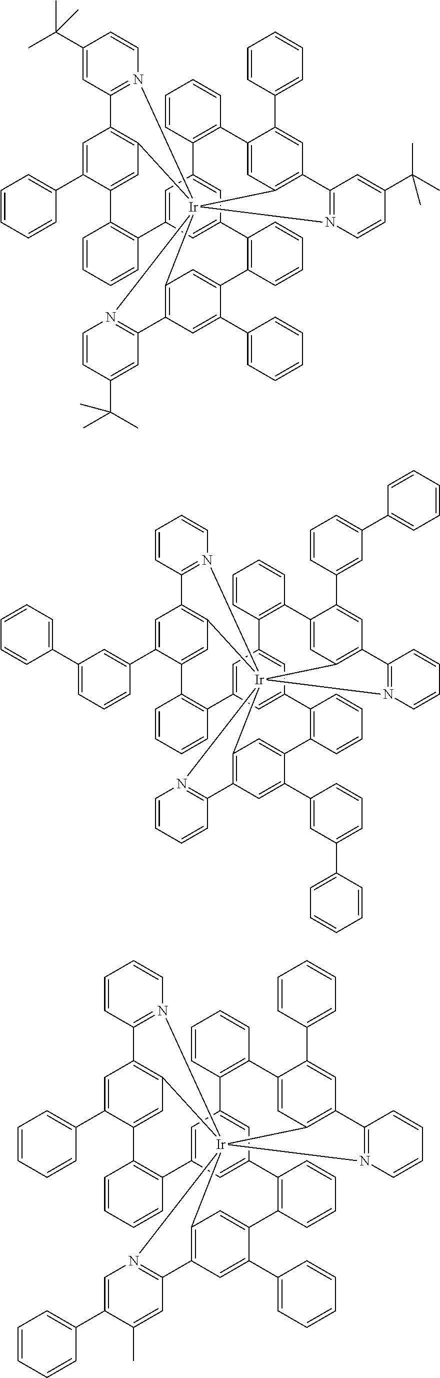

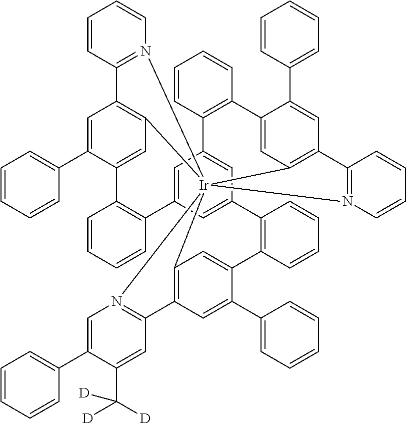

In a preferred embodiment of the invention, the compounds of formula (I) are used as hole-transporting material. The compounds are then preferably in a hole-transporting layer. Preferred embodiments of hole-transporting layers are hole transport layers, electron blocker layers and hole injection layers.

A hole transport layer according to the present application is a layer having a hole-transporting function between the anode and emitting layer. More particularly, it is a hole-transporting layer which is not a hole injection layer and not an electron blocker layer.

Hole injection layers and electron blocker layers are understood in the context of the present application to be specific embodiments of hole-transporting layers. A hole injection layer, in the case of a plurality of hole-transporting layers between the anode and emitting layer, is a hole-transporting layer which directly adjoins the anode or is separated therefrom only by a single coating of the anode. An electron blocker layer, in the case of a plurality of hole-transporting layers between the anode and emitting layer, is that hole-transporting layer which directly adjoins the emitting layer on the anode side. Preferably, the OLED of the invention comprises two, three or four hole-transporting layers between the anode and emitting layer, at least one of which preferably contains a compound of formula (I), and more preferably exactly one or two contain a compound of formula (I).

If the compound of formula (I) is used as hole transport material in a hole transport layer, a hole injection layer or an electron blocker layer, the compound can be used as pure material, i.e. in a proportion of 100%, in the hole transport layer, or it can be used in combination with one or more further compounds. In a preferred embodiment, the organic layer comprising the compound of the formula (I) then additionally contains one or more p-dopants. p-Dopants used according to the present invention are preferably those organic electron acceptor compounds capable of oxidizing one or more of the other compounds in the mixture.

Particularly preferred embodiments of p-dopants are the compounds disclosed in WO 2011/073149, EP 1968131, EP 2276085, EP 2213662, EP 1722602, EP 2045848, DE 102007031220, U.S. Pat. Nos. 8,044,390, 8,057,712, WO 2009/003455, WO 2010/094378, WO 2011/120709, US 2010/0096600, WO 2012/095143 and DE 102012209523.

Particularly preferred p-dopants are quinodimethane compounds, azaindenofluorenediones, azaphenalenes, azatriphenylenes, 12, metal halides, preferably transition metal halides, metal oxides, preferably metal oxides containing at least one transition metal or a metal of main group 3, and transition metal complexes, preferably complexes of Cu, Co, Ni, Pd and Pt with ligands containing at least one oxygen atom as bonding site. Preference is further given to transition metal oxides as dopants, preferably oxides of rhenium, molybdenum and tungsten, more preferably Re₂O₇, MoO₃, WO₃ and ReO₃.

The p-dopants are preferably in substantially homogeneous distribution in the p-doped layers. This can be achieved, for example, by coevaporation of the p-dopant and the hole transport material matrix.

Preferred p-dopants are especially the following compounds:

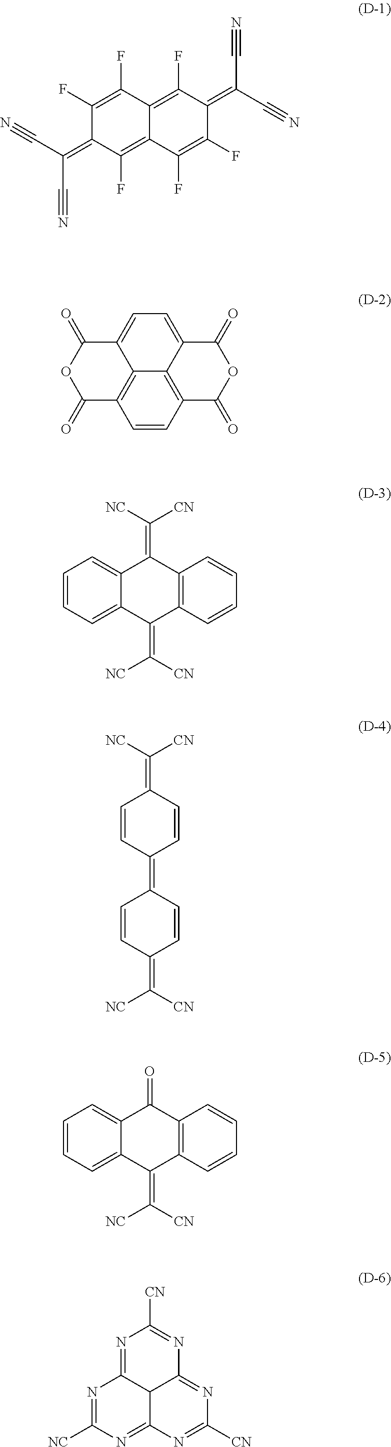 [SMILES: N#CC(C#N)=c1c(F)c(F)c2c(F)c(=C(C#N)C#N)c(F)c(F)c2c1F ; O=C1OC(=O)c2ccc3c4c(ccc1c24)C(=O)OC3=O ; N#CC(C#N)=c1c2ccccc2c(=C(C#N)C#N)c2ccccc12 ; N#CC(C#N)=c1ccc(=c2ccc(=C(C#N)C#N)cc2)cc1 ; N#CC(C#N)=C1c2ccccc2C(=O)c2ccccc21 ; N#CC1=NC2=NC(C#N)=NC3=NC(C#N)=NC(=N1)C23]

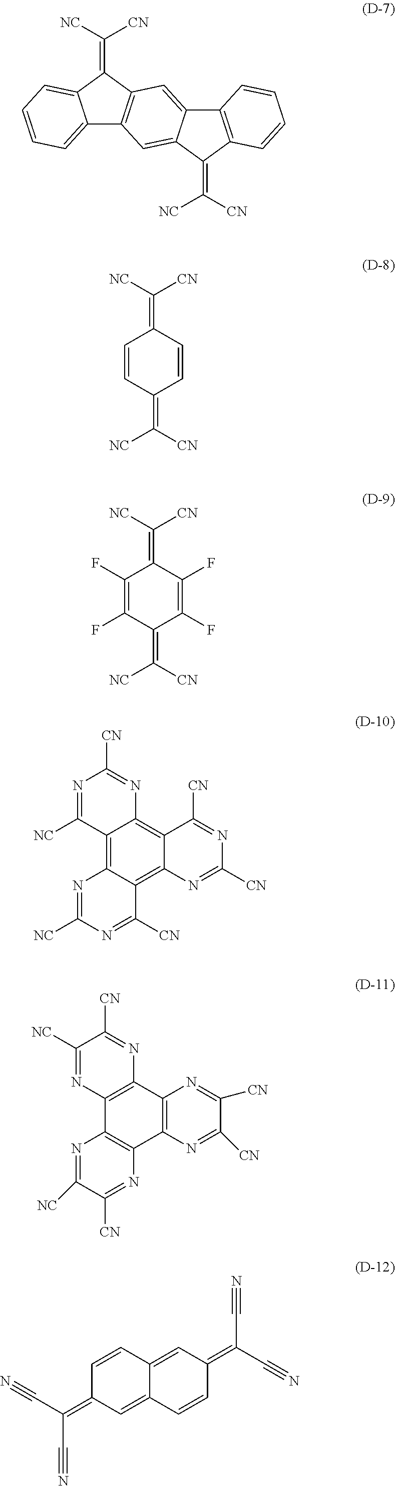 [SMILES: N#CC(C#N)=C1c2ccccc2-c2cc3c(cc21)-c1ccccc1C3=C(C#N)C#N ; N#CC(C#N)=c1ccc(=C(C#N)C#N)cc1 ; N#CC(C#N)=c1c(F)c(F)c(=C(C#N)C#N)c(F)c1F ; N#Cc1nc(C#N)c2c(n1)c1c(C#N)nc(C#N)nc1c1c(C#N)nc(C#N)nc21 ; N#Cc1nc2c3nc(C#N)c(C#N)nc3c3nc(C#N)c(C#N)nc3c2nc1C#N ; N#CC(C#N)=c1ccc2cc(=C(C#N)C#N)ccc2c1]

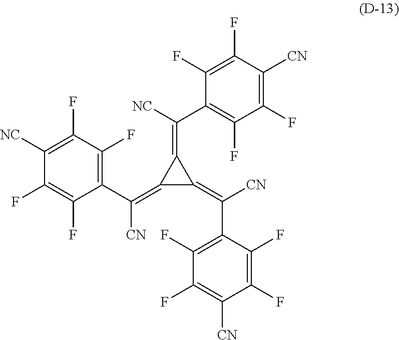 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

In a further preferred embodiment of the invention, the compound of formula (I) is used as hole transport material in combination with a hexaazatriphenylene derivative as described in US 2007/0092755 in an OLED. Particular preference is given here to using the hexaazatriphenylene derivative in a separate layer.

In a preferred embodiment of the present invention, the compound of the formula (I) is used in an emitting layer as matrix material in combination with one or more emitting compounds, preferably phosphorescent emitting compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also comprise systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (I) are used as a component of mixed matrix systems, preferably for phosphorescent emitters. The mixed matrix systems preferably comprise two or three different matrix materials, more preferably two different matrix materials. Preferably, in this case, one of the two materials is a material having hole-transporting properties and the other material is a material having electron-transporting properties. The compound of the formula (I) is preferably the matrix material having hole-transporting properties. Correspondingly, when the compound of the formula (I) is used as matrix material for a phosphorescent emitter in the emitting layer of an OLED, a second matrix compound having electron-transporting properties is present in the emitting layer. The two different matrix materials may be present in a ratio of 1:50 to 1:1, preferably 1:20 to 1:1, more preferably 1:10 to 1:1 and most preferably 1:4 to 1:1. More specific details relating to mixed matrix systems are given inter alia in the application WO 2010/108579, the corresponding technical teaching of which is incorporated by reference in this connection.

The desired electron-transporting and hole-transporting properties of the mixed matrix components may, however, also be combined mainly or entirely in a single mixed matrix component, in which case the further mixed matrix component(s) fulfil(s) other functions.

The mixed matrix systems may comprise one or more emitting compounds, preferably one or more phosphorescent emitting compounds. In general, mixed matrix systems are preferably used in phosphorescent organic electroluminescent devices.

Particularly suitable matrix materials which can be used in combination with the inventive compounds as matrix components of a mixed matrix system are selected from the preferred matrix materials specified below for phosphorescent emitting compounds, and among these especially from those having electron-transporting properties.

Preferred embodiments of the different functional materials in the electronic device are listed hereinafter.

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 positions. Further preferred emitting compounds are indenofluoreneamines or -diamines, for example according to WO 2006/108497 or WO 2006/122630, benzoindenofluoreneamines or -diamines, for example according to WO 2008/006449, and dibenzoindenofluoreneamines or -diamines, for example according to WO 2007/140847, and the indenofluorene derivatives having fused aryl groups disclosed in WO 2010/012328. Likewise preferred are the pyrenearylamines disclosed in WO 2012/048780 and in WO 2013/185871. Likewise preferred are the benzoindenofluoreneamines disclosed in WO 2014/037077, the benzofluoreneamines disclosed in WO 2014/106522, the extended benzoindenofluorenes disclosed in WO 2014/111269 and in WO 2017/036574, the phenoxazines disclosed in WO 2017/028940 and WO 2017/028941, and the fluorene derivatives bonded to furan units or to thiophene units that are disclosed in WO 2016/150544.

Useful matrix materials, preferably for fluorescent emitting compounds, include materials of various substance classes. Preferred matrix materials are selected from the classes of the oligoarylenes (e.g. 2,2′,7,7′-tetraphenylspirobifluorene according to EP 676461 or dinaphthylanthracene), especially of the oligoarylenes containing fused aromatic groups, the oligoarylenevinylenes (e.g. DPVBi or spiro-DPVBi according to EP 676461), the polypodal metal complexes (for example according to WO 2004/081017), the hole-conducting compounds (for example according to WO 2004/058911), the electron-conducting compounds, especially ketones, phosphine oxides, sulfoxides, etc. (for example according to WO 2005/084081 and WO 2005/084082), the atropisomers (for example according to WO 2006/048268), the boronic acid derivatives (for example according to WO 2006/117052) or the benzanthracenes (for example according to WO 2008/145239). Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds.

An oligoarylene in the context of this invention shall be understood to mean a compound in which at least three aryl or arylene groups are bonded to one another. Preference is further given to the anthracene derivatives disclosed in WO 2006/097208, WO 2006/131192, WO 2007/065550, WO 2007/110129, WO 2007/065678, WO 2008/145239, WO 2009/100925, WO 2011/054442 and EP 1553154, the pyrene compounds disclosed in EP 1749809, EP 1905754 and US 2012/0187826, the benzanthracenylanthracene compounds disclosed in WO 2015/158409, the indenobenzofurans disclosed in WO 2017/025165, and the phenanthrylanthracenes disclosed in WO 2017/036573.

Preferred matrix materials for phosphorescent emitting compounds are, as well as the compounds of the formula (I), aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527 or WO 2008/086851, indolocarbazole derivatives, for example according to WO 2007/063754 or WO 2008/056746, indenocarbazole derivatives, for example according to WO 2010/136109, WO 2011/000455 or WO 2013/041176, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2010/015306, WO 2007/063754 or WO 2008/056746, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to US 2009/0136779, WO 2010/050778, WO 2011/042107, WO 2011/088877 or WO 2012/143080, triphenylene derivatives, for example according to WO 2012/048781, or lactams, for example according to WO 2011/116865 or WO 2011/137951.

Suitable charge transport materials as usable in the hole injection or hole transport layer or electron blocker layer or in the electron transport layer of the electronic device of the invention are, as well as the compounds of the formula (I), for example, the compounds disclosed in Y. Shirota et al., Chem. Rev. 2007, 107(4), 953-1010, or other materials as used in these layers according to the prior art.

Preferred materials having a hole-transporting properties which can be used, for example, in hole injection layers, hole transport layers, electron blocker layers and/or emitting layers of OLEDs are depicted in the following table:

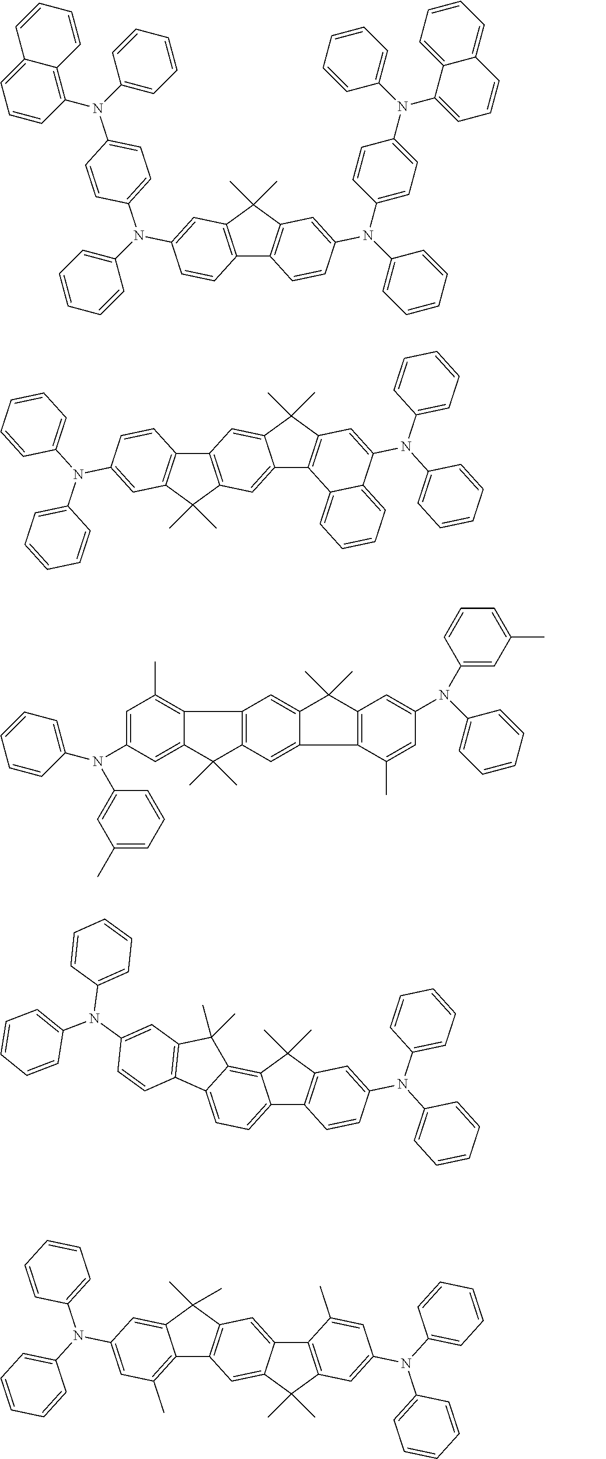

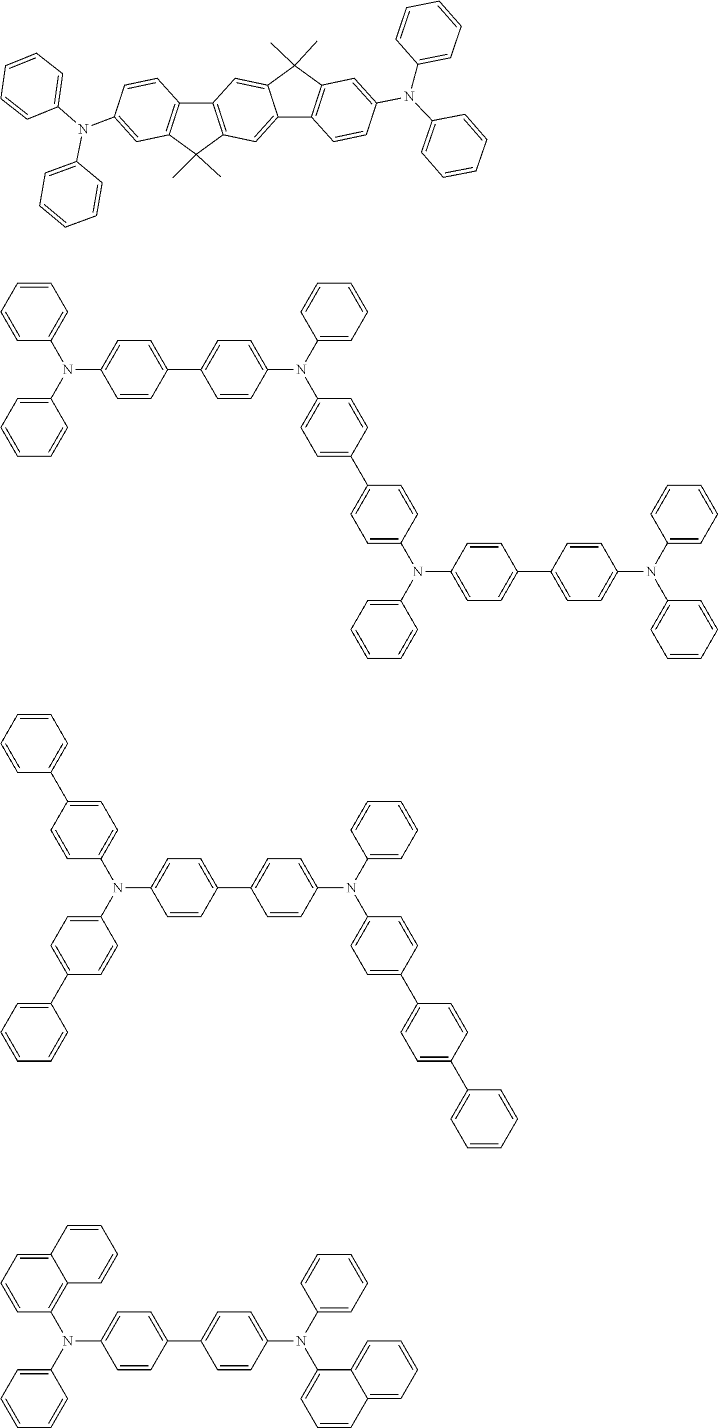 [SMILES: CC1(C)c2cc(N(c3ccccc3)c3ccccc3)ccc2-c2cc3c(cc21)-c1ccc(N(c2ccccc2)c2ccccc2)cc1C3(C)C ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4cccc5ccccc45)cc3)cc2)c2cccc3ccccc23)cc1]

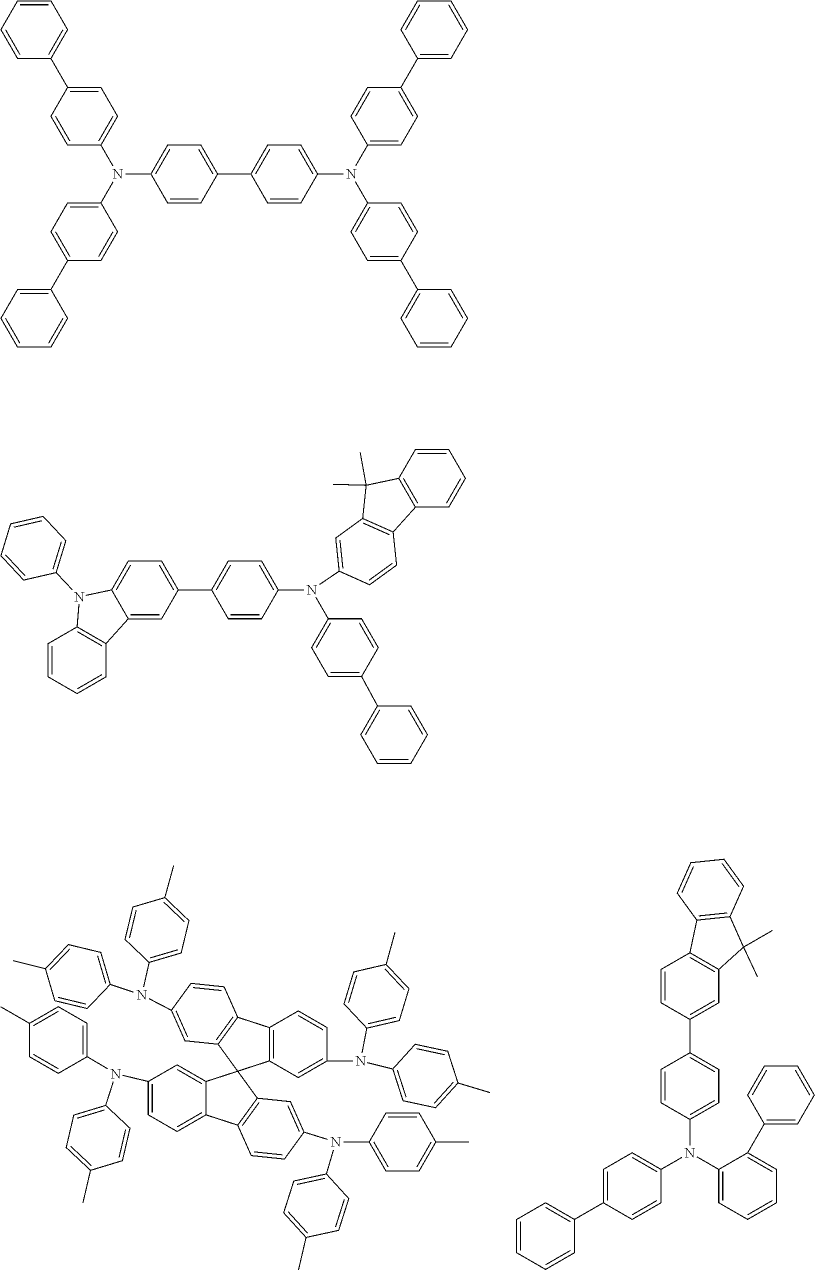

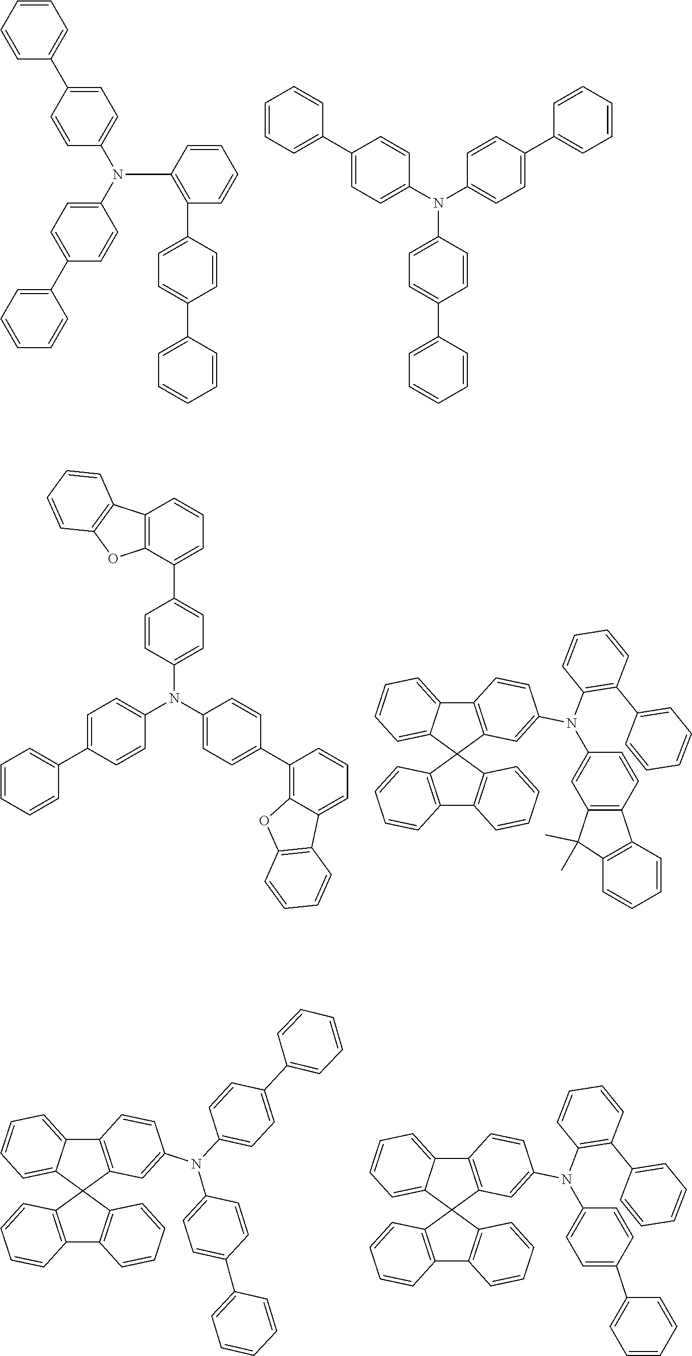

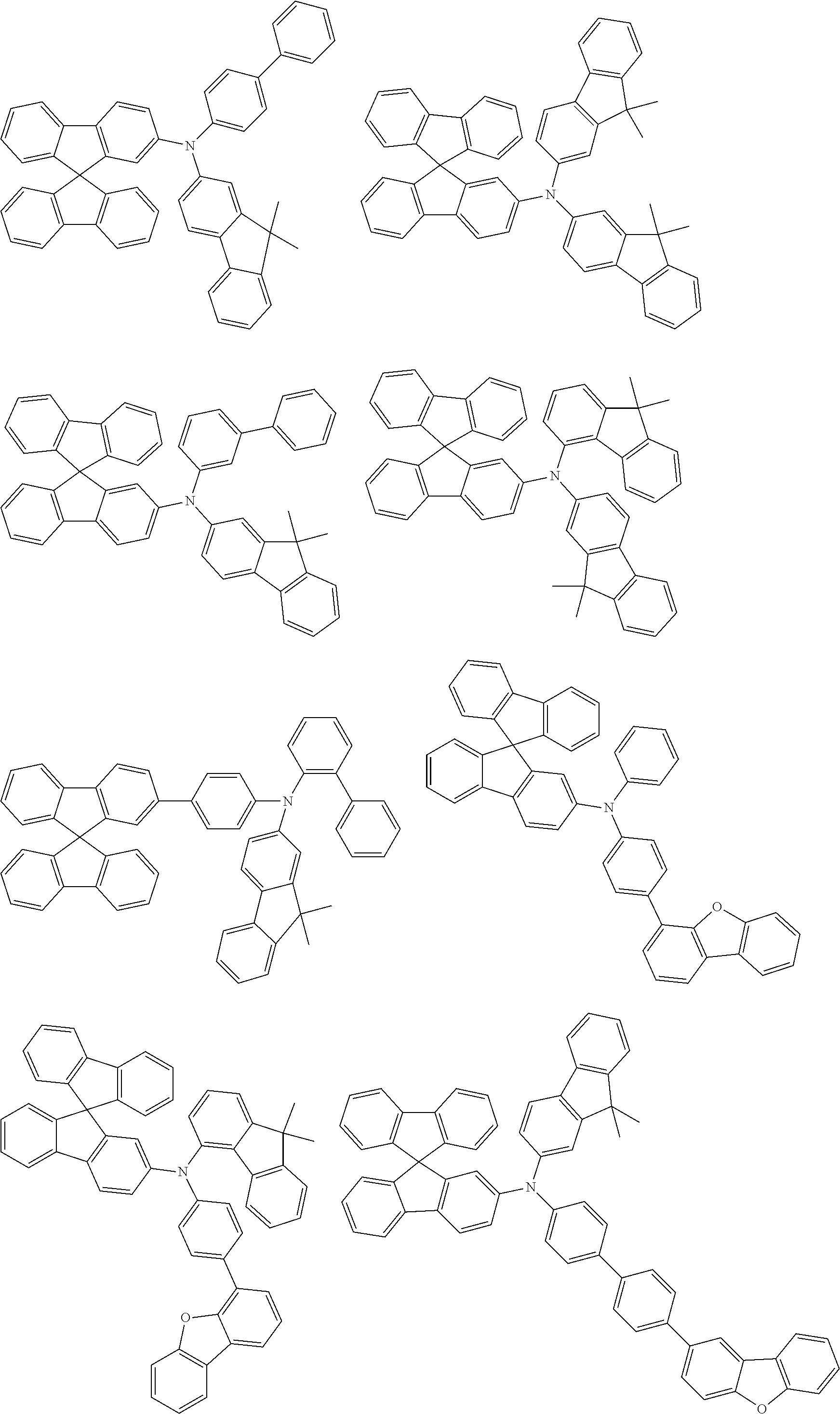

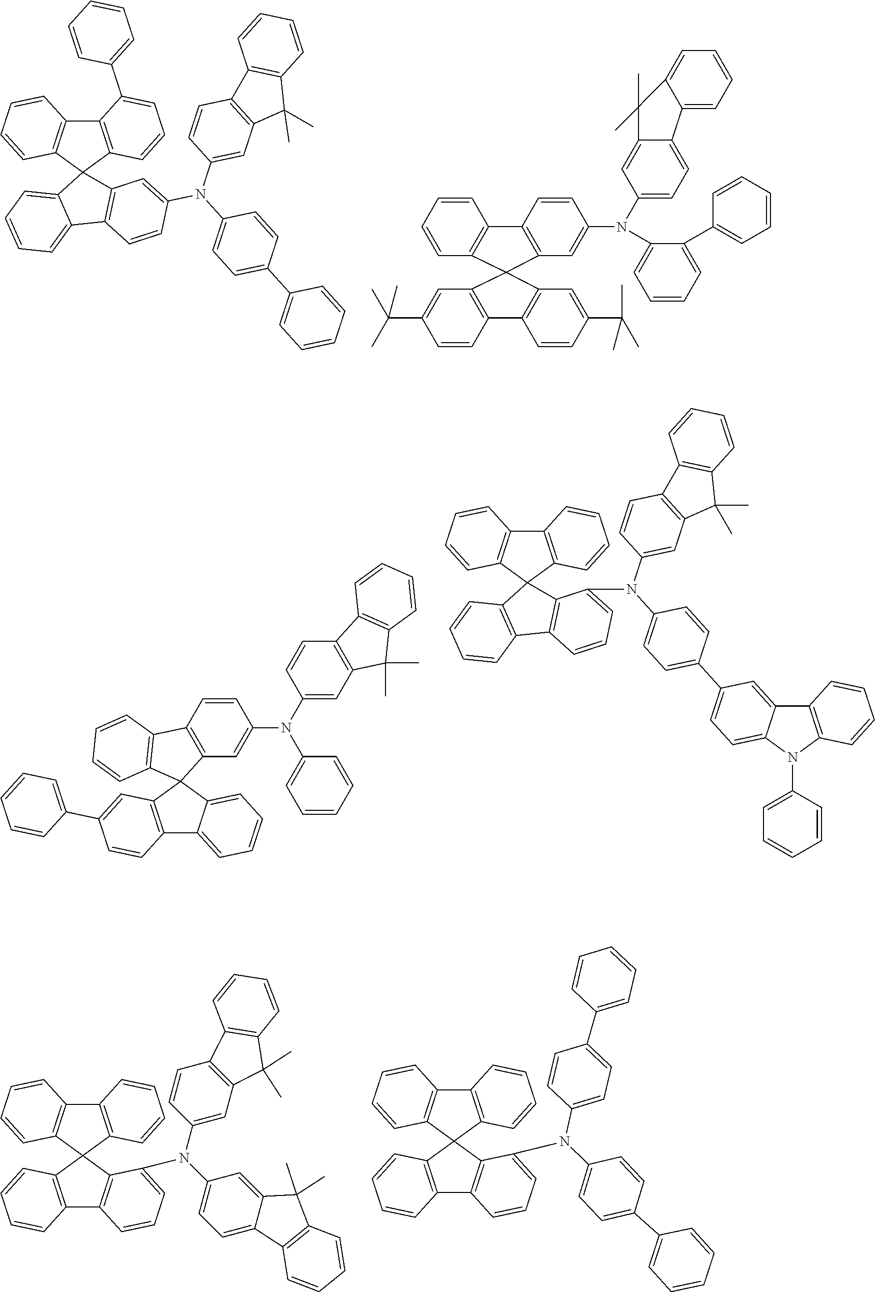

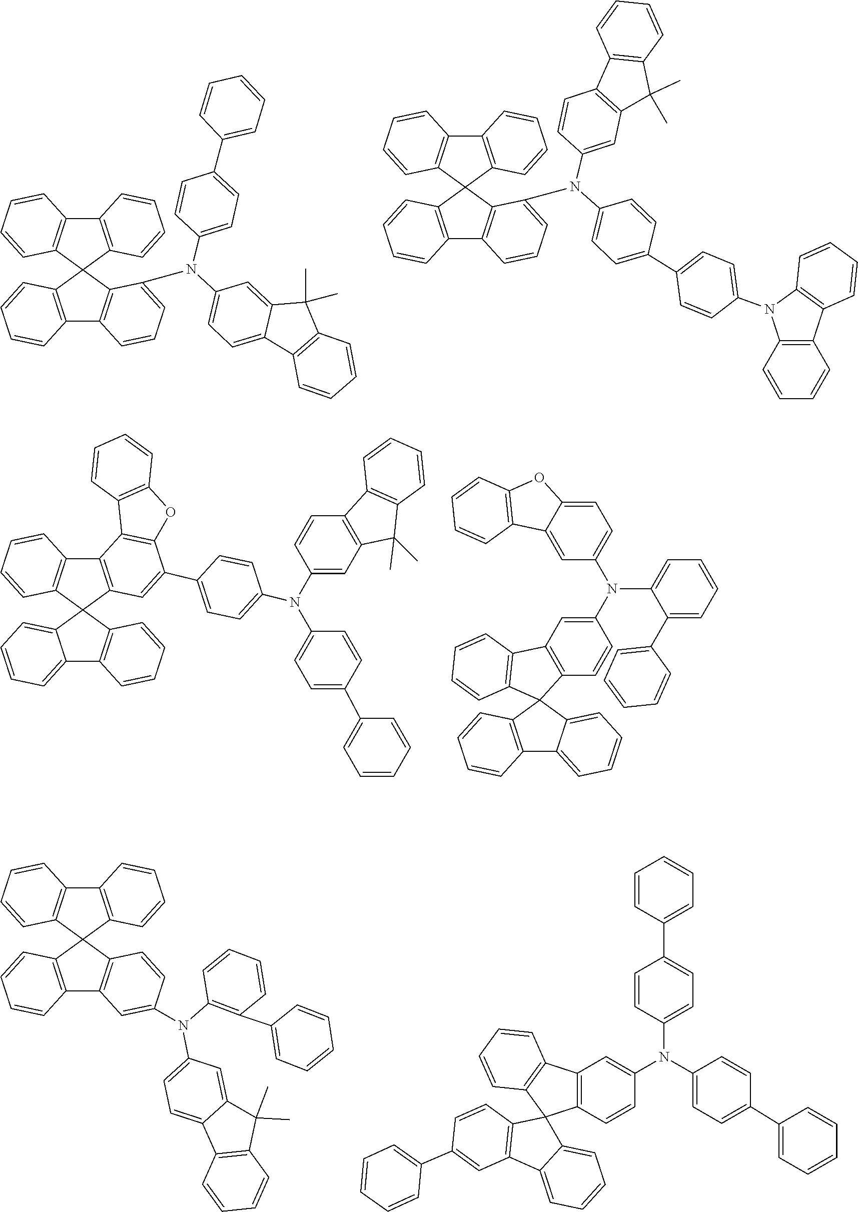

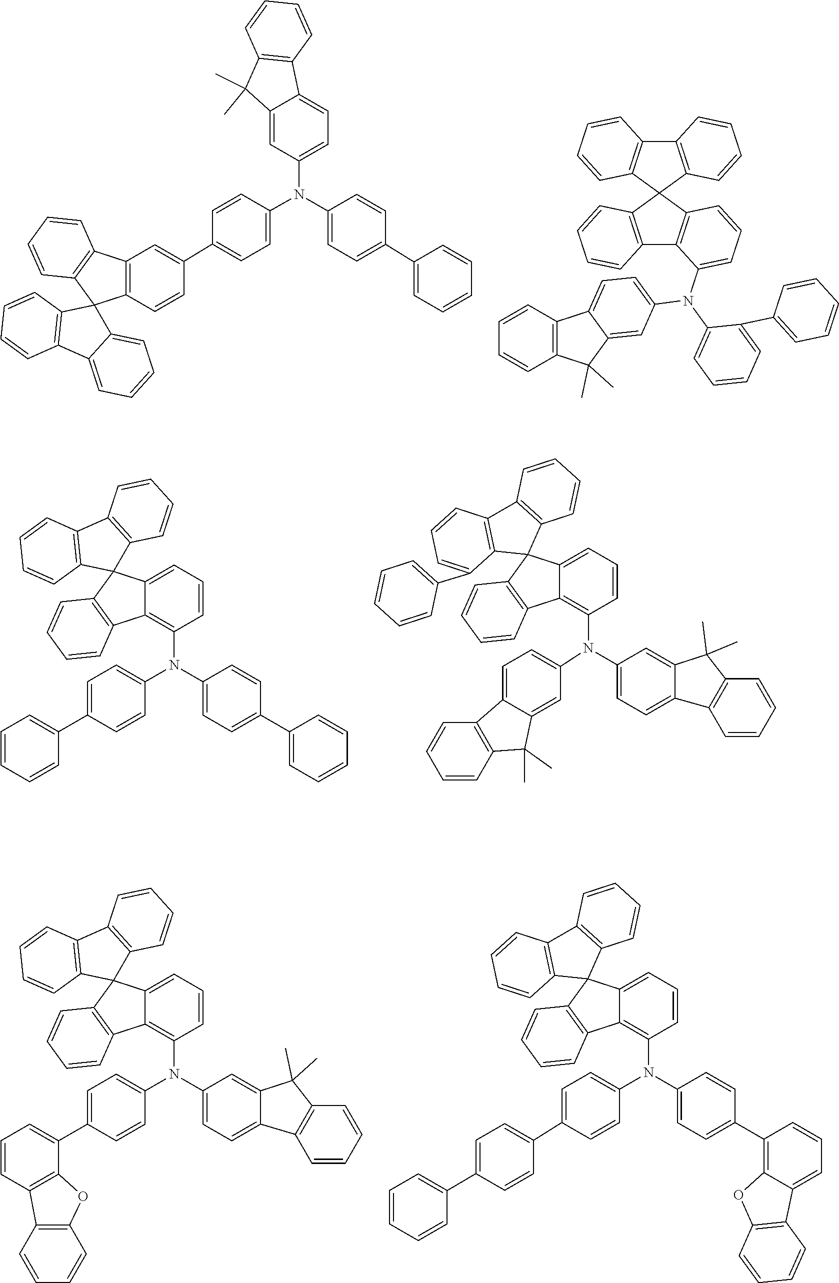

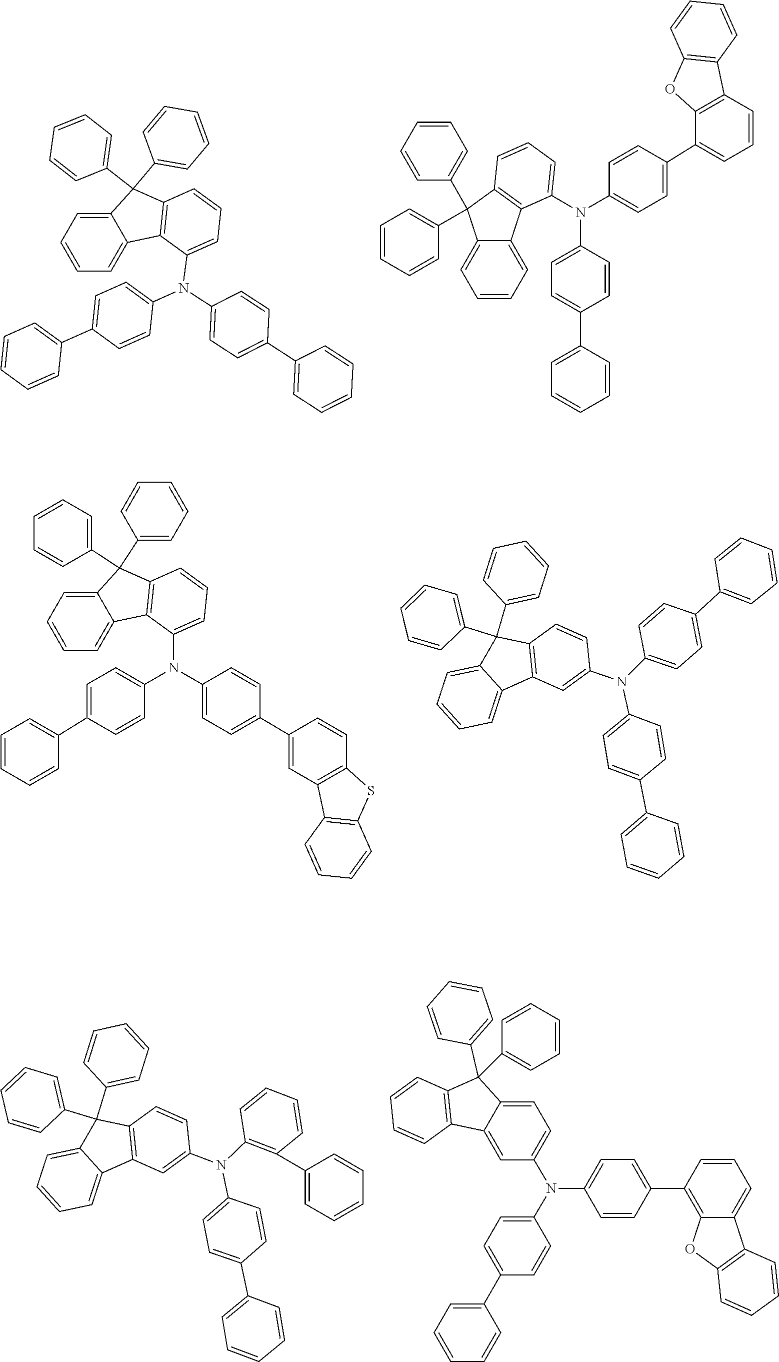

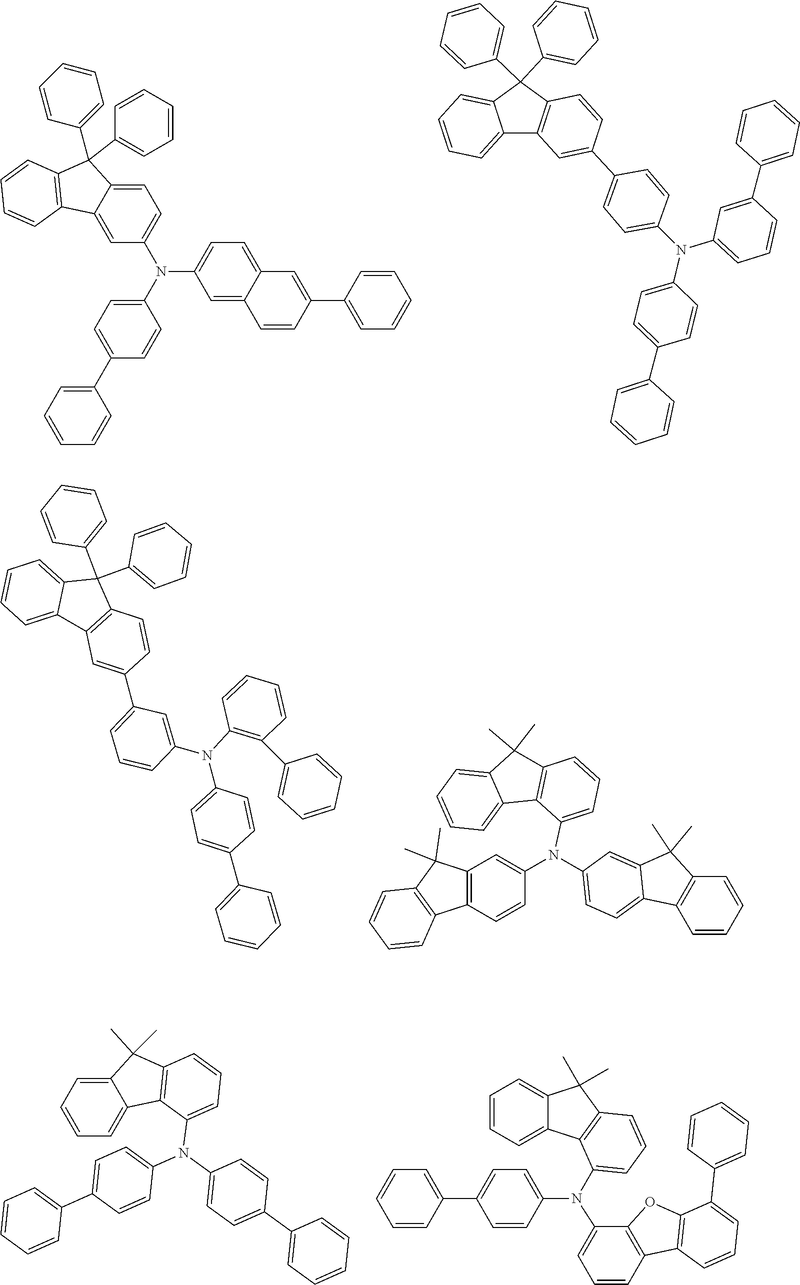

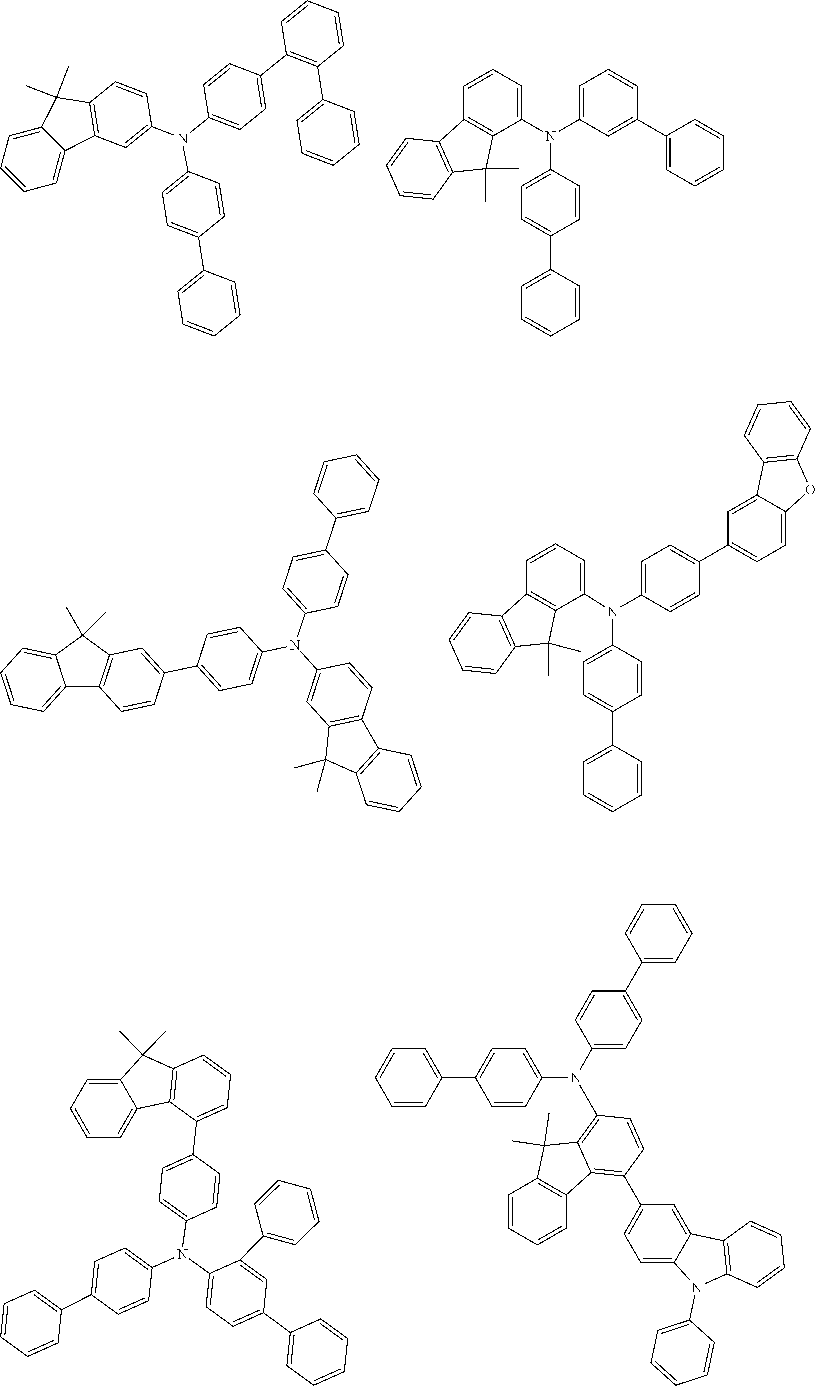

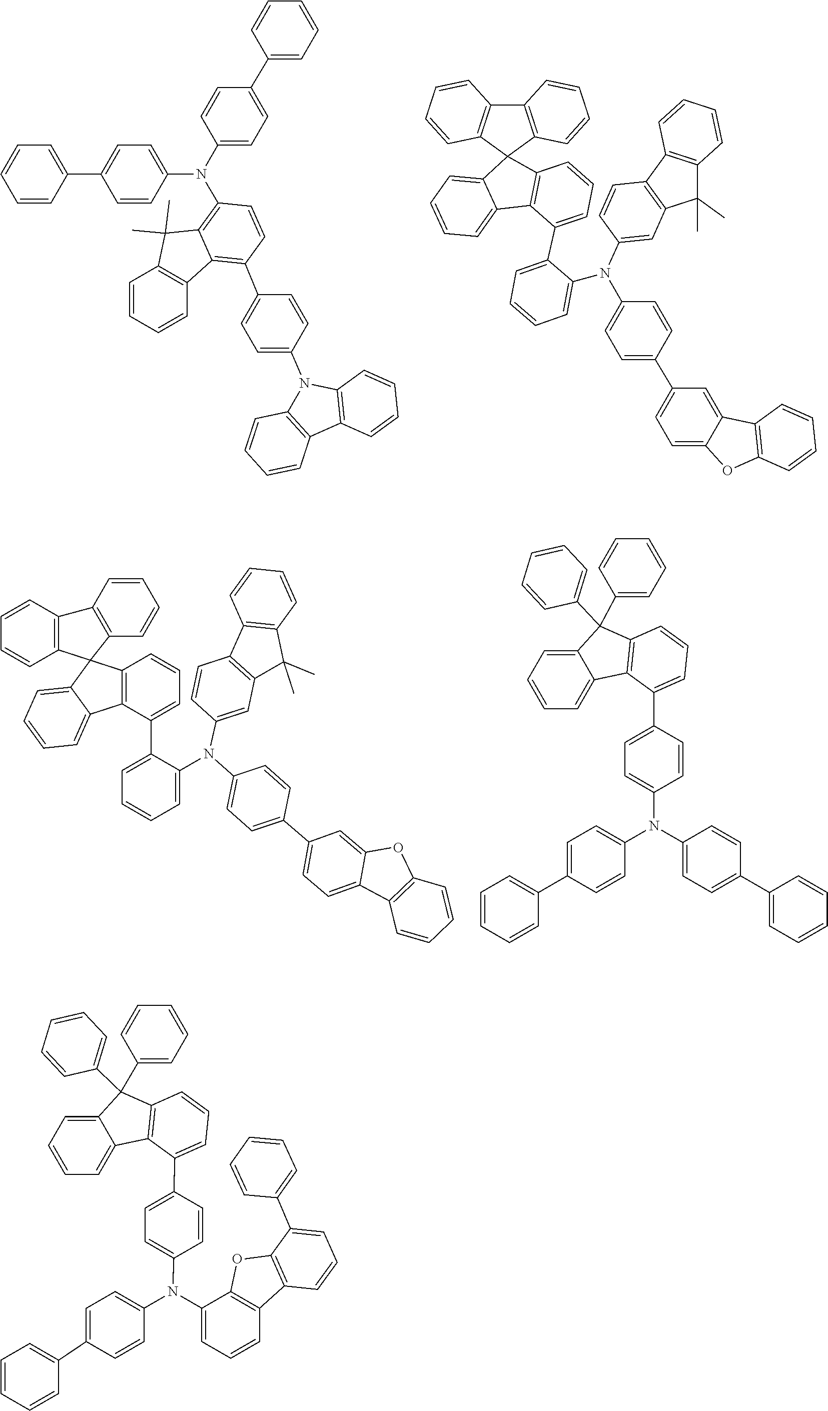

Preferably, the inventive OLED comprises two or more different hole-transporting layers. The compound of the formula (I) may be used here in one or more of or in all the hole-transporting layers. In a preferred embodiment, the compound of the formula (I) is used in exactly one or exactly two hole-transporting layers, and other compounds, preferably aromatic amine compounds, are used in the further hole-transporting layers present. Further compounds which are used alongside the compounds of the formula (I), preferably in hole-transporting layers of the OLEDs of the invention, are especially indenofluoreneamine derivatives (for example according to WO 06/122630 or WO 06/100896), the amine derivatives disclosed in EP 1661888, hexaazatriphenylene derivatives (for example according to WO 01/049806), amine derivatives with fused aromatics (for example according to U.S. Pat. No. 5,061,569), the amine derivatives disclosed in WO 95/09147, monobenzoindenofluoreneamines (for example according to WO 08/006449), dibenzoindenofluoreneamines (for example according to WO 07/140847), spirobifluoreneamines (for example according to WO 2012/034627 or WO 2013/120577), fluoreneamines (for example according to WO 2014/015937, WO 2014/015938, WO 2014/015935 and WO 2015/082056), spirodibenzopyranamines (for example according to WO 2013/083216), dihydroacridine derivatives (for example according to WO 2012/150001), spirodibenzofurans and spirodibenzothiophenes, for example according to WO 2015/022051 and WO 2016/102048 and WO 2016/131521, phenanthrenediarylamines, for example according to WO 2015/131976, spirotribenzotropolones, for example according to WO 2016/087017, spirobifluorenes with meta-phenyldiamine groups, for example according to WO 2016/078738, spirobisacridines, for example according to WO 2015/158411, xanthenediarylamines, for example according to WO 2014/072017, and 9,10-dihydroanthracene spiro compounds with diarylamino groups according to WO 2015/086108.

Materials used for the electron transport layer may be any materials as used according to the prior art as electron transport materials in the electron transport layer. Especially suitable are aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Further suitable materials are derivatives of the abovementioned compounds as disclosed in JP 2000/053957, WO 2003/060956, WO 2004/028217, WO 2004/080975 and WO 2010/072300.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable the irradiation of the organic material (organic solar cell) or the emission of light (OLED, O-laser). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

The device is structured appropriately (according to the application), contact-connected and finally sealed, in order to rule out damaging effects by water and air.

In a preferred embodiment, the electronic device is characterized in that one or more layers are coated by a sublimation process. In this case, the materials are applied by vapour deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapourjet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I) are needed. High solubility can be achieved by suitable substitution of the compounds.

It is further preferable that an electronic device of the invention is produced by applying one or more layers from solution and one or more layers by a sublimation method.

According to the invention, the electronic devices comprising one or more compounds of formula (I) can be used in displays, as light sources in lighting applications and as light sources in medical and/or cosmetic applications (e.g. light therapy).

### EXAMPLES

### A) Synthesis Examples

### Synthesis of the compound (9,9-dimethyl-9H-fluoren-2-yl)(9,9-spirobifluoren-2-yl)(3′-pyridin-3-ylbiphenyl-2-yl)amine (1-1) and of the compounds (I-2) to (I-15)

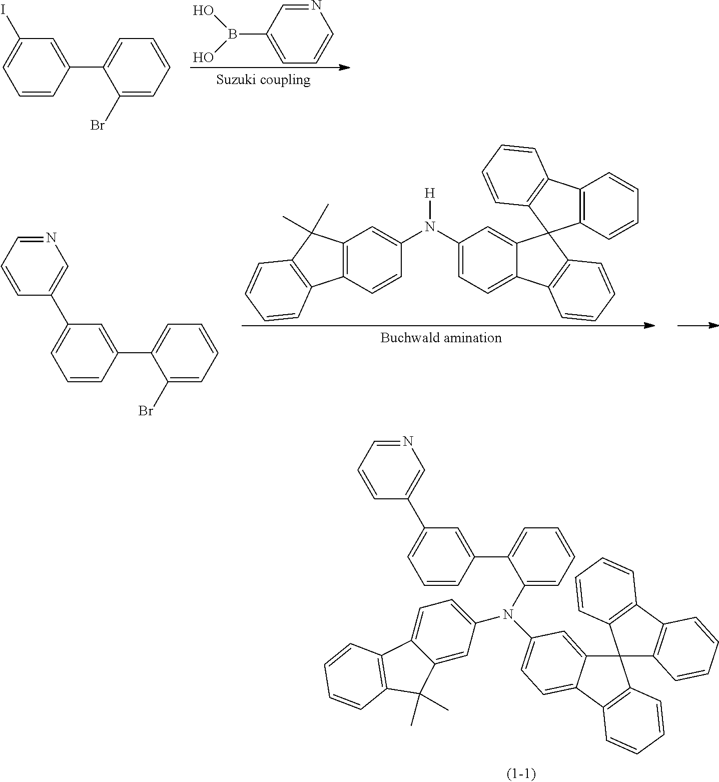

### Synthesis of intermediate I-1: 3-(2′-Bromobiphenyl-3-yl)pyridine

10.0 g (81.4 mmol) of pyridine-3-boronic acid (CAS No.: 1692-25-7), 29.2 g (81.4 mmol) of 2-bromo-3′-iodobiphenyl (CAS No.: 1776936-09-4) and 93 ml of an aqueous 2 M Na₂CO₃ solution (186 mmol) are suspended in 75 ml of ethanol and 120 ml of toluene. To this suspension is added 0.94 g (0.82 mmol) of tetrakis(triphenyl)phosphinepalladium(0). The reaction mixture is heated under reflux for 16 h. After cooling, the organic phase is removed, filtered through silica gel, washed three times with 150 ml of water and then concentrated to dryness. After the crude product has been filtered through silica gel with heptane/ethyl acetate, 19 g (79%) of 3-(2′-bromobiphenyl-3-yl)pyridine are obtained.

The following compounds are prepared in an analogous manner:

| | Reactant 1 | Reactant 2 | Product |
|---|---|---|---|
| I-2 | 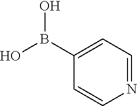 (1692-15-5) | 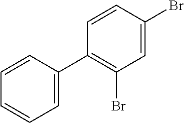 [SMILES: Brc1ccc(-c2ccccc2)c(Br)c1] (53592-10-2) | 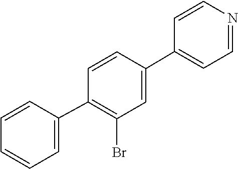 [SMILES: Brc1cc(-c2ccncc2)ccc1-c1ccccc1] |
| I-3 | 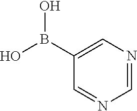 (109299-78-7) | 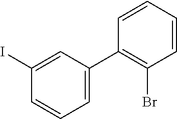 [SMILES: Brc1ccccc1-c1cccc(I)c1] (1776936-09-4) | 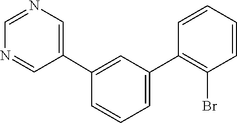 [SMILES: Brc1ccccc1-c1cccc(-c2cncnc2)c1] |
| I-4 | 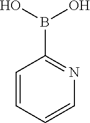 (197958-29-5) | 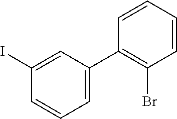 [SMILES: Brc1ccccc1-c1cccc(I)c1] (1776936-09-4) | 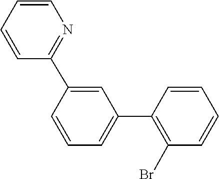 [SMILES: Brc1ccccc1-c1cccc(-c2ccccn2)c1] |
| I-5 | 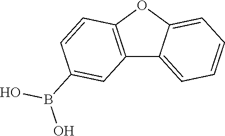 [SMILES: OB(O)c1ccc2oc3ccccc3c2c1] (402936-15-6) | 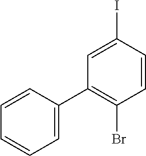 [SMILES: Brc1ccc(I)cc1-c1ccccc1] (1226452-23-8) | 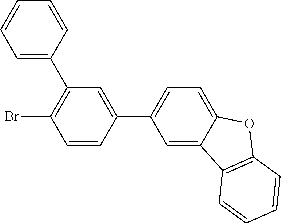 [SMILES: Brc1ccc(-c2ccc3oc4ccccc4c3c2)cc1-c1ccccc1] |
| I-6 | 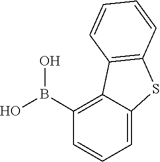 [SMILES: OB(O)c1cccc2sc3ccccc3c12] (1245943-60-5) | 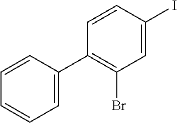 [SMILES: Brc1cc(I)ccc1-c1ccccc1] (1613576-54-7) | 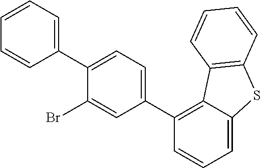 [SMILES: Brc1cc(-c2cccc3sc4ccccc4c23)ccc1-c1ccccc1] |
| I-7 | 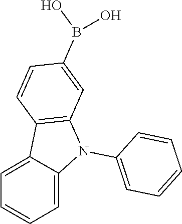 [SMILES: OB(O)c1ccc2c3ccccc3n(-c3ccccc3)c2c1] (1245943-60-5) | 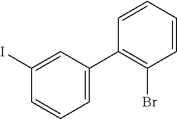 [SMILES: Brc1ccccc1-c1cccc(I)c1] (1776936-09-4) | 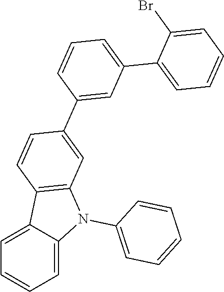 [SMILES: Brc1ccccc1-c1cccc(-c2ccc3c4ccccc4n(-c4ccccc4)c3c2)c1] |
| I-8 | 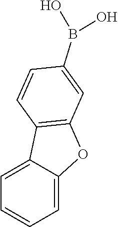 [SMILES: OB(O)c1ccc2c(c1)oc1ccccc12] (395087-89-5) | 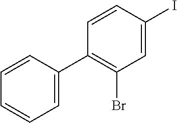 [SMILES: Brc1cc(I)ccc1-c1ccccc1] (1613576-54-7) | 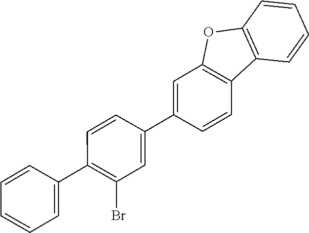 [SMILES: Brc1cc(-c2ccc3c(c2)oc2ccccc23)ccc1-c1ccccc1] |
| I-9 | 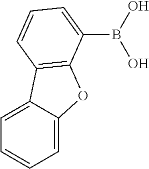 [SMILES: OB(O)c1cccc2c1oc1ccccc12] (100124-06-9) | 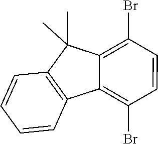 [SMILES: CC1(C)c2ccccc2-c2c(Br)ccc(Br)c21] (11813537-41-5) | 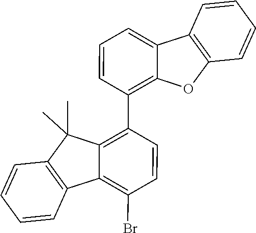 [SMILES: CC1(C)c2ccccc2-c2c(Br)ccc(-c3cccc4c3oc3ccccc34)c21] |
| I-10 | 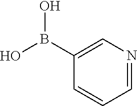 (1692-25-7) | 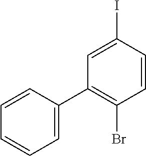 [SMILES: Brc1ccc(I)cc1-c1ccccc1] (1226452-23-8) | 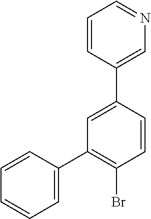 [SMILES: Brc1ccc(-c2cccnc2)cc1-c1ccccc1] |
| I-11 | 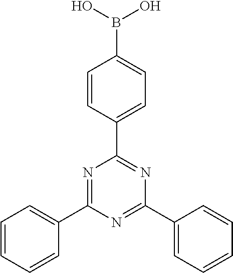 [SMILES: OB(O)c1ccc(-c2nc(-c3ccccc3)nc(-c3ccccc3)n2)cc1] (1313018-07-3) | 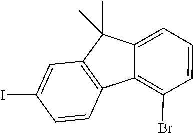 [SMILES: CC1(C)c2cc(I)ccc2-c2c(Br)cccc21] (1822311-14-7) | 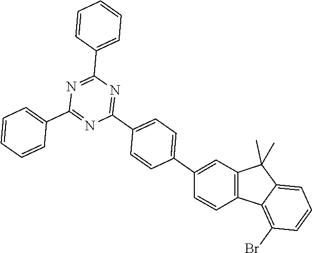 [SMILES: CC1(C)c2cc(-c3ccc(-c4nc(-c5ccccc5)nc(-c5ccccc5)n4)cc3)ccc2-c2c(Br)cccc21] |
| I-12 | 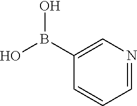 (1692-25-7) | 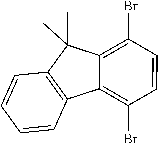 [SMILES: CC1(C)c2ccccc2-c2c(Br)ccc(Br)c21] (11813537-41-5) | 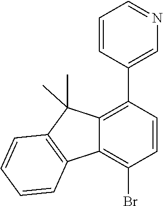 [SMILES: CC1(C)c2ccccc2-c2c(Br)ccc(-c3cccnc3)c21] |
| I-13 | 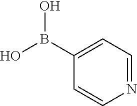 (1692-15-5) | 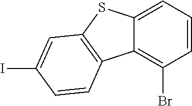 [SMILES: Brc1cccc2sc3cc(I)ccc3c12] (1807910-82-2) | 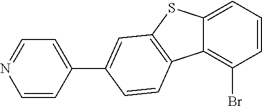 [SMILES: Brc1cccc2sc3cc(-c4ccncc4)ccc3c12] |
| I-14 | 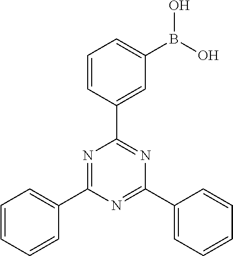 [SMILES: OB(O)c1cccc(-c2nc(-c3ccccc3)nc(-c3ccccc3)n2)c1] (1612243-82-9) | 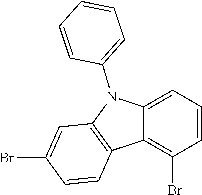 [SMILES: Brc1ccc2c3c(Br)cccc3n(-c3ccccc3)c2c1] (1507368-39-9) | 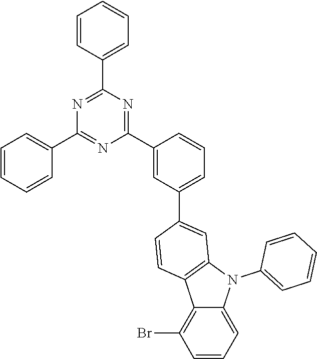 [SMILES: Brc1cccc2c1c1ccc(-c3cccc(-c4nc(-c5ccccc5)nc(-c5ccccc5)n4)c3)cc1n2-c1ccccc1] |
| I-15 | 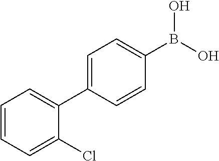 [SMILES: OB(O)c1ccc(-c2ccccc2Cl)cc1] (1383531-51-8) | 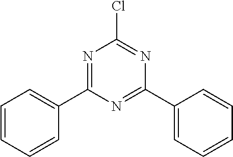 [SMILES: Clc1nc(-c2ccccc2)nc(-c2ccccc2)n1] (3842-55-5) | 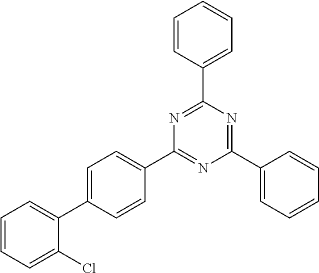 [SMILES: Clc1ccccc1-c1ccc(-c2nc(-c3ccccc3)nc(-c3ccccc3)n2)cc1] |
| I-16 | 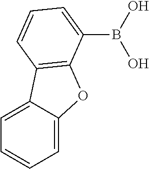 [SMILES: OB(O)c1cccc2c1oc1ccccc12] (100124-06-9) | 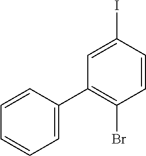 [SMILES: Brc1ccc(I)cc1-c1ccccc1] (1226452-23-8) | 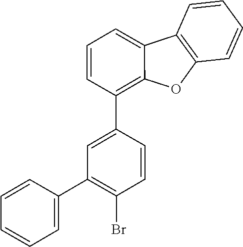 [SMILES: Brc1ccc(-c2cccc3c2oc2ccccc23)cc1-c1ccccc1] |
| I-17 | 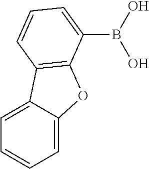 [SMILES: OB(O)c1cccc2c1oc1ccccc12] (100124-06-9) | 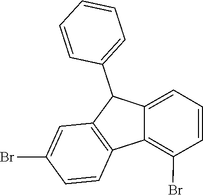 [SMILES: Brc1ccc2c(c1)C(c1ccccc1)c1cccc(Br)c1-2] (1822311-14-7) | 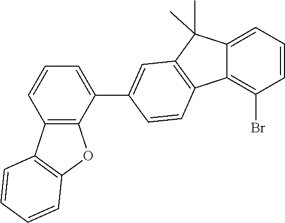 [SMILES: CC1(C)c2cc(-c3cccc4c3oc3ccccc34)ccc2-c2c(Br)cccc21] |
| I-18 | 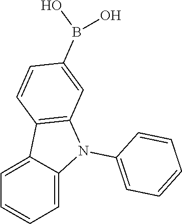 [SMILES: OB(O)c1ccc2c3ccccc3n(-c3ccccc3)c2c1] (1245943-60-5) | 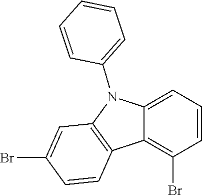 [SMILES: Brc1ccc2c3c(Br)cccc3n(-c3ccccc3)c2c1] (1507368-39-9) | 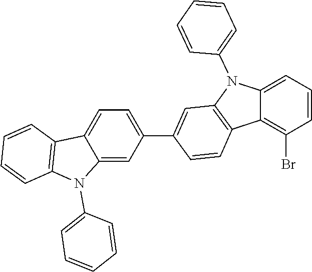 [SMILES: Brc1cccc2c1c1ccc(-c3ccc4c5ccccc5n(-c5ccccc5)c4c3)cc1n2-c1ccccc1] |
| I-19 | 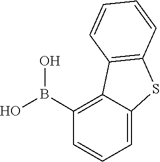 [SMILES: OB(O)c1cccc2sc3ccccc3c12] (1245943-60-5) | 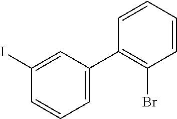 [SMILES: Brc1ccccc1-c1cccc(I)c1] (1776936-09-4) | 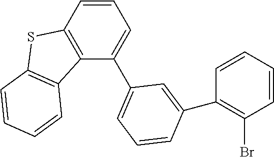 [SMILES: Brc1ccccc1-c1cccc(-c2cccc3sc4ccccc4c23)c1] |

### Synthesis of (9,9-dimethyl-9H-fluoren-2-yl)(9,9-spirobifluoren-2-yl)(3′-pyridin-3-ylbiphenyl-2-yl)amine (1-1) and of the compounds (I-2) to (I-15)

25.3 g of (9,9-dimethyl-9H-fluoren-2-yl)(9,9-spirobifluoren-2-yl)amine (48.4 mmol) and 20 g of 3-(2′-bromobiphenyl-3-yl)pyridine (48.4 mmol) are dissolved in 300 ml of toluene. The solution is degassed and saturated with N₂. Thereafter, 1.95 ml (2.17 mmol) of a 1 M tri-tert-butylphosphine solution and 0.217 g (0.97 mmol) of palladium(II) acetate are added thereto. Subsequently, 11.2 g of sodium pentoxide (96.7 mmol) are added. The reaction mixture is heated to boiling under a protective atmosphere for 4 h. The mixture is subsequently partitioned between toluene and water, and the organic phase is washed three times with water, dried over Na₂SO₄ and concentrated by rotary evaporation. After the crude product has been filtered through silica gel with toluene, the remaining residue is recrystallized from heptane/toluene. The residue of 22.9 g (70% of theory) is finally sublimed under high vacuum.

The following compounds are prepared in an analogous manner:

| | | |
|---|---|---|
| Reactant 1 | Reactant 2 | Product |

| | | | |
|---|---|---|---|
| 1-2 | 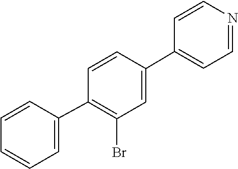 [SMILES: Brc1cc(-c2ccncc2)ccc1-c1ccccc1] | 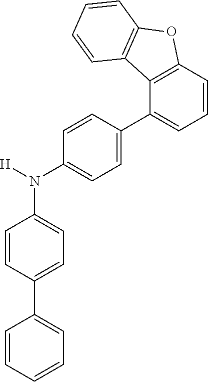 [SMILES: c1ccc(-c2ccc(Nc3ccc(-c4cccc5oc6ccccc6c45)cc3)cc2)cc1] | 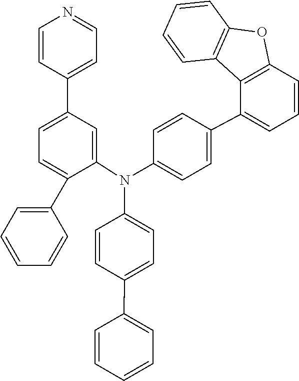 [SMILES: c1ccc(-c2ccc(N(c3ccc(-c4cccc5oc6ccccc6c45)cc3)c3cc(-c4ccncc4)ccc3-c3ccccc3)cc2)cc1] |
| 1-3 | 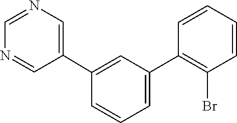 [SMILES: Brc1ccccc1-c1cccc(-c2cncnc2)c1] | 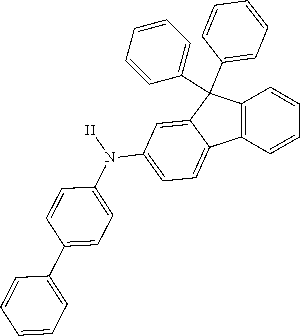 [SMILES: c1ccc(-c2ccc(Nc3ccc4c(c3)C(c3ccccc3)(c3ccccc3)c3ccccc3-4)cc2)cc1] | 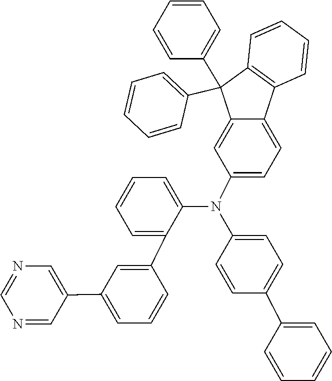 [SMILES: c1ccc(-c2ccc(N(c3ccc4c(c3)C(c3ccccc3)(c3ccccc3)c3ccccc3-4)c3ccccc3-c3cccc(-c4cncnc4)c3)cc2)cc1] |
| 1-4 | 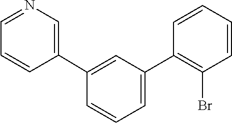 [SMILES: Brc1ccccc1-c1cccc(-c2cccnc2)c1] | 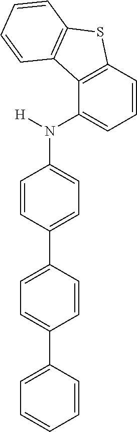 [SMILES: c1ccc(-c2ccc(-c3ccc(Nc4cccc5sc6ccccc6c45)cc3)cc2)cc1] | 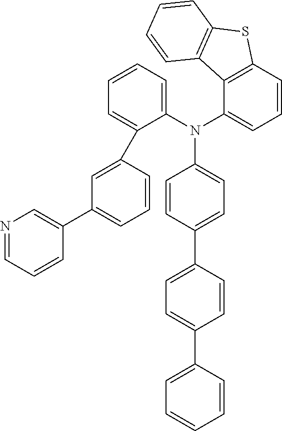 [SMILES: c1ccc(-c2ccc(-c3ccc(N(c4ccccc4-c4cccc(-c5cccnc5)c4)c4cccc5sc6ccccc6c45)cc3)cc2)cc1] |
| 1-5 | 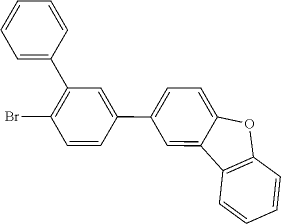 [SMILES: Brc1ccc(-c2ccc3oc4ccccc4c3c2)cc1-c1ccccc1] | 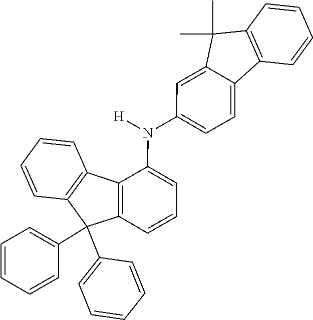 [SMILES: CC1(C)c2ccccc2-c2ccc(Nc3cccc4c3-c3ccccc3C4(c3ccccc3)c3ccccc3)cc21] | 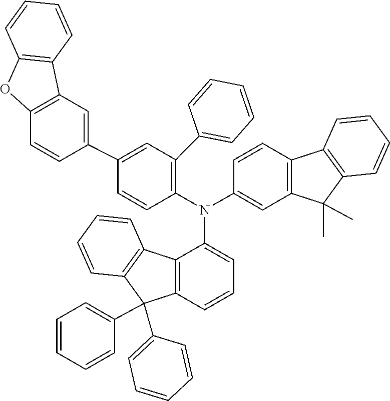 [SMILES: CC1(C)c2ccccc2-c2ccc(N(c3ccc(-c4ccc5oc6ccccc6c5c4)cc3-c3ccccc3)c3cccc4c3-c3ccccc3C4(c3ccccc3)c3ccccc3)cc21] |
| 1-6 | 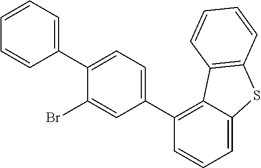 [SMILES: Brc1cc(-c2cccc3sc4ccccc4c23)ccc1-c1ccccc1] | 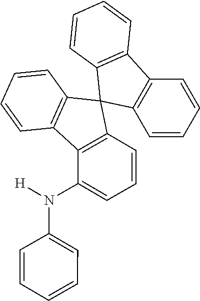 [SMILES: c1ccc(Nc2cccc3c2-c2ccccc2C32c3ccccc3-c3ccccc32)cc1] | 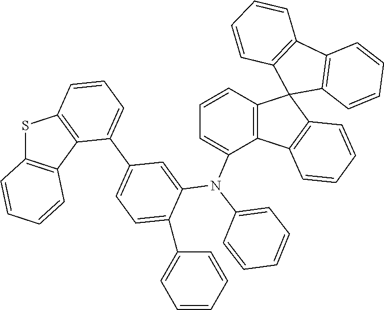 [SMILES: c1ccc(-c2ccc(-c3cccc4sc5ccccc5c34)cc2N(c2ccccc2)c2cccc3c2-c2ccccc2C32c3ccccc3-c3ccccc32)cc1] |
| 1-7 | 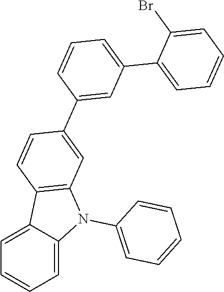 [SMILES: Brc1ccccc1-c1cccc(-c2ccc3c4ccccc4n(-c4ccccc4)c3c2)c1] | 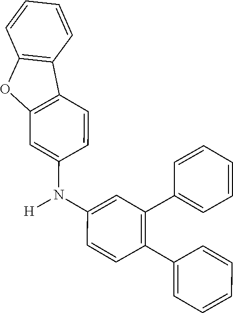 [SMILES: c1ccc(-c2ccc(Nc3ccc4c(c3)oc3ccccc34)cc2-c2ccccc2)cc1] | 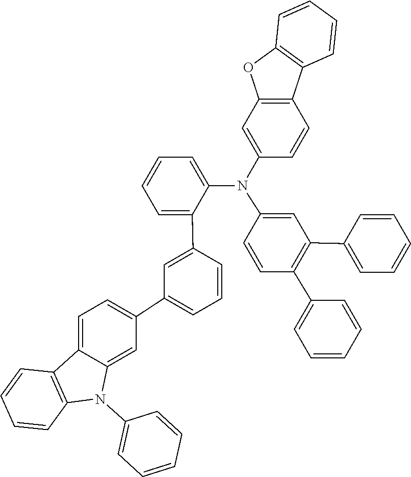 [SMILES: c1ccc(-c2ccc(N(c3ccc4c(c3)oc3ccccc34)c3ccccc3-c3cccc(-c4ccc5c6ccccc6n(-c6ccccc6)c5c4)c3)cc2-c2ccccc2)cc1] |
| 1-8 | 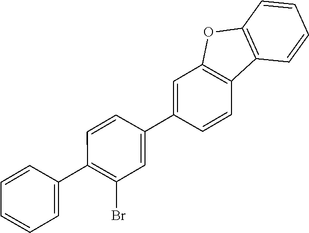 [SMILES: Brc1cc(-c2ccc3c(c2)oc2ccccc23)ccc1-c1ccccc1] | 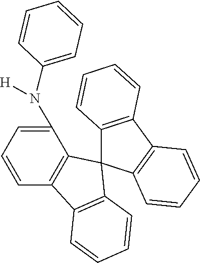 [SMILES: c1ccc(Nc2cccc3c2C2(c4ccccc4-c4ccccc42)c2ccccc2-3)cc1] | 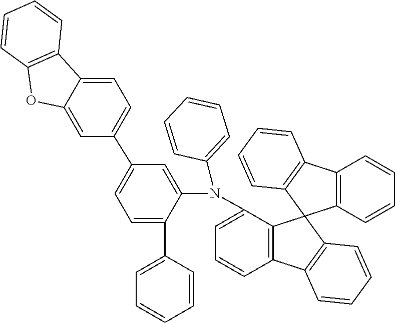 [SMILES: c1ccc(-c2ccc(-c3ccc4c(c3)oc3ccccc34)cc2N(c2ccccc2)c2cccc3c2C2(c4ccccc4-c4ccccc42)c2ccccc2-3)cc1] |
| 1-9 | 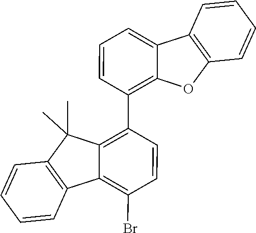 [SMILES: CC1(C)c2ccccc2-c2c(Br)ccc(-c3cccc4c3oc3ccccc34)c21] | 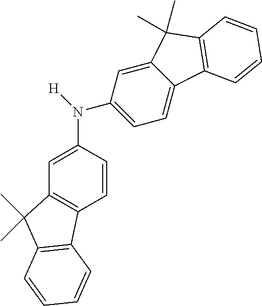 [SMILES: CC1(C)c2ccccc2-c2ccc(Nc3ccc4c(c3)C(C)(C)c3ccccc3-4)cc21] | 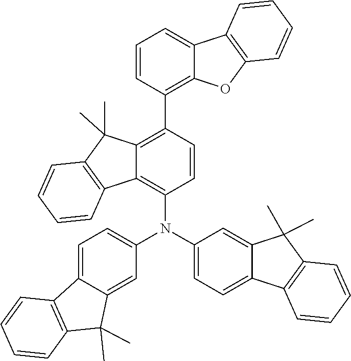 [SMILES: CC1(C)c2ccccc2-c2ccc(N(c3ccc4c(c3)C(C)(C)c3ccccc3-4)c3ccc(-c4cccc5c4oc4ccccc45)c4c3-c3ccccc3C4(C)C)cc21] |
| 1-10 | 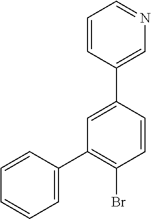 [SMILES: Brc1ccc(-c2cccnc2)cc1-c1ccccc1] | 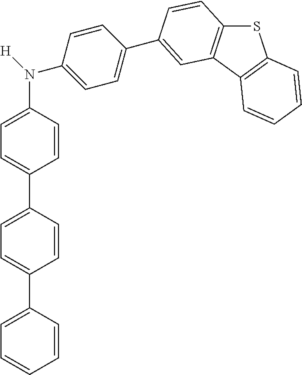 [SMILES: c1ccc(-c2ccc(-c3ccc(Nc4ccc(-c5ccc6sc7ccccc7c6c5)cc4)cc3)cc2)cc1] | 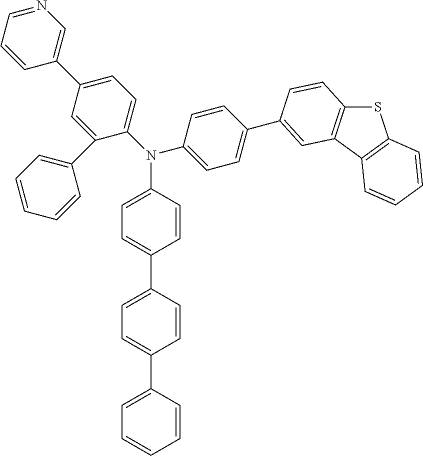 [SMILES: c1ccc(-c2ccc(-c3ccc(N(c4ccc(-c5ccc6sc7ccccc7c6c5)cc4)c4ccc(-c5cccnc5)cc4-c4ccccc4)cc3)cc2)cc1] |
| 1-11 | 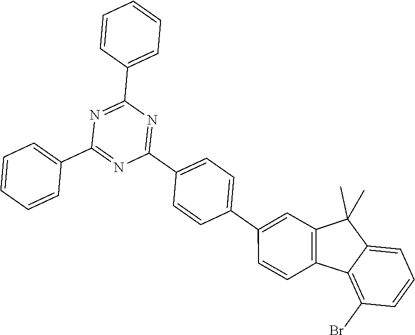 [SMILES: CC1(C)c2cc(-c3ccc(-c4nc(-c5ccccc5)nc(-c5ccccc5)n4)cc3)ccc2-c2c(Br)cccc21] | 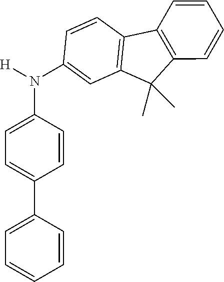 [SMILES: CC1(C)c2ccccc2-c2ccc(Nc3ccc(-c4ccccc4)cc3)cc21] | 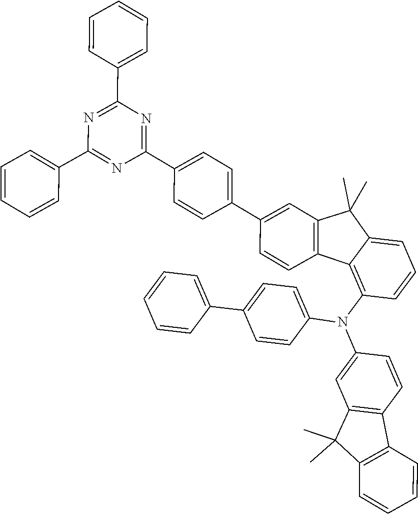 [SMILES: CC1(C)c2ccccc2-c2ccc(N(c3ccc(-c4ccccc4)cc3)c3cccc4c3-c3ccc(-c5ccc(-c6nc(-c7ccccc7)nc(-c7ccccc7)n6)cc5)cc3C4(C)C)cc21] |
| 1-12 | 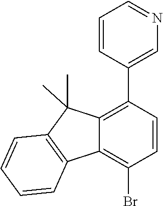 [SMILES: CC1(C)c2ccccc2-c2c(Br)ccc(-c3cccnc3)c21] | 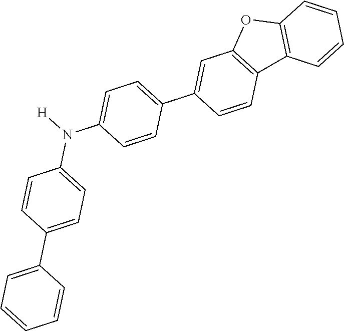 [SMILES: c1ccc(-c2ccc(Nc3ccc(-c4ccc5c(c4)oc4ccccc45)cc3)cc2)cc1] | 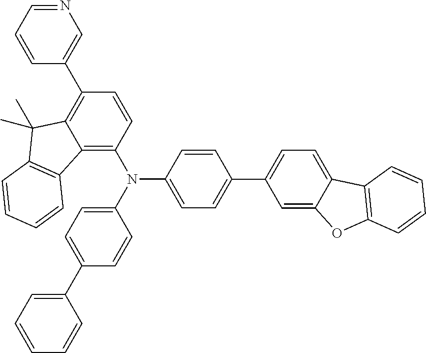 [SMILES: CC1(C)c2ccccc2-c2c(N(c3ccc(-c4ccccc4)cc3)c3ccc(-c4ccc5c(c4)oc4ccccc45)cc3)ccc(-c3cccnc3)c21] |
| 1-13 | 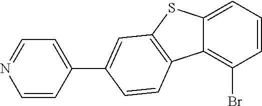 [SMILES: Brc1cccc2sc3cc(-c4ccncc4)ccc3c12] | 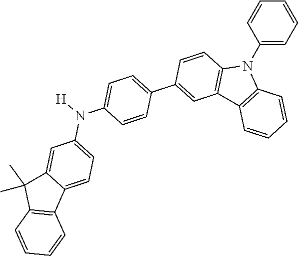 [SMILES: CC1(C)c2ccccc2-c2ccc(Nc3ccc(-c4ccc5c(c4)c4ccccc4n5-c4ccccc4)cc3)cc21] | 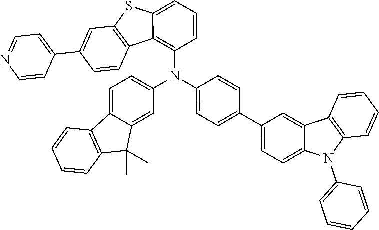 [SMILES: CC1(C)c2ccccc2-c2ccc(N(c3ccc(-c4ccc5c(c4)c4ccccc4n5-c4ccccc4)cc3)c3cccc4sc5cc(-c6ccncc6)ccc5c34)cc21] |
| 1-14 | 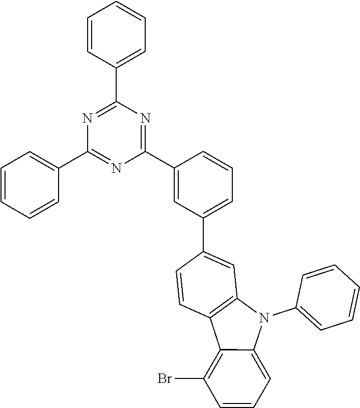 [SMILES: Brc1cccc2c1c1ccc(-c3cccc(-c4nc(-c5ccccc5)nc(-c5ccccc5)n4)c3)cc1n2-c1ccccc1] | 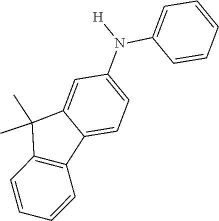 [SMILES: CC1(C)c2ccccc2-c2ccc(Nc3ccccc3)cc21] | 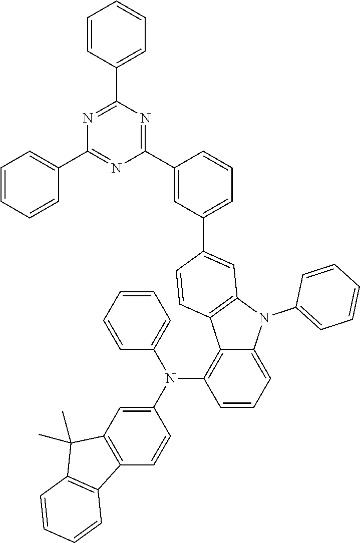 [SMILES: CC1(C)c2ccccc2-c2ccc(N(c3ccccc3)c3cccc4c3c3ccc(-c5cccc(-c6nc(-c7ccccc7)nc(-c7ccccc7)n6)c5)cc3n4-c3ccccc3)cc21] |
| 1-15 | 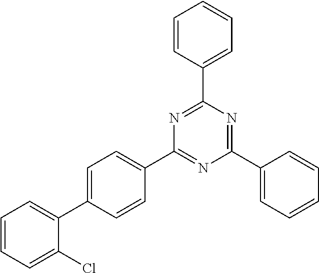 [SMILES: Clc1ccccc1-c1ccc(-c2nc(-c3ccccc3)nc(-c3ccccc3)n2)cc1] | 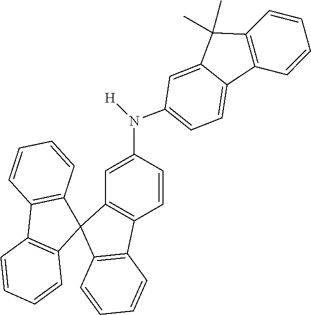 [SMILES: CC1(C)c2ccccc2-c2ccc(Nc3ccc4c(c3)C3(c5ccccc5-c5ccccc53)c3ccccc3-4)cc21] | 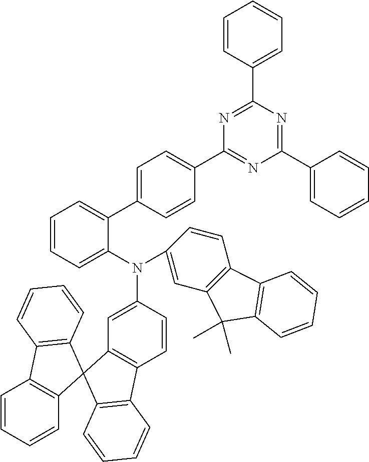 [SMILES: CC1(C)c2ccccc2-c2ccc(N(c3ccc4c(c3)C3(c5ccccc5-c5ccccc53)c3ccccc3-4)c3ccccc3-c3ccc(-c4nc(-c5ccccc5)nc(-c5ccccc5)n4)cc3)cc21] |
| 1-16 | 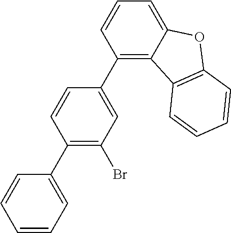 [SMILES: Brc1cc(-c2cccc3oc4ccccc4c23)ccc1-c1ccccc1] | 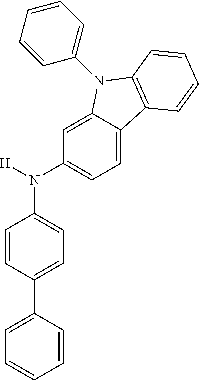 [SMILES: c1ccc(-c2ccc(Nc3ccc4c5ccccc5n(-c5ccccc5)c4c3)cc2)cc1] | 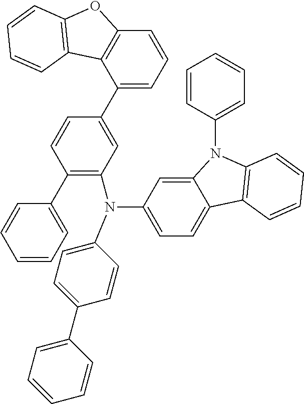 [SMILES: c1ccc(-c2ccc(N(c3ccc4c5ccccc5n(-c5ccccc5)c4c3)c3cc(-c4cccc5oc6ccccc6c45)ccc3-c3ccccc3)cc2)cc1] |
| 1-17 | 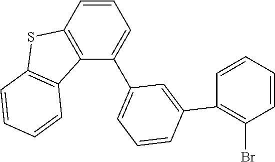 [SMILES: Brc1ccccc1-c1cccc(-c2cccc3sc4ccccc4c23)c1] | 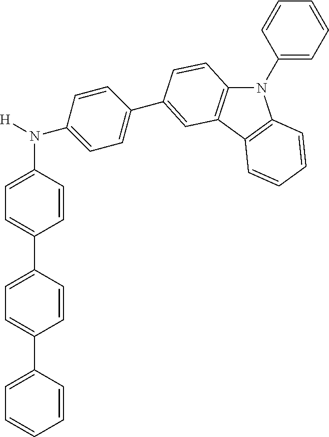 [SMILES: c1ccc(-c2ccc(-c3ccc(Nc4ccc(-c5ccc6c(c5)c5ccccc5n6-c5ccccc5)cc4)cc3)cc2)cc1] | 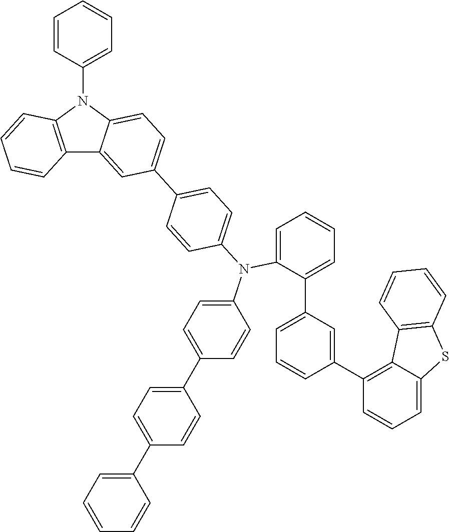 [SMILES: c1ccc(-c2ccc(-c3ccc(N(c4ccc(-c5ccc6c(c5)c5ccccc5n6-c5ccccc5)cc4)c4ccccc4-c4cccc(-c5cccc6sc7ccccc7c56)c4)cc3)cc2)cc1] |
| 1-18 | 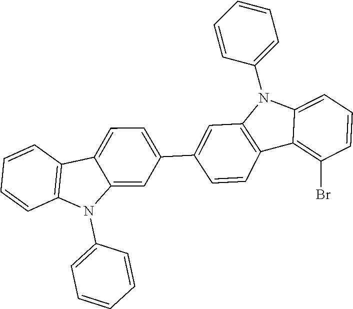 [SMILES: Brc1cccc2c1c1ccc(-c3ccc4c5ccccc5n(-c5ccccc5)c4c3)cc1n2-c1ccccc1] | 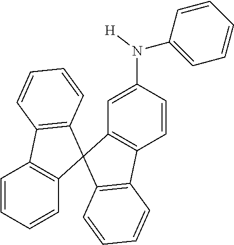 [SMILES: c1ccc(Nc2ccc3c(c2)C2(c4ccccc4-c4ccccc42)c2ccccc2-3)cc1] | 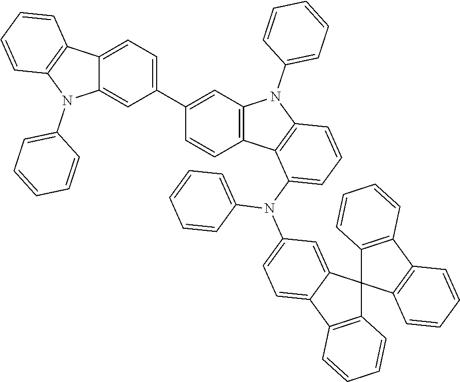 [SMILES: c1ccc(N(c2ccc3c(c2)C2(c4ccccc4-c4ccccc42)c2ccccc2-3)c2cccc3c2c2ccc(-c4ccc5c6ccccc6n(-c6ccccc6)c5c4)cc2n3-c2ccccc2)cc1] |
| 1-19 | 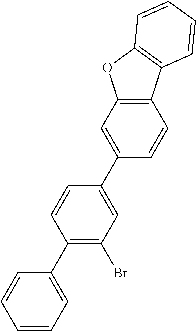 [SMILES: Brc1cc(-c2ccc3c(c2)oc2ccccc23)ccc1-c1ccccc1] | 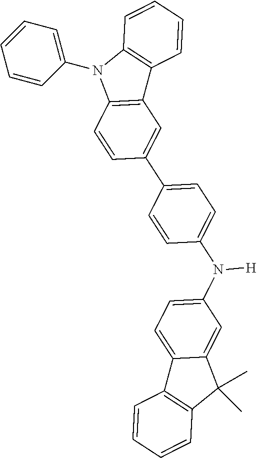 [SMILES: CC1(C)c2ccccc2-c2ccc(Nc3ccc(-c4ccc5c(c4)c4ccccc4n5-c4ccccc4)cc3)cc21] | 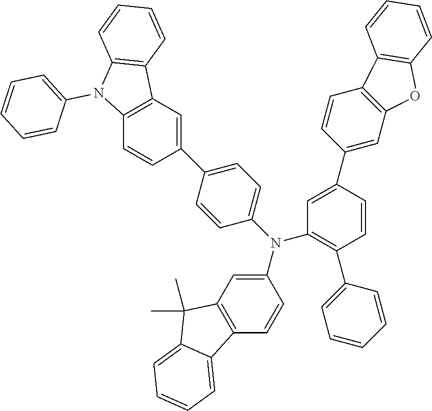 [SMILES: CC1(C)c2ccccc2-c2ccc(N(c3ccc(-c4ccc5c(c4)c4ccccc4n5-c4ccccc4)cc3)c3cc(-c4ccc5c(c4)oc4ccccc45)ccc3-c3ccccc3)cc21] |
| 1-20 | 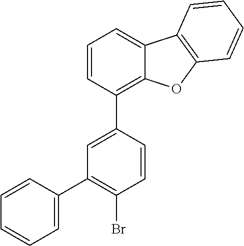 [SMILES: Brc1ccc(-c2cccc3c2oc2ccccc23)cc1-c1ccccc1] | 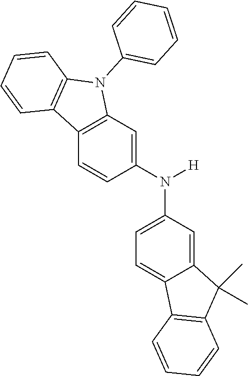 [SMILES: CC1(C)c2ccccc2-c2ccc(Nc3ccc4c5ccccc5n(-c5ccccc5)c4c3)cc21] | 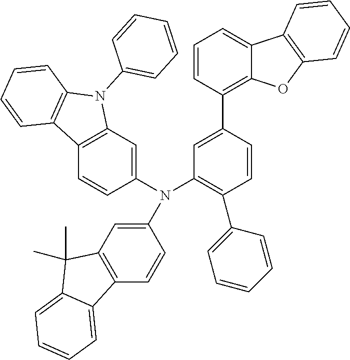 [SMILES: CC1(C)c2ccccc2-c2ccc(N(c3ccc4c5ccccc5n(-c5ccccc5)c4c3)c3cc(-c4cccc5c4oc4ccccc45)ccc3-c3ccccc3)cc21] |
| 1-21 | 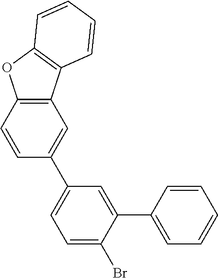 [SMILES: Brc1ccc(-c2ccc3oc4ccccc4c3c2)cc1-c1ccccc1] | 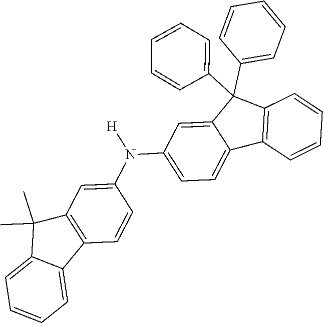 [SMILES: CC1(C)c2ccccc2-c2ccc(Nc3ccc4c(c3)C(c3ccccc3)(c3ccccc3)c3ccccc3-4)cc21] | 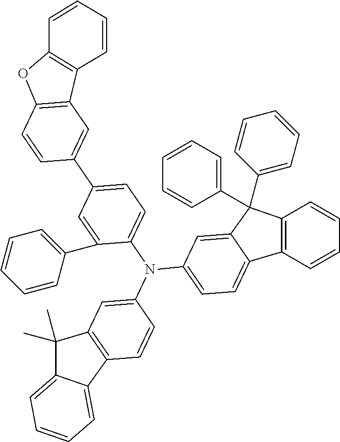 [SMILES: CC1(C)c2ccccc2-c2ccc(N(c3ccc4c(c3)C(c3ccccc3)(c3ccccc3)c3ccccc3-4)c3ccc(-c4ccc5oc6ccccc6c5c4)cc3-c3ccccc3)cc21] |
| 1-22 | 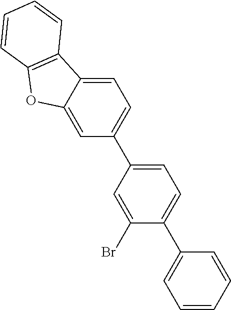 [SMILES: Brc1cc(-c2ccc3c(c2)oc2ccccc23)ccc1-c1ccccc1] | 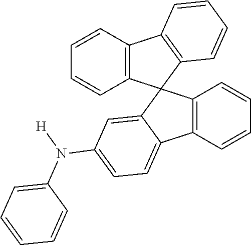 [SMILES: c1ccc(Nc2ccc3c(c2)C2(c4ccccc4-c4ccccc42)c2ccccc2-3)cc1] | 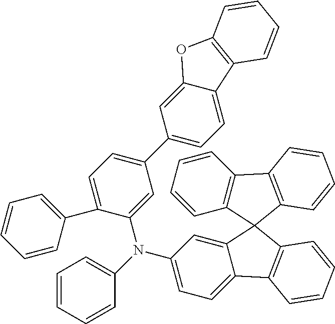 [SMILES: c1ccc(-c2ccc(-c3ccc4c(c3)oc3ccccc34)cc2N(c2ccccc2)c2ccc3c(c2)C2(c4ccccc4-c4ccccc42)c2ccccc2-3)cc1] |

### B) Device Examples

OLEDs containing compounds of the formula (I) are produced by methods that are common knowledge in the prior art. Subsequently, the OLEDs are put into operation, and the properties of the OLEDs are examined.

In the production of the OLEDs, the following general method is employed: The substrates used are glass plaques coated with structured ITO (indium tin oxide) in a layer thickness of 50 nm. The ITO layer forms the anode. To this are applied the following layers in the sequence specified: hole injection layer (HIL), optional hole transport layer (HTL), electron blocker layer (EBL), emitting layer (EML), optional hole blocker layer (HBL), electron transport layer (ETL), electron injection layer (EIL) and cathode.

The materials used in the layers are shown correspondingly in the tables below. The cathode is formed by an aluminium layer having a thickness of 100 nm.

The materials are each applied by thermal deposition from the gas phase. As shown below, the layers may consist of a single material, or of a mixture of two or three different materials. If they consist of a mixture, they are produced by co-evaporation of the materials present. If, as shown below, information is given in the form of H1:SEB(3%), this means that H1 is present in the layer in a proportion by volume of 97% and SEB in a proportion by volume of 3%.

All the OLEDs produced are put into operation. It is determined here that the OLEDs produced are functional, i.e. emit light of the expected colour.

Finally, the OLEDs produced are examined for their properties. The parameters determined here are the operating voltage U, the external quantum efficiency EQE and the lifetime LT80. For each of the values U, EQE and LT80, the luminance in cd/m² or the current density in mA/cm² at which the corresponding values are determined is reported. LT80 is the time that elapses before the value for the OLED in question has dropped from 100% to 80%, based in each case on the luminance or current density reported. In the corresponding calculation, an acceleration factor of 1.8 is employed.

### 1st Experimental Setup:

Blue-fluorescing OLEDs with the structure specified in the table below are produced. The inventive compounds 1-21, 1-22, 1-5 and 1-8 are used here in the EBL.

| | HIL | HTL | EBL | EML | ETL | EIL |
|---|---|---|---|---|---|---|
| | Thickness/ | Thickness/ | Thickness/ | Thickness/ | Thickness/ | Thickness/ |
| Ex. | nm | nm | nm | nm | nm | nm |
| I1 | HIM2: | HIM2 | 1-21 | H1:SEB | ETM:LiQ | LiQ |
| | F4TCNQ | 170 nm | 10 nm | (3%) | (50%) | 1 nm |
| | (5%) | | | 20 nm | 30 nm | |
| | 10 nm | | | | | |
| I2 | as above | as above | 1-22 | as above | as above | as above |
| | | | 10 nm | | | |
| I3 | as above | as above | 1-5 | as above | as above | as above |
| | | | 10 nm | | | |
| I4 | as above | as above | 1-8 | as above | as above | as above |
| | | | 10 nm | | | |

The following results are obtained:

| | U | EQE | LT80 |
|---|---|---|---|
| | @ 10 mA/cm² | @ 10 mA/cm² | @ 60 mA/cm² |
| Ex. | [V] | % | [h] |
| I1 | 4.0 | 5.7 | 324 |
| I2 | 4.0 | 6.1 | 360 |
| I3 | 4.0 | 6.0 | 396 |
| I4 | 3.8 | 6.7 | 432 |

This shows that OLEDs comprising compounds of the invention show good performance data in the EBL.

### 2nd Experimental Setup:

Blue-fluorescing OLEDs with the structure specified in the table below are produced. The inventive compounds 1-1, 1-2, 1-3, 1-4, 1-7, 1-10, 1-12 and 1-13 are used here in the HTL and, having been doped with F4TCNQ, in the HIL.

| | HIL | HTL | EBL | EML | ETL | EIL |
|---|---|---|---|---|---|---|
| | Thickness/ | Thickness/ | Thickness/ | Thickness/ | Thickness/ | Thickness/ |
| Ex. | nm | nm | nm | nm | nm | nm |
| I5 | 1-1: | 1-1 | HTM3 | H1:SEB | ETM:LiQ | LiQ |
| | F4TCNQ | 170 nm | 10 nm | (3%) | (50%) | 1 nm |
| | (5%) | | | 20 nm | 30 nm | |
| | 10 nm | | | | | |
| I6 | 1-2: | 1-2 | as above | as above | as above | as above |
| | F4TCNQ | 170 nm | | | | |
| | (5%) | | | | | |
| | 10 nm | | | | | |
| I7 | 1-3: | 1-3 | as above | as above | as above | as above |
| | F4TCNQ | 170 nm | | | | |
| | (5%) | | | | | |
| | 10 nm | | | | | |
| I8 | 1-4: | 1-4 | as above | as above | as above | as above |
| | F4TCNQ | 170 nm | | | | |
| | (5%) | | | | | |
| | 10 nm | | | | | |
| I9 | 1-7: | 1-7 | as above | as above | as above | as above |
| | F4TCNQ | 170 nm | | | | |
| | (5%) | | | | | |
| | 10 nm | | | | | |
| I9-1 | 1-10: | 1-10 | as above | as above | as above | as above |
| | F4TCNQ | 170 nm | | | | |
| | (5%) | | | | | |
| | 10 nm | | | | | |
| I9-2 | 1-12: | 1-12 | as above | as above | as above | as above |
| | F4TCNQ | 170 nm | | | | |
| | (5%) | | | | | |
| | 10 nm | | | | | |
| I9-3 | 1-13: | 1-13 | as above | as above | as above | as above |
| | F4TCNQ | 170 nm | | | | |
| | (5%) | | | | | |
| | 10 nm | | | | | |

The following results are obtained:

| | U | EQE | LT80 |
|---|---|---|---|
| | @ 10 mA/cm² | @ 10 mA/cm² | @ 60 mA/cm² |
| Ex. | [V] | % | [h] |
| I5 | 4.0 | 6.4 | 415 |
| I6 | 4.1 | 6.1 | 427 |
| I7 | 4.0 | 6.6 | 375 |
| I8 | 4.2 | 6.2 | 340 |
| I9 | 4.6 | 6.2 | 380 |

This shows that OLEDs comprising compounds of the invention show good performance data in the HIL and the HTL.

In experiments I9-1, I9-2 and I9-3, satisfactory results for lifetime and EQE are obtained.

### 3rd Experimental Setup:

Blue-fluorescing OLEDs with the structure specified in the table below are produced. The inventive compound 1-6 is used here in the EBL.

| | HIL | HTL | EBL | EML | ETL | EIL |
|---|---|---|---|---|---|---|
| | Thickness/ | Thickness/ | Thickness/ | Thickness/ | Thickness/ | Thickness/ |
| Ex. | nm | nm | nm | nm | nm | nm |
| I10 | HIM1: | HIM1 | 1-6 | H1:SEB | ETM:LiQ | LiQ |
| | F4TCNQ | 170 nm | 10 nm | (3%) | (50%) | 1 nm |
| | (5%) | | | 20 nm | 30 nm | |
| | 10 nm | | | | | |

The following results are obtained:

| | U | EQE | LT80 |
|---|---|---|---|
| | @ 10 mA/cm² | @ 10 mA/cm² | @ 60 mA/cm² |
| Ex. | [V] | % | [h] |
| I10 | 4.6 | 6.8 | 300 |

Like the 1st experimental setup, this shows that OLEDs comprising compounds of the invention show good performance data in the EBL.

### 4th Experimental Setup:

Green-fluorescing OLEDs with the structure specified in the table below are produced. The inventive compounds 1-11, 1-14 and 1-15 are used here in the EML as matrix material.

| | HIL | HTL | EBL | EML | HBL | ETL | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| I11 | HTM3: | — | HTM3 | H2(29%): | ETM | ETM: | LiQ |
| | F4TCNQ | | 40 nm | (1-11) | (10 nm) | LiQ | 1 nm |
| | (5%) | | | (59%): | | (50%) | |
| | 20 nm | | | TEG | | 30 nm | |
| | | | | (12%) | | | |
| | | | | 30 nm | | | |
| I12 | as above | — | as | H2(29%): | as | as | as |
| | | | above | (1-14) | above | above | above |
| | | | | (59%): | | | |
| | | | | TEG | | | |
| | | | | (12%) | | | |
| | | | | 30 nm | | | |
| I13 | as above | — | as | H2(29%): | as | as | as |
| | | | above | (1-15) | above | above | above |
| | | | | (59%): | | | |
| | | | | TEG | | | |
| | | | | (12%) | | | |
| | | | | 30 nm | | | |

The following results are obtained:

| | U | EQE | LT80 |
|---|---|---|---|
| | @ 1000 cd/m² | @ 1000 cd/m² | @ 40 mA/cm² |
| Ex. | [V] | % | [h] |
| I11 | 3.5 | 21.4 | 87 |
| I12 | 3.8 | 22.3 | 112 |
| I13 | 3.9 | 21.9 | 143 |

This shows that OLEDs comprising compounds of the invention show good performance data as matrix materials for triplet emitters.

### 5th Experimental Setup:

Green-fluorescing OLEDs with the structure specified in the table below are produced. The inventive compound 1-9 is used here in the EML as matrix material and in the EBL.

| | HIL | HTL | EBL | EML | HBL | ETL | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| I14 | HIM1: | HIM1 | 1-9 | H2(59%): | ETM | ETM: | LiQ |
| | F4TCNQ | 215 nm | 40 nm | (1-9) | (10 nm) | LiQ | 1 nm |
| | (5%) | | | (29%) | | (50%) | |
| | 20 nm | | | TEG | | 30 nm | |
| | | | | (12%) | | | |
| | | | | 30 nm | | | |

The following results are obtained:

| | U | EQE | LT80 |
|---|---|---|---|
| | @ 1000 cd/m² | @ 1000 cd/m² | @ 40 mA/cm² |
| Ex. | [V] | % | [h] |
| 114 | 2.9 | 17.6 | 199 |

This shows that OLEDs comprising compounds of the invention show good performance data as matrix materials for triplet emitters and as electron blocker materials.

Structures of the materials used 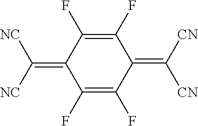 [SMILES: N#CC(C#N)=c1c(F)c(F)c(=C(C#N)C#N)c(F)c1F]
F4TCNQ 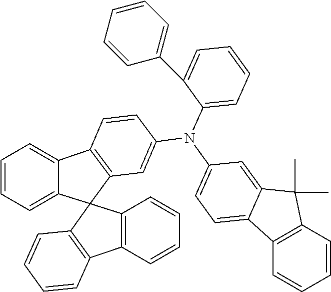 [SMILES: CC1(C)c2ccccc2-c2ccc(N(c3ccc4c(c3)C3(c5ccccc5-c5ccccc53)c3ccccc3-4)c3ccccc3-c3ccccc3)cc21]
HIM1 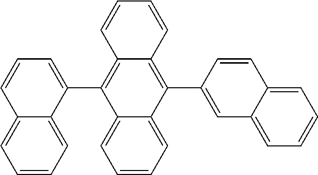 [SMILES: c1ccc2cc(-c3c4ccccc4c(-c4cccc5ccccc45)c4ccccc34)ccc2c1]
H1 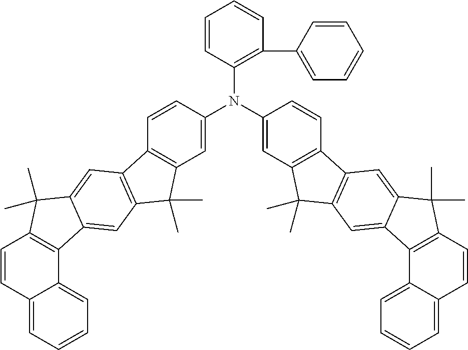 [SMILES: CC1(C)c2cc(N(c3ccc4c(c3)C(C)(C)c3cc5c(cc3-4)C(C)(C)c3ccc4ccccc4c3-5)c3ccccc3-c3ccccc3)ccc2-c2cc3c(cc21)-c1c(ccc2ccccc12)C3(C)C]
SEB 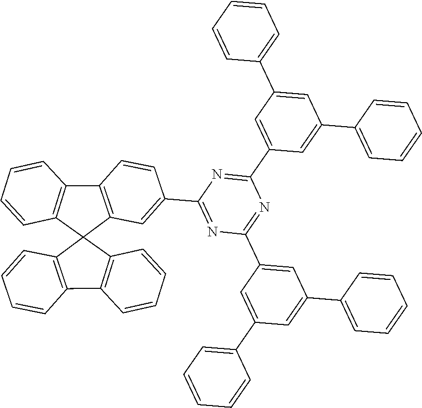 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]
ETM 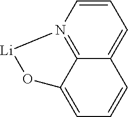
LiQ 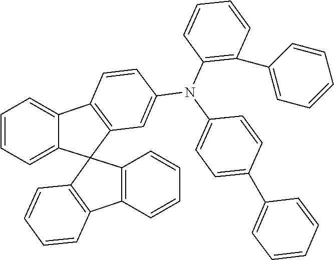 [SMILES: c1ccc(-c2ccc(N(c3ccc4c(c3)C3(c5ccccc5-c5ccccc53)c3ccccc3-4)c3ccccc3-c3ccccc3)cc2)cc1]
HIM2 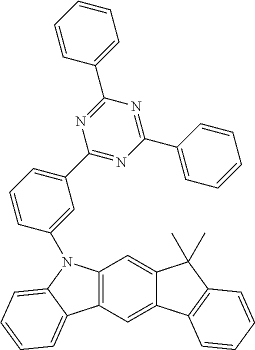 [SMILES: CC1(C)c2ccccc2-c2cc3c4ccccc4n(-c4cccc(-c5nc(-c6ccccc6)nc(-c6ccccc6)n5)c4)c3cc21]
H2 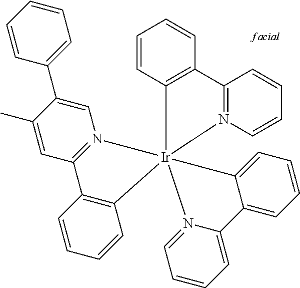
TEG 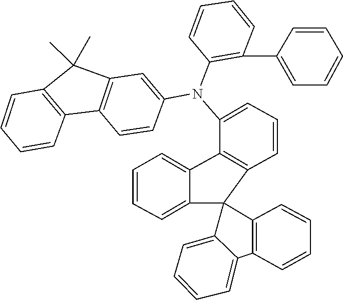 [SMILES: CC1(C)c2ccccc2-c2ccc(N(c3ccccc3-c3ccccc3)c3cccc4c3-c3ccccc3C43c4ccccc4-c4ccccc43)cc21]
HTM3 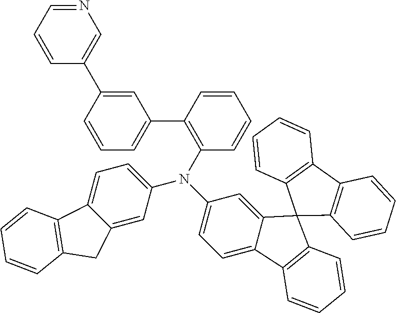 [SMILES: c1cncc(-c2cccc(-c3ccccc3N(c3ccc4c(c3)Cc3ccccc3-4)c3ccc4c(c3)C3(c5ccccc5-c5ccccc53)c3ccccc3-4)c2)c1]
1-1 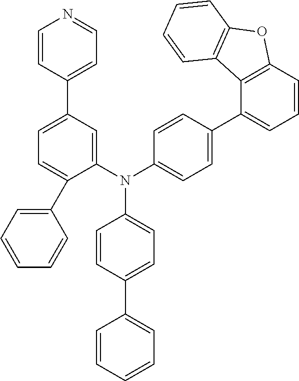 [SMILES: c1ccc(-c2ccc(N(c3ccc(-c4cccc5oc6ccccc6c45)cc3)c3cc(-c4ccncc4)ccc3-c3ccccc3)cc2)cc1]
1-2 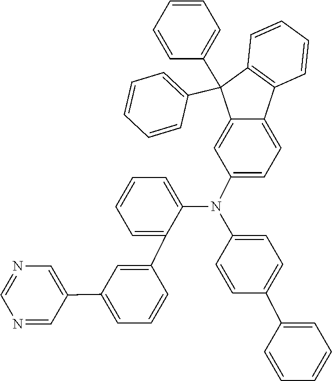 [SMILES: c1ccc(-c2ccc(N(c3ccc4c(c3)C(c3ccccc3)(c3ccccc3)c3ccccc3-4)c3ccccc3-c3cccc(-c4cncnc4)c3)cc2)cc1]
1-3 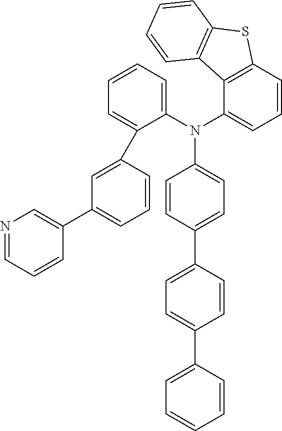 [SMILES: c1ccc(-c2ccc(-c3ccc(N(c4ccccc4-c4cccc(-c5cccnc5)c4)c4cccc5sc6ccccc6c45)cc3)cc2)cc1]
1-4 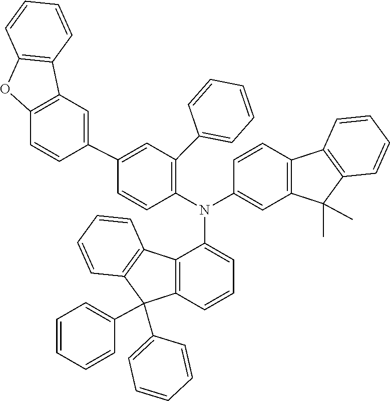 [SMILES: CC1(C)c2ccccc2-c2ccc(N(c3ccc(-c4ccc5oc6ccccc6c5c4)cc3-c3ccccc3)c3cccc4c3-c3ccccc3C4(c3ccccc3)c3ccccc3)cc21]
1-5 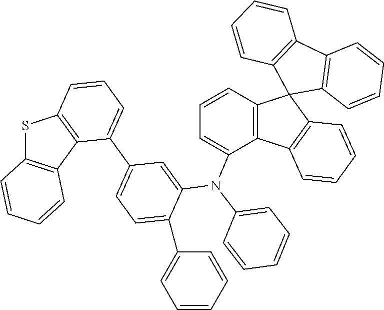 [SMILES: c1ccc(-c2ccc(-c3cccc4sc5ccccc5c34)cc2N(c2ccccc2)c2cccc3c2-c2ccccc2C32c3ccccc3-c3ccccc32)cc1]
1-6 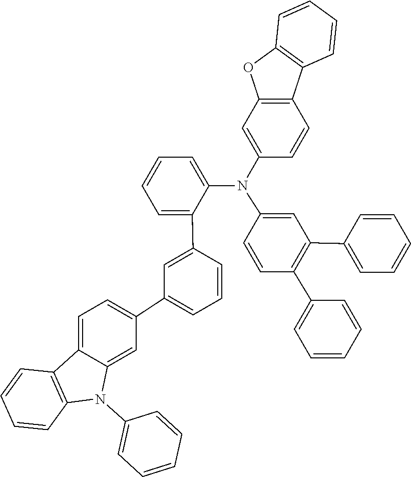 [SMILES: c1ccc(-c2ccc(N(c3ccc4c(c3)oc3ccccc34)c3ccccc3-c3cccc(-c4ccc5c6ccccc6n(-c6ccccc6)c5c4)c3)cc2-c2ccccc2)cc1]
1-7 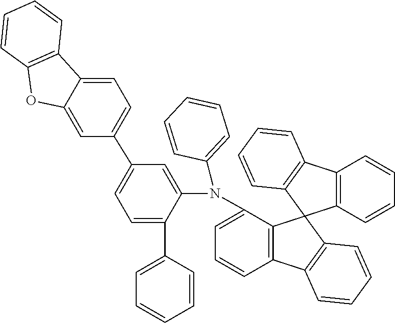 [SMILES: c1ccc(-c2ccc(-c3ccc4c(c3)oc3ccccc34)cc2N(c2ccccc2)c2cccc3c2C2(c4ccccc4-c4ccccc42)c2ccccc2-3)cc1]
1-8 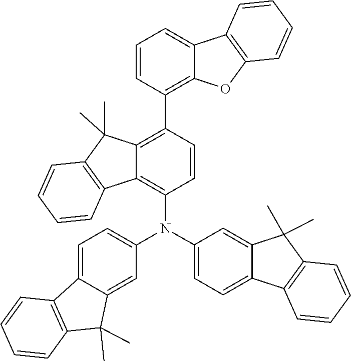 [SMILES: CC1(C)c2ccccc2-c2ccc(N(c3ccc4c(c3)C(C)(C)c3ccccc3-4)c3ccc(-c4cccc5c4oc4ccccc45)c4c3-c3ccccc3C4(C)C)cc21]
1-9 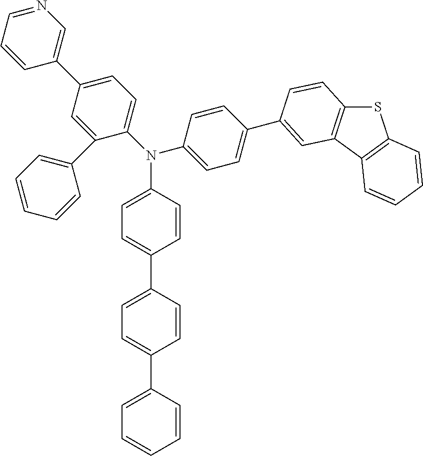 [SMILES: c1ccc(-c2ccc(-c3ccc(N(c4ccc(-c5ccc6sc7ccccc7c6c5)cc4)c4ccc(-c5cccnc5)cc4-c4ccccc4)cc3)cc2)cc1]
1-10 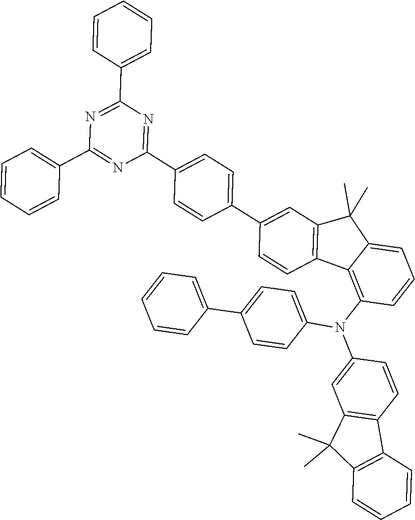 [SMILES: CC1(C)c2ccccc2-c2ccc(N(c3ccc(-c4ccccc4)cc3)c3cccc4c3-c3ccc(-c5ccc(-c6nc(-c7ccccc7)nc(-c7ccccc7)n6)cc5)cc3C4(C)C)cc21]
1-11 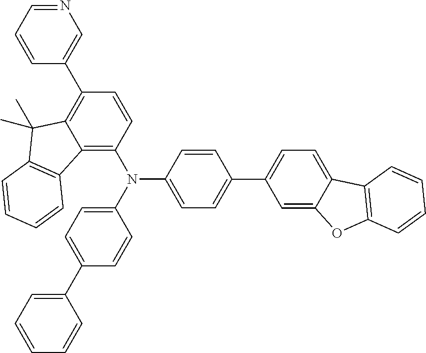 [SMILES: CC1(C)c2ccccc2-c2c(N(c3ccc(-c4ccccc4)cc3)c3ccc(-c4ccc5c(c4)oc4ccccc45)cc3)ccc(-c3cccnc3)c21]
1-12 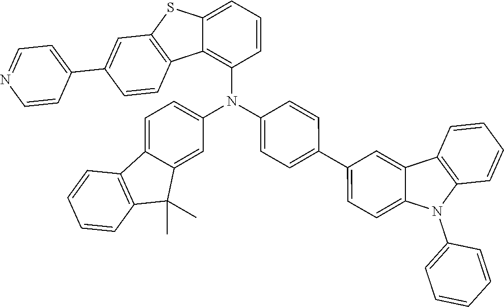 [SMILES: CC1(C)c2ccccc2-c2ccc(N(c3ccc(-c4ccc5c(c4)c4ccccc4n5-c4ccccc4)cc3)c3cccc4sc5cc(-c6ccncc6)ccc5c34)cc21]
1-13 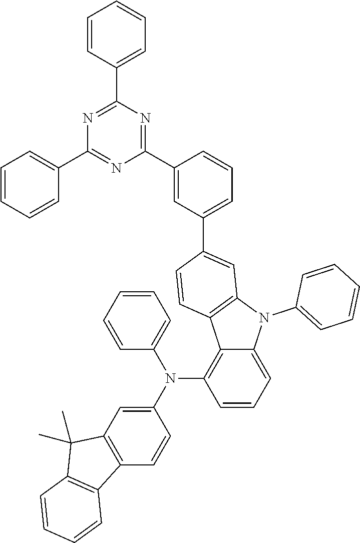 [SMILES: CC1(C)c2ccccc2-c2ccc(N(c3ccccc3)c3cccc4c3c3ccc(-c5cccc(-c6nc(-c7ccccc7)nc(-c7ccccc7)n6)c5)cc3n4-c3ccccc3)cc21]
1-14 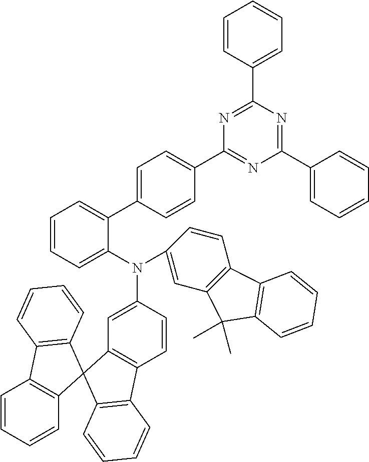 [SMILES: CC1(C)c2ccccc2-c2ccc(N(c3ccc4c(c3)C3(c5ccccc5-c5ccccc53)c3ccccc3-4)c3ccccc3-c3ccc(-c4nc(-c5ccccc5)nc(-c5ccccc5)n4)cc3)cc21]
1-15 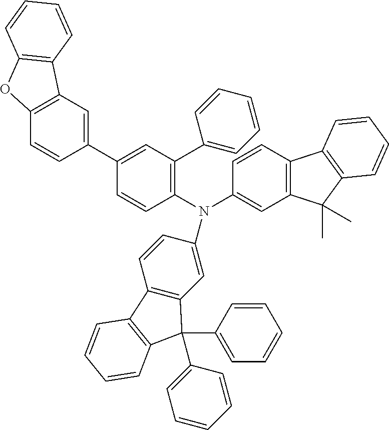 [SMILES: CC1(C)c2ccccc2-c2ccc(N(c3ccc4c(c3)C(c3ccccc3)(c3ccccc3)c3ccccc3-4)c3ccc(-c4ccc5oc6ccccc6c5c4)cc3-c3ccccc3)cc21]
1-21 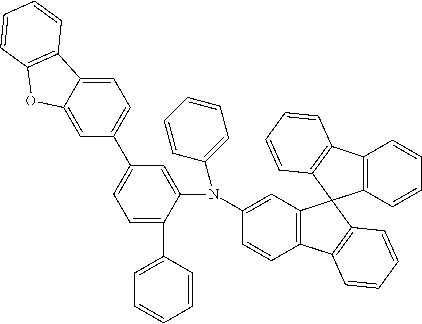 [SMILES: c1ccc(-c2ccc(-c3ccc4c(c3)oc3ccccc34)cc2N(c2ccccc2)c2ccc3c(c2)C2(c4ccccc4-c4ccccc42)c2ccccc2-3)cc1]
1-22

1.-22. (canceled)

23. A compound of formula (I) 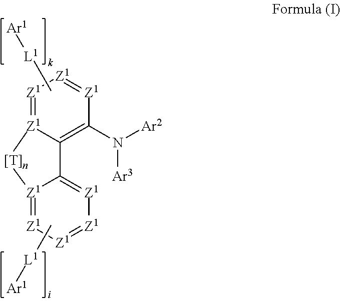
where the variables that occur are as follows:
Z¹ is the same or different at each instance and is selected from CR¹ and N, where Z¹ is C when an Ar¹ or L¹ or T group is attached;
Ar¹ is the same or different at each instance and is selected from dibenzofuran, dibenzothiophene, carbazole, benzimidazole, benzoxazole and benzothiazole, which may each be substituted by one or more R² radicals;
L¹ is a single bond, or an aromatic ring system which has 6 to 30 aromatic ring atoms and may be substituted by one or more R² radicals;
Ar² corresponds to a formula (A) 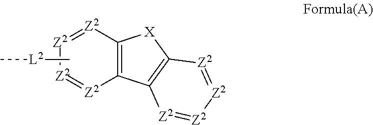
Z² is the same or different at each instance and is CR³ or N, where Z² is C when an L² group is bonded thereto;
L² is a single bond, or an aromatic ring system which has 6 to 30 aromatic ring atoms and may be substituted by one or more R³ radicals;
X is C(R³)₂;
Ar³ is an aromatic ring system which has 6 to 20 aromatic ring atoms and may be substituted by one or more R⁴ radicals, or a heteroaromatic ring system which has 5 to 30 aromatic ring atoms and may be substituted by one or more R⁴ radicals;
T is C(R¹)₂, O or S;
R¹, R², R³, R⁴ are the same or different at each instance and are selected from H, D, F, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned may each be substituted by one or more R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned may each be substituted by one or more R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; and where the alkyl, alkoxy, alkenyl and alkynyl groups, aromatic ring systems and heteroaromatic ring systems mentioned may be substituted by F or CN;
n is 1;
i is 0, 1, 2, 3, 4 or 5, where the -L¹-Ar¹ group having the index i is absent when i is 0;
k is 0, 1, 2, 3 or 4, where the -L¹-Ar¹ group having the index k is absent when k is 0;
where the sum of k and i is at least 1.

24. The compound according to claim 23, wherein Z¹ is CR¹, where Z¹ is C when an Ar¹ or L¹ or T group is bonded thereto.

25. The compound according to claim 23, wherein Ar¹ is the same or different at each instance and is selected from the group consisting of dibenzofuran, dibenzothiophene, where the groups mentioned may each be substituted by one or more R² radicals.

26. The compound according to claim 23, wherein L¹ is a single bond.

27. The compound according to claim 23, wherein Z² is CR³, where Z² is C when an L² group is bonded thereto.

28. The compound according to claim 23, wherein L² is a single bond.

29. The compound according to claim 23, wherein Ar³ is selected from the group consisting of phenyl, biphenyl, terphenyl, fluorenyl, naphthyl, pyridyl, pyrimidyl, triazinyl, dibenzofuranyl, benzofused dibenzofuranyl, dibenzothiophenyl, benzofused dibenzothiophenyl, carbazolyl, and benzofused carbazolyl, and combinations of two, three or four of these groups, where the groups mentioned may each be substituted by one or more R⁴ radicals.

30. The compound according to claim 23, wherein R¹ groups that are not bonded to a T group which is C(R¹)₂ are H.

31. The compound according to claim 23, wherein R³ groups that are not bonded to an X group which is C(R³)₂ are H.

32. The compound according to claim 23, wherein the sum total of i and k is 1.

33. Compound according to claim 23, characterized in that Ar³ is fluorenyl, which may be substituted by one or more R⁴ radicals.

34. Compound according to claim 23, characterized in that T is C(R¹)₂.

35. Compound according to claim 23, characterized in that the sum total of i and k is 1; and Ar³ is selected from the group consisting of phenyl, biphenyl, terphenyl, fluorenyl, naphthyl, pyridyl, pyrimidyl, triazinyl, dibenzofuranyl, benzofused dibenzofuranyl, dibenzothiophenyl, benzofused dibenzothiophenyl, carbazolyl, and benzofused carbazolyl, and combinations of two, three or four of these groups, where the groups mentioned may each be substituted by one or more R⁴ radicals; and L¹ is a single bond; and L² is a single bond; and Z¹ is CR¹, where Z¹ is C when an Ar¹ or L¹ or T group is bonded thereto; and Z² is CR³, where Z² is C when an L² group is bonded thereto; and Ar¹ is dibenzofuran, which may be substituted by one or more R² radicals; and T is C(R¹)₂.

36. Compound according to claim 23, having the following chemical structure:

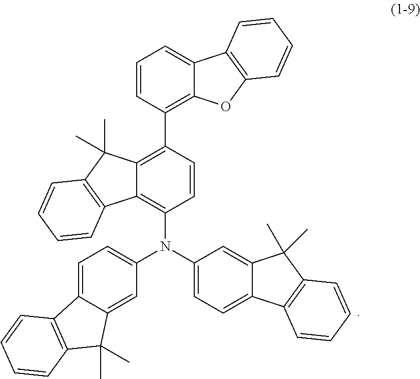 [SMILES: CC1(C)c2ccccc2-c2ccc(N(c3ccc4c(c3)C(C)(C)c3ccccc3-4)c3ccc(-c4cccc5c4oc4ccccc45)c4c3-c3ccccc3C4(C)C)cc21]

37. A process for preparing the compound according to claim 23, which comprises reacting a diarylamine which is a secondary amine with a halogen-substituted aromatic or heteroaromatic ring system to give a triarylamine compound which is a tertiary amine.

38. An oligomer, polymer or dendrimer containing one or more compounds according to claim 23, wherein the bond(s) to the polymer, oligomer or dendrimer may be localized at any position substituted by R¹, R², R³ or R⁴ in formula (I).

39. A formulation comprising at least one compound according to claim 23 and at least one solvent.

40. An electronic device comprising at least one compound according to claim 23.

41. The electronic device according to claim 42, wherein the device is an organic electroluminescent device comprising anode, cathode and at least one emitting layer, where it is at least one organic layer of the device, which may be an emitting layer or a hole-transporting layer, that contains the at least one compound.
